(19) United States
(12) Patent Application Publication
(10) Pub. No.: US 2022/0111028 A1
(43) Pub. Date: Apr. 14, 2022

(54) Combination Of A STING Agonist And A Complex Comprising A Cell Penetrating Peptide, A Cargo And A TLR Peptide Agonist

(71) Applicant: Boehringer Ingelheim International GmbH, Ingelheim Am Rhein (DE)

(72) Inventors: Matteo ROSSI, Geneva (CH); Elodie BELNOUE, Geneva (CH); Madiha DEROUAZI, Geneva (CH)

(21) Appl. No.: 17/501,155

(22) Filed: Oct. 14, 2021

(30) Foreign Application Priority Data
Oct. 14, 2020 (EP) PCT/EP2020/078961
Apr. 16, 2021 (EP) PCT/EP2021/059934

(51) Int. Cl. A61K 39/00; A61P 35/00

(52) U.S. Cl. CPC A61K 39/001182; A61K 39/00117; A61K 39/001166; A61K 39/001106; A61K 39/001109; A61K 39/001186; A61K 2039/6075; A61K 39/001189; A61K 39/001152; A61K 39/001119; A61K 39/00115; A61P 35/00; A61K 2039/55561; A61K 39/001188

(57) ABSTRACT

The present invention provides a combination of an agonist of stimulator of interferon response cGAMP interactor 1 (STING) and a vaccine including specific antigens or antigenic epitopes, namely, a complex comprising a cell penetrating peptide, at least one antigen or antigenic epitope, and a TLR peptide agonist. Such a combination is particularly useful in medicine, in particular in the prevention and/or treatment of cancer. Moreover, the present invention also provides compositions, such as a pharmaceutical compositions and vaccines, which are useful, for example, in the prevention and/or treatment of cancer.

The present invention relates to the field of vaccination and immunotherapy, in particular to cancer immunotherapy.

The immune system can recognize and to some extent eliminate tumor cells, however, this anti-tumor response is often of low amplitude and inefficient. Boosting this weak anti-tumor response with therapeutic vaccination has been a long sought goal for cancer therapy. Modulating the immune system to enhance immune responses has thus become a promising therapeutic approach in oncology as it can be combined with standard of care treatments.

Cancer vaccines can be divided into two principal categories: personalized (autologous) and standardized vaccines, and further classified depending on the technology platform. Current personalized vaccines include tumor lysate vaccines as well as dendritic cells based vaccine (hereinafter cell based). For the latter, antigen loading can occur either with a pulse using tumor lysates, or transfection with RNA extracted from the tumors. In this case, the antigens are tumor specific or associated, but are not clearly defined. Dendritic cells can also be loaded with defined antigens either with peptide pulse or using a protein such as the Prostatic Acid Phosphatase (PAP) used to engineer the Provenge® vaccine. However, the manufacturing process of these cell-based therapies is time-consuming and labor-intensive while quality standards are difficult to reach and maintain. Immunomonitoring creates further complications. Moreover, the majority of the autologous cancer vaccines do not allow the identities or quantities of antigens used to be controlled, unlike defined and standardized vaccines.

In contrast to cell-based therapy (such as antigen-presenting cells (APCs), T cells, CARs, lysates), subunits vaccines (protein or peptides) allow the development of a standardized vaccine with an easier production and significantly better batch to batch reproducibility that can be administered to a broad range of patients. Furthermore, the antigens are fully defined allowing for better immune-monitoring and reducing the risk of unwanted effects of vaccine component.

The different approaches which were evaluated in pre-clinical and clinical development include short peptide vaccines (Slingluff C L, Jr. The present and future of peptide vaccines for cancer: single or multiple, long or short, alone or in combination? Cancer journal 2011; 17(5):343-50), long-peptide vaccines (Melief C J, van der Burg S H. Immunotherapy of established (pre)malignant disease by synthetic long peptide vaccines. Nature reviews Cancer 2008; 8(5):351-60) and proteins. In contrast to long peptide and protein vaccines, short peptide vaccines have a very short half-life and can have negative consequences on the immune response.

In general, a cancer vaccine is administered to cancer patients to strengthen the capability of their immune system to recognize and kill the tumor cells. The main goal of a therapeutic cancer vaccine is to generate killer T cells (also called cytotoxic T lymphocytes) specific for the tumor cells. To this end, and to achieve a potent immune response, the vaccine usually contains antigens or antigenic epitopes that are also present in the tumor and that need to be delivered to Antigen Presenting Cells (APCs), especially dendritic cells (DCs), to allow cancer immunity to be initiated. The DCs process these tumor antigens into small peptides that are presented on cell surface expressed MHC class I or MHC class II molecules to T cells. Peptides that are then recognized by T cells and thereby induce their stimulation are called epitopes. Presentation by MHC class I and MHC class II molecules allows activation of two classes of T cells, CD8⁺ cytotoxic T lymphocytes (CTLs) and CD4⁺ helper T (Tₕ) cells, respectively. In addition, to become fully activated, beside antigen recognition T cells require a second signal, the co-stimulatory signal, which is antigen non-specific and is provided by the interaction between co-stimulatory molecules expressed on the surface of APCs and the T cell. Therefore two major requirements for an efficient therapeutic cancer vaccine are the specificity of the tumor antigens and the ability to deliver them efficiently to DCs.

Taken together, induction of a tumor specific immune response thus requires three main steps: (i) an antigen being delivered to dendritic cells, which will process it into epitopes, (ii) dendritic cells should receive a suitable activation signal, and (iii) activated tumor antigen-loaded dendritic cells must generate T-cell mediated immune responses in the lymphoid organs.

Since tumor cells can escape the immune system by down-regulating expression of individual antigens (passive immune escape), multi-epitopic antigen delivery provides an advantage. Indeed, protein based vaccines allow multi-epitopic antigen delivery to antigen presenting cells (APCs) such as dendritic cells (DCs) without the limitation of restriction to a single MHC allele. Another strength is long-lasting epitope presentation recently described in dendritic cells loaded with proteins (van Montfoort N, Camps M G, Khan S, Filippov D V, Weterings J J, Griffith J M, et al. Antigen storage compartments in mature dendritic cells facilitate prolonged cytotoxic T lymphocyte cross-priming capacity. Proceedings of the National Academy of Sciences of the United States of America 2009; 106(16):6730-5). Furthermore, proteins require uptake and processing by DCs to achieve MHC restricted presentation of their constituent epitopes. This reduces the risk of inducing peripheral tolerance as has been shown after vaccination with short peptides that do not have such stringent processing requirements (Toes R E, Offringa R, Blom R J, Melief C J, Kast W M. Peptide vaccination can lead to enhanced tumor growth through specific T-cell tolerance induction. Proceedings of the National Academy of Sciences of the United States of America 1996; 93(15):7855-60).

However, most soluble proteins are generally degraded in endolysosomes and are poorly cross-presented on MHC class I molecules and are therefore poorly immunogenic for CD8⁺ T cell responses (Rosalia R A, Quakkelaar E D, Redeker A, Khan S, Camps M, Drijfhout J W, et al. Dendritic cells process synthetic long peptides better than whole protein, improving antigen presentation and T-cell activation. European journal of immunology 2013; 43(10):2554-65). Moreover, although mature DCs are more potent than immature DCs in priming and eliciting T-cell responses (Apetoh L, Locher C, Ghiringhelli F, Kroemer G, Zitvogel L. Harnessing dendritic cells in cancer. Semin Immunol. 2011; 23:42-49), they lose the ability to efficiently take up exogenous antigens, particularly for MHC class II restricted antigens (Banchereau J, Steinman R M. Dendritic cells and the control of immunity. Nature. 1998; 392:245-252). As a result, peptide-pulsed DCs as vaccines have several limitations. For example, peptide degradation, rapid MHC class I turnover, and the disassociation of peptide from MHC class I molecules during the preparation and injection of DC/peptides may result in short half-lives of MHC class I/peptide complexes on the DC surface, leading to weak T-cell responses.

To improve the efficacy of protein-based vaccine delivery, the use of cell penetrating peptides for intracellular delivery of cancer peptides into DCs has been proposed (Wang R F, Wang H Y. Enhancement of antitumor immunity by prolonging antigen presentation on dendritic cells. Nat Biotechnol. 2002; 20:149-156). Cell penetrating peptides (CPPs) are peptides that have the ability to cross the cell membrane and enter into most cell types (Copolovici D M, Langel K, Eriste E, Langel U. Cell-penetrating peptides: design, synthesis, and applications. ACS nano 2014; 8(3):1972-94, Milletti F. Cell-penetrating peptides: classes, origin, and current landscape. Drug Discov Today 2012). Alternatively, they are also called protein transduction domain (PTDs) reflecting their origin as occurring in natural proteins. Several potent CPPs have been identified from proteins, including the Tat protein of human immunodeficiency virus, the VP22 protein of herpes simplex virus, and fibroblast growth factor (Berry C C. Intracellular delivery of nanoparticles via the HIV-1 tat peptide. Nanomedicine. 2008; 3:357-365; Deshayes S, Morris M C, Divita G, Heitz F. Cell-penetrating peptides: Tools for intracellular delivery of therapeutics. Cell Mol Life Sci. 2005; 62:1839-1849; Edenhofer F. Protein transduction revisited: Novel insights into the mechanism underlying intracellular delivery of proteins. Curr Pharm Des. 2008; 14:3628-3636; Gupta B, Levchenko T S, Torchilin V P. Intracellular delivery of large molecules and small particles by cell-penetrating proteins and peptides. Adv Drug Deliv Rev. 2005; 57:637-651; Torchilin V P. Recent approaches to intracellular delivery of drugs and DNA and organelle targeting. Annu Rev Biomed Eng. 2006; 8:343-375). It was found that T-cell activity elicited by DC/TAT-TRP2 was 3- to 10-fold higher than that induced by DC/TRP2 (Wang H Y, Fu T, Wang G, Gang Z, Donna M P L, Yang J C, Restifo N P, Hwu P, Wang R F. Induction of CD4+ T cell-dependent antitumor immunity by TAT-mediated tumor antigen delivery into dendritic cells. J Clin Invest. 2002a; 109:1463-1470).

In order to increase the level of co-stimulatory molecules on DCs and to augment the immune system's response to the target antigens, adjuvants may be used. Adjuvants may accomplish this task by mimicking conserved microbial components that are naturally recognized by the immune system. They include, for example, lipopolysaccharide (LPS), components of bacterial cell walls, and nucleic acids such as double-stranded RNA (dsRNA), single-stranded DNA (ssDNA), and unmethylated CpG dinucleotide-containing DNA. Their presence can increase the innate immune response to the antigen. Furthermore, this adjuvant should promote an adaptive immune response with CTLs and type polarized Tₕ1 rather than a humoral immune response resulting in antibody production. Different adjuvants have been evaluated, with a limited number having gained regulatory approval for human use. These include Alum, MPL (monophosphoryl lipid A) and ASO₄ (Alum and MPL) in the US, and MF59 (oil-in-water emulsion), ASO₄, liposomes in Europe (Lim, Y. T., Vaccine adjuvant materials for cancer immunotherapy and control of infectious disease. Clin Exp Vaccine Res, 2015. 4(1): p. 54-8).

Recently, Toll Like Receptor (TLR) ligands are emerging as promising class of adjuvants (Baxevanis, C. N., I. F. Voutsas, and O. E. Tsitsilonis, Toll-like receptor agonists: current status and future perspective on their utility as adjuvants in improving anticancer vaccination strategies. Immunotherapy, 2013.5(5): p. 497-511). A significant development of cancer vaccine studies was thus to include various TLR agonists to vaccine formulations, including TLR-3 (poly I:C), TLR-4 (monophosphoryl lipid A; MPL), TLR-5 (flagellin), TLR-7 (imiquimod), and TLR-9 (CpG) (Duthie M S, Windish H P, Fox C B, Reed S G. Use of defined TLR ligands as adjuvants within human vaccines. Immunol Rev. 2011; 239:178-196). The types of signaling and cytokines produced by immune cells after TLR stimulation control CD4+ T-cell differentiation into Th1, Th2, Th17, and Treg cells. Stimulation of immune cells such as DCs and T cells by most TLR-based adjuvants produces proinflammatory cytokines and promotes Th1 and CD8+T responses (Manicassamy S, Pulendran B. Modulation of adaptive immunity with Toll-like receptors. Semin Immunol. 2009; 21:185-193).

Conjugating the vaccine to a TLR ligand is an attractive approach that offers several advantages over non-conjugated vaccines including (i) preferential uptake by the immune cells expressing the TLR, (ii) higher immune response and (iii) reduced risk of inducing peripheral tolerance. Indeed, all the antigen presenting cells loaded with the antigen will be simultaneously activated. Different groups explored this approach with various TLR ligands being mainly linked chemically to the peptide or protein vaccine (Zom G G, Khan S, Filippov D V, Ossendorp F. TLR ligand-peptide conjugate vaccines: toward clinical application. Adv Immunol. 2012; 114:177-201). As the chemical linkage to peptide is easily performed, the most highly investigated TLR ligands for conjugate vaccine are the TLR2 agonist Pam2Cys and Pam3Cys (Fujita, Y. and H. Taguchi, Overview and outlook of Toll-like receptor ligand-antigen conjugate vaccines. Ther Deliv, 2012. 3(6): p. 749-60).

Recently, a chimeric protein vaccine platform was described, which provides a complex composed of three elements: a cell-penetrating peptide, an antigenic cargo and a TLR agonist conferring self-adjuvanticity (Belnoue, E., et al. (2016). “Enhancing Antitumor Immune Responses by Optimized Combinations of Cell-penetrating Peptide-based Vaccines and Adjuvants.” Mol Ther 24(9): 1675-1685). This vaccine platform was shown to elicit both CD8 and CD4 antigen-specific immune responses in preclinical tumor models leading to immunological memory and high vaccine efficacy together with increased intratumoral leukocyte infiltration (Derouazi, M., et al. (2015). “Novel Cell-Penetrating Peptide-Based Vaccine Induces Robust CD4+ and CD8+ T Cell-Mediated Antitumor Immunity.” Cancer Res 75(15): 3020-3031; Belnoue, E., et al. (2019). “Targeting self and neo-epitopes with a modular self-adjuvanting cancer vaccine.” JCI Insight 5).

Another very promising strategy is the targeting of the stimulator of interferon genes/stimulator of interferon response cGAMP interactor 1 (STING) pathway. STING is an adaptor protein activated by the binding to cyclic GAMP, a by-product of viral or bacterial DNA degradation by cytosolic DNA sensors (Ishikawa, H. and G. N. Barber (2008). “STING is an endoplasmic reticulum adaptor that facilitates innate immune signalling.” Nature 455(7213): 674-678; Ablasser, A., et al. (2013). “cGAS produces a 2′-5′-linked cyclic dinucleotide second messenger that activates STING.” Nature 498(7454): 380-384), which upon activation, induces the secretion of high levels of type I interferons and other pro-inflammatory cytokines such as IL-6 and TNF [symbol] (Ishikawa, H. and G. N. Barber (2008). “STING is an endoplasmic reticulum adaptor that facilitates innate immune signalling.” Nature 455(7213): 674-678; Saitoh, T., et al. (2009). “Atg9a controls dsDNA-driven dynamic translocation of STING and the innate immune response.” Proc Natl Acad Sci USA 106(49): 20842-20846; Sokolowska, O. and D. Nowis (2018). “STING Signaling in Cancer Cells: Important or Not?” Arch Immunol Ther Exp (Warsz) 66(2): 125-132). Furthermore, STING signaling was shown to enhance NK cells recruitment and activation (Takashima, K., et al. (2016). “STING in tumor and host cells cooperatively work for NK cell-mediated tumor growth retardation.” Biochem Biophys Res Commun 478(4): 1764-1771), and to promote CD4 and CD8 T cells chemotaxis (Parkes, E. E., et al. (2017). “Activation of STING-Dependent Innate Immune Signaling By S-Phase-Specific DNA Damage in Breast Cancer.” J Natl Cancer Inst 109(1)). In addition, STING signaling was found to be inhibited in patient derived colorectal adenocarcinoma cells, supporting its anti-tumoral role (Xia, X., et al. (2015). “Porous silicon microparticle potentiates anti-tumor immunity by enhancing cross-presentation and inducing type I interferon response.” Cell Rep 11(6): 957-966). Due to these properties, synthetic STING agonists have been tested in pre-clinical tumor models and in clinical studies with the intent of inflame the tumor and elicit an anti-tumoral immune response. Intra-tumoral injection of STING agonist was shown to induce regression of different murine tumor models, while also inducing a systemic tumor-specific immune response as underscored by the resistance to re-challenge of tumor-cleared mice (Corrales, L., et al. (2015). “Direct Activation of STING in the Tumor Microenvironment Leads to Potent and Systemic Tumor Regression and Immunity.” Cell Rep 11(7): 1018-1030). Moreover, STING agonist formulated within a GM-CSF-producing cancer cells vaccine was shown to delay the progression of several murine tumor models, demonstrating that intra-tumoral administration is not the only effective route. Currently, multiple phase ½ clinical trials investigates the use of STING agonists in different solid tumors and lymphoma patients.

In view of the above, it is the object of the present invention to overcome the drawbacks of current cancer vaccines outlined above. In particular, it is an object of the present invention to provide a combination of a STING agonist with a vaccine containing an antigen or an antigenic epitope, which provides specificity against a certain tumor. It is also an object of the present invention to provide a vaccine, which enhances or prolongs the antitumor effects of each of its components (e.g., when administered as stand-alone therapy). Accordingly, such a combination represents a more potent vaccine for cancer immunotherapy applications, in particular with improved anti-tumor activity. The present invention thus relates to a combination therapy to initiate, enable, enhance and/or improve an anti-tumor immune response.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and/or combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

### Combination of a STING Agonist and a Complex Comprising a Cell Penetrating Peptide, at Least One Antigen or Antigenic Epitope and a TLR Peptide Agonist

In a first aspect the present invention provides a combination of
(i) a STING agonist and
(ii) a complex comprising:
   a) a cell penetrating peptide;
   b) at least one antigen or antigenic epitope; and
   c) a TLR peptide agonist,
   wherein the components a)-c) (i.e. the cell penetrating peptide, the at least one antigen or antigenic epitope and the TLR peptide agonist) are covalently linked.

The present inventors surprisingly found that a combination of (i) a STING agonist and (ii) a complex comprising a cell penetrating peptide, at least one antigen or antigenic epitope, and a TLR peptide agonist improves both CD4 and CD8 T cells response boosting antigen-specific CD8 T cells, increases intra-tumoral immunogenicity and results in considerably increased survival rates and reduced tumor growth. This indicates a synergistic effect of the STING agonist and the complex acting together, which considerably increases the anti-tumor effects of each of its components administered as stand-alone therapy.

As used herein, the term “combination” refers to any kind of combination of its components, in particular, to any kind of combination of (i) the STING agonist and (ii) the complex as described herein, and, optionally, any further components. In particular, the components of a combination are provided together (i.e., in a combined manner). In some embodiments, the combination may be a kit (e.g., comprising the components in an (at least partially) separated manner). In other embodiments, the combination may be a composition (e.g., the components may be comprised in a single composition).

Accordingly, each of the components (i) (STING agonist) and (ii) (complex) (and any further optional components) of the combination may be comprised in a separate composition. In other embodiments, some (but not all) of the components (i) (STING agonist) and (ii) (complex) (and any further optional components) of the combination may be comprised in the same composition. Alternatively, all components (i) (STING agonist) and (ii) (complex) (and any further optional components) of the combination may be comprised in the same composition. Accordingly, the each of the components (i) (STING agonist) and (ii) (complex) (and any further optional components) of the combination may be comprised in a separate container (e.g., a syringe). In other embodiments, some (but not all) of the components (i) (STING agonist) and (ii) (complex) (and any further optional components) of the combination may be comprised in the same container (e.g., a syringe). Alternatively, all components (i) (STING agonist) and (ii) (complex) (and any further optional components) of the combination may be comprised in the same container (e.g., a syringe).

More specifically, (i) the STING agonist and (ii) the complex may be comprised in the same composition and/or in the same container (e.g., a syringe). In some embodiments, (i) the STING agonist and (iii) an optional third component (other than the complex and the STING agonist) may be comprised in the same composition and/or in the same container (e.g., a syringe). In some embodiments, (ii) the complex and (iii) an optional third component (other than the complex and the STING agonist) may be comprised in the same composition and/or in the same container (e.g., a syringe). For example, (i) the STING agonist; (ii) the complex and (iii) an optional third component (other than the complex and the STING agonist) may be comprised in the same composition and/or in the same container (e.g., a syringe).

In some embodiments, (i) the STING agonist and (ii) the complex may be provided in distinct compositions and/or in distinct containers (e.g., distinct syringes). In some embodiments, (i) the STING agonist and (iii) an optional third component (other than the complex and the STING agonist) may be provided in distinct compositions and/or in distinct containers (e.g., distinct syringes). In some embodiments, (ii) the complex and (iii) an optional third component (other than the complex and the STING agonist) may be provided in distinct compositions and/or in distinct containers (e.g., distinct syringes). For example, (i) the STING agonist; (ii) the complex and (iii) an optional third component (other than the complex and the STING agonist) may be provided in distinct compositions and/or in distinct containers (e.g., distinct syringes).

In the following, the components of the combination according to the present invention, i.e. the STING agonist and the complex comprising cell penetrating peptide, the at least one antigen or antigenic epitope and the at least one TLR peptide agonist, and embodiments thereof, are described in detail. It is understood that (i) a preferred embodiment of the combination according to the present invention comprises a preferred embodiment of the STING agonist; (ii) a preferred embodiment of the combination according to the present invention comprises a preferred embodiment of the complex comprising cell penetrating peptide, the at least one antigen or antigenic epitope and the at least one TLR peptide agonist; and (iii) a more preferred embodiment of the combination according to the present invention comprises a preferred embodiment of the STING agonist and a preferred embodiment of the complex comprising cell penetrating peptide, the at least one antigen or antigenic epitope and the at least one TLR peptide agonist.

In some embodiments, the combination according to the present invention, i.e. the STING agonist and the complex comprising cell penetrating peptide, the at least one antigen or antigenic epitope and the at least one TLR peptide agonist may be administered in combination with further, additional active compounds (e.g., in the context of tumor/cancer treatment). In other embodiments, the combination according to the present invention, i.e. the STING agonist and the complex comprising cell penetrating peptide, the at least one antigen or antigenic epitope and the at least one TLR peptide agonist is not administered in combination with further, additional active compounds (e.g., in the context of tumor/cancer treatment). In other words, the inventive combination may also be useful as “stand-alone” therapy.

### Complex Comprising a Cell Penetrating Peptide, at Least One Antigen or Antigenic Epitope

The combination according to the present invention comprises a complex comprising:
a) a cell penetrating peptide;
b) at least one antigen or antigenic epitope; and
c) at least one TLR peptide agonist,
   wherein the components a)-c), i.e. the cell penetrating peptide, the at least one antigen or antigenic epitope and the at least one TLR peptide agonist, are covalently linked. In the following, it is also referred to such a complex comprising a cell penetrating peptide, at least one antigen or antigenic epitope and at least one TLR peptide agonist, which are covalently linked, by using the term “the complex” or “the complex comprised by the combination according to the present invention”.

Such a complex comprised by the combination according to the present invention provides simultaneous (i) stimulation of multi-epitopic cytotoxic T cell-mediated immunity, (ii) induction of Tₕ cells and (iii) promotion of immunological memory. Thereby, a complex comprised by the combination according to the present invention provides a potent vaccine, in particular having improved anti-tumor activity.

Preferably, the complex comprised by the combination according to the present invention is a polypeptide or a protein, in particular a recombinant polypeptide or a recombinant protein, preferably a recombinant fusion protein or a recombinant fusion polypeptide.

The term “recombinant” as used herein (i.e. throughout the specification) means that it (here: the polypeptide or the protein) does not occur naturally. Accordingly, the complex comprised by the combination according to the present invention, which is a recombinant polypeptide or a recombinant protein, typically comprises components a) to c), wherein components a) to c) may be of different origins, i.e. do not naturally occur in this combination. In some embodiments, the term “recombinant” refers to peptides, polypeptides or proteins, which are semisynthetic or synthetic origin. A recombinant peptide, polypeptide or protein may result from the expression of a combination of DNA molecules of different origin that may be joined using recombinant DNA technologies. In some instances, a recombinant peptide, polypeptide or protein may—by virtue of its origin or manipulation—not be associated with all or a portion of a protein with which it is associated in nature. Moreover, a recombinant peptide, polypeptide or protein may be linked to a polypeptide other than that to which it is linked in nature. Recombinant peptides, polypeptides or proteins may be produced by any method known in the art, such as, e.g., prokaryotic and eukaryotic expression systems using well established protocols (see e.g. LaVallie, Current Protocols in Protein Science (1995) 5.1.1-5.1.8; Chen et al., Current Protocols in Protein Science (1998) 5.10.1-5.10.41). For example, in the complex comprised in the combination of the invention, components a)-c) may be of different origin, i.e. components a)-c) of the complex usually do not occur together in nature (such that the complex may be “recombinant” due to the combination of its components a)-c)).

In the context of the present invention, i.e. throughout the present application, the terms “peptide”, “polypeptide”, “protein” and variations of these terms refer to peptide, oligopeptide, oligomer or protein including fusion protein, respectively, comprising at least two amino acids joined to each other, preferably by a normal peptide bond, or, alternatively, by a modified peptide bond, such as for example in the cases of isosteric peptides. A peptide, polypeptide or protein can be composed of L-amino acids and/or D-amino acids. Preferably, a peptide, polypeptide or protein is either (entirely) composed of L-amino acids or (entirely) of D-amino acids, thereby forming “retro-inverso peptide sequences”. The term “retro-inverso (peptide) sequences” refers to an isomer of a linear peptide sequence in which the direction of the sequence is reversed and the chirality of each amino acid residue is inverted (see e.g. Jameson et al., Nature, 368, 744-746 (1994); Brady et al., Nature, 368, 692-693 (1994)). In particular, the terms “peptide”, “polypeptide”, “protein also include “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide or protein can comprise amino acids other than the 20 amino acids defined by the genetic code in addition to these amino acids, or it can be composed of amino acids other than the 20 amino acids defined by the genetic code. In particular, a peptide, polypeptide or protein in the context of the present invention can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, “protein” in the context of the present invention in particular also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62). Accordingly, the terms “peptide”, “polypeptide”, “protein” preferably include for example lipopeptides, lipoproteins, glycopeptides, glycoproteins and the like.

However, in a particularly preferred embodiment, the complex as described herein is a “classical” peptide, polypeptide or protein, whereby a “classical” peptide, polypeptide or protein is typically composed of amino acids selected from the 20 amino acids defined by the genetic code, linked to each other by a normal peptide bond.

If the complex comprised by the combination according to the present invention is a polypeptide or a protein, it is preferred that it comprises at least 50, at least 60, at least 70, preferably at least 80, at least 90, more preferably at least 100, at least 110, even more preferably at least 120, at least 130, particularly preferably at least 140, or most preferably at least 150 amino acid residues.

As used herein (i.e. throughout the present application), the term “sequence variant” refers to any alteration in a reference sequence. The term “sequence variant” includes nucleotide sequence variants and amino acid sequence variants. Preferably, a reference sequence is any of the sequences listed in the “Table of Sequences and SEQ ID Numbers” (Sequence listing), i.e. SEQ ID NO: 1 to SEQ ID NO: 55. In particular, a sequence variant shares (over the whole length of the sequence) at least 70% or at least 75%, preferably at least 80% or at least 85%, more preferably at least 90% or at least 95%, even more preferably at least 97% or at least 98%, particularly preferably at least 99% sequence identity with a reference sequence. Sequence identity may be calculated as described below. In particular, a sequence variant preserves the specific function of the reference sequence. In particular, an amino acid sequence variant has an altered sequence in which one or more of the amino acids in the reference sequence is deleted or substituted, or one or more amino acids are inserted into the sequence of the reference amino acid sequence. As a result of the alterations, the amino acid sequence variant has an amino acid sequence which is at least 70% or at least 75%, preferably at least 80% or at least 85%, more preferably at least 90% or at least 95%, even more preferably at least 97% or at least 98%, particularly preferably at least 99% identical to the reference sequence. For example, variant sequences which are at least 90% identical have no more than 10 alterations, i.e. any combination of deletions, insertions or substitutions, per 100 amino acids of the reference sequence.

In the context of the present invention, an amino acid sequence “sharing a sequence identity” of at least, for example, 95% to a query amino acid sequence of the present invention, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted, preferably within the above definitions of variants or fragments. The same, of course, also applies similarly to nucleic acid sequences.

For (amino acid or nucleic acid) sequences without exact correspondence, a “% identity” of a first sequence may be determined with respect to a second sequence. In general, these two sequences to be compared may be aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length.

Methods for comparing the identity and homology of two or more sequences are well known in the art. The percentage to which two sequences are identical can e.g. be determined using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs, e.g. BLAST or NBLAST program (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 183, 63-98; Pearson and Lipman (1988), Proc. Natl. Acad. Sci. U.S.A 85, 2444-2448.). Sequences which are identical to other sequences to a certain extent can be identified by these programmes. Furthermore, programs available in the Wisconsin Sequence Analysis Package, version 9.1 (Devereux et al., 1984, Nucleic Acids Res., 387-395), for example the programs BESTFIT and GAP, may be used to determine the % identity between two polynucleotides and the % identity and the % homology or identity between two polypeptide sequences. BESTFIT uses the “local homology” algorithm of (Smith and Waterman (1981), J. Mol. Biol. 147, 195-197.) and finds the best single region of similarity between two sequences.

In general, substitutions for one or more amino acids present in the referenced amino acid sequence are preferably made conservatively. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity properties, are well known (Kyte and Doolittle, 1982, J. Mol. Biol. 157(1):105-132). Substitutions of one or more L-amino acids with one or more D-amino acids are to be considered as conservative substitutions in the context of the present invention. Exemplary amino acid substitutions are presented in Table 1 below:

TABLE 1

| Original residues | Examples of substitutions |
|---|---|
| Ala (A) | Val, Leu, Ile, Gly |
| Arg (R) | His, Lys |
| Asn (N) | Gln |
| Asp (D) | Glu |
| Cys (C) | Ser |
| Gln (Q) | Asn |
| Glu (E) | Asp |
| Gly (G) | Pro, Ala |
| His (H) | Lys, Arg |
| Ile (I) | Leu, Val, Met, Ala, Phe |
| Leu (L) | Ile, Val, Met, Ala, Phe |
| Lys (K) | Arg, His |
| Met (M) | Leu, Ile, Phe |
| Phe (F) | Leu, Val, Ile, Tyr, Trp, Met |
| Pro (P) | Ala, Gly |
| Ser (S) | Thr |
| Thr (T) | Ser |
| Trp (W) | Tyr, Phe |
| Tyr (Y) | Trp, Phe |
| Val (V) | Ile, Met, Leu, Phe, Ala |

Component a)—Cell Penetrating Peptide

The cell penetrating peptide (CPP) allows for efficient delivery, i.e. transport and loading, in particular of at least one antigen or antigenic epitope, into the antigen presenting cells (APCs), in particular into the dendritic cells (DCs) and thus to the dendritic cells' antigen processing machinery.

The term “cell penetrating peptide” (“CPP”) is generally used to designate short peptides that are able to transport different types of cargo molecules across plasma membrane, and, thus, facilitate cellular uptake of various molecular cargoes (from nanosize particles to small chemical molecules and large fragments of DNA). “Cellular internalization” of the cargo molecule linked to the cell penetrating peptide generally means transport of the cargo molecule across the plasma membrane and thus entry of the cargo molecule into the cell. Depending on the particular case, the cargo molecule can, then, be released in the cytoplasm, directed to an intracellular organelle, or further presented at the cell surface. Cell penetrating ability, or internalization, of the cell penetrating peptide or of the complex (comprising said cell penetrating peptide) comprised by the combination according to the invention can be checked by standard methods known to one skilled in the art, including flow cytometry or fluorescence microscopy of live and fixed cells, immunocytochemistry of cells transduced with said peptide or complex, and Western blot.

Cell penetrating peptides typically have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. Cell-Penetrating peptides are of different sizes, amino acid sequences, and charges but all CPPs have a common characteristic that is the ability to translocate the plasma membrane and facilitate the delivery of various molecular cargoes to the cytoplasm or to an organelle of a cell. At present, the theories of CPP translocation distinguish three main entry mechanisms: direct penetration in the membrane, endocytosis-mediated entry, and translocation through the formation of a transitory structure. CPP transduction is an area of ongoing research. Cell-penetrating peptides have found numerous applications in medicine as drug delivery agents in the treatment of different diseases including cancer and virus inhibitors, as well as contrast agents for cell labeling and imaging.

Typically, cell penetrating peptides (CPPs) are peptides of 8 to 50 residues that have the ability to cross the cell membrane and enter into most cell types. Alternatively, they are also called protein transduction domain (PTDs) reflecting their origin as occurring in natural proteins. Frankel and Pabo simultaneously to Green and Lowenstein described the ability of the trans-activating transcriptional activator from the human immunodeficiency virus 1 (HIV-TAT) to penetrate into cells (Frankel, A. D. and C. O. Pabo, Cellular uptake of the tat protein from human immunodeficiency virus. Cell, 1988. 55(6): p. 1189-93). In 1991, transduction into neural cells of the Antennapedia homeodomain (DNA-binding domain) from Drosophila melanogaster was described (Joliot, A., et al., Antennapedia homeobox peptide regulates neural morphogenesis. Proc Natl Acad Sci USA, 1991. 88(5): p. 1864-8). In 1994, the first 16-mer peptide CPP called Penetratin, having the amino acid sequence RQIKIYFQNRRMKWKK (SEQ ID NO: 1) was characterized from the third helix of the homeodomain of Antennapedia (Derossi, D., et al., The third helix of the Antennapedia homeodomain translocates through biological membranes. J Biol Chem, 1994. 269(14): p. 10444-50), followed in 1998 by the identification of the minimal domain of TAT, having the amino acid sequence YGRKKRRQRRR (SEQ ID NO: 2) required for protein transduction (Vives, E., P. Brodin, and B. Lebleu, A truncated HIV-1 Tat protein basic domain rapidly translocates through the plasma membrane and accumulates in the cell nucleus. J Biol Chem, 1997. 272(25): p. 16010-7). Over the past two decades, dozens of peptides were described from different origins including viral proteins, e.g. VP22 (Elliott, G. and P. O'Hare, Intercellular trafficking and protein delivery by a herpesvirus structural protein. Cell, 1997. 88(2): p. 223-33) and ZEBRA (Rothe, R., et al., Characterization of the cell-penetrating properties of the Epstein-Barr virus ZEBRA trans-activator. J Biol Chem, 2010. 285(26): p. 20224-33), or from venoms, e.g. melittin (Dempsey, C. E., The actions of melittin on membranes. Biochim Biophys Acta, 1990. 1031(2): p. 143-61), mastoporan (Konno, K., et al., Structure and biological activities of eumenine mastoparan-AF (EMP-AF), a new mast cell degranulating peptide in the venom of the solitary wasp (Anterhynchium flavomarginatum micado). Toxicon, 2000. 38(11): p. 1505-15), maurocalcin (Esteve, E., et al., Transduction of the scorpion toxin maurocalcine into cells. Evidence that the toxin crosses the plasma membrane. J Biol Chem, 2005. 280(13): p. 12833-9), crotamine (Nascimento, F. D., et al., Crotamine mediates gene delivery into cells through the binding to heparan sulfate proteoglycans. J Biol Chem, 2007. 282(29): p. 21349-60) or buforin (Kobayashi, S., et al., Membrane translocation mechanism of the antimicrobial peptide buforin 2. Biochemistry, 2004. 43(49): p. 15610-6). Synthetic CPPs were also designed including the poly-arginine (R8, R9, R10 and R12) (Futaki, S., et al., Arginine-rich peptides. An abundant source of membrane-permeable peptides having potential as carriers for intracellular protein delivery. J Biol Chem, 2001. 276(8): p. 5836-40) or transportan (Pooga, M., et al., Cell penetration by transportan. FASEB J, 1998. 12(1): p. 67-77). Any of the above described CPPs may be used as cell penetrating peptide, i.e. as component a), in the complex comprised by the combination according to the present invention. In particular, the component a), i.e. the CPP, in the complex comprised by the combination according to the present invention may comprise the minimal domain of TAT, having the amino acid sequence YGRKKRRQRRR (SEQ ID NO: 2). In some embodiments, the component a), i.e. the CPP, in the complex comprised by the combination according to the present invention, may comprise Penetratin having the amino acid sequence RQIKIYFQNRRMKWKK (SEQ ID NO: 1).

Various CPPs, which can be used as cell penetrating peptide, i.e. as component a), in the complex comprised by the composition according to the present invention, are also disclosed in the review: Milletti, F., Cell-penetrating peptides: classes, origin, and current landscape. Drug Discov Today 17 (15-16): 850-60, 2012. In other words, the CPPs disclosed in Milletti, F., 2012, Cell-penetrating peptides: classes, origin, and current landscape. Drug Discov Today 17 (15-16): 850-60 can be used as cell penetrating peptide, i.e. as component a), in the complex comprised by the combination according to the present invention. This includes in particular cationic CPPs, amphipatic CPPs, and hydrophobic CPPs as well as CPPs derived from heparan-, RNA- and DNA-binding proteins (cf. Table 1 of Milletti, F., Cell-penetrating peptides: classes, origin, and current landscape. Drug Discov Today 17 (15-16): 850-60, 2012), CPPs derived from signal peptides (cf. Table 2 of Milletti, F., Cell-penetrating peptides: classes, origin, and current landscape. Drug Discov Today 17 (15-16): 850-60, 2012), CPPs derived from antimicrobial peptides (cf. Table 3 of Milletti, F., Cell-penetrating peptides: classes, origin, and current landscape. Drug Discov Today 17 (15-16): 850-60, 2012), CPPs derived from viral proteins (cf. Table 4 of Milletti, F., Cell-penetrating peptides: classes, origin, and current landscape. Drug Discov Today 17 (15-16): 850-60, 2012), CPPs derived from various natural proteins (cf. Table 5 of Milletti, F., Cell-penetrating peptides: classes, origin, and current landscape. Drug Discov Today 17 (15-16): 850-60, 2012), and Designed CPPs and CPPs derived from peptide libraries (cf. Table 6 of Milletti, F., Cell-penetrating peptides: classes, origin, and current landscape. Drug Discov Today 17 (15-16): 850-60, 2012).

Preferably, the cell penetrating peptide is derived from the “ZEBRA” protein of the Epstein-Barr virus (EBV). “ZEBRA” (also known as Zta, Z, EB1, or BZLF1) generally refers to the basic-leucine zipper (bZIP) transcriptional activator of the Epstein-Barr virus (EBV). The minimal domain of ZEBRA, which exhibits cell penetrating properties, has been identified as spanning from residue 170 to residue 220 of ZEBRA. The amino acid sequence of ZEBRA is disclosed under NCBI accession number YP_401673 and comprises 245 amino acids represented in SEQ ID NO: 3:

MMDPNSTSEDVKFTPDPYQVPFVQAFDQATRVYQDLGGPSQAPLPCVLW
PVLPEPLPQGQLTAYHVSTAPTGSWFSAPQPAPENAYQAYAAPQLFPVS
DITQNQQTNQAGGEAPQPGDNSTVQTAAAVVFACPGANQGQQLADIGVP
QPAPVAAPARRTRKPQQPESLEECDSELEIKRYKNRVASRKCRAKFKQL
LQHYREVAAAKSSENDRLRLLLKQMCPSLDVDSIIPRTPDVLHEDLLNF
(SEQ ID NO: 3-ZEBRA amino acid sequence (natural
sequence from Epstein-Barr virus (EBV))
(YP_401673))

Preferably, the cell penetrating peptide
i) has a length of the amino acid sequence of said peptide of 5 to 50 amino acids in total, preferably of 10 to 45 amino acids in total, more preferably of 15 to 45 amino acids in total; and/or
ii) has an amino acid sequence comprising a fragment of the minimal domain of ZEBRA, said minimal domain extending from residue 170 to residue 220 of the ZEBRA amino acid sequence according to SEQ ID NO: 3, wherein, optionally, 1, 2, 3, 4, or 5 amino acids have been substituted, deleted, and/or added without abrogating said peptide's cell penetrating ability, or a sequence variant of such a fragment.

Thereby, it is preferred that the cell penetrating peptide
i) has a length of the amino acid sequence of said peptide of 5 to 50 amino acids in total, preferably of 10 to 45 amino acids in total, more preferably of 15 to 45 amino acids in total; and
ii) has an amino acid sequence comprising a fragment of the minimal domain of ZEBRA, said minimal domain extending from residue 170 to residue 220 of the ZEBRA amino acid sequence according to SEQ ID NO: 3, wherein, optionally, 1, 2, 3, 4, or 5 amino acids have been substituted, deleted, and/or added without abrogating said peptide's cell penetrating ability, or a sequence variant of such a fragment.

Such preferred CPPs are disclosed, for example, in WO 2014/041505.

Recently, a CPP derived from the viral protein ZEBRA was described to transduce protein cargoes across biological membranes by both (i) direct translocation and (ii) lipid raft-mediated endocytosis (Rothe R, Liguori L, Villegas-Mendez A, Marques B, Grunwald D, Drouet E, et al. Characterization of the cell-penetrating properties of the Epstein-Barr virus ZEBRA trans-activator. The Journal of biological chemistry 2010; 285(26):20224-33). The present inventors assume that these two mechanisms of entry should promote both MHC class I and II restricted presentation of cargo antigens to CD8⁺ and CD4⁺ T cells, respectively. Accordingly, such a CPP can deliver multi-epitopic peptides to dendritic cells (DCs), and subsequently to promote CTL and Th cell activation and anti-tumor function. Such a CPP can thus efficiently deliver the complex comprised by the combination according to the present invention to antigen presenting cells (APCs) and lead to multi-epitopic MHC class I and II restricted presentation.

In the context of the present invention, the term “MHC class I” designates one of the two primary classes of the Major Histocompatibility Complex molecules. The MHC class I (also noted “MHC I”) molecules are found on every nucleated cell of the body. The function of MHC class I is to display an epitope to cytotoxic cells (CTLs). In humans, MHC class I molecules consist of two polypeptide chains, α- and β2-microglobulin (b2m). Only the a chain is polymorphic and encoded by a HLA gene, while the b2m subunit is not polymorphic and encoded by the Beta-2 microglobulin gene. In the context of the present invention, the term “MHC class II” designates the other primary class of the Major Histocompatibility Complex molecules. The MHC class II (also noted “MHC II”) molecules are found only on a few specialized cell types, including macrophages, dendritic cells and B cells, all of which are dedicated antigen-presenting cells (APCs).

Preferably, the sequence variant of a fragment of the minimal domain of ZEBRA as described above shares, in particular over the whole length, at least 70% or at least 75%, preferably at least 80% or at least 85%, more preferably at least 90% or at least 95%, even more preferably at least 97% or at least 98%, particularly preferably at least 99% amino acid sequence identity with the fragment of the minimal domain of ZEBRA (residue 170 to residue 220 of SEQ ID NO: 3) without abrogating the cell penetrating ability of the cell penetrating peptide. In particular, a “fragment” of the minimal domain of ZEBRA as defined above is preferably to be understood as a truncated sequence thereof, i.e. an amino acid sequence, which is N-terminally, C-terminally and/or intrasequentially truncated compared to the amino acid sequence of the native sequence. Moreover, such a “fragment” of the minimal domain of ZEBRA has preferably a length of 5 to 50 amino acids in total, preferably of 10 to 45 amino acids in total, more preferably of 15 to 45 amino acids in total.

More preferably, the fragments of the cell penetrating peptide or the variants thereof as described above further retain said peptide's ability to present a cargo molecule such as antigens or antigenic epitopes at the surface of a cell, such as an antigen-presenting cell, in the context of MHC class I and/or MHC class II molecules. The ability of a cell penetrating peptide or complex comprising said cell penetrating peptide to present a cargo molecule such as antigens or antigenic epitopes at the surface of a cell in the context of MHC class I and/or MHC class II molecules can be checked by standard methods known to one skilled in the art, including capacity to stimulate proliferation and/or function of MHC-restricted CD4⁺ or CD8⁺ T cells with specificity for these epitopes.

The preferred cell penetrating peptide, which
i) has a length of the amino acid sequence of said peptide of 5 to 50 amino acids in total, preferably of 10 to 45 amino acids in total, more preferably of 15 to 45 amino acids in total; and/or
ii) has an amino acid sequence comprising a fragment of the minimal domain of ZEBRA, said minimal domain extending from residue 170 to residue 220 of the ZEBRA amino acid sequence according to SEQ ID NO: 3, wherein, optionally, 1, 2, 3, 4, or 5 amino acids have been substituted, deleted, and/or added without abrogating said peptide's cell penetrating ability, or a variant of such a fragment
   preferably comprises an amino acid sequence having at least one conservatively substituted amino acid compared to the referenced sequence, meaning that a given amino acid residue is replaced by a residue having similar physiochemical characteristics.

Particularly preferably, the preferred cell penetrating peptide, which
i) has a length of the amino acid sequence of said peptide of 5 to 50 amino acids in total, preferably of 10 to 45 amino acids in total, more preferably of 15 to 45 amino acids in total; and/or
ii) has an amino acid sequence comprising a fragment of the minimal domain of ZEBRA, said minimal domain extending from residue 170 to residue 220 of the ZEBRA amino acid sequence according to SEQ ID NO: 3, wherein, optionally, 1, 2, 3, 4, or 5 amino acids have been substituted, deleted, and/or added without abrogating said peptide's cell penetrating ability, or a variant of such a fragment
   comprises a Cys (C) substituted into a Ser (S), at the equivalent of position 189 relative to ZEBRA amino acid sequence of SEQ ID NO: 3.

Thereby, it is preferred that such a preferred cell penetrating peptide has an amino acid sequence comprising a sequence according to the following general formula (A):

X₁X₂X₃X₄X₅X₆X₇X₈X₉X₁₀X₁₁SX₁₃X₁₄X₁₅X₁₆X₁₇

with 0, 1, 2, 3, 4, or 5 amino acids which are substituted, deleted, and/or added without abrogating said peptide's cell penetrating ability, wherein
X₁ is K, R, or H, preferably X₁ is K or R;
X₂ is R, K, or H, preferably X₂ is R or K;
X₃ is Y, W, or F, preferably X₃ is Y, W, or F;
X₄ is K, R, or H, preferably X₄ is K or R;
X₅ is N or Q;
X₆ is R, K, or H, preferably X₆ is R or K;
X₇ is V, I, M, L, F, or A, preferably X₇ is V, I, M or L;
X₈ is A, V, L, I, or G, preferably X₈ is A or G;
X₉ is S or T;
X₁₀ is R, K, or H, preferably X₁₀ is R or K;
X₁₁ is K, R, or H, preferably X₁₁ is K or R;
X₁₃ is R, K, or H, preferably X₁₃ is R or K;
X₁₄ is A, V, L, I, or G, preferably X₁₄ is A or G;
X₁₅ is K, R, or H, preferably X₁₅ is K or R;
X₁₆ is F, L, V, I, Y, W, or M, preferably X₁₆ is F, Y or W; and
X₁₇ is K, R, or H, preferably X₁₇ is K or R.

Preferably, such a peptide, polypeptide or protein is either (entirely) composed of L-amino acids or (entirely) of D-amino acids, thereby forming “retro-inverso peptide sequences”. The term “retro-inverso (peptide) sequences” refers to an isomer of a linear peptide sequence in which the direction of the sequence is reversed and the chirality of each amino acid residue is inverted (see e.g. Jameson et al., Nature, 368, 744-746 (1994); Brady et al., Nature, 368, 692-693 (1994)).

In a particular embodiment, the cell penetrating peptide is as generically defined above by general formula (A), wherein X₁ is K.

In a particular embodiment, the cell penetrating peptide is as generically defined above by general formula (A), wherein X₂ is R.

In a particular embodiment, the cell penetrating peptide is as generically defined above by general formula (A), wherein X₃ is Y.

In a particular embodiment, the cell penetrating peptide is as generically defined above by general formula (A), wherein X₄ is K.

In a particular embodiment, the cell penetrating peptide is as generically defined above by general formula (A), wherein X₅ is N.

In a particular embodiment, the cell penetrating peptide is as generically defined above by general formula (A), wherein X₆ is R.

In a particular embodiment, the cell penetrating peptide is as generically defined above by general formula (A), wherein X₇ is V.

In a particular embodiment, the cell penetrating peptide is as generically defined above by general formula (A), wherein X₉ is A.

In a particular embodiment, the cell penetrating peptide is as generically defined above by general formula (A), wherein X₉ is S.

In a particular embodiment, the cell penetrating peptide is as generically defined above by general formula (A), wherein X₁₀ is R.

In a particular embodiment, the cell penetrating peptide is as generically defined above by general formula (A), wherein X₁₁ is K.

In a particular embodiment, the cell penetrating peptide is as generically defined above by general formula (A), wherein X₁₃ is R.

In a particular embodiment, the cell penetrating peptide is as generically defined above by general formula (A), wherein X₁₄ is A.

In a particular embodiment, the cell penetrating peptide is as generically defined above by general formula (A), wherein X₁₅ is K.

In a particular embodiment, the cell penetrating peptide is as generically defined above by general formula (A), wherein X₁₆ is F.

In a particular embodiment, the cell penetrating peptide is as generically defined above by general formula (A), wherein X₁₇ is K.

In a particular embodiment, the cell penetrating peptide is as generically defined above by general formula (A), wherein the amino acid at position equivalent to position 12 relative to general formula (A) is a Ser (S).

It is also particularly preferred, that the preferred cell penetrating peptide, which
i) has a length of the amino acid sequence of said peptide of 5 to 50 amino acids in total, preferably of 10 to 45 amino acids in total, more preferably of 15 to 45 amino acids in total; and/or
ii) has an amino acid sequence comprising a fragment of the minimal domain of ZEBRA, said minimal domain extending from residue 170 to residue 220 of the ZEBRA amino acid sequence according to SEQ ID NO: 3, wherein, optionally, 1, 2, 3, 4, or 5 amino acids have been substituted, deleted, and/or added without abrogating said peptide's cell penetrating ability, or a variant of such a fragment
   comprises or consists of an amino acid sequence selected from the group consisting of amino acid sequences according to SEQ ID NO: 4-13, or sequence variants thereof without abrogating said peptide's cell penetrating ability, preferably sequence variants having 0, 1, 2, 3, 4, or 5 amino acids substituted, deleted and/or added without abrogating said peptide's cell penetrating ability.

CPP1 (Z11):
(SEQ ID NO: 4)
KRYKNRVASRKCRAKFKQLLQHYREVAAAKSSENDRLRLLLKQMC
CPP2 (Z12):
(SEQ ID NO: 5)
KRYKNRVASRKCRAKFKQLLQHYREVAAAKSSENDRLRLLLK
CPP3 (Z13):
(SEQ ID NO: 6)
KRYKNRVASRKSRAKFKQLLQHYREVAAAKSSENDRLRLLLK
CPP4 (Z14):
(SEQ ID NO: 7)
KRYKNRVASRKSRAKFKQLLQHYREVAAAK
CPP5 (Z15):
(SEQ ID NO: 8)
KRYKNRVASRKSRAKFK
CPP6 (Z16):
(SEQ ID NO: 9)
QHYREVAAAKSSEND
CPP7 (Z17):
(SEQ ID NO: 10)
QLLQHYREVAAAK
CPP8 (Z18):
(SEQ ID NO: 11)
REVAAAKSS END RLRLLLK
CPP9 (Z19):
(SEQ ID NO: 12)
KRYKNRVA
CPP10 (Z20):
(SEQ ID NO: 13)
VASRKSRAKFK

Thereby, a cell penetrating peptide is particularly preferred, which has an amino acid sequence comprising or consisting of an amino acid sequence according to SEQ ID NO: 6 (CPP3/Z13), SEQ ID NO: 7 (CPP4/Z14), SEQ ID NO: 8 (CPP5/Z15), or SEQ ID NO: 11 (CPP8/Z18), or sequence variants thereof without abrogating said peptide's cell penetrating ability, preferably sequence variants having 0, 1, 2, 3, 4, or 5 amino acids substituted, deleted and/or added without abrogating said peptide's cell penetrating ability. Moreover, a cell penetrating peptide is more preferred, which has an amino acid sequence comprising or consisting of an amino acid sequence according to SEQ ID NO: 6 (CPP3/Z13) or SEQ ID NO: 7 (CPP4/Z14) or sequence variants thereof without abrogating said peptide's cell penetrating ability, preferably sequence variants having 0, 1, 2, 3, 4, or 5 amino acids substituted, deleted and/or added without abrogating said peptide's cell penetrating ability. Moreover, a cell penetrating peptide is most preferred, which has an amino acid sequence comprising or consisting of an amino acid sequence according to SEQ ID NO: 6 (CPP3/Z13) or sequence variants thereof without abrogating said peptide's cell penetrating ability, preferably sequence variants having 0, 1, 2, 3, 4, or 5 amino acids substituted, deleted and/or added without abrogating said peptide's cell penetrating ability.

In one preferred embodiment, the cell penetrating peptide according to the invention has an amino acid sequence comprising or consisting of SEQ ID NO: 6 (CPP3/Z13).

In another preferred embodiment, the cell penetrating peptide according to the invention has an amino acid sequence comprising or consisting of SEQ ID NO: 7 (CPP4/Z14).

In another preferred embodiment, the cell penetrating peptide according to the invention has an amino acid sequence comprising or consisting of SEQ ID NO: 8 (CPP5/Z15).

In another preferred embodiment, the cell penetrating peptide according to the invention has an amino acid sequence comprising or consisting of SEQ ID NO: 11 (CPP8/Z18).

It will be understood by one skilled in the art that the primary amino acid sequence of the cell penetrating peptide may further be post-translationally modified, such as by glycosylation or phosphorylation, without departing from the invention.

In certain embodiments, the cell penetrating peptide optionally further comprises, in addition to its amino acid sequence as described above, any one of, or any combination of:
(i) a nuclear localization signal (NLS). Such signals are well known to the skilled person and are described in Nair et al. (2003, Nucleic Acids Res. 31(1): 397-399)
(ii) a targeting peptide, including tumor homing peptides such as those described in Kapoor et al. (2012, PLoS ONE 7(4): e35187) and listed in http://crdd.osdd.net/raghava/tumorhope/general.php?

Preferably, the cell penetrating peptide is linked to an antigen or antigenic epitope and facilitates the cellular internalization of said antigen or antigenic epitope.

The complex comprised in the combination according to the present invention may comprise one single cell penetrating peptide or more than one cell penetrating peptides. Preferably, the complex comprised by the combination according to the present invention comprises no more than five cell penetrating peptides, more preferably the complex comprised by the combination according to the present invention comprises no more than four cell penetrating peptides, even more preferably the complex comprised by the combination according to the present invention comprises no more than three cell penetrating peptides, particularly preferably the complex comprised by the combination according to the present invention comprises no more than two cell penetrating peptides and most preferably the complex comprised by the combination according to the present invention comprises a single cell penetrating peptide.

Component b)—Antigen/Antigenic Epitope

The complex comprised by the combination according to the present invention comprises as component b) at least one antigen or antigenic epitope.

In general, the at least one antigen or antigenic epitope may be of any nature, for example it may be selected from the group consisting of: (i) a peptide, a polypeptide, or a protein, (ii) a polysaccharide, (iii) a lipid, (iv) a lipoprotein or a lipopeptide, (v) a glycolipid, (vi) a nucleic acid, and (vii) a small molecule drug or a toxin. Thus, the at least one antigen or antigenic epitope may be a peptide, a protein, a polysaccharide, a lipid, a combination thereof including lipoproteins and glycolipids, a nucleic acid (e.g. DNA, siRNA, shRNA, antisense oligonucleotides, decoy DNA, plasmid), or a small molecule drug (e.g. cyclosporine A, paclitaxel, doxorubicin, methotrexate, 5-aminolevulinic acid), or any combination thereof (in particular if more than one antigen or antigenic epitope is comprised by the complex comprised by the combination according to the present invention). Preferably, the at least one antigen or antigenic epitope comprised by the complex is a (poly)peptide.

As used herein, an “antigen” is any structural substance which serves as a target for the receptors of an adaptive immune response, in particular as a target for antibodies, T cell receptors, and/or B cell receptors. An “epitope”, also known as “antigenic determinant”, is the part (or fragment) of an antigen that is recognized by the immune system, in particular by antibodies, T cell receptors, and/or B cell receptors. Thus, one antigen has at least one epitope, i.e. a single antigen has one or more epitopes. In the context of the present invention, the term “epitope” is mainly used to designate T cell epitopes, which are presented on the surface of an antigen-presenting cell, where they are bound to Major Histocompatibility Complex (MHC). T cell epitopes presented by MHC class I molecules are typically, but not exclusively, peptides between 8 and 11 amino acids in length, whereas MHC class II molecules present longer peptides, generally, but not exclusively, between 12 and 25 amino acids in length.

Preferably, the complex comprises at least one fragment of an antigen, said fragment comprising at least one epitope of said antigen. As used herein, a “fragment” of an antigen comprises at least 10 consecutive amino acids of the antigen, preferably at least 15 consecutive amino acids of the antigen, more preferably at least 20 consecutive amino acids of the antigen, even more preferably at least 25 consecutive amino acids of the antigen and most preferably at least 30 consecutive amino acids of the antigen. A “sequence variant” of an antigen or antigenic epitope (or fragment) is as defined above, namely a sequence variant has an (amino acid) sequence which is at least 70% or at least 75%, preferably at least 80% or at least 85%, more preferably at least 90% or at least 95%, even more preferably at least 97% or at least 98%, particularly preferably at least 99% identical to the reference sequence. A “functional” sequence variant means in the context of an antigen/antigen fragment/epitope, that the function of the epitope(s), e.g. comprised by the antigen (fragment), is not impaired or abolished, i.e. that it is immunogenic, preferably has the same immunogenicity as the epitope comprised in the full length antigen. In some embodiments, the amino acid sequence of the epitope(s), e.g. comprised by the cancer/tumor antigen (fragment) as described herein, is not mutated and, thus, identical to a (naturally occurring) reference epitope sequence.

Preferably, the complex comprised by the combination according to the present invention comprises more than one antigen or antigenic epitope, in particular 2, 3, 4, 5, 6, 7, 8, 9, 10 or more antigens or antigenic epitopes, more preferably the complex comprised by the combination according to the present invention comprises (at least) two or three antigens or antigenic epitopes, even more preferably the complex comprised by the combination according to the present invention comprises (at least) four or five antigens or antigenic epitopes. If more than one antigen or antigenic epitope is comprised by the complex comprised by the combination according to the present invention it is understood that said antigen or antigenic epitope is in particular also covalently linked in the complex comprised by the combination according to the present invention, e.g. to another antigen or antigenic epitope and/or to a component a), i.e. a cell penetrating peptide, and/or to a component c), i.e. a TLR peptide agonist.

The various antigens or antigenic epitopes comprised by the complex may be the same or different. Preferably, the various antigens or antigenic epitopes comprised by the complex are different from each other, thus providing a multi-antigenic and/or multi-epitopic complex.

Moreover, it is preferred that the more than one antigen or antigenic epitope, in particular 2, 3, 4, 5, 6, 7, 8, 9, 10 or more antigens or antigenic epitopes, are positioned consecutively in the complex comprised by the combination according to the present invention. This means in particular that all antigens and/or antigenic epitopes comprised by the complex are positioned in a stretch, which is neither interrupted by component a), i.e. a cell penetrating peptide, nor by component c), i.e. a TLR peptide agonist. Rather, component a) and component c) are positioned in the complex for example before or after such a stretch of all antigens and/or antigenic epitopes. Thereby, a “multi-antigenic domain” may be formed. As used herein the term “multiantigenic domain” refers to a domain, such as a (poly)peptide, comprising (fragments of) at least two (e.g., 2, 3, 4, 5, 6, 7, 8, 9 or more) distinct antigens or antigenic epitopes of at least two (e.g., 2, 3, 4, 5, 6, 7, 8, 9 or more) distinct antigens. Preferably, the “multiantigenic domain” comprises fragments of at least two (e.g., 2, 3, 4, 5, 6, 7, 8, 9 or more) distinct antigens, wherein each fragment comprises at least one antigenic epitope. More preferably, the “multiantigenic domain” comprises fragments of two to five distinct antigens, wherein each fragment comprises at least one antigenic epitope. Even more preferably, the “multiantigenic domain” comprises fragments of (exactly) three or four distinct antigens, wherein each fragment comprises at least one antigenic epitope.

The antigens and/or antigenic epitopes positioned consecutively in such a way may optionally be linked to each other for example by a spacer or linker (e.g., as described below), which is neither component a), i.e. a cell penetrating peptide, nor component c), i.e. a TLR peptide agonist.

Alternatively, however, the various antigens and/or antigenic epitopes may also be positioned in any other way in the complex comprised by the combination according to the present invention, for example with component a) and/or component c) positioned in between two or more antigens and/or antigenic epitopes, i.e. with one or more antigens and/or antigenic epitopes positioned between component a) and component c) (or vice versa) and, optionally, one or more antigens and/or antigenic epitopes positioned at the respective other end of component a) and/or component c).

It is understood that a number of different antigens or antigenic epitopes relating to the same kind of disease, in particular to the same kind of tumor, may be advantageously comprised by a single complex. Alternatively, a number of different antigens or antigenic epitopes relating to the same kind of disease, in particular to the same kind of tumor, may be distributed to subsets of different antigens or antigenic epitopes, in particular subsets complementing each other in the context of a certain kind of disease, e.g. tumor, which are comprised by different complexes, whereby such different complexes comprising different subsets may advantageously be administered simultaneously, e.g. in a single vaccine, to a subject in need thereof.

Preferably, the at least one antigen or antigenic epitope will be presented at the cell surface in an MHC class I and/or MHC class II context and/or in a CD1 context, whereby presentation at the cell surface in an MHC class I and/or MHC class II context is preferred. The phrase “epitope presentation in the MHC class I context” refers in particular to a CD8⁺ epitope lying in the groove of a MHC class I molecule at the surface of a cell. The phrase “epitope presentation in the MHC class II context” refers in particular to a CD4⁺ epitope lying in the groove of a MHC class II molecule at the surface of a cell. The phrase “epitope presentation in the CD1 context” refers in particular to a lipidic epitope lying in the groove of a cluster of differentiation 1 molecule at the surface of a cell.

Advantageously, the complex comprised by the combination according to the invention comprises a cell penetrating peptide and at least one antigen or antigenic epitope, and allows the transport and presentation of said epitopes at the cell surface of antigen presenting cells in an MHC class I and MHC class II context, and is, thus, useful in vaccination and immunotherapy.

Preferably, the complex comprised by the combination according to the present invention comprises at least one antigen or antigenic epitope, which is at least one CD4⁺ epitope and/or at least one CD8⁺ epitope.

The terms “CD4⁺ epitope” or “CD4⁺-restricted epitope”, as used herein, designate an epitope recognized by a CD4⁺ T cell, said epitope in particular consisting of an antigen fragment lying in the groove of a MHC class II molecule. A single CD4⁺ epitope comprised in the complex comprised by the combination according to the present invention preferably consists of about 12-25 amino acids. It can also consist of, for example, about 8-25 amino acids or about 6-100 amino acids.

The terms “CD8⁺ epitope” or “CD8⁺-restricted epitope”, as used herein, designate an epitope recognized by a CD8⁺ T cell, said epitope in particular consisting of an antigen fragment lying in the groove of a MHC class I molecule. A single CD8⁺ epitope comprised in the complex comprised by the combination according to the present invention preferably consists of about 8-11 amino acids. It can also consist of, for example, about 8-15 amino acids or about 6-100 amino acids.

Preferably, the at least one antigen can comprise or the at least one antigenic epitope can consist of a CD4⁺ epitope and/or a CD8⁺ epitope corresponding to antigenic determinant(s) of a cancer/tumor-associated antigen, a cancer/tumor-specific antigen, or an antigenic protein from a pathogen. More preferably, the at least one antigen can comprise or the at least one antigenic epitope can consist of a CD4⁺ epitope and/or a CD8⁺ epitope corresponding to antigenic determinant(s) of a cancer/tumor-associated antigen or a cancer/tumor-specific antigen. Even more preferably, the at least one antigen can comprise or the at least one antigenic epitope can consist of a CD4⁺ epitope and/or a CD8⁺ epitope corresponding to antigenic determinant(s) of a tumor-associated antigen or a tumor-specific antigen.

It is also preferred that the complex comprised by the combination according to the present invention comprises at least two antigens or antigenic epitopes, wherein at least one antigen or antigenic epitope comprises or consists a CD4⁺ epitope and at least one antigen or antigenic epitope comprises or consists a CD8⁺ epitope. It is now established that Tₕ cells (CD4⁺) play a central role in the anti-tumor immune response both in DC licensing and in the recruitment and maintenance of CTLs (CD8⁺) at the tumor site. Therefore, a complex comprised by the combination according to the present invention comprising at least two antigens or antigenic epitopes, wherein at least one antigen or antigenic epitope comprises or consists of a CD4⁺ epitope and at least one antigen or antigenic epitope comprises or consists a CD8⁺ epitope, provides an integrated immune response allowing simultaneous priming of CTLs and Tₕ cells and is thus preferable to immunity against only one CD8⁺ epitope or only one CD4⁺ epitope.

Preferably, the complex comprised by the combination according to the present invention comprises at least two antigens or antigenic epitopes, wherein the at least two antigens or antigenic epitopes comprise or consist of at least two, e.g. 2, 3, 4, 5, 6, 7, 8, 9, or more, CD4⁺ epitopes and/or at least two, e.g. 2, 3, 4, 5, 6, 7, 8, 9, or more, CD8⁺ epitopes. Thereby, the at least two antigens or antigenic epitopes are preferably different antigens or antigenic epitopes, more preferably the at least two antigens or antigenic epitopes are different from each other but relating to the same kind of tumor. A multi-antigenic vaccine will (i) avoid outgrowth of antigen-loss variants, (ii) target different tumor cells within a heterogeneous tumor mass and (iii) circumvent patient-to-patient tumor variability. Thus, the complex comprised by the combination according to the present invention particularly preferably comprises at least four antigens or antigenic epitopes, in particular with at least two CD8⁺ epitopes and at least two CD4⁺ epitopes. Such a complex comprised by the combination according to the present invention induces multi-epitopic CD8 CTLs and CD4 Tₕ cells to function synergistically to counter tumor cells and promote efficient anti-tumor immunity. Tₕ cells are also involved in the maintenance of long-lasting cellular immunity that was monitored after vaccination. Such a complex comprised by the combination according to the present invention induces polyclonal, multi-epitopic immune responses and poly-functional CD8⁺ and CD4⁺ T cells, and thus efficacious anti-tumor activity.

Preferably, the complex comprised by the combination according to the present invention comprises at least two antigens or antigenic epitopes, more preferably the complex comprised by the combination according to the present invention comprises at least three antigens or antigenic epitopes, even more preferably the complex comprised by the combination according to the present invention comprises at least four antigens or antigenic epitopes, particularly preferably the complex comprised by the combination according to the present invention comprises at least five antigens or antigenic epitopes and most preferably the complex comprised by the combination according to the present invention comprises at least six antigens or antigenic epitopes. The antigens or antigenic epitopes comprised by the complex may be the same or different, preferably the antigens or antigenic epitopes comprised by the complex are different from each other. Preferably, the complex comprises at least one CD4⁺ epitope and at least one CD8⁺ epitope.

Preferably, the complex comprised by the combination according to the present invention comprises more than one CD4⁺ epitope, e.g. two or more CD4⁺ epitopes from the same antigen or from different antigens, and preferably no CD8⁺ epitope. It is also preferred that the complex comprised by the combination according to the present invention comprises more than one CD8⁺ epitope, e.g. two or more CD8⁺ epitopes from the same antigen or from different antigens, and preferably no CD4⁺ epitope. Most preferably, however, the complex comprised by the combination according to the present invention comprises (i) at least one CD4⁺ epitope, e.g. two or more CD4⁺ epitopes from the same antigen or from different antigens, and (ii) at least one CD8⁺ epitope, e.g. two or more CD8⁺ epitopes from the same antigen or from different antigens.

While the at least one antigen or antigenic epitope may comprise any kind of antigen or antigenic epitope, for example, (one or more epitope(s) from) a cancer/tumor-associated antigen, a cancer/tumor-specific antigen, and/or an antigenic protein from a pathogen, including viral, bacterial, fungal, protozoal and multicellular parasitic antigenic protein, cancer or tumor epitopes are preferred.

It is understood, that the skilled person usually selects the antigen or antigenic epitope in view of the disease to be treated. Accordingly, the antigen or antigenic epitope is usually associated with (or related to) the disease to be treated. A large number of antigens is known in the art in the context of specific diseases. For example, to treat a tumor/cancer, the skilled person selects a tumor/cancer antigen (or antigenic epitope), in particular a tumor/cancer antigen (or antigenic epitope), which is useful for the specific type of tumor/cancer. In some embodiments, the patient may be tested/screened for specific antigens (e.g., by using an isolated sample to identify whether or not the cancer/tumor expresses the specific antigen) in order to determine whether or not the specific antigen in question is useful for the treatment (or to identify a useful antigen or antigenic epitope for the treatment).

Preferably, the at least one antigen or antigenic epitope comprises or consists of at least one cancer or tumor epitope. More preferably, the at least one antigen or antigenic epitope preferably comprises or consists of at least one epitope of a cancer/tumor-associated antigen or a cancer/tumor-specific antigen.

As used herein, “cancer/tumor antigens/epitopes” are antigens/epitopes produced by cancer/tumor cells. Such epitopes are typically specific for (or associated with) a certain kind of cancer/tumor. For instance, cancer/tumor epitopes include glioma epitopes. In particular, cancer/tumor-associated (also cancer/tumor-related) antigens (TAAs) are antigens, which are expressed by both, cancer/tumor cells and normal cells. For example, a TAA may be one or more surface proteins or polypeptides, nuclear proteins or glycoproteins, or fragments thereof, expressed by a tumor cell. For example, human tumor-associated antigens include differentiation antigens (such as melanocyte differentiation antigens), mutational antigens (such as p53), overexpressed cellular antigens (such as HER2), viral antigens (such as human papillomavirus proteins), and cancer/testis (CT) antigens that are expressed in germ cells of the testis and ovary but are silent in normal somatic cells (such as MAGE and NY-ESO-1). Many TAAs are not cancer- or tumor-specific and may also be found on normal tissues. Accordingly, those antigens may be present since birth (or even before). Therefore, there is a chance that the immune system developed self-tolerance to those antigens.

Cancer/tumor-specific antigens (TSAs), in contrast, are antigens, which are expressed specifically by cancer/tumor cells, but not by normal cells. TSA can be specifically recognized by neoantigen-specific T cell receptors (TCRs) in the context of major histocompatibility complexes (MHCs) molecules. Accordingly, TSA include in particular neoantigens. In general neoantigens are antigens, which were not present before and are, thus, “new” to the immune system. Neoantigens are typically due to somatic mutations. In the context of cancer/tumors, cancer/tumor-specific neoantigens were typically not present before the cancer/tumor developed and cancer/tumor-specific neoantigens are usually encoded by somatic gene mutations in the cancerous cells/tumor cells. From an immunological perspective, tumor neoantigen is the truly foreign protein and entirely absent from normal human organs/tissues. For most human tumors without a viral etiology, tumor neoantigens can e.g. derive from a variety of nonsynonymous genetic alterations including single-nucleotide variants (SNVs), insertions and deletions (indel), gene fusions, frameshift mutations, and structural variants (SVs). For example, tumor-neoantigens may be identified using in silico prediction tools known in the art as disclosed in Trends in Molecular Medicine, November 2019, Pages 980-992. Since neoantigens are new to the immune system, the risk of self-tolerance of those antigens is considerably lower as compared to cancer/tumor-associated antigens. However, every cancer's set of tumor-specific mutations appears to be unique. Accordingly, cancer/tumor-specific antigens, in particular neoantigens, may be identified in a subject diagnosed with a cancer by methods known to the skilled person, e.g., cancer genome sequencing. Potential neoantigens may be predicted by methods known to the skilled person, such as cancer genome sequencing or deep-sequencing technologies identifying mutations within the protein-coding part of the (cancer) genome. After identification, the respective cancer/tumor-specific neoantigens and/or cancer/tumor-specific neoantigenic epitopes may be used in the complex comprised by the combination according to the present invention.

In some embodiments, a complex comprised by the combination according to the present invention comprises one or more cancer/tumor-associated epitopes and/or one or more cancer/tumor-associated antigens (but preferably no cancer/tumor-specific epitopes). In other embodiments, the complex comprised by the combination according to the present invention comprises one or more cancer/tumor-specific epitopes and/or one or more cancer/tumor-specific antigens (but preferably no cancer/tumor-associated epitopes). A complex comprised by the combination according to the present invention may also comprise both, (i) one or more cancer/tumor-associated epitopes and/or one or more cancer/tumor-associated antigens and (ii) one or more cancer/tumor-specific epitopes and/or one or more cancer/tumor-specific antigens.

Suitable cancer/tumor epitopes can be retrieved for example from cancer/tumor epitope databases, e.g. from van der Bruggen P, Stroobant V, Vigneron N, Van den Eynde B. Peptide database: T cell-defined tumor antigens. Cancer Immun 2013; URL: http://www.cancerimmunity.org/peptide/, wherein human tumor antigens recognized by CD4+ or CD8+ T cells are classified into four major groups on the basis of their expression pattern, or from the database “Tantigen” (TANTIGEN version 1.0, Dec. 1, 2009; developed by Bioinformatics Core at Cancer Vaccine Center, Dana-Farber Cancer Institute; URL: http://cvc.dfci.harvard.edu/tadb/).

Specific examples of cancer/tumor antigens useful in a complex comprised by the combination according to the present invention include, but are not limited to, the following antigens: Prostate: prostate-specific antigen (PSA), prostate-specific membrane antigen (PSMA), PAP, PSCA (PNAS 95(4) 1735-1740 1998), prostate mucin antigen (PMA) (Beckett and Wright, 1995, Int. J. Cancer 62: 703-710), Prostase, Her-2neu, SPAS-1; Melanoma: TRP-2, tyrosinase, Melan A/Mart-1, gplOO, BAGE, GAGE, GM2 ganglioside; Breast: Her2-neu, kinesin 2, TATA element modulatory factor 1, tumor protein D52, MAGE D, ING2, HIP-55, TGF-1 anti-apoptotic factor, HOM-Mel-40/SSX2, epithelial antigen (LEA 135), DF31MUC1 antigen (Apostolopoulos et al., 1996 Immunol. Cell. Biol. 74: 457-464; Pandey et al., 1995, Cancer Res. 55: 4000-4003); Testis: MAGE-1, HOM-Mel-40/SSX2, NY-ESO-1; Colorectal: EGFR, CEA; Lung: MAGE D, EGFR Ovarian Her-2neu; Baldder: transitional cell carcinoma (TCC) (Jones et al., 1997, Anticancer Res. 17: 685-687), Several cancers: Epha2, Epha4, PCDGF, HAAH, Mesothelin; EPCAM; NY-ESO-1, glycoprotein MUC1 and NIUC10 mucins p5 (especially mutated versions), EGFR; Miscellaneous tumor: cancer-associated serum antigen (CASA) and cancer antigen 125 (CA 125) (Kierkegaard et al., 1995, Gynecol. Oncol. 59: 251-254), the epithelial glycoprotein 40 (EGP40) (Kievit et al., 1997, Int. J. Cancer 71: 237-245), squamous cell carcinoma antigen (SCC) (Lozza et al., 1997 Anticancer Res. 17: 525-529), cathepsin E (Mota et al., 1997, Am. J Pathol. 150: 1223-1229), tyrosinase in melanoma (Fishman et al., 1997 Cancer 79: 1461-1464), cell nuclear antigen (PCNA) of cerebral cavernomas (Notelet et al., 1997 Surg. Neurol. 47: 364-370), a 35 kD tumor-associated autoantigen in papillary thyroid carcinoma (Lucas et al., 1996 Anticancer Res. 16: 2493-2496), CDC27 (including the mutated form of the protein), antigens triosephosphate isomerase, 707-AP, A60 mycobacterial antigen (Macs et al., 1996, J. Cancer Res. Clin. Oncol. 122: 296-300), Annexin II, AFP, ART-4, BAGE, β-catenin/m, BCL-2, bcr-abl, bcr-abl p190, bcr-abl p210, BRCA-1, BRCA-2, CA 19-9 (Tolliver and O'Brien, 1997, South Med. J. 90: 89-90; Tsuruta at al., 1997 Urol. Int. 58: 20-24), CAMEL, CAP-1, CASP-8, CDC27/m, CDK-4/m, CEA (Huang et al., Exper Rev. Vaccines (2002) 1:49-63), CT9, CT10, Cyp-B, Dek-cain, DAM-6 (MAGE-B2), DAM-10 (MAGE-B1), EphA2 (Zantek et al., Cell Growth Differ. (1999) 10:629-38; Carles-Kinch et al., Cancer Res. (2002) 62:2840-7), EphA4 (Cheng at al., 2002, Cytokine Growth Factor Rev. 13:75-85), tumor associated Thomsen-Friedenreich antigen (Dahlenborg et al., 1997, Int. J Cancer 70: 63-71), ELF2M, ETV6-AML1, G250, GAGE-1, GAGE-2, GAGE-3, GAGE-4, GAGE-5, GAGE-6, GAGE-7B, GAGE-8, GnT-V, gp100 (Zajac et al., 1997, Int. J Cancer 71: 491-496), HAGE, HER2/neu, HLA-A*0201-R170I, HPV-E7, HSP70-2M, HST-2, hTERT, hTRT, iCE, inhibitors of apoptosis (e.g., survivin), KH-1 adenocarcinoma antigen (Deshpande and Danishefsky, 1997, Nature 387: 164-166), KIAA0205, Kras, LAGE, LAGE-1, LDLR/FUT, MAGE-1, MAGE-2, MAGE-3, MAGE-6, MAGE-A1, MAGE-A2, MAGE-A3, MAGE-A4, MAGE-A6, MAGE-A10, MAGE-A12, MAGE-B5, MAGE-B6, MAGE-C2, MAGE-C3, MAGE D, MART-1, MART-1/Melan-A (Kawakami and Rosenberg, 1997, Int. Rev. Immunol. 14: 173-192), MC1R, MDM-2, Myosin/m, MUC1, MUC2, MUM-1, MUM-2, MUM-3, neo-polyA polymerase, NA88-A, NY-ESO-1, NY-ESO-1a (CAG-3), PAGE-4, PAP, Proteinase 3 (Molldrem et al., Blood (1996) 88:2450-7; Molldrem et al., Blood (1997) 90:2529-34), P15, p190, Pm1/RARα, PRAME, PSA, PSM, PSMA, RAGE, RAS, RCAS1, RU1, RU2, SAGE, SART-1, SART-2, SART-3, SP17, SPAS-1, TEL/AML1, TPI/m, Tyrosinase, TARP, TRP-1 (gp75), TRP-2, TRP-2/INT2, WT-1, and alternatively translated NY-ESO-ORF2 and CAMEL proteins, derived from the NY-ESO-1 and LAGE-1 genes. Numerous other cancer antigens are well known in the art.

In some embodiments, the cancer/tumor antigen or the cancer/tumor epitope may be a recombinant cancer/tumor antigen or a recombinant cancer/tumor epitope. Such a recombinant cancer/tumor antigen or a recombinant cancer/tumor epitope may be designed by introducing mutations that change (add, delete or substitute) particular amino acids in the overall amino acid sequence of the native cancer/tumor antigen or the native cancer/tumor epitope. The introduction of mutations does not alter the cancer/tumor antigen or the cancer/tumor epitope so much that it cannot be universally applied across a mammalian subject, and preferably a human or dog subject, but changes it enough that the resulting amino acid sequence breaks tolerance or is considered a foreign antigen in order to generate an immune response. Another manner may be creating a consensus recombinant cancer/tumor antigen or cancer/tumor epitope that has at least 85% and up to 99% amino acid sequence identity to its' corresponding native cancer/tumor antigen or native cancer/tumor epitope; preferably at least 90% and up to 98% sequence identity; more preferably at least 93% and up to 98% sequence identity; or even more preferably at least 95% and up to 98% sequence identity. In some instances the recombinant cancer/tumor antigen or the recombinant cancer/tumor epitope has 95%, 96%, 97%, 98%, or 99% amino acid sequence identity to its' corresponding native cancer/tumor antigen or cancer/tumor epitope. The native cancer/tumor antigen is the antigen normally associated with the particular cancer or cancer tumor. Depending upon the cancer/tumor antigen, the consensus sequence of the cancer/tumor antigen can be across mammalian species or within subtypes of a species or across viral strains or serotypes. Some cancer/tumor antigen do not vary greatly from the wild type amino acid sequence of the cancer/tumor antigen. The aforementioned approaches can be combined so that the final recombinant cancer/tumor antigen or cancer/tumor epitope has a percent similarity to native cancer antigen amino acid sequence as discussed above. In other embodiments, the amino acid sequence of an epitope of a cancer/tumor antigen as described herein is not mutated and, thus, identical to the reference epitope sequence.

Preferably, the at least one cancer/tumor antigen or epitope (e.g. comprised in the multi-antigenic domain), is selected from the group of tumors or cancers comprising endocrine tumors, gastrointestinal tumors, genitourinary and gynecologic tumors, head and neck tumors, hematopoietic tumors, skin tumors, thoracic and respiratory tumors. Preferably, the at least one tumor epitope, or the at least one TAA, or the at least one TSA of the multi-antigenic domain of the invention is selected from the group of tumors and/or cancers comprising breast cancer, including triple-negative breast cancer, biliary tract cancer; bladder cancer; brain cancer including glioblastomas and medulloblastomas; cervical cancer; choriocarcinoma; colon cancer; endometrial cancer; esophageal cancer; gastric cancer; gastrointestinal stromal tumor (GIST), appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, colorectal cancer, or metastatic colorectal cancer, hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer, including non-small cell lung cancer, lymphomas including Hodgkin's disease and lymphocytic lymphomas; neuroblastomas; glioblastoma, oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

In particular, the at least one cancer/tumor antigen or epitope (e.g. comprised in the multi-antigenic domain) is preferably selected from the group of tumors or cancers comprising colorectal cancer, metastatic colorectal cancer, pancreatic cancer, or breast cancer, including triple-negative breast cancer (TN BC). The term “triple negative breast cancer” as used herein refers to breast cancer that lacks the expression of estrogen receptor (ER), progesterone receptor (PgR) and HER2, all of which are molecular targets of therapeutic agents. TNBC accounts for 10-20% of invasive breast cancer cases and encompasses more than one molecular subtype. Typically, patients afflicted with TNBC have a relatively poorer outcome compared with those with other breast cancer subtypes owing to an inherently aggressive clinical behavior and a lack of recognized molecular targets for therapy. Triple negative breast cancer is a phenotype and its major components in molecular assays are the basal-like tumors, normal breast-like tumors and the more recently recognized, the uncommon but intriguing, claudin-low molecular subtypes and also includes BRCA1-deficient subtypes. TAAs that are expressed by TNBC comprise for example MAGE-A3, MUC-1, PRAME, ASCL2, and NY-ESO-1.

The term “pancreas cancer” or “pancreatic cancer” as used herein relates to cancer which is derived from pancreatic cells. Preferably, pancreatic cancer as used herein refers to pancreatic adenocarcinoma, including pancreatic ductal adenocarcinoma and ist morphological variants, e.g. adenosquamous carcinoma, colloid/mucinous carcinoma, undifferentiated/anaplastic carcinoma, signet ring cell carcinoma, medullary carcinoma, hepatoid carcinoma. Pancreatic adenocarcinoma is a lethal condition with poor outcomes and an increasing incidence. Pancreatic cancer is typically a disease of the elderly. It is extremely rare for patients to be diagnosed before the age of 30, and 90% of newly diagnosed patients are aged over 55 years of age, with the majority in their 7th and 8th decade of life, with a higher incidence in males compared to females. Pancreatic cancer is characterized by the expression of tumor-associated antigens comprising mesothelin, survivin, and NY-ESO-1.

As used herein, colorectal cancer (CRC, also known as “bowel cancer”) is a cancer that comprises colon cancers and rectal cancers (CC). Both individual cancers have many features in common, but the cancer starting point. According to Siegel, R., C. Desantis, and A. Jemal, Colorectal cancer statistics, 2014. CA Cancer J Clin, 2014. 64(2): p. 104-17, in the United States between 2006 and 2010, the incidence by tumor site is slightly more important in the proximal colon (first and middle parts of the colon). With about 19 cases on 100,000 people, it represents 42% of the cases. It is followed by the rectal cancer, with 28% of the cases and the distal colon (bottom part of the colon) with an incidence of 10 cases on 100,000 people. Anatomically, the term “colorectal cancer” includes (i) cancers of colon, such as cancers of cecum (including cancers the ileocecal valve), appendix, ascending colon, hepatic flexure, transverse colon, splenic flexure, descending colon, sigmoid colon (including cancers of sigmoid (flexure)) as well as cancers of overlapping sites of colon; (ii) cancers of recto-sigmoid junction, such as cancers of colon and rectum and cancers of rectosigmoid; and (iii) cancers of rectum, such as cancers of rectal ampulla.

Preferably, the colorectal cancer is a cancer of colon, such as a cancer of cecum (including cancer the ileocecal valve), cancer of appendix, cancer of ascending colon, cancer of hepatic flexure, cancer of transverse colon, cancer of splenic flexure, cancer of descending colon, cancer of sigmoid colon (including cancers of sigmoid (flexure)) or a combination thereof.

It is also preferred that the colorectal cancer is a cancer of rectosigmoid junction, such as (i) a cancer of colon and rectum or (ii) a cancer of rectosigmoid. Furthermore, it is also preferred that the colorectal cancer is a cancer of rectum, such as a cancer of rectal ampulla.

Colorectal cancer comprises different cell types such as e.g. the cell type, colorectal cancers include colorectal adenocarcinoma, colorectal stromal tumors, primary colorectal lymphoma, colorectal leiomyosarcoma, colorectal melanoma, colorectal squamous cell carcinoma and colorectal carcinoid tumors, such as, for example, carcinoid tumors of cecum, appendix, ascending colon, transverse colon, descending colon, sigmoid colon and/or rectum. Thus, preferred types of colorectal cancers include colorectal adenocarcinoma, colorectal stromal tumors, primary colorectal lymphoma, colorectal leiomyosarcoma, colorectal melanoma, colorectal squamous cell carcinoma and colorectal carcinoid tumors, such as, for example, carcinoid tumors of cecum, appendix, ascending colon, transverse colon, descending coloncom, sigmoid colon and/or rectum. More preferably, the colorectal cancer is a colorectal adenocarcinoma or a colorectal carcinoid carcinoma. Even more preferably, the colorectal cancer is a colorectal adenocarcinoma. Accordingly, the at least one tumor or cancer epitope of the complex may be selected from any of the colorectal cancer cell types disclosed above.

Since colorectal cancer expresses different TAAs, or TSAs depending on the staging of the tumor according to the TMN staging system, the at least one tumor or cancer epitope (of the multi-antigenic domain) of the complex preferably includes TAAs, or TSAs of for example the following stages for primary tumors (“T” stages): TX—Primary tumour cannot be assessed, T0—No evidence of primary tumour, Ta—Non-invasive papillary carcinoma, Tis—Carcinoma in situ: intraepithelial or invasion of lamina propria, T1—Tumour invades submucosa, T2—Tumour invades muscularis propria, T3—Tumour invades through the muscularis propria into the pericolorectal tissues, T4a—Tumour penetrates to the surface of the visceral peritoneum and T4b—Tumour directly invades or is adherent to other organs or structures; following stages for lymph nodes (“N” stages): NX—Regional lymph nodes cannot be assessed, N0—No regional lymph node metastasis, N1—Metastasis in 1-3 regional lymph nodes with N1a—Metastasis in 1 regional lymph node, N1b—Metastasis in 2-3 regional lymph nodes and N1c—Tumor deposit(s) in the subserosa, mesentery, or nonperitonealized pericolic or perirectal tissues without regional nodal metastasis, N2—Metastasis in 4 or more lymph nodes with N2a—Metastasis in 4-6 regional lymph nodes and N2b—Metastasis in 7 or more regional lymph nodes; and the following stages for distant metastasis (“M” stages): M0—No distant metastasis and M1—Distant metastasis with M1a—Metastasis confined to 1 organ or site (eg, liver, lung, ovary, nonregional node) and M1b—Metastases in more than 1 organ/site or the peritoneum. The stages can be integrated into the following numerical staging of colorectal cancer: Stage 0: Tis, N0, M0; Stage I: T1, N0, M0 or T2, N0, M0; Stage IIA: T3, N0, M0; Stage IIB: T4a, N0, M0; Stage IIC: T4b, N0, M0; Stage IIIA: T1-T2, N1/N1c, M0 or T1, N2a, M0; Stage IIIB: T3-T4a, N1/N1c, M0 or T2-T3, N2a, M0 or T1-T2, N2b, M0; Stage IIIC: T4a, N2a, M0 or T3-T4a, N2b, M0 or T4b, N1-N2, M0; Stage IVA: any T, any N, M1a and Stage IVB: any T, any N, M1b. Briefly, in Stage 0, the cancer has not grown beyond the inner layer of the colon or rectum; in Stage I the cancer has spread from the mucosa to the muscle layer; in Stage II the cancer has spread through the muscle layer to the serosa nearby organs; in Stage III the cancer has spread to nearby lymph node(s) or cancer cells have spread to tissues near the lymph nodes; and in Stage IV the cancer has spread through the blood and lymph nodes to other parts of the body.

Various tumor associated antigens of the above colorectal cancer cell types and stages have been reported and comprise e.g. CEA, MAGE, MUC1, survivin, WT1, RNF43, TOMM34, VEGFR-1, VEGFR-2, KOC1, ART4, KRas, EpCAM, HER-2, COA-1 SAP, TGF-βRII, p53, ASCL2, and SART 1-3 (see e.g. World J Gastroenterol 2018 Dec. 28; 24(48): 5418-5432). Accordingly, the at least one cancer/tumor epitope/antigen of the complex is preferably (an epitope of) an antigen selected from the group consisting of EpCAM, HER-2, MUC-1, TOMM34, RNF 43, KOC1, VEGFR, βhCG, survivin, CEA, ASCL2, TGFβR2, p53, KRas, OGT, mesothelin, CASP5, COA-1, MAGE, SART, IL13Ralpha2, ASCL2, NY-ESO-1, MAGE-A3, PRAME, WT1.

### Melanoma-Associated Antigen (MAGE)

The mammalian members of the MAGE (melanoma-associated antigen) gene family were originally described as completely silent in normal adult tissues, with the exception of male germ cells and, for some of them, placenta. By contrast, these genes were expressed in various kinds of tumors. Therefore, the complex preferably comprises an antigen of the MAGE-family (a “MAGE” antigen) or an epitope thereof. Of the MAGE family, in particular MAGE-A3 and MAGE-D4 are preferred, and MAGE-A3 is particularly preferred. The normal function of MAGE-A3 in healthy cells is unknown. MAGE-A3 which may e.g. also be referred to as Cancer/Testis Antigen 1.3, is a tumor-specific protein, and has been identified on many tumors. The amino acid sequence of MAGE-A3 is shown in the following:

[SEQ ID NO: 14]
MPLEQRSQHCKPEEGLEARGEALGLVGAQAPATEEQEAASSSSTLVEVT
LGEVPAAESPDPPQSPQGASSLPTTMNYPLWSQSYEDSSNQEEEGPSTF
PDLESEFQAALSRKVAELVHFLLLKYRAREPVTKAEMLGSVVGNWQYFF
PVIFSKAFSSLQLVFGIELMEVDPIGHLYIFATCLGLSYDGLLGDNQIM
PKAGLLIIVLAIIAREGDCAPEEKIWEELSVLEVFEGREDSILGDPKKL
LTQHFVQENYLEYRQVPGSDPACYEFLWGPRALVETSYVKVLHHMVKIS
GGPHISYPPLHEWVLREGEE

Accordingly, the complex preferably comprises the amino acid sequence according to SEQ ID NO: 14, or a fragment or variant thereof as described herein.

### Mesothelin

Mesothelin, which was initially identified in ovarian cancer as a protein reacting with an antibody termed “mAb K1” is a tumor antigen that is highly expressed in many human cancers, including malignant mesothelioma and pancreatic, ovarian, and lung adenocarcinomas. The amino acid sequence of mesothelin according to UniProtKB Q13421 is shown below:

[SEQ ID NO: 15]
MALPTARPLLGSCGTPALGSLLFLLFSLGWVQPSRTLAGETGQEAAPLD
GVLANPPNISSLSPRQLLGFPCAEVSGLSTERVRELAVALAQKNVKLST
EQLRCLAHRLSEPPEDLDALPLDLLLFLNPDAFSGPQACTRFFSRITKA
NVDLLPRGAPERQRLLPAALACWGVRGSLLSEADVRALGGLACDLPGRF
VAESAEVLLPRLVSCPGPLDQDQQEAARAALQGGGPPYGPPSTVVSVST
MDALRGLLPVLGQPIIRSIPQGIVAAWRQRSSRDPSWRQPERTILRPRF
RREVEKTACPSGKKAREIDESLIFYKKWELEACVDAALLATQMDRVNAI
PFTYEQLDVLKHKLDELYPQGYPESVIQHLGYLFLKMSPEDIRKWNVTS
LETLKALLEVNKGHEMSPQAPRRPLPQVATLIDRFVKGRGQLDKDTLDT
LTAFYPGYLCSLSPEELSSVPPSSIWAVRPQDLDTCDPRQLDVLYPKAR
LAFQNMNGSEYFVKIQSFLGGAPTEDLKALSQQNVSMDLATFMKLRTDA
VLPLTVAEVQKLLGPHVEGLKAEERHRPVRDWILRQRQDDLDTLGLGLQ
GGIPNGYLVLDLSMQEALSGTPCLLGPGPVLTVLALLLASTLA

Accordingly, the complex preferably comprises the amino acid sequence according to SEQ ID NO: 15, or a fragment or variant thereof as described herein.

### Survivin

Survivin, also called baculoviral inhibitor of apoptosis repeat-containing 5 or BIRC5 (UniProtKB 015392), is a member of the inhibitor of apoptosis (IAP) family. The survivin protein functions to inhibit caspase activation, thereby leading to negative regulation of apoptosis or programmed cell death. The amino acid sequence of survivin is shown in the following:

[SEQ ID NO: 16]
MGAPTLPPAWQPFLKDHRISTFKNWPFLEGCACTPERMAEAGFIHCPTE
NEPDLAQCFFCFKELEGWEPDDDPIEEHKKHSSGCAFLSVKKQFEELTL
GEFLKLDRERAKNKIAKETNNKKKEFEETAKKVRRAIEQLAAMD

Accordingly, the complex preferably comprises an amino acid sequence according to SEQ ID NO: 16 or a fragment or a variant thereof as described herein.

Several epitopes of survivin are known to the skilled person. A preferred survivin epitope, which is preferably comprised by the complex, includes the following epitope (the epitope sequence shown in the following is a fragment of the above survivin sequence; the following epitope sequence may refer to one epitope or more than one (overlapping) epitopes):

[SEQ ID NO: 17]
RISTFKNWPF

Accordingly, the complex preferably comprises an amino acid sequence according to SEQ ID NO: 17.

Accordingly, it is preferred that the complex comprises an epitope of survivin. More preferably, the complex comprises a peptide having an amino acid sequence according to SEQ ID NO: 16, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least at least 70% or at least 75%, preferably at least 80% or at least 85%, more preferably at least 90% or at least 95%, even more preferably at least 97% or at least 98%, particularly preferably at least 99% sequence identity. Even more preferably, the complex comprises a peptide having an amino acid sequence according to SEQ ID NO: 17.

The complex may also comprise a fragment of survivin comprising at least one epitope, such as SEQ ID NO: 18:

APTLPPAWQPFLKDHRISTFKNWPFLEGSAVKKQFEELTLGEFLKLDRE
R

Particularly preferably, the complex comprises a peptide having an amino acid sequence according to SEQ ID NO: 18 or a functional sequence variant thereof having at least at least 70% or at least 75%, preferably at least 80% or at least 85%, more preferably at least 90% or at least 95%, even more preferably at least 97% or at least 98%, particularly preferably at least 99% sequence identity.

### NY-ESO-1

NY-ESO-1 (also referred to as “Cancer/testis antigen 1”, or “New York esophageal squamous cell carcinoma 1”, UniProtKB P78358) is a well-known cancer-testis antigen (CTAs) with re-expression in numerous cancer types. NY-ESO-1 elicits spontaneous humoral and cellular immune responses and is characterized by a restricted expression pattern, render it a good candidate target for cancer immunotherapy. NY-ESO-1-specific immune responses have been observed in various cancer types. The amino acid sequence of NY-ESO-1 is shown in the following:

[SEQ ID NO: 19]
MQAEGRGTGGSTGDADGPGGPGIPDGPGGNAGGPGEAGATGGRGPRGAG
AARASGPGGGAPRGPHGGAASGLNGCCRCGARGPESRLLEFYLAMPFAT
PMEAELARRSLAQDAPPLPVPGVLLKEFTVSGNILTIRLTAADHRQLQL
SISSCLQQLSLLMWITQCFLPVFLAQPPSGQRR

Preferably, the at least one tumor epitope of the complex is an epitope of an antigen selected from the group consisting of mesothelin, survivin, and NY-ESO-1. For example, the at least one tumor epitope of the complex is an epitope selected from mesothelin, survivin, or mesothelin and NY-ESO-1, or survivin and NY-ESO-1. In some embodiments, the at least one tumor antigen/epitope of the complex comprises an epitope of the antigen mesothelin, or NY-ESO-1, or survivin, or a fragment thereof, or a sequence variant thereof.

For example, a complex comprising a multi-antigenic domain which comprises at least one, e.g. one, two, three, four, five, six, seven, eight, nine, ten or more epitopes selected from at least one, two or all of the antigens as disclosed above, e.g. mesothelin, survivin, and NY-ESO-1, may be particularly useful in the context of pancreatic cancer.

### PRAME

PRAME (Melanoma antigen preferentially expressed in tumors, UniProtKB P78395) otherwise known as cancer testis antigen 130 (CT130), MAPE (melanoma antigen preferentially expressed in tumors) and OIP4 (OPA-interacting protein 4) is a member of the cancer testis antigen (CTA) family. PRAME expression in normal somatic tissues is epigenetically restricted to adult germ cells with low expression in the testis, epididymis, endometrium, ovaries and adrenal glands. Similar to the CTA member NY-ESO-1, PRAME was identified as an immunogenic tumor-associated antigen in melanoma, and since its discovery its expression has been demonstrated in a variety of solid and hematological malignancies including triple negative breast cancer. The amino acid sequence of PRAME is shown below:

[SEQ ID NO: 20]
MERRRLWGSIQSRYISMSVWTSPRRLVELAGQSLLKDEALAIAALELLP
RELFPPLFMAAFDGRHSQTLKAMVQAWPFTCLPLGVLMKGQHLHLETFK
AVLDGLDVLLAQEVRPRRWKLQVLDLRKNSHQDFWTVWSGNRASLYSFP
EPEAAQPMTKKRKVDGLSTEAEQPFIPVEVLVDLFLKEGACDELFSYLI
EKVKRKKNVLRLCCKKLKIFAMPMQDIKMILKMVQLDSIEDLEVTCTWK
LPTLAKFSPYLGQMINLRRLLLSHIHASSYISPEKEEQYIAQFTSQFLS
LQCLQALYVDSLFFLRGRLDQLLRHVMNPLETLSITNCRLSEGDVMHLS
QSPSVSQLSVLSLSGVMLTDVSPEPLQALLERASATLQDLVFDECGITD
DQLLALLPSLSHCSQLTTLSFYGNSISISALQSLLQHLIGLSNLTHVLY
PVPLESYEDIHGTLHLERLAYLHARLRELLCELGRPSMVWLSANPCPHC
GDRTFYDPEPILCPCFMPN

Accordingly, the complex preferably comprises an amino acid sequence according to SEQ ID NO: 20 or a fragment or a variant thereof as described herein.

### ASCL2 (ACHAETE-Scute Homolog 2)

ASCL2 is a basic helix-loop-helix transcription factor essential for the maintenance of proliferating trophoblasts during placental development. ASCL2 was found to be a putative regulator of proliferation that is overexpressed in intestinal neoplasia. The amino acid sequence of ASCL2 is shown in the following:

[SEQ ID NO: 21]
MDGGTLPRSAPPAPPVPVGCAARRRPASPELLRCSRRRRPATAETGGGA
AAVARRNERERNRVKLVNLGFQALRQHVPHGGASKKLSKVETLRSAVEY
IRALQRLLAEHDAVRNALAGGLRPQAVRPSAPRGPPGTTPVAASPSRAS
SSPGRGGSSEPGSPRSAYSSDDSGCEGALSPAERELLDFSSWLGGY

Accordingly, the complex preferably comprises an amino acid sequence according to SEQ ID NO: 21 or a fragment or a variant thereof as described herein.

Several epitopes of ASCL2 are known to the skilled person. Preferred ASCL2 epitopes, which are preferably comprised by the complex, include the following epitopes (the epitope sequences shown in the following are fragments of the above ASCL2 sequence and are, thus, shown in the above ASCL2 sequence underlined; each of the following epitope sequences may refer to one epitope or more than one (overlapping) epitopes):

[SEQ ID NO: 22]
SAVEYIRALQ
[SEQ ID NO: 23]
ERELLDFSSW

Accordingly, the complex preferably comprises an amino acid sequence according to SEQ ID NO: 22 and/or an amino acid sequence according to SEQ ID NO: 23.

Accordingly, it is preferred that the complex comprises an epitope of ASCL2. More preferably, the complex comprises a peptide having an amino acid sequence according to SEQ ID NO: 21, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% or at least 75%, preferably at least 80% or at least 85%, more preferably at least 90% or at least 95%, even more preferably at least 97% or at least 98%, particularly preferably at least 99% sequence identity). Even more preferably, the complex comprises a peptide having an amino acid sequence according to SEQ ID NO: 22 and/or a peptide having an amino acid sequence according to SEQ ID NO: 23.

The complex may also comprise a fragment of ASCL2 comprising at least one epitope, such as SEQ ID NO: 24:

AAVARRNERERNRVKLVNLGFQALRQHVPHGGASKKLSKVETLRSAVEY
IRALQRLLAEHDAVRNALAGGLRPQAVRPSAPRGPSEGALSPAERELLD
FSSWLGGY

Particularly preferably, the complex comprises a peptide having an amino acid sequence according to SEQ ID NO: 24 or a functional sequence variant thereof having at least 70% or at least 75%, preferably at least 80% or at least 85%, more preferably at least 90% or at least 95%, even more preferably at least 97% or at least 98%, particularly preferably at least 99% sequence identity).

### Mucin-1 (MUC-1)

MUC-1 (UniProtKB P15941) is a human epithelial mucin, acting on cell adhesion. The amino acid sequence of MUC-1 is shown in the following:

[SEQ ID NO: 25]
MTPGTQSPFFLLLLLTVLTVVTGSGHASSTPGGEKETSATQRSSVPSST
EKNAVSMTSSVLSSHSPGSGSSTTQGQDVTLAPATEPASGSAATWGQDV
TSVPVTRPALGSTTPPAHDVTSAPDNKPAPGSTAPPAHGVTSAPDTRPA
PGSTAPPAHGVISAPDTRPAPGSTAPPAHGVTSAPDTRPAPGSTAPPAH
GVTSAPDTRPAPGSTAPPAHGVTSAPDTRPAPGSTAPPAHGVTSAPDTR
PAPGSTAPPAHGVTSAPDTRPAPGSTAPPAHGVISAPDTRPAPGSTAPP
AHGVTSAPDTRPAPGSTAPPAHGVTSAPDTRPAPGSTAPPAHGVTSAPD
TRPAPGSTAPPAHGVISAPDTRPAPGSTAPPAHGVTSAPDTRPAPGSTA
PPAHGVTSAPDTRPAPGSTAPPAHGVTSAPDTRPAPGSTAPPAHGVTSA
PDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVT
SAPDTRPAPGSTAPPAHGVTSAPDTRPAPGSTAPPAHGVTSAPDTRPAP
GSTAPPAHGVTSAPDTRPAPGSTAPPAHGVTSAPDTRPAPGSTAPPAHG
VTSAPDTRPAPGSTAPPAHGVTSAPDTRPAPGSTAPPAHGVTSAPDTRP
APGSTAPPAHGVTSAPDTRPAPGSTAPPAHGVISAPDTRPAPGSTAPPA
HGVTSAPDTRPAPGSTAPPAHGVTSAPDTRPAPGSTAPPAHGVTSAPDT
RPAPGSTAPPAHGVTSAPDTRPAPGSTAPPAHGVTSAPDTRPAPGSTAP
PAHGVTSAPDTRPAPGSTAPPAHGVTSAPDTRPAPGSTAPPAHGVTSAP
DTRPAPGSTAPPAHGVTSAPDTRPAPGSTAPPAHGVTSAPDTRPAPGST
APPAHGVTSAPDNRPALGSTAPPVHNVTSASGSASGSASTLVHNGTSAR
ATTTPASKSTPFSIPSHHSDTPTTLASHSTKTDASSTHHSSVPPLTSSN
HSTSPQLSTGVSFFFLSFHISNLQFNSSLEDPSTDYYQELQRDISEMFL
QIYKQGGFLGLSNIKFRPGSVVVQLTLAFREGTINVHDVETQFNQYKTE
AASRYNLTISDVSVSDVPFPFSAQSGAGVPGWGIALLVLVCVLVALAIV
YLIALAVCQCRRKNYGQLDIFPARDTYHPMSEYPTYHTHGRYVPPSSTD
RSPYEKVSAGNGGSSLSYTNPAVAATSANL

Accordingly, the complex preferably comprises an amino acid sequence according to SEQ ID NO: 25 or a fragment or a variant thereof as described herein.

Several epitopes of MUC-1 are known to the skilled person. Preferred MUC-1 epitopes, which are preferably comprised by the complex, include the following epitopes (the epitope sequences shown in the following are fragments of the above MUC-1 sequence and are, thus, shown in the above MUC-1 sequence underlined; each of the following epitope sequences may refer to one epitope or more than one (overlapping) epitopes):

[SEQ ID NO: 26]
GSTAPPVHN
[SEQ ID NO: 27]
TAPPAHGVTS

Accordingly, the complex preferably comprises an amino acid sequence according to SEQ ID NO: 26 and/or an amino acid sequence according to SEQ ID NO: 27.

### Transforming Growth Factor Beta Receptor 2 (TGFβR2)

TGFβ receptors are single pass serine/threonine kinase receptors. They exist in several different isoforms. TGFβR2 (UniProtKB P37137) is a transmembrane protein that has a protein kinase domain, forms a heterodimeric complex with another receptor protein, and binds TGF-beta. This receptor/ligand complex phosphorylates proteins, which then enter the nucleus and regulate the transcription of a subset of genes related to cell proliferation.

[SEQ ID NO: 28]
MGRGLLRGLWPLHIVLWTRIASTIPPHVQKSVNNDMIVTDNNGAVKFPQ
LCKFCDVRFSTCDNQKSCMSNCSITSICEKPQEVCVAVWRKNDENITLE
TVCHDPKLPYHDFILEDAASPKCIMKEKKKPGETFFMCSCSSDECNDNI
IFSEEYNTSNPDLLLVIFQVTGISLLPPLGVAISVIIIFYCYRVNRQQK
LSSTWETGKTRKLMEFSEHCAIILEDDRSDISSTCANNINHNTELLPIE
LDTLVGKGRFAEVYKAKLKQNTSEQFETVAVKIFPYEEYASWKTEKDIF
SDINLKHENILQFLTAEERKTELGKQYWLITAFHAKGNLQEYLTRHVIS
WEDLRKLGSSLARGIAHLHSDHTPCGRPKMPIVHRDLKSSNILVKNDLT
CCLCDFGLSLRLDPTLSVDDLANSGQVGTARYMAPEVLESRMNLENVES
FKQTDVYSMALVLWEMTSRCNAVGEVKDYEPPFGSKVREHPCVESMKDN
VLRDRGRPEIPSFWLNHQGIQMVCETLTECWDHDPEARLTAQCVAERFS
ELEHLDRLSGRSCSEEKIPEDGSLNTTK

Accordingly, the complex preferably comprises an amino acid sequence according to SEQ ID NO: 28 or a fragment or a variant thereof as described herein.

### Carcino-Embryonic Antigen (CEA)

CEA is an intracellular adhesion glycoprotein. CEA is normally produced in gastrointestinal tissue during fetal development, but the production stops before birth. Therefore, CEA is usually present only at very low levels in the blood of healthy adults. The amino acid sequence of CEA is shown in the following:

[SEQ ID NO: 29]
MESPSAPPHRWCIPWQRLLLTASLLTFWNPPTTAKLTIESTPFNVAEGK
EVLLLVHNLPQHLFGYSWYKGERVDGNRQIIGYVIGTQQATPGPAYSGR
EIIYPNASLLIQNIIQNDTGFYTLHVIKSDLVNEEATGQFRVYPELPKP
SISSNNSKPVEDKDAVAFTCEPETQDATYLWWVNNQSLPVSPRLQLSNG
NRTLTLFNVTRNDTASYKCETQNPVSARRSDSVILNVLYGPDAPTISPL
NTSYRSGENLNLSCHAASNPPAQYSWFVNGTFQQSTQELF1PNITVNNS
GSYTCQAHNSDTGLNRTTVTTITVYAEPPKPFITSNNSNPVEDEDAVAL
TCEPEIQNTTYLWWVNNQSLPVSPRLQLSNDNRTLTLLSVTRNDVGPYE
CGIQNKLSVDHSDPVILNVLYGPDDPTISPSYTYYRPGVNLSLSCHAAS
NPPAQYSWLIDGNIQQHTQELFISNITEKNSGLYTCQANNSASGHSRTT
VKTITVSAELPKPSISSNNSKPVEDKDAVAFTCEPEAQNTTYLWWVNGQ
SLPVSPRLQLSNGNRTLTLFNVTRNDARAYVCGIQNSVSANRSDPVTLD
VLYGPDTPIISPPDSSYLSGANLNLSCHSASNPSPQYSWRINGIPQQHT
QVLFIAKITPNNNGTYACFVSNLATGRNNSIVKSITVSASGTSPGLSAG
ATVGIMIGVLVGVAL

Accordingly, the complex preferably comprises an amino acid sequence according to SEQ ID NO: 29 or a fragment or a variant thereof as described herein.

Several epitopes of CEA are known to the skilled person. Preferred CEA epitopes, which are preferably comprised by the complex, include the following epitopes (the epitope sequences shown in the following are fragments of the above CEA sequence and are, thus, shown in the above CEA sequence underlined; each of the following epitope sequences may refer to one epitope or more than one (overlapping) epitopes):

[SEQ ID NO: 30]
YLSGANLNLS
[SEQ ID NO: 31]
SWRINGIPQQ

Accordingly, a preferred complex comprises an amino acid sequence according to SEQ ID NO: 30 and/or an amino acid sequence according to SEQ ID NO: 31.

Accordingly, it is preferred that the complex comprises an epitope of CEA. More preferably, the complex comprises a peptide having an amino acid sequence according to SEQ ID NO: 29, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% or at least 75%, preferably at least 80% or at least 85%, more preferably at least 90% or at least 95%, even more preferably at least 97% or at least 98%, particularly preferably at least 99% sequence identity. Even more preferably, the complex comprises a peptide having an amino acid sequence according to SEQ ID NO: 30 and/or a peptide having an amino acid sequence according to SEQ ID NO: 31.

The complex may also comprise a fragment of CEA comprising at least one epitope, such as SEQ ID NO: 32:

NRTLTLFNVTRNDARAYVSGIQNSVSANRSDPVTLDVLPDSSYLSGANL
NLSCHSASPQYSWRINGIPQQHTQVLFIAKITPNNNGTYACFVSNLATG
RNNSIVKSITVSASGTSPGLSA

Particularly preferably, the complex comprises a peptide having an amino acid sequence according to SEQ ID NO: 32 or a functional sequence variant thereof having at least 70% or at least 75%, preferably at least 80% or at least 85%, more preferably at least 90% or at least 95%, even more preferably at least 97% or at least 98%, particularly preferably at least 99% sequence identity).

### P53

P53 (UniProtKB P04637) is a tumor suppressor protein having a role in preventing genome mutation. P53 has many mechanisms of anticancer function and plays a role in apoptosis, genomic stability, and inhibition of angiogenesis. In its anti-cancer role, p53 works through several mechanisms: it an activate DNA repair proteins when DNA has sustained damage; it can arrest growth by holding the cell cycle at the G1/S regulation point on DNA damage recognition; and it can initiate apoptosis.

[SEQ ID NO: 33]
MEEPQSDPSVEPPLSQETFSDLWKLLPENNVLSPLPSQAMDDLMLSPDD
IEQWFTEDPGPDEAPRMPEAAPPVAPAPAAPTPAAPAPAPSWPLSSSVP
SQKTYQGSYGFRLGFLHSGTAKSVTCTYSPALNKMFCQLAKTCPVQLWV
DSTPPPGTRVRAMAIYKQSQHMTEVVRRCPHHERCSDSDGLAPPQHLIR
VEGNLRVEYLDDRNTFRHSVVVPYEPPEVGSDCTTIHYNYMCNSSCMGG
MNRRPILTIITLEDSSGNLLGRNSFEVRVCACPGRDRRTEEENLRKKGE
PHHELPPGSTKRALPNNTSSSPQPKKKPLDGEYFTLQIRGRERFEMFRE
LNEALELKDAQAGKEPGGSRAHSSHLKSKKGQSTSRHKKLMFKTEGPDS
D

Accordingly, the complex preferably comprises an amino acid sequence according to SEQ ID NO: 33 or a fragment or a variant thereof as described herein.

### Kirsten Ras (KRas)

GTPase KRas also known as V-Ki-ras2 Kirsten rat sarcoma viral oncogene homolog and KRAS, performs an essential function in normal tissue signaling, and the mutation of a KRAS gene is an essential step in the development of many cancers. Like other members of the ras subfamily, the KRAS protein is a GTPase and is an early player in many signal transduction pathways. KRAS is usually tethered to cell membranes because of the presence of an isoprene group on its C-terminus. The amino acid sequence of KRas is shown in the following:

[SEQ ID NO: 34]
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQRVEDAFYTLVREIRQYRLKKISKEEKTPGCVKIKKCIIM

Accordingly, the complex preferably comprises an amino acid sequence according to SEQ ID NO: 34 or a fragment or a variant thereof as described herein.

Several epitopes of Kirsten Ras are known to the skilled person. A preferred Kirsten Ras epitope, which is preferably comprised by the complex, includes the following epitope (the epitope sequence shown in the following is a fragment of the above Kirsten Ras sequence and is, thus, shown in the above Kirsten Ras sequence underlined; the following epitope sequence may refer to one epitope or more than one (overlapping) epitopes):

[SEQ ID NO: 35]
VVVGAGGVG

Accordingly, a preferred complex comprises an amino acid sequence according to SEQ ID NO: 35.

### O-Linked N-Acetylglucosamine (GlcNAc) Transferase (OGT)

OGT (O-Linked N-Acetylglucosamine (GlcNAc) Transferase, O-GlcNAc transferase, OGTase, O-linked N-acetylglucosaminyltransferase, uridine diphospho-N-acetylglucosamine:polypeptide beta-N-acetylglucosaminyltransferase, protein O-linked beta-N-acetylglucosamine transferase, UniProtKB O15294) is an enzyme with system name UDP-N-acetyl-D-glucosamine:protein-O-beta-N-acetyl-D-glucosaminyl transferase) is an enzyme with system name “UDP-N-acetyl-D-glucosamine:protein-O-beta-N-acetyl-D-glucosaminyl transferase”. OGT catalyzes the addition of a single N-acetylglucosamine in O-glycosidic linkage to serine or threonine residues of intracellular proteins. OGT is a part of a host of biological functions within the human body. OGT is involved in the resistance of insulin in muscle cells and adipocytes by inhibiting the Threonine 308 phosphorylation of AKT1, increasing the rate of IRS1 phosphorylation (at Serine 307 and Serine 632/635), reducing insulin signaling, and glycosylating components of insulin signals. Additionally, OGT catalyzes intracellular glycosylation of serine and threonine residues with the addition of N-acetylglucosamine. Studies show that OGT alleles are vital for embryogenesis, and that OGT is necessary for intracellular glycosylation and embryonic stem cell vitality. OGT also catalyzes the posttranslational modification that modifies transcription factors and RNA polymerase II, however the specific function of this modification is mostly unknown. The sequence of OGT is shown below:

[SEQ ID NO: 36]
MASSVGNVADSTEPTKRMLSFQGLAELAHREYQAGDFEAAERHCMQLWR
QEPDNTGVLLLLSSIHFQCRRLDRSAHFSTLAIKQNPLLAEAYSNLGNV
YKERGQLQEAIEHYRHALRLKPDFIDGYINLAAALVAAGDMEGAVQAYV
SALQYNPDLYCVRSDLGNLLKALGRLEEAKACYLKAIETQPNFAVAWSN
LGCVFNAQGEIWLAIHHFEKAVTLDPNFLDAYINLGNVLKEARIFDRAV
AAYLRALSLSPNHAVVHGNLACVYYEQGLIDLAIDTYRRAIELQPHFPD
AYCNLANALKEKGSVAEAEDCYNTALRLCPTHADSLNNLANIKREQGNI
EEAVRLYRKALEVFPEFAAAHSNLASVLQQQGKLQEALMHYKEAIRISP
TFADAYSNMGNTLKEMQDVQGALQCYTRAIQINPAFADAHSNLASIHKD
SGNIPEAIASYRTALKLKPDFPDAYCNLAHCLQIVCDWTDYDERMKKLV
SIVADQLEKNRLPSVHPHHSMLYPLSHGFRKAIAERHGNLCLDKINVLH
KPPYEHPKDLKLSDGRLRVGYVSSDFGNHPTSHLMQSIPGMHNPDKFEV
FCYALSPDDGTNFRVKVMAEANHFIDLSQIPCNGKAADRIHQDGIHILV
NMNGYTKGARNELFALRPAPIQAMWLGYPGTSGALFMDYIITDQETSPA
EVAEQYSEKLAYMPHTFFIGDHANMFPHLKKKAVIDFKSNGHIYDNRIV
LNGIDLKAFLDSLPDVKIVKMKCPDGGDNADSSNTALNMPVIPMNTIAE
AVIEMINRGQIQITINGFSISNGLATTQINNKAATGEEVPRTIIVTTRS
QYGLPEDAIVYCNFNQLYKIDPSTLQMWANILKRVPNSVLWLLRFPAVG
EPNIQQYAQNMGLPQNRIIFSPVAPKEEHVRRGQLADVCLDTPLCNGHT
TGMDVLWAGTPMVTMPGETLASRVAASQLTCLGCLELIAKNRQEYEDIA
VKLGTDLEYLKKVRGKVWKQRISSPLFNTKQYTMELERLYLQMWEHYAA
GNKPDHMIKPVEVTESA

Accordingly, the complex preferably comprises an amino acid sequence according to SEQ ID NO: 36 or a fragment or a variant thereof as described herein.

### Caspase 5 (CASP5)

Caspase 5 (UniProtKB P51878) is an enzyme that proteolytically cleaves other proteins at an aspartic acid residue, and belongs to a family of cysteine proteases called caspases. It is an inflammatory caspase, along with caspase 1, caspase 4 and the murine caspase 4 homolog caspase 11, and has a role in the immune system. The amino acid sequence of CASP5 is shown below:

[SEQ ID NO: 37]
MAEDSGKKKRRKNFEAMFKGILQSGLDNFVINHMLKNNVAGQTSIQTLV
PNTDQKSTSVKKDNHKKKTVKMLEYLGKDVLHGVFNYLAKHDVLTLKEE
EKKKYYDTKIEDKALILVDSLRKNRVAHQMFTQTLLNMDQKITSVKPLL
QIEAGPPESAESTNILKLCPREEFLRLCKKNHDEIYPIKKREDRRRLAL
IICNTKFDHLPARNGAHYDIVGMKRLLQGLGYTVVDEKNLTARDMESVL
RAFAARPEHKSSDSTFLVLMSHGILEGICGTAHKKKKPDVLLYDTIFQI
FNNRNCLSLKDKPKVIIVQACRGEKHGELWVRDSPASLALISSQSSENL
EADSVCKIHEEKDFIAFCSSTPHNVSWRDRTRGSIFITELITCFQKYSC
CCHLMEIFRKVQKSFEVPQAKAQMPTIERATLTRDFYLFPGN

Accordingly, the complex preferably comprises an amino acid sequence according to SEQ ID NO: 37 or a fragment or a variant thereof as described herein.

### Colorectal Tumor-Associated Antigen-1 (COA-1)

COA-1 was identified in 2003 by Maccalli et al. (Maccalli, C., et al., Identification of a colorectal tumor-associated antigen (COA-1) recognized by CD4(+) T lymphocytes. Cancer Res, 2003. 63(20): p. 6735-43) as strongly expressed by colorectal and melanoma cells (no data available). Its mutation may interfere with the differential recognition of tumor and normal cells. The amino acid sequence of COA-1 (UniProtKB Q5T124) is shown below:

[SEQ ID NO: 38]
MSSPLASLSKTRKVPLPSEPMNPGRRGIRIYGDEDEVDMLSDGCGSEEK
ISVPSCYGGIGAPVSRQVPASHDSELMAFMTRKLWDLEQQVKAQTDEIL
SKDQKIAALEDLVQTLRPHPAEATLQRQEELETMCVQLQRQVREMERFL
SDYGLQWVGEPMDQEDSESKTVSEHGERDWMTAKKFWKPGDSLAPPEVD
FDRLLASLQDLSELVVEGDTQVTPVPGGARLRTLEPIPLKLYRNGIMMF
DGPFQPFYDPSTQRCLRDILDGFFPSELQRLYPNGVPFKVSDLRNQVYL
EDGLDPFPGEGRVVGRQLMHKALDRVEEHPGSRMTAEKFLNRLPKFVIR
QGEVIDIRGPIRDTLQNCCPLPARIQEIVVETPTLAAERERSQESPNTP
APPLSMLRIKSENGEQAFLLMMQPDNTIGDVRALLAQARVMDASAFEIF
STFPPTLYQDDTLTLQAAGLVPKAALLLRARRAPKSSLKFSPGPCPGPG
PGPSPGPGPGPSPGPGPGPSPCPGPSPSPQ

Accordingly, the complex preferably comprises an amino acid sequence according to SEQ ID NO: 38 or a fragment or a variant thereof as described herein.

### Squamous Cell Carcinoma Antigen Recognized by T-Cells (SART)

Within the SART family, SART-3 is most preferred. Thus, the complex preferably comprises an antigen of the SART-family (a “SART” antigen) or an epitope thereof; the complex more preferably comprises SART-3 or an epitope thereof. Squamous cell carcinoma antigen recognized by T-cells 3 possesses tumor epitopes capable of inducing HLA-A24-restricted and tumor-specific cytotoxic T lymphocytes in cancer patients. SART-3 is thought to be involved in the regulation of mRNA splicing.

### IL13Ralpha2

IL13Ralpha2 binds interleukin 13 (IL-13) with very high affinity (and can therefore sequester it) but does not allow IL-4 binding. It acts as a negative regulator of both IL-13 and IL-4, however the mechanism of this is still undetermined. The amino acid sequence of IL13Ralpha2 is shown in the following:

[SEQ ID NO: 39]
MAFVCLAIGCLYTFLISTTFGCTSSSDTEIKVNPPQDFEIVDPGYLGYL
YLQWQPPLSLDHFKECTVEYELKYRNIGSETWKTIITKNLHYKDGFDLN
KGIEAKIHTLLPWQCTNGSEVQSSWAETTYWISPQGIPETKVQDMDCVY
YNWQYLLCSWKPGIGVLLDTNYNLFYWYEGLDHALQCVDYIKADGQNIG
CRFPYLEASDYKDFYICVNGSSENKPIRSSYFTFQLQNIVKPLPPVYLT
FTRESSCEIKLKWSIPLGPIPARCFDYEIEIREDDTTLVTATVENETYT
LKTTNETRQLCFVVRSKVNIYCSDDGIWSEWSDKQCWEGEDLSKKTLLR
FWLPFGFILILVIFVTGLLLRKPNTYPKMIPEFFCDT

Accordingly, the complex preferably comprises an amino acid sequence according to SEQ ID NO: 39 or a fragment or a variant thereof as described herein.

Several epitopes of IL13Ralpha2 are known to the skilled person. A preferred IL13Ralpha2 epitope, which is preferably comprised by the complex, includes the following epitope (the epitope sequence shown in the following is a fragment of the above IL13Ralpha2 sequence and is, thus, shown in the above IL13Ralpha2 sequence underlined; the following epitope sequence may refer to one epitope or more than one (overlapping) epitopes):

[SEQ ID NO: 40]
LPFGFIL

Accordingly, the complex preferably comprises an amino acid sequence according to SEQ ID NO: 40.

### KOC1

KOC1 (UniProtKB O00425), also known as insulin-like growth factor 2 mRNA-binding protein 3 (IGF2BP3), IMP3, KOC1, VICKZ3 is an mRNA binding protein. No expression data are however available, the sequence of which is as depicted below:

[SEQ ID NO: 41]
MNKLYIGNLSENAAPSDLESIFKDAKIPVSGPFLVKTGYAFVDCPDESWA
LKAIEALSGKIELHGKPIEVEHSVPKRQRIRKLQIRNIPPHLQWEVLDSL
LVQYGVVESCEQVNTDSETAVVNVTYSSKDQARQALDKLNGFQLENFTLK
VAYIPDEMAAQQNPLQQPRGRRGLGQRGSSRQGSPGSVSKQKPCDLPLRL
LVPTQFVGAIIGKEGATIRNITKQTQSKIDVHRKENAGAAEKSITILSTP
EGTSAACKSILEIMHKEAQDIKFTEEIPLKILAHNNFVGRLIGKEGRNLK
KIEQDTDTKITISPLQELTLYNPERTITVKGNVETCAKAEEEIMKKIRES
YENDIASMNLQAHLIPGLNLNALGLFPPTSGMPPPTSGPPSAMTPPYPQF
EQSETETVHLFIPALSVGAIIGKQGQHIKQLSRFAGASIKIAPAEAPDAK
VRMVIITGPPEAQFKAQGRIYGKIKEENFVSPKEEVKLEAHIRVPSFAAG
RVIGKGGKTVNELQNLSSAEVVVPRDQTPDENDQVVVKITGHFYACQVAQ
RKIQEILTQVKQHQQQKALQSGPPQSRRK

Accordingly, the complex preferably comprises an amino acid sequence according to SEQ ID NO: 41 or a fragment or a variant thereof as described herein.

### TOMM34

TOMM34 (UniProtKB Q15785) is involved in the import of precursor proteins into mitochondria. Many epitopes thereof are known to the skilled person, which can selected from the amino acid sequence shown below:

[SEQ ID NO: 42]
MAPKFPDSVEELRAAGNESFRNGQYAEASALYGRALRVLQAQGSSDPEEE
SVLYSNRAACHLKDGNCRDCIKDCTSALALVPFSIKPLLRRASAYEALEK
YPMAYVDYKTVLQIDDNVTSAVEGINRMTRALMDSLGPEWRLKLPSIPLV
PVSAQKRWNSLPSENHKEMAKSKSKETTATKNRVPSAGDVEKARVLKEEG
NELVKKGNHKKAIEKYSESLLCSNLESATYSNRALCYLVLKQYTEAVKDC
TEALKLDGKNVKAFYRRAQAHKALKDYKSSFADISNLLQIEPRNGPAQKL
RQEVKQNLH

Accordingly, the complex preferably comprises an amino acid sequence according to SEQ ID NO: 42 or a fragment or a variant thereof as described herein.

### RNF 43

RNF43 (UniProtKB Q68DV7) is a RING-type E3 ubiquitin ligase and is predicted to contain a transmembrane domain, a protease-associated domain, an ectodomain, and a cytoplasmic RING domain. RNF43 is thought to negatively regulate Wnt signaling, and expression of RNF43 results in an increase in ubiquitination of frizzled receptors, an alteration in their subcellular distribution, resulting in reduced surface levels of these receptors. Many epitopes thereof are known to the skilled person, with the amino acid sequence of RNF43 shown below:

[SEQ ID NO: 43]
MSGGHQLQLAALWPWLLMATLQAGFGRTGLVLAAAVESERSAEQKAIIRV
IPLKMDPTGKLNLTLEGVFAGVAEITPAEGKLMQSHPLYLCNASDDDNLE
PGFISIVKLESPRRAPRPCLSLASKARAGERGASAVLFDITEDRAAAEQL
QQPLGLTWPVVLIWGNDAEKLMEFVYKNQKAHVRIELKEPPAWPDYDVWI
LMTVVGTIFVIILASVLRIRCRPRHSRPDPLQQRTAWAISQLATRRYQAS
CRQARGEWPDSGSSCSSAPVCAICLEEFSEGQELRVISCLHEFHRNCVDP
WLHQHRTCPLCMFNITEGDSFSQSLGPSRSYQEPGRRLHLIRQHPGHAHY
HLPAAYLLGPSRSAVARPPRPGPFLPSQEPGMGPRHHRFPRAAHPRAPGE
QQRLAGAQHPYAQGWGLSHLQSTSQHPAACPVPLRRARPPDSSGSGESYC
TERSGYLADGPASDSSSGPCHGSSSDSVVNCTDISLQGVHGSSSTFCSSL
SSDFDPLVYCSPKGDPQRVDMQPSVTSRPRSLDSVVPTGETQVSSHVHYH
RHRHHHYKKRFQWHGRKPGPETGVPQSRPPIPRTQPQPEPPSPDQQVTRS
NSAAPSGRLSNPQCPRALPEPAPGPVDASSICPSTSSLFNLQKSSLSARH
PQRKRRGGPSEPTPGSRPQDATVHPACQIFPHYTPSVAYPWSPEAHPLIC
GPPGLDKRLLPETPGPCYSNSQPVWLCLTPRQPLEPHPPGEGPSEWSSDT
AEGRPCPYPHCQVLSAQPGSEEELEELCEQAV

Accordingly, the complex preferably comprises an amino acid sequence according to SEQ ID NO: 43 or a fragment or a variant thereof as described herein.

### Vascular Endothelial Growth Factor (VEGF)/Vascular Endothelial Growth Factor Receptor (VEGFR)

Vascular endothelial growth factor (VEGF, UniProtKB P15692), originally known as vascular permeability factor (VPF), is a signal protein produced by cells that stimulates vasculogenesis and angiogenesis. It is part of the system that restores the oxygen supply to tissues when blood circulation is inadequate. VEGF's normal function is to create new blood vessels during embryonic development, new blood vessels after injury, muscle following exercise, and new vessels (collateral circulation) to bypass blocked vessels. There are three main subtypes of the receptors for VEGF (VEGFR), namely VEGFR1 (UniProtKB P17948), VEGFR2 (UniProtKB P35968) and VEGFR3 (UniProtKB P35916). The sequences of VEGF, VEGFR1, VEGFR2 and VEGFR3 are hereby incorporated by reference. Accordingly, the complex preferably comprises an amino acid sequence of VEGF, VEGFR1, VEGFR2 and VEGFR3 or a fragment or a variant thereof as described herein.

Beta Subunit of Human Chorionic Gonadotropin (βhCG)

Human chorionic gonadotropin (hCG) is a hormone produced by the embryo following implantation. Some cancerous tumors produce this hormone; therefore, elevated levels measured when the patient is not pregnant can lead to a cancer diagnosis. hCG is heterodimeric with an α (alpha) subunit identical to that of luteinizing hormone (LH), follicle-stimulating hormone (FSH), thyroid-stimulating hormone (TSH), and β (beta) subunit that is unique to hCG. The β-subunit of hCG gonadotropin (beta-hCG) contains 145 amino acids and is encoded by six highly homologous genes. Accordingly, the complex preferably comprises an amino acid sequence of beta-hCG or a fragment or a variant thereof as described herein.

### EpCAM

EpCAM (UniProtKB P16422) is a glycoprotein mediating cellular adhesion. The amino acid sequence of EpCAM is shown in the following:

[SEQ ID NO: 44]
MAPPQVLAFGLLLAAATATFAAAQEECVCENYKLAVNCFVNNNRQCQCTS
VGAQNTVICSKLAAKCLVMKAEMNGSKLGRRAKPEGALQNNDGLYDPDCD
ESGLFKAKQCNGTSMCWCVNTAGVRRTDKDTEITCSERVRTYWIIIELKH
KAREKPYDSKSLRTALQKEITTRYQLDPKFITSILYENNVITIDLVQNSS
QKTQNDVDIADVAYYFEKDVKGESLFHSKKMDLTVNGEQLDLDPGQTLIY
YVDEKAPEFSMQGLKAGVIAVIVVVVIAVVAGIVVLVISRKKRMAKYEKA
EIKEMGEMHRELNA

Accordingly, the complex preferably comprises an amino acid sequence according to SEQ ID NO: 44 or a fragment or a variant thereof as described herein.

Several epitopes of EpCAM are known to the skilled person. A preferred EpCAM epitope, which is preferably comprised by the complex, includes the following epitope (the epitope sequence shown in the following is a fragment of the above EpCAM sequence and is, thus, shown in the above EpCAM sequence underlined; the following epitope sequence may refer to one epitope or more than one (overlapping) epitopes):

[SEQ ID NO: 45]
GLKAGVIAV

Accordingly, the complex preferably comprises an amino acid sequence according to SEQ ID NO: 45 or a fragment or a variant thereof as described herein.

### HER-2/neu

Her-2 belongs to the EGFR (epidermal growth factor receptor) family. Many HLA-A epitopes are known to the skilled person. The amino acid sequence of HER2 is shown in the following:

[SEQ ID NO: 46]
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV

Accordingly, the complex preferably comprises an amino acid sequence according to SEQ ID NO: 46 or a fragment or a variant thereof as described herein.

As described above, suitable cancer/tumor epitopes of Her-2 are known from the literature or can be identified by using cancer/tumor epitope databases, e.g. from van der Bruggen P, Stroobant V, Vigneron N, Van den Eynde B. Peptide database: T cell-defined tumor antigens. Cancer Immun 2013; URL: www.cancerimmunity.org/peptide/, wherein human tumor antigens recognized by CD4+ or CD8+ T cells are classified into four major groups on the basis of their expression pattern, or from the database “Tantigen” (TANTIGEN version 1.0, Dec. 1, 2009; developed by Bioinformatics Core at Cancer Vaccine Center, Dana-Farber Cancer Institute; URL: cvc.dfci.harvard.edu/tadb/).

### WT1

WT1 (Wilms tumor protein, UniProtKB P19544) Transcription factor that plays an important role in cellular development and cell survival. The gene encoding WT1 is characterized by an complex structure, is located on chromosome 11. It is involved in cell growth and differentiation, and has a strong impact on consecutive stages of the functioning of the body. The WT1 gene may e.g. undergo many different mutations, as well as may be overexpressed without a mutation. The molecular basis of diseases such as Wilms tumor are congenital WT1 mutations, while somatic mutations of this gene occur in acute and chronic myeloid leukemia, myelodysplastic syndrome and also in some other blood neoplasms, as acute lymphoblood leukemia. Increased expression of this gene without its mutation is observed in leukemias and solid tumors. The amino acid sequence of WT 1 is shown below:

[SEQ ID NO: 47]
MGSDVRDLNALLPAVPSLGGGGGCALPVSGAAQWAPVLDFAPPGASAYGS
LGGPAPPPAPPPPPPPPPHSFIKQEPSWGGAEPHEEQCLSAFTVHFSGQF
TGTAGACRYGPFGPPPPSQASSGQARMFPNAPYLPSCLESQPAIRNQGYS
TVTFDGTPSYGHTPSHHAAQFPNHSFKHEDPMGQQGSLGEQQYSVPPPVY
GCHTPTDSCTGSQALLLRTPYSSDNLYQMTSQLECMTWNQMNLGATLKGV
AAGSSSSVKWTEGQSNHSTGYESDNHTTPILCGAQYRIHTHGVFRGIQDV
RRVPGVAPTLVRSASETSEKRPFMCAYPGCNKRYFKLSHLQMHSRKHTGE
KPYQCDFKDCERRFSRSDQLKRHQRRHTGVKPFQCKTCQRKFSRSDHLKT
HTRTHTGKTSEKPFSCRWPSCQKKFARSDELVRHHNMHQRNMTKLQLAL

Accordingly, the complex preferably comprises an amino acid sequence according to SEQ ID NO: 47 or a fragment or a variant thereof as described herein.

Preferably, the complex comprises at least one tumor epitope, which is an epitope of an antigen selected from the group consisting of EpCAM, HER-2, MUC-1, TOMM34, RNF 43, KOC1, VEGFR, βhCG, survivin, CEA, TGFβR2, p53, KRas, OGT, CASP5, COA-1, MAGE, SART and IL13Ralpha2. More preferably, the complex comprises at least one tumor epitope, which is an epitope of an antigen selected from the group consisting of ASCL2, EpCAM, HER-2, MUC-1, TOMM34, RNF 43, KOC1, VEGFR, βhCG, survivin, CEA, TGFβR2, p53, KRas, OGT, CASP5, COA-1, MAGE, SART and IL13Ralpha2. Those antigens are particularly useful in the context of colorectal cancer. It is also preferred that the complex comprises at least one tumor antigen selected from the group consisting of EpCAM, HER-2, MUC-1, TOMM34, RNF 43, KOC1, VEGFR, βhCG, survivin, CEA, TGFβR2, p53, KRas, OGT, CASP5, COA-1, MAGE, SART and IL13Ralpha2, or a fragment thereof, or a sequence variant of a tumor antigen or a sequence variant of a fragment thereof. It is also preferred that the complex comprises at least one tumor antigen selected from the group consisting of ASCL2, EpCAM, HER-2, MUC-1, TOM M34, RNF 43, KOC1, VEGFR, βhCG, survivin, CEA, TGFβR2, p53, KRas, OGT, CASP5, COA-1, MAGE, SART and IL13Ralpha2, or a fragment thereof, or a sequence variant of a tumor antigen or a sequence variant of a fragment thereof.

Preferably, the complex comprises at least one tumor epitope, which is an epitope of an antigen selected from the group consisting of EpCAM, MUC-1, survivin, CEA, KRas, MAGE-A3, IL13Ralpha2, and ASCL2, such as an epitope according to any of SEQ ID NOs 45, 26, 27, 17, 30, 31, 35, 40, 22 and 23; more preferably the at least one tumor epitope is an epitope of an antigen selected from the group consisting of EpCAM, MUC-1, survivin, CEA, KRas, MAGE-A3, and ASCL2, such as an epitope according to any of SEQ ID NOs 45, 26, 27, 17, 30, 31, 35, 22 and 23; even more preferably the at least one tumor epitope is an epitope of an antigen selected from the group consisting of EpCAM, MUC-1, survivin, CEA, and ASCL2 such as an epitope according to any of SEQ ID NOs 45, 26, 27, 17, 30, 31, 22 and 23; and most preferably the at least one tumor epitope is an epitope of an antigen selected from the group consisting of EpCAM, survivin, CEA, and ASCL2 such as an epitope according to any of SEQ ID NOs 45, 17, 30, 31, 22 and 23.

In some embodiments, the at least one tumor epitope of the complex is an epitope of an antigen selected from the group consisting of MAGE-A3, MUC-1, PRAME, ASCL2, and NY-ESO-1, preferably the at least one tumor epitope complex is an epitope of an antigen selected from the group consisting of MAGE-A3, MUC-1, PRAME, ASCL2, preferably the at least one tumor epitope complex is an epitope of an antigen selected from the group consisting of MAGE-A3, MUC-1, PRAME, preferably the at least one tumor epitope of the complex is an epitope of an antigen selected from the group consisting of MAGE-A3, MUC-1, ASCL2, preferably the at least one tumor epitope of the complex is an epitope of an antigen selected from the group consisting of MAGE-A3, ASCL2, PRAME, preferably the at least one tumor epitope of the complex is an epitope of an antigen selected from the group consisting of MAGE-A3, MUC-1, NY-ESO-1, preferably the at least one tumor epitope of the complex is an epitope of an antigen selected from the group consisting of MAGE-A3, ASCL2, NY-ESO-1. In one embodiment, it is more preferred that the multi-antigenic domain of the complex comprises at least one epitope of the antigen MAGE-A3, or ASCL2, or MUC1, or PRAME, or NY-ESO-1.

It is also preferred that the complex comprises
i) one or more epitopes of EpCAM (such as the epitope according to SEQ ID NO: 45) or functional sequence variants thereof;
ii) one or more epitopes of MUC-1 (such as the epitope according to SEQ ID NO: 26 and/or the epitope according to SEQ ID NO: 27) or functional sequence variants thereof;
iii) one or more epitopes of survivin (such as the epitope according to SEQ ID NO: 17) or functional sequence variants thereof;
iv) one or more epitopes of CEA (such as the epitope according to SEQ ID NO: 30 and/or the epitope according to SEQ ID NO: 31) or functional sequence variants thereof;
v) one or more epitopes of KRas (such as the epitope according to SEQ ID NO: 31) or functional sequence variants thereof; and/or
vi) one or more epitopes of MAGE-A3 or functional sequence variants thereof.

It is also preferred that the complex comprises
i) one or more epitopes of EpCAM (such as the epitope according to SEQ ID NO: 45) or functional sequence variants thereof;
ii) one or more epitopes of MUC-1 (such as the epitope according to SEQ ID NO: 26 and/or the epitope according to SEQ ID NO: 27) or functional sequence variants thereof;
iii) one or more epitopes of survivin (such as the epitope according to SEQ ID NO: 17) or functional sequence variants thereof;
iv) one or more epitopes of CEA (such as the epitope according to SEQ ID NO: 30 and/or the epitope according to SEQ ID NO: 31) or functional sequence variants thereof;
v) one or more epitopes of KRas (such as the epitope according to SEQ ID NO: 35) or functional sequence variants thereof;
vi) one or more epitopes of MAGE-A3 or functional sequence variants thereof; and/or
vii) one or more epitopes of ASCL2 (such as the epitope according to SEQ ID NO: 22 and/or the epitope according to SEQ ID NO: 23) or functional sequence variants thereof.

It is also preferred that the complex comprises
a fragment of EpCAM comprising one or more epitopes or a functional sequence variant thereof;
a fragment of MUC-1 comprising one or more epitopes or a functional sequence variant thereof;
a fragment of survivin comprising one or more epitopes or a functional sequence variant thereof;
a fragment of CEA comprising one or more epitopes or a functional sequence variant thereof;
a fragment of KRas comprising one or more epitopes or a functional sequence variant thereof; and/or
a fragment of MAGE-A3 comprising one or more epitopes or a functional sequence variant thereof.

It is also preferred that the complex comprises
a fragment of EpCAM comprising one or more epitopes or a functional sequence variant thereof;
a fragment of MUC-1 comprising one or more epitopes or a functional sequence variant thereof;
a fragment of survivin comprising one or more epitopes or a functional sequence variant thereof;
a fragment of CEA comprising one or more epitopes or a functional sequence variant thereof;
a fragment of KRas comprising one or more epitopes or a functional sequence variant thereof;
a fragment of MAGE-A3 comprising one or more epitopes or a functional sequence variant thereof; and/or
a fragment of ASCL2 comprising one or more epitopes or a functional sequence variant thereof.

As used herein, a “fragment” of an antigen comprises at least 10 consecutive amino acids of the antigen, preferably at least 15 consecutive amino acids of the antigen, more preferably at least 20 consecutive amino acids of the antigen, even more preferably at least 25 consecutive amino acids of the antigen and most preferably at least 30 consecutive amino acids of the antigen. Accordingly, a fragment of EpCAM comprises at least 10 consecutive amino acids of EpCAM (SEQ ID NO: 44), preferably at least 15 consecutive amino acids of EpCAM (SEQ ID NO: 44), more preferably at least 20 consecutive amino acids of EpCAM (SEQ ID NO: 44), even more preferably at least 25 consecutive amino acids of EpCAM (SEQ ID NO: 44) and most preferably at least 30 consecutive amino acids of EpCAM (SEQ ID NO: 44); a fragment of MUC-1 comprises at least 10 consecutive amino acids of MUC-1 (SEQ ID NO: 25), preferably at least 15 consecutive amino acids of MUC-1 (SEQ ID NO: 25), more preferably at least 20 consecutive amino acids of MUC-1 (SEQ ID NO: 25), even more preferably at least 25 consecutive amino acids of MUC-1 (SEQ ID NO: 25) and most preferably at least 30 consecutive amino acids of MUC-1 (SEQ ID NO: 25); a fragment of survivin comprises at least 10 consecutive amino acids of survivin (SEQ ID NO: 16), preferably at least 15 consecutive amino acids of survivin (SEQ ID NO: 16), more preferably at least 20 consecutive amino acids of survivin (SEQ ID NO: 16), even more preferably at least 25 consecutive amino acids of survivin (SEQ ID NO: 16) and most preferably at least 30 consecutive amino acids of survivin (SEQ ID NO: 16); a fragment of CEA comprises at least 10 consecutive amino acids of CEA (SEQ ID NO: 29), preferably at least 15 consecutive amino acids of CEA (SEQ ID NO: 29), more preferably at least 20 consecutive amino acids of CEA (SEQ ID NO: 29), even more preferably at least 25 consecutive amino acids of CEA (SEQ ID NO: 29) and most preferably at least 30 consecutive amino acids of CEA (SEQ ID NO: 29); a fragment of KRas comprises at least 10 consecutive amino acids of KRas (SEQ ID NO: 34), preferably at least 15 consecutive amino acids of KRas (SEQ ID NO: 34), more preferably at least 20 consecutive amino acids of KRas (SEQ ID NO: 34), even more preferably at least 25 consecutive amino acids of KRas (SEQ ID NO: 34) and most preferably at least 30 consecutive amino acids of KRas (SEQ ID NO: 34); and a fragment of MAGE-A3 comprises at least 10 consecutive amino acids of MAGE-A3 (SEQ ID NO: 14), preferably at least 15 consecutive amino acids of MAGE-A3 (SEQ ID NO: 14), more preferably at least 20 consecutive amino acids of MAGE-A3 (SEQ ID NO: 14), even more preferably at least 25 consecutive amino acids of MAGE-A3 (SEQ ID NO: 14) and most preferably at least 30 consecutive amino acids of MAGE-A3 (SEQ ID NO: 14). Moreover, a fragment of ASCL2 comprises at least 10 consecutive amino acids of ASCL2 (SEQ ID NO: 21), preferably at least 15 consecutive amino acids of ASCL2 (SEQ ID NO: 21), more preferably at least 20 consecutive amino acids of ASCL2 (SEQ ID NO: 21), even more preferably at least 25 consecutive amino acids of ASCL2 (SEQ ID NO: 21) and most preferably at least 30 consecutive amino acids of ASCL2 (SEQ ID NO: 21).

A functional sequence variant of such a fragment has an (amino acid) sequence, which is at least 70% or at least 75%, preferably at least 80% or at least 85%, more preferably at least 90% or at least 95%, even more preferably at least 97% or at least 98%, particularly preferably at least 99% identical to the reference sequence, and the epitope function of at least one, preferably all, epitope(s) comprised by the fragment is maintained.

In a preferred embodiment, the complex does not comprise any epitope of HER-2, MUC-1, TOMM34, RNF 43, KOC1, VEGFR, βhCG, survivin, TGFβR2, p53, KRas, OGT, CASP5, COA-1, SART or IL13Ralpha2. In a more preferred embodiment, such a complex does not comprise any epitope of ASCL2, HER-2, MUC-1, TOMM34, RNF 43, KOC1, VEGFR, βhCG, survivin, TGFβR2, p53, KRas, OGT, CASP5, COA-1, SART or IL13Ralpha2.

In a preferred embodiment the complex comprises
one or more epitopes of EpCAM (such as the epitope according to SEQ ID NO: 45) or functional sequence variants thereof;
one or more epitopes of MUC-1 (such as the epitope according to SEQ ID NO: 26 and/or the epitope according to SEQ ID NO: 27) or functional sequence variants thereof;
one or more epitopes of CEA (such as the epitope according to SEQ ID NO: 30 and/or the epitope according to SEQ ID NO: 31) or functional sequence variants thereof; and
one or more epitopes of MAGE-A3 or functional sequence variants thereof.

In this preferred embodiment, the complex does preferably not comprise any epitope of HER-2, TOMM34, RNF 43, KOC1, VEGFR, βhCG, survivin, TGFβR2, p53, KRas, OGT, CASP5, COA-1, SART or IL13Ralpha2. More preferably, in this preferred embodiment the complex does not comprise any epitope of ASCL2, HER-2, TOMM34, RNF 43, KOC1, VEGFR, βhCG, survivin, TGFβR2, p53, KRas, OGT, CASP5, COA-1, SART or IL13Ralpha2.

In another preferred embodiment the complex comprises
one or more epitopes of EpCAM (such as the epitope according to SEQ ID NO: 45) or functional sequence variants thereof;
one or more epitopes of MUC-1 (such as the epitope according to SEQ ID NO: 26 and/or the epitope according to SEQ ID NO: 27) or functional sequence variants thereof;
one or more epitopes of CEA (such as the epitope according to SEQ ID NO: 30 and/or the epitope according to SEQ ID NO: 31) or functional sequence variants thereof; and
one or more epitopes of KRas (such as the epitope according to SEQ ID NO: 35) or functional sequence variants thereof.

In this preferred embodiment, the a complex does preferably not comprise any epitope of HER-2, TOMM34, RNF 43, KOC1, VEGFR, βhCG, survivin, TGFβR2, p53, OGT, CASP5, COA-1, MAGE, SART or IL13Ralpha2. More preferably, in this preferred embodiment the complex does not comprise any epitope of ASCL2, HER-2, TOMM34, RNF 43, KOC1, VEGFR, βhCG, survivin, TGFβR2, p53, OGT, CASP5, COA-1, MAGE, SART or IL13Ralpha2.

In a different preferred embodiment the complex comprises
one or more epitopes of EpCAM (such as the epitope according to SEQ ID NO: 45) or functional sequence variants thereof;
one or more epitopes of survivin (such as the epitope according to SEQ ID NO: 17) or functional sequence variants thereof;
one or more epitopes of CEA (such as the epitope according to SEQ ID NO: 30 and/or the epitope according to SEQ ID NO: 31) or functional sequence variants thereof; and
one or more epitopes of MAGE-A3 5 or functional sequence variants thereof.

In this preferred embodiment, the a complex does preferably not comprise any epitope of HER-2, MUC-1, TOMM34, RNF 43, KOC1, VEGFR, βhCG, TGFβR2, p53, KRas, OGT, CASP5, COA-1, SART or IL13Ralpha2. More preferably, in this preferred embodiment the complex does not comprise any epitope of ASCL2, HER-2, MUC-1, TOM M34, RNF 43, KOC1, VEGFR, βhCG, TGFβR2, p53, KRas, OGT, CASP5, COA-1, SART or IL13Ralpha2.

In a preferred embodiment the complex comprises
one or more epitopes of MUC-1 (such as the epitope according to SEQ ID NO: 26 and/or the epitope according to SEQ ID NO: 27) or functional sequence variants thereof;
one or more epitopes of survivin (such as the epitope according to SEQ ID NO: 17) or functional sequence variants thereof; and
one or more epitopes of MAGE-A3 or functional sequence variants thereof.

Such a complex does preferably not comprise any epitope of EpCAM, HER-2, TOMM34, RNF 43, KOC1, VEGFR, βhCG, CEA, TGFβR2, p53, KRas, OGT, CASP5, COA-1, SART or IL13Ralpha2. More preferably, such a complex does not comprise any epitope of ASCL2, EpCAM, HER-2, TOMM34, RNF 43, KOC1, VEGFR, βhCG, CEA, TGFβR2, p53, KRas, OGT, CASP5, COA-1, SART or IL13Ralpha2.

More preferably, the complex comprises
one or more epitopes of EpCAM (such as the epitope according to SEQ ID NO: 45) or functional sequence variants thereof;
one or more epitopes of MUC-1 (such as the epitope according to SEQ ID NO: 26 and/or the epitope according to SEQ ID NO: 27) or functional sequence variants thereof;
one or more epitopes of survivin (such as the epitope according to SEQ ID NO: 17) or functional sequence variants thereof; and/or
one or more epitopes of CEA (such as the epitope according to SEQ ID NO: 30 and/or the epitope according to SEQ ID NO: 31) or functional sequence variants thereof.

Such a complex does preferably not comprise any epitope of HER-2, TOMM34, RNF 43, KOC1, VEGFR, βhCG, TGFβR2, p53, KRas, OGT, CASP5, COA-1, MAGE, SART or IL13Ralpha2. More preferably, such a complex does not comprise any epitope of ASCL2, HER-2, TOMM34, RNF 43, KOC1, VEGFR, βhCG, TGFβR2, p53, KRas, OGT, CASP5, COA-1, MAGE, SART or IL13Ralpha2.

More preferably, the complex comprises
one or more epitopes of EpCAM (such as the epitope according to SEQ ID NO: 45) or functional sequence variants thereof;
one or more epitopes of MUC-1 (such as the epitope according to SEQ ID NO: 26 and/or the epitope according to SEQ ID NO: 27) or functional sequence variants thereof;
one or more epitopes of survivin (such as the epitope according to SEQ ID NO: 17) or functional sequence variants thereof;
one or more epitopes of ASCL2 (such as the epitope according to SEQ ID NO: 22 and/or the epitope according to SEQ ID NO: 23) or functional sequence variants thereof; and/or
one or more epitopes of CEA (such as the epitope according to SEQ ID NO: 30 and/or the epitope according to SEQ ID NO: 31) or functional sequence variants thereof.

Such a complex does preferably not comprise any epitope of HER-2, TOMM34, RNF 43, KOC1, VEGFR, βhCG, TGFβR2, p53, KRas, OGT, CASP5, COA-1, MAGE, SART or IL13Ralpha2.

More preferably, the complex comprises
one or more epitopes of EpCAM (such as the epitope according to SEQ ID NO: 45) or functional sequence variants thereof;
one or more epitopes of survivin (such as the epitope according to SEQ ID NO: 17) or functional sequence variants thereof;
one or more epitopes of ASCL2 (such as the epitope according to SEQ ID NO: 22 and/or the epitope according to SEQ ID NO: 23) or functional sequence variants thereof; and/or
one or more epitopes of CEA (such as the epitope according to SEQ ID NO: 30 and/or the epitope according to SEQ ID NO: 31) or functional sequence variants thereof.

Such a complex does preferably not comprise any epitope of HER-2, MUC-1, TOMM34, RNF 43, KOC1, VEGFR, βhCG, TGFβR2, p53, KRas, OGT, CASP5, COA-1, MAGE, SART or IL13Ralpha2.

Particularly preferably, such a complex comprises
one or more epitopes of EpCAM (such as the epitope according to SEQ ID NO: 45) or functional sequence variants thereof;
one or more epitopes of MUC-1 (such as the epitope according to SEQ ID NO: 26 and/or the epitope according to SEQ ID NO: 27) or functional sequence variants thereof;
one or more epitopes of survivin (such as the epitope according to SEQ ID NO: 17) or functional sequence variants thereof; and
one or more epitopes of CEA (such as the epitope according to SEQ ID NO: 30 and/or the epitope according to SEQ ID NO: 31) or functional sequence variants thereof.

Such a complex does preferably not comprise any epitope of HER-2, TOMM34, RNF 43, KOC1, VEGFR, βhCG, TGFβR2, p53, KRas, OGT, CASP5, COA-1, MAGE, SART or IL13Ralpha2. More preferably, such a complex does not comprise any epitope of ASCL2, HER-2, TOMM34, RNF 43, KOC1, VEGFR, βhCG, TGFβR2, p53, KRas, OGT, CASP5, COA-1, MAGE, SART or IL13Ralpha2.

It is also particularly preferred that such a complex comprises
one or more epitopes of EpCAM (such as the epitope according to SEQ ID NO: 45) or functional sequence variants thereof;
one or more epitopes of MUC-1 (such as the epitope according to SEQ ID NO: 26 and/or the epitope according to SEQ ID NO: 27) or functional sequence variants thereof; and
one or more epitopes of CEA (such as the epitope according to SEQ ID NO: 30 and/or the epitope according to SEQ ID NO: 31) or functional sequence variants thereof.

Such a complex does preferably not comprise any epitope of HER-2, TOMM34, RNF 43, KOC1, VEGFR, βhCG, survivin, TGFβR2, p53, KRas, OGT, CASP5, COA-1, MAGE, SART or IL13Ralpha2. More preferably, such a complex does not comprise any epitope of ASCL2, HER-2, TOMM34, RNF 43, KOC1, VEGFR, βhCG, survivin, TGFβR2, p53, KRas, OGT, CASP5, COA-1, MAGE, SART or IL13Ralpha2.

In a most preferred embodiment, the complex comprises
one or more epitopes of CEA (such as the epitope according to SEQ ID NO: 30 and/or the epitope according to SEQ ID NO: 31) or functional sequence variants thereof;
one or more epitopes of survivin (such as the epitope according to SEQ ID NO: 17) or functional sequence variants thereof;
one or more epitopes of EpCAM (such as the epitope according to SEQ ID NO: 41) or functional sequence variants thereof; and
one or more epitopes of ASCL2 (such as the epitope according to SEQ ID NO: 22 and/or the epitope according to SEQ ID NO: 23) or functional sequence variants thereof.

Such a complex does preferably not comprise any epitope of HER-2, MUC-1, TOMM34, RNF 43, KOC1, VEGFR, βhCG, TGFβR2, p53, KRas, OGT, CASP5, COA-1, MAGE, SART or IL13Ralpha2.

In a further most preferred embodiment, the complex comprises in N- to C-terminal direction:
one or more epitopes of CEA (such as the epitope according to SEQ ID NO: 30 and/or the epitope according to SEQ ID NO: 31) or functional sequence variants thereof;
one or more epitopes of survivin (such as the epitope according to SEQ ID NO: 17) or functional sequence variants thereof; and
one or more epitopes of ASCL2 (such as the epitope according to SEQ ID NO: 22 and/or the epitope according to SEQ ID NO: 23) or functional sequence variants thereof.

Such a complex does preferably not comprise any epitope of HER-2, EpCAM, MUC-1, TOMM34, RNF 43, KOC1, VEGFR, βhCG, TGFβR2, p53, KRas, OGT, CASP5, COA-1, MAGE, SART or IL13Ralpha2

Even more preferably, the complex comprises in N- to C-terminal direction:
i) a peptide having an amino acid sequence according to SEQ ID NO: 29, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% or at least 75%, preferably at least 80% or at least 85%, more preferably at least 90% or at least 95%, even more preferably at least 97% or at least 98%, particularly preferably at least 99% sequence identity;
ii) a peptide having an amino acid sequence according to SEQ ID NO: 16, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% or at least 75%, preferably at least 80% or at least 85%, more preferably at least 90% or at least 95%, even more preferably at least 97% or at least 98%, particularly preferably at least 99% sequence identity; and
iii) a peptide having an amino acid sequence according to SEQ ID NO: 21, or a fragment thereof having a length of at least 10 amino acids (preferably at least 15 amino acids, more preferably at least 20 amino acids, even more preferably at least 25 amino acids and most preferably at least 30 amino acids), or a functional sequence variant thereof having at least 70% or at least 75%, preferably at least 80% or at least 85%, more preferably at least 90% or at least 95%, even more preferably at least 97% or at least 98%, particularly preferably at least 99% sequence identity.

Such a complex does preferably not comprise any further antigen or further epitopes of antigens other than CEA, survivin and ASCL2, more preferably such a complex does not comprise any other (tumor) epitope.

Preferably, in such a complex, the C-terminus of (i) the peptide having an amino acid sequence according to SEQ ID NO: 29 or the fragment or variant thereof is directly linked to the N-terminus of (ii) the peptide having an amino acid sequence according to SEQ ID NO: 16 or the fragment or variant thereof; and the C-terminus of (ii) the peptide having an amino acid sequence according to SEQ ID NO: 16 or the fragment or variant thereof is directly linked to the N-terminus of (iii) the peptide having an amino acid sequence according to SEQ ID NO: 21 or the fragment or variant thereof.

Still more preferably, the complex comprises in N- to C-terminal direction:
i) a peptide having an amino acid sequence according to SEQ ID NO: 32, or a functional sequence variant thereof having at least at least 70% or at least 75%, preferably at least 80% or at least 85%, more preferably at least 90% or at least 95%, even more preferably at least 97% or at least 98%, particularly preferably at least 99% sequence identity;
ii) a peptide having an amino acid sequence according to SEQ ID NO: 18, or a functional sequence variant thereof having at least 70% or at least 75%, preferably at least 80% or at least 85%, more preferably at least 90% or at least 95%, even more preferably at least 97% or at least 98%, particularly preferably at least 99% sequence identity; and
iii) a peptide having an amino acid sequence according to SEQ ID NO: 24, or a functional sequence variant thereof having at least 70% or at least 75%, preferably at least 80% or at least 85%, more preferably at least 90% or at least 95%, even more preferably at least 97% or at least 98%, particularly preferably at least 99% sequence identity.

Such a complex does preferably not comprise any further antigen or further epitopes of antigens other than CEA, survivin and ASCL2, more preferably such a complex does not comprise any other (tumor) epitope.

Preferably, in such a complex, the C-terminus of (i) the peptide having an amino acid sequence according to SEQ ID NO: 32 or the variant thereof is directly linked to the N-terminus of (ii) the peptide having an amino acid sequence according to SEQ ID NO: 18 or the variant thereof; and the C-terminus of (ii) the peptide having an amino acid sequence according to SEQ ID NO: 18 or the variant thereof is directly linked to the N-terminus of (iii) the peptide having an amino acid sequence according to SEQ ID NO: 24 or the variant thereof.

Most preferably, the multi-antigenic domain of the complex comprises or consists of a peptide having an amino acid sequence according to SEQ ID NO: 48 or a (functional) sequence variant thereof having at least 70% or at least 75%, preferably at least 80% or at least 85%, more preferably at least 90% or at least 95%, even more preferably at least 97% or at least 98%, particularly preferably at least 99% sequence identity. In a most preferred embodiment, the complex does not comprise any further antigen or further epitopes of antigens other than CEA, survivin and ASCL2, yet more preferably does not comprise any other (tumor) epitope.

Component c)—TLR Peptide Agonist

In the complex comprised by the combination according to the present invention, the TLR peptide agonist allows an increased targeting of the vaccine towards dendritic cells along with self-adjuvancity. Physical linkage of a TLR peptide agonist to the CPP and the at least one antigen or antigenic epitope in the complex comprised by the combination according to the present invention provides an enhanced immune response by simultaneous stimulation of antigen presenting cells, in particular dendritic cells, that internalize, metabolize and display antigen(s).

As used in the context of the present invention, a “TLR peptide agonist” is an agonist of a Toll-like receptor (TLR), i.e. it binds to a TLR and activates the TLR, in particular to produce a biological response. Moreover, the TLR peptide agonist is a peptide, a polypeptide or a protein as defined above. Preferably, the TLR peptide agonist comprises from 10 to 150 amino acids, more preferably from 15 to 130 amino acids, even more preferably from 20 to 120 amino acids, particularly preferably from 25 to 110 amino acids, and most preferably from 30 to 100 amino acids.

Toll like receptors (TLRs) are transmembrane proteins that are characterized by extracellular, transmembrane, and cytosolic domains. The extracellular domains containing leucine-rich repeats (LRRs) with horseshoe-like shapes are involved in recognition of common molecular patterns derived from diverse microbes. Toll like receptors include TLRs1-10. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LP A, LTA, fimbriae, outer membrane proteins, heat shock proteins from bacteria or from the host, and Mycobacterial lipoarabinomannans. TLR3 may be activated by dsRNA, in particular of viral origin, or by the chemical compound poly(LC). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLR5 may be activated with bacterial flagellae or flagellin. TLR6 may be activated by mycobacterial lipoproteins and group B streptococcus heat labile soluble factor (GBS-F) or staphylococcus modulins. TLR7 may be activated by imidazoquinolines. TLR9 may be activated by unmethylated CpG DNA or chromatin-IgG complexes.

Preferably, the TLR peptide agonist comprised by the complex comprised by the combination according to the present invention is an agonist of TLR1, 2, 4, 5, 6, and/or 10. TLRs are expressed either on the cell surface (TLR1, 2, 4, 5, 6, and 10) or on membranes of intracellular organelles, such as endosomes (TLR3, 4, 7, 8, and 9). The natural ligands for the endosomal receptors turned out to be nucleic acid-based molecules (except for TLR4). The cell surface-expressed TLR1, 2, 4, 5, 6, and 10 recognize molecular patterns of extracellular microbes (Monie, T. P., Bryant, C. E., et al. 2009: Activating immunity: Lessons from the TLRs and NLRs. Trends Biochem. Sci. 34(11), 553-561). TLRs are expressed on several cell types but virtually all TLRs are expressed on DCs allowing these specialized cells to sense all possible pathogens and danger signals.

However, TLR2, 4, and 5 are constitutively expressed at the surface of DCs. Accordingly, the TLR peptide agonist comprised by the complex comprised by the combination according to the present invention is more preferably a peptide agonist of TLR2, TLR4 and/or TLR5. Even more preferably, the TLR peptide agonist is a TLR2 peptide agonist and/or a TLR4 peptide agonist. Particularly preferably, the TLR peptide agonist is a TLR4 peptide agonist. Most preferably, the TLR peptide agonist is one TLR peptide agonist, which is both, a TLR2 and a TLR4 agonist. TLR2 can detect a wide variety of ligands derived from bacteria, viruses, parasites, and fungi. The ligand specificity is often determined by the interaction of TLR2 with other TLRs, such as TLR1, 6, or 10, or non-TLR molecules, such as dectin-1, CD14, or CD36. The formation of a heterodimer with TLR1 enables TLR2 to identify triacyl lipoproteins or lipopeptides from (myco)bacterial origin, such as Pam3CSK4 and peptidoglycan (PGA; Gay, N. J., and Gangloff, M. (2007): Structure and function of Toll receptors and their ligands. Annu. Rev. Biochem. 76, 141-165; Spohn, R., Buwitt-Beckmann, U., et al. (2004): Synthetic lipopeptide adjuvants and Toll-like receptor 2—Structure-activity relationships. Vaccine 22(19), 2494-2499). Heterodimerization of TLR2 and 6 enables the detection of diacyl lipopeptides and zymosan. Lipopolysaccharide (LPS) and its derivatives are ligands for TLR4 and flagellin (Bryant, C. E., Spring, D. R., et al. (2010) or entolimod (CBLB502) for TLR5. The molecular basis of the host response to lipopolysaccharide. Nat. Rev. Microbiol. 8(1), 8-14).

TLR2 interacts with a broad and structurally diverse range of ligands, including molecules expressed by microbes and fungi. Multiple TLR2 agonists have been identified, including natural and synthetic lipopeptides (e.g. Mycoplasma fermentas macrophage-activating lipopeptide (MALP-2)), peptidoglycans (PG such as those from S. aureus), lipopolysaccharides from various bacterial strains (LPS), polysaccharides (e.g. zymosan), glycosylphosphatidyl-inositol-anchored structures from gram positive bacteria (e.g. lipoteichoic acid (LTA) and lipo-arabinomannan from mycobacteria and lipomannas from M. tuberculosis). Certain viral determinants may also trigger via TLR2 (Barbalat R, Lau L, Locksley R M, Barton G M. Toll-like receptor 2 on inflammatory monocytes induces type I interferon in response to viral but not bacterial ligands. Nat Immunol. 2009: 10(11):1200-7). Bacterial lipopeptides are structural components of cell walls. They consist of an acylated s-glycerylcysteine moiety to which a peptide can be conjugated via the cysteine residue. Examples of TLR2 agonists, which are bacterial lipopeptides, include MALP-2 and it's synthetic analogue di-palmitoyl-S-glyceryl cysteine (Pam₂Cys) or tri-palmitoyl-S-glyceryl cysteine (Pam₃Cys).

A diversity of ligands interact with TLR4, including Monophosphoryl Lipid A from Salmonella minnesota R595 (MPLA), lipopolysaccharides (LPS), mannans (Candida albicans), glycoinositolphospholipids (Trypanosoma), viral envelope proteins (RSV and MMTV) and endogenous antigens including fibrinogen and heat-shock proteins. Such agonists of TLR4 are for example described in Akira S, Uematsu S, Takeuchi O. Pathogen recognition and innate immunity. Cell. Feb. 24; 2006: 124(4):783-801 or in Kumar H, Kawai T, Akira S. Toll-like receptors and innate immunity. Biochem Biophys Res Commun. October 30; 2009 388(4):621-5. LPS, which is found in the outer membrane of gram negative bacteria, is the most widely studied of the TLR4 ligands. Suitable LPS-derived TLR4 agonist peptides are described for example in WO 2013/120073 (A1).

TLR5 is triggered (i) by a region of the flagellin molecule expressed by nearly all motile bacteria; or (ii) by entolimod (CBLB502). Thus, (i) flagellin, or peptides or proteins derived from flagellin and/or variants or fragments of flagellin; or (ii) entolimod (CBLB502) are also suitable as TLR peptide agonists comprised by the complex.

Examples of TLR peptide agonists thus include the TLR2 lipopeptide agonists MALP-2, Pam₂Cys and Pam₃Cys or modifications thereof, different forms of the TLR4 agonist LPS, e.g. N. meningitidis wild-type L3-LPS and mutant penta-acylated LpxL1-LPS, and the TLR5 agonist flagellin.

However, it is preferred that the TLR peptide agonist comprised by the complex is neither a lipopeptide nor a lipoprotein, neither a glycopeptide nor a glycoprotein, more preferably, the TLR peptide agonist comprised by the complex is a classical peptide, polypeptide or protein as defined herein.

In some embodiments, the TLR peptide agonist is a fragment of a (naturally occurring) protein, or a variant thereof, which shares at least 70% or at least 75%, preferably at least 80% or at least 85%, more preferably at least 90% or at least 95%, even more preferably at least 97% or at least 98%, particularly preferably at least 99% sequence identity. Such fragments may have a minimum length of at least 20 or 25, preferably at least 30 or 35, more preferably at least 40 or 50, even more preferably 60 or 70, still more preferably at least 80 or 90, such as at least 100, amino acids. In particular, the fragment exhibits TLR agonist functionality. The fragment of the protein may advantageously be selected such that it provides the “TLR agonist domain” of the protein, but preferably does not include any other domain (other than the TLR agonist domain) of the protein.

Therefore, in some embodiments, the TLR agonist does not comprise another immunological active domain (other than the TLR agonist domain), more preferably the TLR agonist does not comprise another biological active domain (other than the TLR agonist domain). For example, in some embodiments, the TLR agonist is not flagellin (which includes further domains in addition to the TLR agonist functionality). However, in some embodiments, the TLR agonist may be a fragment of flagellin including the TLR agonist domain of flagellin (but no other domain of flagellin).

A preferred TLR2 peptide agonist is annexin II or an immunomodulatory fragment thereof (having TLR agonist functionality), which is described in detail in WO 2012/048190 A1 and U.S. patent application Ser. No. 13/033,1546, in particular a TLR2 peptide agonist comprising an amino acid sequence according to SEQ ID NO: 7 of WO 2012/048190 A1 or fragments or variants thereof are preferred.

Thereby, a TLR2 peptide agonist comprising or consisting of an amino acid sequence according to SEQ ID NO: 49 or a sequence variant thereof, which is at least 70% or at least 75%, preferably at least 80% or at least 85%, more preferably at least 90% or at least 95%, even more preferably at least 97% or at least 98%, particularly preferably at least 99% identical to SEQ ID NO: 49 is preferred as component c), i.e. as the TLR peptide agonist, comprised by the complex.

(TLR2 peptide agonist Anaxa)
SEQ ID NO: 49
STVHEILCKLSLEGDHSTPPSAYGSVKPYTNFDAE

A particularly preferred functional sequence variant of the TLR peptide agonist according to SEQ ID NO: 49 is the TLR peptide agonist according to SEQ ID NO: 50:

SEQ ID NO: 50
STVHEILSKLSLEGDHSTPPSAYGSVKPYTNFDAE

Accordingly, a TLR2 peptide agonist comprising or consisting of an amino acid sequence according to SEQ ID NO: 50 or a sequence variant thereof as described above is particularly preferred as component c), i.e. as the at least one TLR peptide agonist, comprised by the complex.

Regarding TLR4, TLR peptides agonists are particularly preferred, which in particular correspond to motifs that bind to TLR4, in particular (i) peptides mimicking the natural LPS ligand (RS01: Gln-Glu-Ile-Asn-Ser-Ser-Tyr and RS09: Ala-Pro-Pro-His-Ala-Leu-Ser) and (ii) Fibronectin derived peptides. The cellular glycoprotein Fibronectin (FN) has multiple isoforms generated from a single gene by alternative splicing of three exons. One of these isoforms is the extra domain A (EDA), which interacts with TLR4.

Further suitable TLR peptide agonists comprise a fibronectin EDA domain or a fragment or variant thereof. Such suitable fibronectin EDA domains or a fragments or variants thereof are disclosed in EP 1 913 954 B1, EP 2 476 440 A1, US 2009/0220532 A1, and WO 2011/101332 A1. Thereby, a TLR4 peptide agonist comprising or consisting of an amino acid sequence according to SEQ ID NO: 40 or a sequence variant thereof as described above is preferred as component c), i.e. as the at least one TLR peptide agonist, comprised by the complex comprised by the combination according to the present invention.

(TLR4 peptide agonist EDA)
SEQ ID NO: 52
NIDRPKGLAFTDVDVDSIKIAWESPQGQVSRYRVTYSSPEDGIRELFPAP
DGEDDTAELQGLRPGSEYTVSVVALHDDMESQPLIGIQST

Another suitable TLR peptide agonists comprises or consists of Hp91, or a fragment or variant thereof as described herein. Hp91 is a TLR4-agonist, as described, e.g., in U.S. Pat. No. 9,539,321 B2 and has the following amino acid sequence:

{SEQ ID NO: 53]
DPNAPKRPPSAFFLFCSEKRYKNRVASRKSRAKFKQLLQHYREVAAAKSS
ENDRLRLLLKESLKISQAVHAAHAEINEAGREVVGVGALKVPRNQDWLGV
PRFAKFASFEAQGALANIAVDKANLDVEQLESIINFEKLTEWTGS

In addition, high-mobility group box 1 protein (HMGB1) and peptide fragments thereof are assumed to act as TLR2 agonist, in particular as an enhancer of TLR2-mediated inflammatory activities. Such HMGB1-derived peptides are for example disclosed in US 2011/0236406 A1. Thereby, a TLR2 peptide agonist comprising or consisting of an amino acid sequence according to SEQ ID NO: 51 or a sequence variant thereof as described above is preferred as component c), i.e. as the at least one TLR peptide agonist, comprised by the complex comprised by the combination according to the present invention:

[SEQ ID NO: 51]
MGKGDPKKPRGKMSSYAFFVQTCREEHKKKHPDASVNFSEFSKKCSERWK
TMSAKEKGKFEDMAKADKARYEREMKTYIPPKGETKKKFKDPNAPKRPPS
AFFLFCSEYRPKIKGEHPGLSIGDVAKKLGEMWNNTAADDKQPYEKKAAK
LKEKYEKDIAAYRAKGKPDAAKKGVVKAEKSKKKK

HMGB1 and peptide fragments thereof, may be used as sole TLR agonist or as an enhancer of TLR2-mediated inflammatory activities in combination with (other) TLR2/TLR4 peptide agonists. Accordingly, in some embodiments, the complex may e.g. comprise as part of the TLR agonist 130 HMGB1 or any immunomodulatory fragment thereof, such as those disclosed in WO2006/083301 A1 in combination with a TLR2/TLR4 peptide agonist, such as e.g. ANAXA (SEQ ID NO: 49) or sequence variants thereof, such as SEQ ID NO: 50. Accordingly, the complex may comprise in addition to the TLR peptide agonists disclosed above Δ30 HMGB1 (SEQ ID NO: 51), any immunomodulatory fragment thereof, or any of the peptides Hp-1-HP-166 as disclosed in WO2006/083301 A1, preferably Hp-31, Hp-46, Hp-106. For example, the complex (may comprise at least the TLR peptide agonists EDA (SEQ ID NO: 52) and A30 HMGB1 (SEQ ID NO: 51), or EDA (SEQ ID NO: 52) and Hp-31, or Hp-46, or Hp-106, preferably the complex comprises at least the TLR peptide agonists ANAXA (SEQ. ID NO: 49 or 50) and A30 HMGB1 (SEQ. ID NO: 51), or ANAXA (SEQ. ID NO: 49 or 50) and Hp-31, or Hp-46, or Hp-106. The use of any such combination may be advantageous if a stronger self-adjuvancy of the complex is desired.

Preferably, the complex comprised by the combination according to the present invention comprises a single TLR agonist. In other embodiments, the complex comprised by the combination according to the present invention may comprise more than one TLR peptide agonist, in particular 2, 3, 4, 5, 6, 7, 8, 9, 10 or more TLR peptide agonists, more preferably the complex comprised by the combination according to the present invention comprises (at least) two or three TLR peptide agonists, even more preferably the complex comprised by the combination according to the present invention comprises (at least) four or five TLR peptide agonists. If more than one TLR peptide agonist is comprised by the complex it is understood that said TLR peptide agonist is in particular also covalently linked in the complex comprised by the combination according to the present invention, e.g. to another TLR peptide agonist and/or to a component a), i.e. a cell penetrating peptide, and/or to a component b), i.e. an antigen or antigenic epitope.

In a particularly preferred embodiment, the complex comprised by the combination according to the present invention comprises one single TLR peptide agonist. In particularly, in this particularly preferred embodiment, the complex comprised by the combination according to the present invention comprises one single TLR peptide agonist and no further component having TLR agonist properties except the one single TLR peptide agonist as described.

The various TLR peptide agonists comprised by the complex may be the same or different. Preferably, the various TLR peptide agonists comprised by the complex are different from each other.

Moreover, it is preferred that the more than one antigen or antigenic epitope, in particular 2, 3, 4, 5, 6, 7, 8, 9, 10 antigens or antigenic epitopes, or more TLR peptide agonists, in particular 2, 3, 4, 5, 6, 7, 8, 9, 10 TLR agonists, are positioned consecutively in the complex comprised by the combination according to the present invention. This means in particular that all TLR peptide agonists comprised by the complex are positioned in a stretch, which is neither interrupted by component a), i.e. a cell penetrating peptide, nor by component b), i.e. at least one antigen or antigenic epitope. Rather, component a) and component b) are positioned in the complex for example before or after such a stretch of all TLR peptide agonists. However, the TLR peptide agonists positioned consecutively in such a way may be linked to each other for example by a spacer or linker as described below, which is neither component a), i.e. a cell penetrating peptide, nor component b), i.e. at least one antigen or antigenic epitope.

Alternatively, however, the various TLR peptide agonists may also be positioned in any other way in the complex comprised by the combination according to the present invention, for example with component a) and/or component b) positioned in between two or more TLR peptide agonists, i.e. with one or more TLR peptide agonist positioned between component a) and component b) (or vice versa) and, optionally, one or more TLR peptide agonists positioned at the respective other end of component a) and/or component b).

It is understood that a number of different TLR peptide agonists activating the same or different TLR receptors may be advantageously comprised by a single complex. Alternatively, a number of different TLR peptide agonists activating the same or different TLR receptors may be distributed to subsets of different TLR peptide agonists activating the same or different TLR receptors, which are comprised by different complexes, whereby such different complexes comprising different subsets may advantageously be administered simultaneously, e.g. in a single vaccine, to a subject in need thereof.

### Linkage and Arrangement of Components a), b), and c) in the Complex

In the complex comprised by the combination according to the present invention, components a), b) and c) are covalently linked, i.e. the linkage between two out of the three components a), b), and c) of the complex is a covalent linkage. Preferably, two out of the three components a), b), and c) of the complex are covalently linked to each other (i.e. the “first” and the “second” component), and the third component out of the three components a), b), and c) is covalently linked either to the first component out of the three components a), b), and c) or to the second component out of the three components a), b), and c). Thereby, preferably a linear molecule is formed. However, it is also conceivable that each of the three components a), b), and c) is covalently linked to both of the other components out of the three components a), b), and c).

A “covalent linkage” (also covalent bond), as used in the context of the present invention, refers to a chemical bond that involves the sharing of electron pairs between atoms. A “covalent linkage” (also covalent bond) in particular involves a stable balance of attractive and repulsive forces between atoms when they share electrons. For many molecules, the sharing of electrons allows each atom to attain the equivalent of a full outer shell, corresponding to a stable electronic configuration. Covalent bonding includes many kinds of interactions, including for example σ-bonding, π-bonding, metal-to-metal bonding, agostic interactions, and three-center two-electron bonds. Accordingly, the complex comprised by the combination according to the present invention, may also be referred to as “compound”, in particular it may be referred to as “molecule”.

Preferably, in the complex comprised by the combination according to the present invention, components a), b), and c) are covalently linked by chemical coupling in any suitable manner known in the art, such as cross-linking methods. However, attention is drawn to the fact that many known chemical cross-linking methods are non-specific, i.e., they do not direct the point of coupling to any particular site on the components a), b), and c). Thus, the use of non-specific cross-linking agents may attack functional sites or sterically block active sites, rendering the fused components of the complex biologically inactive. It is referred to the knowledge of the skilled artisan to block potentially reactive groups by using appropriate protecting groups. Alternatively, the use of the powerful and versatile oxime and hydrazone ligation techniques, which are chemo-selective entities that can be applied for the cross-linking of components a), b), and c) may be employed. This linking technology is described e.g. by Rose et al. (1994), JACS 116, 30.

The linkage between two out of the three components a), b), and c) of the complex comprised by the combination according to the present invention may be directly or indirectly, i.e. two components directly adjoin or they may be linked by an additional component of the complex, e.g. a spacer or a linker.

The complex comprised by the combination according to the present invention may optionally comprise a spacer or linker, which are usually non-immunologic moieties, which are preferably cleavable, and which link component a) and b) and/or component a) and c), and/or component b) and c), and/or link consecutive antigens or antigenic epitopes, and/or link consecutive TLR peptide agonists, and/or link consecutive cell penetrating peptides, and/or which can be placed at the C-terminal part of components b) and/or c). A linker or spacer may preferably provide further functionalities in addition to linking of the components, and preferably being cleavable, more preferably naturally cleavable inside the target cell, e.g. by enzymatic cleavage. However, such further functionalities do in particular not include any immunological functionalities. Examples of further functionalities, in particular regarding linkers in fusion proteins, can be found in Chen X. et al., 2013: Fusion Protein Linkers: Property, Design and Functionality. Adv Drug Deliv Rev. 65(10): 1357-1369, wherein for example also in vivo cleavable linkers are disclosed. Moreover, Chen X. et al., 2013: Fusion Protein Linkers: Property, Design and Functionality. Adv Drug Deliv Rev. 65(10): 1357-1369 also discloses various linkers, e.g. flexible linkers and rigid linkers, and linker designing tools and databases, which can be useful in the complex comprised by the combination according to the present invention or to design a linker to be used in the complex.

Said spacer may be peptidic or non-peptidic, preferably the spacer is peptidic. Preferably, a peptidic spacer consists of about 1, 2, 3, 4, 5, 6, 7, 8, 9, or 10 amino acids, more preferably of about 1, 2, 3, 4, or 5 amino acids. The amino acid sequence of the peptidic spacer may be identical to that of the N-terminal or C-terminal flanking region of any of the components a), b), or c). Alternatively a peptidic spacer can consist of non-natural amino acid sequences such as an amino acid sequence resulting from conservative amino acid substitutions of said natural flanking regions or sequences of known cleavage sites for proteases. In some embodiments, the peptidic spacer does not contain any Cys (C) residues. In some embodiments the linker sequence contains at least 20%, more preferably at least 40% and even more preferably at least 50% Gly or β-alanine residues. Appropriate linker sequences can be easily selected and prepared by a person skilled in the art. They may be composed of D and/or L amino acids.

In some embodiments, the complex comprised by the combination according to the invention may comprise a spacer or linker, in particular a peptidic spacer, placed between component a) and b) and/or between component a) and c), and/or between component b) and c). This peptidic spacer can be chosen by one skilled in the art so that it may be cut by the cell machinery once the complex comprising the cell penetrating peptide and the cargo molecule has been internalized.

The technics for linking two of the three components a), b), and c) are well documented in the literature and can depend on the nature of the at least one antigen or antigenic epitope. For instance, linkages between two of the three components a), b), and c) can be achieved via cleavable disulphide linkages through total stepwise solid-phase synthesis or solution-phase or solid-phase fragment coupling, stable amide, thiazolidine, oxime and hydrazine linkage, disulphide linkage, stable thiomaleimide linkage, peptide bond (including peptide bonds between amino acids of a fusion protein), or electrostatic or hydrophobic interactions.

Preferably, the at least one antigen or antigenic epitope comprised by the complex as well as any optional spacer or linker comprised by the complex are of peptidic nature. More preferably, all components of the complex comprised by the combination according to the present invention, e.g. the cell penetrating peptide, the at least one antigen or antigenic epitope, which is a peptide, polypeptide or protein, the at least one TLR peptide agonist and any optional peptidic linker or spacer are linked in the complex comprised by the combination according to the present invention by a peptide bond. Most preferably, the complex comprised by the combination according to the present invention is thus a peptide, polypeptide or protein, such as a fusion protein, e.g. a recombinant fusion protein.

The components a), b), and c) may be arranged in the complex comprised by the combination according to the present invention in any way.

In particular if more than one cell penetrating peptide and/or more than one antigen or antigenic epitope and/or more than one TLR peptide agonist are comprised by the complex, the more than one cell penetrating peptide may be positioned in a non-consecutive manner, i.e. at least one antigen or antigenic epitope (component b)) and/or at least one TLR peptide agonist (component c)) may interrupt a stretch of consecutively positioned cell penetrating peptides and/or the cell penetrating peptides may be positioned with component b) and/or with component c) in an alternating manner. Similarly, the more than one antigen or antigenic epitope may be positioned in a non-consecutive manner, i.e. at least one cell penetrating peptide (component a)) and/or at least one TLR peptide agonist (component c)) may interrupt a stretch of consecutively positioned antigens or antigenic epitopes and/or the antigens or antigenic epitopes may be positioned with component a) and/or with component c) in an alternating manner. Similarly, the more than one TLR peptide agonist may be positioned in a non-consecutive manner, i.e. at least one cell penetrating peptide (component a)) and/or at least one antigen or antigenic epitope (component b)) may interrupt a stretch of consecutively positioned TLR peptide agonists and/or the TLR peptide agonists may be positioned with component a) and/or with component b) in an alternating manner.

However, it is preferred that the more than one cell penetrating peptide is positioned in the complex comprised by the combination according to the present invention in a consecutive manner and/or the more than one antigen or antigenic epitope is positioned in the complex comprised by the combination according to the present invention in a consecutive manner and/or the more than one TLR peptide agonist is positioned in the complex comprised by the combination according to the present invention in a consecutive manner. This means in particular that all single units of a certain component, i.e. all cell penetrating peptides, all antigens or antigenic epitopes or all TLR peptide agonists, which are comprised by the complex are positioned in a stretch, which is not interrupted by any of the other two components. Rather, the other two components are positioned in the complex for example before or after such a stretch of all single units of said certain component. However, the single units of said certain component positioned consecutively in such a way may be linked to each other for example by a spacer or linker as described herein, which is not of the other two components.

It is particularly preferred that each of the components a), b), and c) is positioned in a consecutive manner.

Preferably, all three components a), b), and c) are linked via main-chain/main-chain linkage, thus resulting in particular in a main chain of the complex, which comprises the main chain of one or more cell penetrating peptide(s), the main chain of one or more antigen(s) or antigenic epitope(s), and the main chain of one or more TLR peptide agonist(s). In other words, the main chain of one or more cell penetrating peptide(s), the main chain of one or more antigen(s) or antigenic epitope(s), and the main chain of one or more TLR peptide agonist(s) constitute the main chain of the complex, optionally together with further components, for example linker(s), spacer(s), etc. Accordingly, the following arrangements of the components a), b), and c) are preferred, in particular if the at least one antigen or antigenic epitope is a peptide, polypeptide or protein, whereby said preferred arrangements are shown below in N-terminus→C-terminus direction of the main chain of the complex and wherein all three components a), b), and c) are linked via main-chain/main-chain linkage and may be optionally linked by a linker, a spacer or another additional component:
(α) component a) (cell penetrating peptide)-component b) (at least one antigen or antigenic epitope)-component c) (at least one TLR peptide agonist);
(β) component c) (at least one TLR peptide agonist)-component a) (cell penetrating peptide)-component b) (at least one antigen or antigenic epitope);
(γ) component a) (cell penetrating peptide)-component c) (at least one TLR peptide agonist)-component b) (at least one antigen or antigenic epitope);
(δ) component c) (at least one TLR peptide agonist)-component b) (at least one antigen or antigenic epitope)-component a) (cell penetrating peptide);
(ε) component b) (at least one antigen or antigenic epitope)-component a) (cell penetrating peptide)-component c) (at least one TLR peptide agonist); or
(ζ) component b) (at least one antigen or antigenic epitope)-component c) (at least one TLR peptide agonist)-component a) (cell penetrating peptide).

In particular if all three components a), b), and c) are linked via main-chain/main-chain linkage, it is preferred that the at least one antigen or antigenic epitope is positioned C-terminally of the cell penetrating peptide, whereby the cell penetrating peptide and the at least one antigen or antigenic epitope are optionally linked by a further component, e.g. a linker, a spacer, or by the at least one TLR peptide agonist. Accordingly, this corresponds to the arrangements (α), (β), and (γ) from the arrangements shown above, i.e. from the above arrangements (α), (β), and (γ) are more preferred.

Even more preferably, the at least one antigen or antigenic epitope is positioned C-terminally of the cell penetrating peptide, whereby the cell penetrating peptide and the at least one antigen or antigenic epitope are optionally linked by a further component, e.g. a linker, a spacer, but not by the at least one TLR peptide agonist. Accordingly, this corresponds to the arrangements (α) and (β) from the arrangements shown above, i.e. from the above arrangements (α) and (β) are even more preferred. Particularly preferably, the complex comprised by the combination according to the present invention is a recombinant polypeptide or a recombinant protein and the components a) to c) are positioned in N-terminus→C-terminus direction (N-terminal→C-terminal direction) of the main chain of said complex in the order:

(α) component a)-component b)-component c); or
(β) component c)-component a)-component b),
wherein the components may be linked by a further component, in particular by a linker or a spacer.

In some embodiments, the at least one antigen or antigenic epitope (or the multiantigenic domain) of the complex is positioned C-terminally of the cell penetrating peptide of the complex, wherein the cell penetrating peptide and the at least one antigen or antigenic epitope (or the multiantigenic domain) are optionally linked by a further component, e.g. a linker, a spacer, or by the TLR peptide agonist of the complex.

A preferred exemplified complex of the inventive combination is a polypeptide or protein, wherein
a) the cell penetrating peptide has an amino acid sequence comprising or consisting of an amino acid sequence according to SEQ ID NO: 6 (CPP3/Z13), SEQ ID NO: 7 (CPP4/Z14), SEQ ID NO: 8 (CPP5/Z15), or SEQ ID NO: 11 (CPP8/Z18), or a (functional) sequence variant thereof having at least 70%, 75%, 80%, 85%, 90% or 95% sequence identity (without abrogating said peptide's cell penetrating ability);
b) the at least one antigen or antigenic epitope is a peptide, polypeptide or protein and, preferably, comprises or consists of at least one cancer/tumor epitope; and
c) the TLR peptide agonist is a TLR2 peptide agonist and/or a TLR4 peptide agonist.

It is particularly preferred for the combination according to the present invention, that the complex comprises or consists of an amino acid sequence according to SEQ ID NO: 54, or a (functional) sequence variant thereof having at least 70% or at least 75%, preferably at least 80% or at least 85%, more preferably at least 90% or at least 95%, even more preferably at least 97% or at least 98%, particularly preferably at least 99% sequence identity.

[SEQ ID NO: 54]
KRYKNRVASRKSRAKFKQLLQHYREVAAAKSSENDRLRLLLKNRTLTLFN
VTRNDARAYVSGIQNSVSANRSDPVTLDVLPDSSYLSGANLNLSCHSASP
QYSWRINGIPQQHTQVLFIAKITPNNNGTYACFVSNLATGRNNSIVKSIT
VSASGTSPGLSAAPTLPPAWQPFLKDHRISTFKNWPFLEGSAVKKQFEEL
TLGEFLKLDRERAAVARRNERERNRVKLVNLGFQALRQHVPHGGASKKLS
KVETLRSAVEYIRALQRLLAEHDAVRNALAGGLRPQAVRPSAPRGPSEGA
LSPAERELLDFSSWLGGYSTVHEILSKLSLEGDHSTPPSAYGSVKPYTNF
DAE

In some embodiments, the complex may comprise or consist of an amino acid sequence according to SEQ ID NO: 55, or a (functional) sequence variant thereof having at least 70% or at least 75%, preferably at least 80% or at least 85%, more preferably at least 90% or at least 95%, even more preferably at least 97% or at least 98%, particularly preferably at least 99% sequence identity.

[SEQ ID NO: 55]
KRYKNRVASRKSRAKFKQLLQHYREVAAAKSSENDRLRLLLKQAEPDRAH
YNIVTFSSKSSTVHEILSKLSLEGDHSTPPSAYGSVKPYTNFDAE

### STING Agonist

The stimulator of interferon (IFN) genes (STING) is a 379-amino-acid protein, which belongs to the family of nucleic acid sensors and is the adaptor for cytosolic DNA signaling. STING is expressed in various endothelial and epithelial cell types, as well as in hematopoietic cells, such as T cells, macrophages and dendritic cells (DCs). STING is part of the innate immune response to cytosolic nucleic acids and functions as a DNA sensor and signaling molecule. STING is essential for controlling the transcription of numerous host defense genes, including type I IFNs and other pro-inflammatory cytokines, following the recognition of cyclic dinucleotides (CDNs) in the cytosol of the cell. In its basal state STING exists as a dimer with its N-terminal domain anchored in the endoplasmic reticulum (ER) and the C-terminal domain residing in the cytosol. Cyclic dinucleotides (CDNs), generated by the protein cyclic GMP-AMP Synthase (cGAS), are the natural ligands of STING (Ablasser et al, Nature 498, 380-384, 2013). Binding of CDNs to STING induces conformational changes, which allow the binding and activation of the TANK binding kinase (TBK1) and interferon regulatory factor 3 (IRF3) and the re-localization from the ER to perinuclear endosomes (Liu et al, Science 347, Issue 6227, 2630-1-2630-14, 2015). Phosphorylation of the transcription factor IRF3 and NF-kB by TBK1 results in expression of multiple cytokines including type I IFN. The production of type I IFNs leads to the activation of DCs, effective cross-priming of CD8+ T cells against tumor antigens and migration of tumor-specific CD8+ T cells into the tumor.

Thus, therapeutic strategies that activate the STING innate immune-sensing pathway to restore type I IFN signaling may have the potential to increase tumor immunogenicity—‘heating up’ immune cold tumors that do not respond to alternative therapies.

As used herein, the term “STING agonist” refers to a compound, which induces, activates, stimulates, enhances or prolongs the activity of STING. An overview is provided, for example, in Ding et al., 2020 (C. Ding, Z. Song, A. Shen, T. Chen, A. Zhang. Small molecules targeting the innate immune cGAS-STING-TBK1 signaling pathway. Acta Pharm. Sin. B (2020), 10.1016/j.apsb.2020.03.001, published online Mar. 13, 2020). In particular, the specific examples of STING agonists described in Ding et al., 2020 (incorporated herein by reference) may be useful as STING agonists in the context of the present invention.

In some embodiments, the term “STING agonist” includes compounds which act indirectly (i.e. which do not directly interact with STING), such as cyclic GMP-AMP Synthase (cGAS) agonists. Activated cGAS then synthesizes 2′,3′-cGAMP, which in turn acts as an agonist for STING. Non-limiting examples of cGAS agonists include
Spherical Nucleic Acids (SNAs) presenting dsDNA at high surface density, in particular SNA nanostructures carrying a 45 bp IFN-simulating dsDNA oligonucleotide, such as those described in Alexander Stegh, DDIS-03. DEVELOPMENT OF A NOVEL CLASS OF cGAS AGONISTS TO TRIGGER STING PATHWAY-DEPENDENT INNATE IMMUNE RESPONSES AGAINST GLIOBLASTOMA, Neuro-Oncology, Volume 21, Issue Supplement_6, November 2019, Page vi65, which is incorporated herein by reference; and
G3-YSD, which is a 26-mer DNA sequence derived from the HIV-1 RNA genome (Herzner A M. et al., 2015. Sequence-specific activation of the DNA sensor cGAS by Y-form DNA structures as found in primary HIV-1 cDNA. Nat Immunol. 16(10):1025-33).

In other embodiments, the term “STING agonist” refers to such compounds only, which directly interact with STING.

Various (direct) STING agonists are known in the art. In general, (direct) STING agonists can be classified as cyclic dinucleotides (CDNs) and non-CDN STING agonists. CDN STING agonists are inspired by the natural ligand of STING, 2′3′-cGAMP. Examples of non-CDN STING agonists include small molecule STING agonists, with “compound 7” described in L. Corrales et al. (L. Corrales, L. H. Glickman, S. M. McWhirter, D. B. Kanne, K. E. Sivick, G. E. Katibah, et al. Direct activation of STING in the tumor microenvironment leads to potent and systemic tumor regression and immunity, Cell Rep, 11 (2015), pp. 1018-1030) being recognized as the prototypic structural model for the development of non-CDN small molecule STING agonists. Preferably, however, the (direct) STING agonist is a cyclic-dinucleotide (CDN) based STING agonist.

Non-limiting examples of STING agonists useful in the context of the present invention include:
the STING agonists as described in WO 2014/093936 A1, WO 2014/189805 and WO 2014/189806, in particular ADU-S100 (Aduro),
the STING agonists as described in WO 2017/027646 A1 and WO 2018/118664 A1, in particular MK-1454 (Merck),
the STING agonists as described in WO 2018/152450 A1, WO 2018/152453 A1 and WO 2020/036199 A1, in particular E-7766 (Eisai),
MK-2118 (Merck)
BMS-986301 (Bristol-Myers Squibb),
IMSA-101 (ImmuneSensor Therapeutics Inc.), an analog of cGAMP,
SB-11285 (Spring Bank Pharmaceuticals), a small molecule-nucleic acid hybrid STING agonist,
SYNB-1891 (Synlogic), a non-pathogenic strain of Escherichia coli (E. coli) bacteria that has been engineered to express STING,
GSK-3745417 (GlaxoSmithKline), which is believed to be a synthetic non-CDN STING agonist with dimeric ABZI scaffold,
non-CDN STING agonist TAK-676 (Takeda), and
non-CDN small molecule STING agonist TTI-10001 (Trillium Therapeutics Inc.).

Accordingly, the STING agonist may be selected from the group consisting of ADU-S100, MK-1454, E-7766, MK-2118, BMS-986301, IMSA-101, SB-11285, SYNB-1891, GSK-3745417, TAK-676, and TTI-10001. Among those STING agonists, ADU-S100 is preferred.

Preferably, the STING agonist is a compound as described in WO 2018/060323 A1, which is incorporated herein by reference, or a compound as described in WO 2018/172206, which is incorporated herein by reference.

More preferably, the STING agonist is a compound of formula I

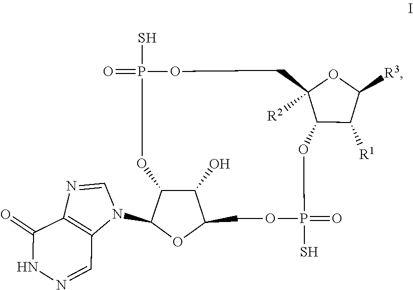

wherein
R¹ is selected from the group consisting of H, F, —O—C₁₋₃alkyl and OH, and
R² is H, or
R² is —CH₂— and R¹ is —O—, forming together a —CH₂—O— bridge, and
R³ is a purine nucleobase selected from the group consisting of purine, adenine, guanine, xanthine, hypoxanthine, connected through its N⁹ nitrogen,
   or a solvate or a hydrate thereof, or a salt thereof.

Even more preferably, the STING agonist is a compound of formula Ia

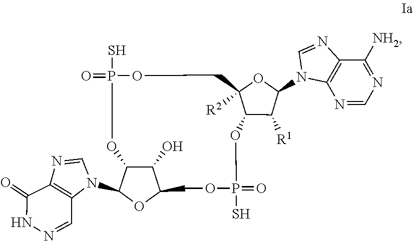

wherein R¹ and R² are defined as disclosed in relation to formula I, or a solvate or a hydrate thereof, or a salt thereof.

It is also even more preferred, that the STING agonist is a compound of formula Ib

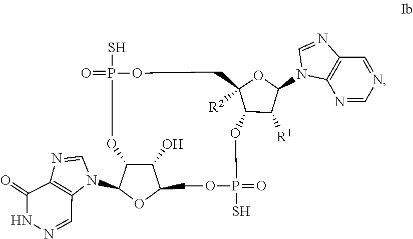

or a solvate or a hydrate thereof, or a salt thereof.

Still more preferably, the STING agonist is a compound of formula Ia.1

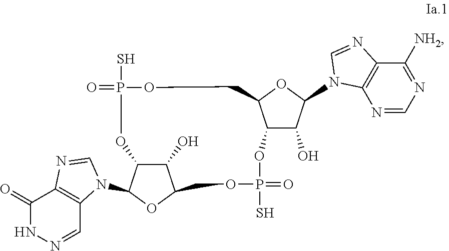 [SMILES: Nc1ncnc2c1ncn2[C@@H]1O[C@@H]2COP(=O)(S)O[C@@H]3[C@H](O)[C@@H](COP(=O)(S)O[C@H]2[C@H]1O)O[C@H]3n1cnc2c(=O)[nH]ncc21]

or a solvate or a hydrate thereof.

It is also still more preferred that the STING agonist is a compound of formula Ia.2 (STINGa 2).

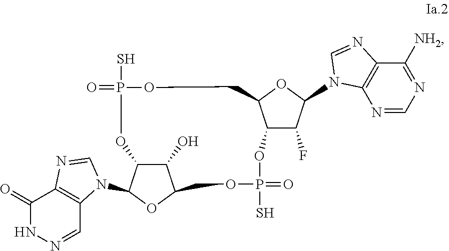 [SMILES: Nc1ncnc2c1ncn2[C@@H]1O[C@@H]2COP(=O)(S)O[C@@H]3[C@H](O)[C@@H](COP(=O)(S)O[C@H]2[C@H]1F)O[C@H]3n1cnc2c(=O)[nH]ncc21]

or a solvate or a hydrate thereof.

Moreover, it is also still more preferred that the STING agonist is a compound of formula Ia.3

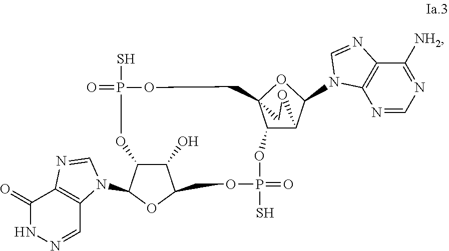 [SMILES: Nc1ncnc2c1ncn2[C@@H]1O[C@]23CO[C@@H]1[C@@H]2OP(=O)(S)OC[C@H]1O[C@@H](n2cnc4c(=O)[nH]ncc42)[C@H](OP(=O)(S)OC3)[C@@H]1O]

or a solvate or a hydrate thereof.

Furthermore, it is also still more preferred that the STING agonist is a compound of formula Ib.1

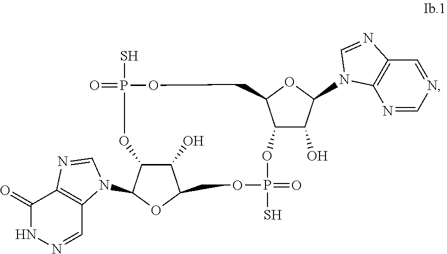 [SMILES: O=c1[nH]ncc2c1ncn2[C@@H]1O[C@@H]2COP(=O)(S)O[C@H]3[C@@H](O)[C@H](n4cnc5cncnc54)O[C@@H]3COP(=O)(S)O[C@@H]1[C@@H]2O]

or a solvate or a hydrate thereof.

It is also preferred that the STING agonist is a compound of formula (II) (as described in WO 2018/172206).

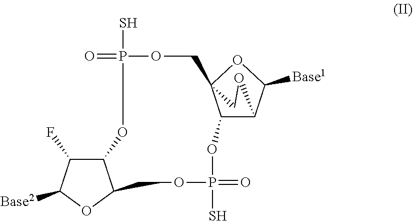

wherein
Base¹ and Base¹ are independently selected from the group consisting of purine, adenine, guanine, xanthine, and hypoxanthine, connected through their N⁹ nitrogen atoms,
   or a salt thereof.

More preferably, in the compound of formula (II) Base¹ and Base² are adenine, such that the STING agonist exhibits the structure of formula (II-1):

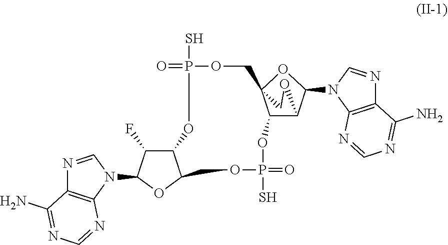 [SMILES: Nc1ncnc2c1ncn2[C@@H]1O[C@@H]2COP(=O)(S)O[C@H]3[C@H]4OC[C@]3(COP(=O)(S)O[C@H]2[C@H]1F)O[C@H]4n1cnc2c(N)ncnc21]

It is also more preferred that in the compound of formula (II) Base¹ is adenine and Base² is guanine, such that the STING agonist exhibits the structure of formula (II-2):

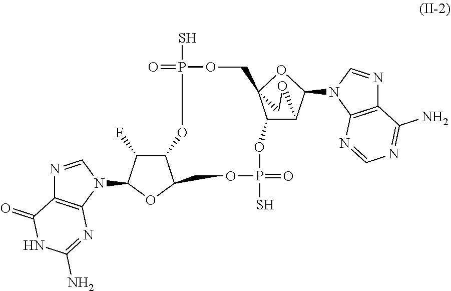 [SMILES: Nc1nc2c(ncn2[C@@H]2O[C@@H]3COP(=O)(S)O[C@H]4[C@H]5OC[C@]4(COP(=O)(S)O[C@H]3[C@H]2F)O[C@H]5n2cnc3c(N)ncnc32)c(=O)[nH]1]

It is also more preferred that in the compound of formula (II) Base¹ is guanine and Base¹ is adenine, such that the STING agonist exhibits the structure of formula (II-3):

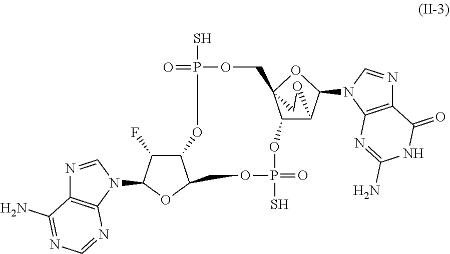 [SMILES: Nc1nc2c(ncn2[C@@H]2O[C@@]34CO[C@@H]2[C@@H]3OP(=O)(S)OC[C@H]2O[C@@H](n3cnc5c(N)ncnc53)[C@H](F)[C@@H]2OP(=O)(S)OC4)c(=O)[nH]1]

It is also more preferred that in the compound of formula (II) Base¹ is adenine and Base¹ is hypoxanthine, such that the STING agonist exhibits the structure of formula (II-4):

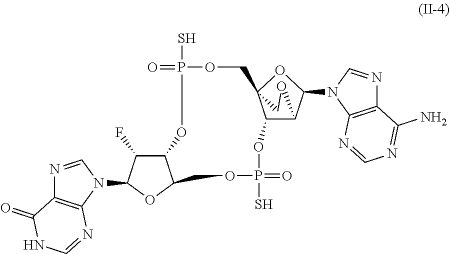 [SMILES: Nc1ncnc2c1ncn2[C@@H]1O[C@@]23CO[C@@H]1[C@@H]2OP(=O)(S)OC[C@H]1O[C@@H](n2cnc4c(=O)[nH]cnc42)[C@H](F)[C@@H]1OP(=O)(S)OC3]

The STING agonist may also be a substantially pure (Sp,Sp), (Rp,Rp), (Sp,Rp), or (Rp,Sp) stereoisomer of a compound shown in any one of the above in structural formulas I, Ia, Ib, Ia.1, Ia.2, Ia.3, Ib.1, II, II-1, II-2, II-3 and II-4, or a salt thereof. Preferably, the STING agonist is a substantially pure (Rp,Rp) stereoisomer of a compound shown in any one of the above in structural formulas I, Ia, Ib, Ia.1, Ia.2, Ia.3, Ib.1, II, II-1, II-2, II-3 and II-4, or a salt thereof. The term “substantially pure” as used herein refers to one (Rp,Rp), (Rp,Sp), (Sp,Rp) or (Sp,Sp) diastereomer which is at least 75% pure relative to the other possible diastereomers with respect to the phosphor atoms. In preferred embodiments, a substantially pure compound is at least 85% pure, at least 90% pure, at least 95% pure, at least 97% pure, and at least 99% pure.

In some embodiments, the STING agonist is a pharmaceutically acceptable salt of a compound shown in any one of the above in structural formulas I, Ia, Ib, Ia.1, Ia.2, Ia.3, Ib.1, II, II-1, II-2, II-3 and II-4. As used herein, the expression “pharmaceutically acceptable” is employed to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio. As used herein, “pharmaceutically acceptable salts” refer to derivatives of the disclosed compounds wherein the parent compound is modified by making salts thereof with bases. The pharmaceutically acceptable salts can be synthesized from the parent compound which contains an acidic moiety by conventional chemical methods. Generally, such salts can be prepared by reacting the free acid forms of these compounds with a sufficient amount of the appropriate base in water or in an organic diluent like ether, ethyl acetate, ethanol, isopropanol, or acetonitrile, or a mixture thereof. Alternatively, salts can be prepared by ion exchange, for example by treating aqueous solutions of the compounds of the invention (free acid or salt form) with a cation exchanger. In some embodiments, the STING agonist is a sodium salt of a compound shown in any one of the above in structural formulas I, Ia, Ib, Ia.1, Ia.2, Ia.3, Ib.1, II, II-1, II-2, II-3 and II-4.

In the combination according to the present invention it is preferred that the complex comprises or consists of an amino acid sequence according to SEQ ID NO: 55 or a (functional) sequence variant thereof having at least 70%, 75%, 80%, 85%, 90% or 95% sequence identity, and the STING agonist is ADU-S100.

It is also preferred in the combination according to the present invention that the complex comprises or consists of an amino acid sequence according to SEQ ID NO: 55 or a (functional) sequence variant thereof having at least 70%, 75%, 80%, 85%, 90% or 95% sequence identity, and the STING agonist is one compound selected from the group of compounds represented by formula Ia.1, Ia.2, Ia.3, Ib.1, II-1, II-2, II-3 and II-4, or a solvate or a hydrate thereof (as described above).

More preferably, the complex comprises or consists of an amino acid sequence according to SEQ ID NO: 54 or a (functional) sequence variant thereof having at least 70%, 75%, 80%, 85%, 90% or 95% sequence identity, and the STING agonist is ADU-S100.

Even more preferably, the complex comprises or consists of an amino acid sequence according to SEQ ID NO: 54 or a (functional) sequence variant thereof having at least 70%, 75%, 80%, 85%, 90% or 95% sequence identity, and the STING agonist is one compound selected from the group of compounds represented by formula Ia.1, Ia.2, Ia.3, Ib.1, II-1, II-2, II-3 and II-4, or a solvate or a hydrate thereof (as described above).

In general, with regard to the sequence variants, the higher the % identity, the more is the sequence variant preferred. Accordingly, 100% identity is most preferred, for example amino acid sequences according to SEQ ID NO: 54 or 55. Similarly, compounds of formula Ia.1, Ia.2, Ia.3, Ib.1, II-1, II-2, II-3 and II-4 are generally more preferred than their solvates or hydrates.

Accordingly, the present invention also provides the complex as described above for use (in particular for use in medicine) in combination with a STING agonist. Preferably, the STING agonist is as described above. Preferred medical uses are described below, such as prevention and/or treatment of a cancer.

Moreover, the present invention also provides a STING agonist for use (in particular for use in medicine) in combination with the complex as described above. Preferably, the STING agonist is as described above. Preferred medical uses are described below, such as prevention and/or treatment of a cancer.

### Medical Use

The combination of (i) the STING agonist and (ii) the complex (and any optional further component) according to the present invention, as described in detail above, may be used in medicine, in particular as medicament.

As described herein and shown in the appended examples, the combination of (i) a STING agonist and (ii) a complex comprising a cell penetrating peptide, at least one antigen or antigenic epitope, and a TLR peptide agonist improves both CD4 and CD8 T cells response boosting antigen-specific CD8 T cells, increases intra-tumoral immunogenicity and results in considerably increased survival rates and reduced tumor growth. This indicates a synergistic effect of the STING agonist and the complex acting together, which considerably increases the anti-tumor effects of each of its components administered as stand-alone therapy.

The combination according to the present invention can be useful in a variety of diseases. Preferably the combination as described herein is for use (for the preparation of a medicament) for the prevention, treatment or stabilization of a disease or disorder, such as those which can be treated by immunotherapy, including cancers, infectious diseases, autoimmunity disorders, hematological diseases and transplant rejections. Accordingly, a combination as described herein for use in the prevention, treatment or stabilization of a disease or disorder, such as those which can be treated by immunotherapy, including cancers, infectious diseases, autoimmunity disorders, hematological diseases and transplant rejections is preferred.

In the context of the present invention, it is particularly preferred that the combination according to the present invention as described herein is used in the prevention and/or treatment of cancer or of a tumor.

Accordingly, the present invention also provides method for treating cancer or initiating, enhancing or prolonging an anti-tumor-response in a subject in need thereof comprising administering to the subject an effective amount of the inventive combination as described above (i.e., an effective amount of (i) the STING agonist; and an effective amount of (ii) the complex comprising a cell penetrating peptide, at least one antigen or antigenic epitope, and a TLR peptide agonist; and, optionally (iii) an effective amount of any optional further active substance). Furthermore, the present invention also provides a method for increasing the infiltration of a tumor with tumor antigen-specific T-cells in a patient, the method comprising administering to a patient afflicted with a tumor or cancer the inventive combination as described above (i.e., (i) the STING agonist; and (ii) the complex comprising a cell penetrating peptide, at least one antigen or antigenic epitope, and a TLR peptide agonist; and, optionally (iii) any optional further active substance). Moreover, the present invention also provides a combination therapy for preventing and/or treating cancer, wherein the combination therapy comprises administration of the inventive combination as described above (i.e., (i) the STING agonist; and (ii) the complex comprising a cell penetrating peptide, at least one antigen or antigenic epitope, and a TLR peptide agonist; and, optionally (iii) any optional further active substance).

The term “disease” as used in the context of the present invention is intended to be generally synonymous, and is used interchangeably with, the terms “disorder” and “condition” (as in medical condition), in that all reflect an abnormal (non-physiological or pathological) condition of the human or animal body or of one of its parts that impairs normal functioning, is typically manifested by distinguishing signs and symptoms, and causes the human or animal to have a reduced duration or quality of life.

Preferred diseases to be treated and/or prevented by use of the complex comprising a cell penetrating peptide, at least one antigen or antigenic epitope and at least one TLR peptide agonist as described herein include cancer, hematological disorders, infectious diseases, autoimmunity disorders and transplant rejections. Thereby, treatment and/or prevention of cancer and infectious diseases is preferred and treatment and/or prevention of cancer is more preferred. For cancer, an endocrine tumor, a gastrointestinal tumor, a genitourinary or gynecologic tumor, breast cancer, head and neck tumor, hematopoietic tumor, skin tumor, thoracic or respiratory tumor, preferably colorectal cancer, such as metastatic colorectal cancer, are preferred.

Preferably, the combination of the STING agonist as described herein and of the complex comprising a cell penetrating peptide, at least one antigen or antigenic epitope and at least one TLR peptide agonist as described herein may be used for (the preparation of a medicament for) the prophylaxis, treatment and/or amelioration of cancer or tumor diseases, including diseases caused by defective apoptosis. The cancer may be a solid tumor, blood cancer, or lymphatic cancer. The cancer may be benign or metastatic. Further preferred examples of cancers to be treated include brain cancer, prostate cancer, breast cancer, ovarian cancer, esophageal cancer, lung cancer, liver cancer, kidney cancer, melanoma, gut carcinoma, lung carcinoma, head and neck squamous cell carcinoma, chronic myeloid leukemia, colorectal carcinoma, gastric carcinoma, endometrial carcinoma, myeloid leukemia, lung squamous cell carcinoma, acute lymphoblastic leukemia, acute myelogenous leukemia, bladder tumor, promyelocytic leukemia, non-small cell lung carcinoma, and sarcoma. Particularly preferably, the complex comprising a cell penetrating peptide, at least one antigen or antigenic epitope and at least one TLR peptide agonist as described herein are used to treat colorectal cancer.

In some embodiments, the cancer/tumor may be selected from breast cancer, including triple-negative breast cancer, biliary tract cancer; bladder cancer; brain cancer including glioblastomas and medulloblastomas; cervical cancer; choriocarcinoma; colon cancer; endometrial cancer; esophageal cancer; gastric cancer; gastrointestinal stromal tumor (GIST), appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, colorectal cancer, or metastatic colorectal cancer, hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer, including non-small cell lung cancer, lymphomas including Hodgkin's disease and lymphocytic lymphomas; neuroblastomas; glioblastoma, oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

In some embodiments, the cancer/tumor may be selected from acusticus neurinoma, anal carcinoma, astrocytoma, basalioma, Behcet's syndrome, bladder cancer, blastomas, bone cancer, brain metastases, brain tumors, brain cancer (glioblastomas), breast cancer (mamma carcinoma), Burkitt's lymphoma, carcinoids, cervical cancer, colon carcinoma, colorectal cancer, corpus carcinoma, craniopharyngeomas, CUP syndrome, endometrial carcinoma, gall bladder cancer, genital tumors, including cancers of the genitourinary tract, glioblastoma, gliomas, head/neck tumors, hepatomas, histocytic lymphoma, Hodgkin's syndromes or lymphomas and non-Hodgkin's lymphomas, hypophysis tumor, intestinal cancer, including tumors of the small intestine, and gastrointestinal tumors, Kaposi's sarcoma, kidney cancer, kidney carcinomas, laryngeal cancer or larynx cancer, leukemia, including acute myeloid leukaemia (AML), erythroleukemia, acute lymphoid leukaemia (ALL), chronic myeloid leukaemia (CML), and chronic lymphocytic leukaemia (CLL), lid tumor, liver cancer, liver metastases, lung carcinomas (=lung cancer=bronchial carcinoma), small cell lung carcinomas and non-small cell lung carcinomas, and lung adenocarcinoma, lymphomas, lymphatic cancer, malignant melanomas, mammary carcinomas (=breast cancer), medulloblastomas, melanomas, meningiomas, Mycosis fungoides, neoplastic diseases neurinoma, oesophageal cancer, oesophageal carcinoma (=oesophageal cancer), oligodendroglioma, ovarian cancer (=ovarian carcinoma), ovarian carcinoma, pancreatic carcinoma (=pancreatic cancer), penile cancer, penis cancer, pharyngeal cancer, pituitary tumour, plasmocytoma, prostate cancer (=prostate tumors), rectal carcinoma, rectal tumors, renal cancer, renal carcinomas, retinoblastoma, sarcomas, Schneeberger's disease, skin cancer, e.g. melanoma or non-melanoma skin cancer, including basal cell and squamous cell carcinomas as well as psoriasis, pemphigus vulgaris, soft tissue tumours, spinalioma, stomach cancer, testicular cancer, throat cancer, thymoma, thyroid carcinoma, tongue cancer, urethral cancer, uterine cancer, vaginal cancer, various virus-induced tumors such as, for example, papilloma virus-induced carcinomas (e.g. cervical carcinoma=cervical cancer), adenocarcinomas, herpes virus-induced tumors (e.g. Burkitt's lymphoma, EBV-induced B-cell lymphoma, cervix carcinoma), hepatitis B-induced tumors (hepatocell carcinomas), HTLV-1- and HTLV-2-induced lymphomas, and vulval cancer.

Preferably, the patient to be treated with the combination of the invention is afflicted with colorectal cancer (CRC), in particular late stage colorectal cancer (CRC), or late stage metastatic colorectal cancer (mCRC), whereby the term “late stage” CRC, mCRC includes Stage IIIC: T4a, N2a, M0 or T3-T4a, N2b, M0 or T4b, N1-N2, M0; Stage IVA: any T, any N, M1a and Stage IVB: any T, any N, M1b (according to TNM staging), whereby the CRC or mCRC tumor may e.g. be “Microsatellite Stable” (MSS), or microsatellite instable” (MSI), preferably the CRC or mCRC tumor is MSS.

In the present context, the terms “therapy” and “therapeutic” preferably mean to have at least some minimal physiological effect upon being administered to a living body. For example, a physiological effect upon administering a “therapeutic” anti-tumor compound may be the inhibition of tumor growth, or decrease in tumor size, or prevention reoccurrence of the tumor. Preferably, in the treatment of cancer or neoplastic disease, a compound which inhibits the growth of a tumor or decreased the size of the tumor or prevents the reoccurrence of the tumor would be considered therapeutically effective. The term “anti-tumor drug” therefore preferably means any therapeutic agent having therapeutic effect against a tumor, neoplastic disease or cancer.

The components of the combination of the present invention as described herein, i.e. (i) the STING agonist and (ii) the complex comprising a cell penetrating peptide, at least one antigen or antigenic epitope, and a TLR peptide agonist (and any optional further component), are usually administered as combination therapy. This means that, even if one component (the STING agonist or the complex) is not administered, e.g., at the same day as the other component (the other of STING agonist or complex), their treatment schedules are typically intertwined. This means that “a combination” in the context of the present invention does in particular not include the start of a therapy with one component (the STING agonist or the complex) after the therapy with the other component (the other of STING agonist or complex) is finished. Thereby, a “finished” therapy means in particular that the active component does not exert its effects anymore—i.e. a “therapy” may in particular be finished several minutes, hours or days after the last administration of the active component, depending on how long the active component exerts its effects. In more general, an “intertwined” treatment schedule of the STING agonist and the complex—and, thus, a combination of the STING agonist and the complex—means that
(i) not every administration of the STING agonist (and therefore the complete STING agonist therapy) is completed for more than one week (preferably for more than 3 days, more preferably for more than 2 days, even more preferably for more than a day) before the first administration of the complex (and therefore the complete therapy with the complex) starts; or
(ii) not every administration of the complex (and therefore the complete therapy with the complex) is completed for more than one week (preferably for more than 3 days, more preferably for more than 2 days, even more preferably for more than a day) before the first administration of the STING agonist (and therefore the complete STING agonist therapy) starts.

For example, in the combination of the STING agonist as described herein and of the complex comprising a cell penetrating peptide, at least one antigen or antigenic epitope and at least one TLR peptide agonist as described herein, one component (the STING agonist or the complex) may be administered once a week and the other component (the other of STING agonist or complex) may be administered once a month. To achieve in this example “a combination” in the sense of the present invention the monthly administered component is to be administered at least once in the same week, in which also the weekly administered other component is administered.

As outlined above, the administration of the STING agonist and/or of the complex comprised by the combination according to the present invention may require multiple successive administrations, e.g. multiple injections. Thus, the administration may be repeated at least two times, for example once as primary immunization injections and, later, as booster injections.

In particular, the STING agonist and/or the complex comprised by the combination according to the present invention may be administered repeatedly (or continuously). The STING agonist and/or the complex comprised by the combination according to the present invention may be administered repeatedly or continuously for a period of at least 1, 2, 3, or 4 weeks; 2, 3, 4, 5, 6, 8, 10, or 12 months; or 2, 3, 4, or 5 years. For example, the STING agonist comprised by the combination according to the present invention may be administered twice per day, once per day, every two days, every three days, once per week, every two weeks, every three weeks, once per month or every two months. For example, the complex comprised by the combination according to the present invention may be administered twice per day, once per day, every two days, every three days, once per week, every two weeks, every three weeks, once per month or every two months. Preferably, the complex and/or the STING agonist comprised by the combination according to the present invention may be administered repeatedly, for example once per week or (once) every two weeks.

Preferably, the STING agonist and/or the complex comprised by the combination according to the present invention may be administered at the same day. For example, the STING agonist and/or the complex comprised by the combination according to the present invention may be administered repeatedly (as described above; e.g., weekly) and at those days, at which the complex is administered, also the STING agonist is administered. Moreover, on such days of combined administration, also an optional third component may be administered.

In the combination of the STING agonist as described herein and of the complex comprising a cell penetrating peptide, at least one antigen or antigenic epitope and at least one TLR peptide agonist as described herein according to the present invention, the STING agonist and the complex are preferably administered at about the same time.

“At about the same time”, as used herein, means in particular simultaneous administration or that directly after administration of the STING agonist the complex is administered or directly after administration of the complex the STING agonist is administered. The skilled person understands that “directly after” includes the time necessary to prepare the second administration—in particular the time necessary for exposing and disinfecting the location for the second administration as well as appropriate preparation of the “administration device” (e.g., syringe, pump, etc.). Simultaneous administration also includes if the periods of administration of the STING agonist and of the complex overlap or if, for example, one component (STING agonist or complex) is administered over a longer period of time, such as 30 min, 1 h, 2 h or even more, e.g. by infusion, and the other component (STING agonist or complex) is administered at some time during such a long period. Administration of the STING agonist and of the complex at about the same time is in particular preferred if different formulations, different routes of administration and/or different administration sites are used.

Preferably, the STING agonist comprised by the combination according to the present invention and the complex comprised by the combination according to the present invention (as well as an optional further active compound) are administered in a therapeutically effective amount. A “therapeutically effective amount”, as used herein, is the amount which is sufficient for the alleviation of the symptoms of the disease or condition being treated and/or for prophylaxis of the symptoms of the disease or condition being prevented. In other words, a “therapeutically effective amount” means an amount of the complex and/or of the STING agonist that is sufficient to significantly induce a positive modification of a disease or disorder, i.e. an amount of the complex and/or of the STING agonist, that elicits the biological or medicinal response in a tissue, system, animal or human that is being sought. The term also includes the amount of the complex and/or of the STING agonist sufficient to reduce the progression of the disease, notably to reduce or inhibit the tumor growth or infection and thereby elicit the response being sought, in particular such response could be an immune response directed against the antigens or antigenic epitopes comprised in by the complex (i.e. an “inhibition effective amount”). At the same time, however, a “therapeutically effective amount” is preferably small enough to avoid serious side-effects, that is to say to permit a sensible relationship between advantage and risk. The determination of these limits typically lies within the scope of sensible medical judgment. A “therapeutically effective amount” of the complex and/or of the STING agonist, will furthermore vary in connection with the particular condition to be treated and also with the age and physical condition of the patient to be treated, the body weight, general health, sex, diet, time of administration, rate of excretion, drug combination, the activity of the specific components (STING agonist and complex), the severity of the condition, the duration of the treatment, the nature of the accompanying therapy, of the particular pharmaceutically acceptable carrier used, and similar factors, within the knowledge and experience of the accompanying doctor.

The dosage administered, as single or multiple doses, to an individual will thus vary depending upon a variety of factors, including pharmacokinetic properties, subject conditions and characteristics (sex, age, body weight, health, size), extent of symptoms, concurrent treatments, frequency of treatment and the effect desired.

The complex comprised by the combination according to the present invention and the STING agonist comprised by the combination according to the present invention (as well as an optional further active compound) can be administered by various routes of administration, for example, systemically or locally (e.g. intratumorally). Routes for systemic administration in general include, for example, transdermal, oral and parenteral routes, which include subcutaneous, intravenous, intramuscular, intraarterial, intradermal and intraperitoneal routes and/or intranasal administration routes. Routes for local administration include, for example, administration at the site of affliction, such as intratumoral administration.

Preferably, the complex comprised by the combination according to the present invention and the STING agonist comprised by the combination according to the present invention are administered systemically. In some embodiments, the STING agonist is administered intratumorally and the complex is administered systemically.

For systemic administration, parenteral routes of administration are preferred. More preferably, the complex comprised by the combination according to the present invention and the STING agonist comprised by the combination according to the present invention are administered via intravenous, intradermal, subcutaneous, intramuscular, intranasal, or intranodal route. Even more preferably, the complex comprised by the combination according to the present invention and the STING agonist comprised by the combination according to the present invention are administered intravenously or subcutaneously. In some embodiments, the complex comprised by the combination according to the present invention and the STING agonist comprised by the combination according to the present invention are administered intramuscularly.

Preferably, the complex comprised by the combination according to the present invention and the STING agonist comprised by the combination according to the present invention are administered via the same route of administration, preferably via the same systemic route of administration, more preferably via the same parenteral route of administration, even more preferably intravenously or subcutaneously.

In some embodiments, the complex comprised by the combination according to the present invention and the STING agonist comprised by the combination according to the present invention are administered via distinct routes of administration. For example, the STING agonist is administered intratumorally and the complex is administered systemically, e.g. intramuscularly or subcutaneously. In some embodiments, the complex comprised by the combination according to the present invention and the STING agonist comprised by the combination according to the present invention are administered via distinct systemic routes of administration. For example, the STING agonist may be administered intravenously and the complex is administered systemically, e.g. intramuscularly or subcutaneously.

### Compositions

As described above, (i) the STING agonist and (ii) the complex may be provided in distinct compositions. In some embodiments, (i) the STING agonist and (iii) an optional third component (other than the complex and the STING agonist) may be provided in distinct compositions. In some embodiments, (ii) the complex and (iii) an optional third component (other than the complex and the STING agonist) may be provided in distinct compositions. For example, (i) the STING agonist; (ii) the complex and (iii) an optional third component (other than the complex and the STING agonist) may be provided in distinct compositions.

In some embodiments, (i) the STING agonist and (ii) the complex may be comprised in the same composition. In some embodiments, (i) the STING agonist and (iii) an optional third component (other than the complex and the STING agonist) may be comprised in the same composition. In some embodiments, (ii) the complex and (iii) an optional third component (other than the complex and the STING agonist) may be comprised in the same composition. For example, (i) the STING agonist; (ii) the complex and (iii) an optional third component (other than the complex and the STING agonist) may be comprised in the same composition.

Accordingly, the present invention also provides a combination of compositions, wherein a first composition comprises the STING agonist as described above (but preferably not the complex as described above); and a second composition comprises the complex as described above (but preferably not the STING agonist as described above). Each of those compositions may optionally comprise an optional third component (other than the complex and the STING agonist). However, in some embodiments, neither the composition comprising the STING agonist nor the composition comprising the complex further comprises the optional third component (other than the complex and the STING agonist). In such cases, the optional third component (other than the complex and the STING agonist) may be comprised in a further distinct composition.

Moreover, the present invention also provides a composition comprising the STING agonist as described above and the complex as described above. Such a composition may optionally further comprise an optional third component (other than the complex and the STING agonist). However, in some embodiments, the optional third component (other than the complex and the STING agonist) may be comprised in a further distinct composition.

Accordingly, in a further aspect, the present invention also provides a composition comprising

(i) a STING agonist and
(ii) a complex comprising:
a) a cell penetrating peptide;
b) at least one antigen or antigenic epitope; and
c) a TLR peptide agonist,
   wherein the components a)-c) comprised by the complex are covalently linked.

In particular, such a composition according to the present invention comprises (i) the STING agonist as described above and (ii) the complex as described above. In other words, preferred embodiments of the STING agonist as described above (in the context of the combination according to the present invention) are also preferred in the composition according to the present invention. Accordingly, preferred embodiments of the complex as described above (in the context of the combination according to the present invention) are also preferred in the composition according to the present invention.

In general, the composition may be a pharmaceutical composition and/or a vaccine composition. In particular, such a composition is preferably a (pharmaceutical) composition which optionally comprises a pharmaceutically acceptable carrier and/or vehicle, or any excipient, buffer, stabilizer or other materials well known to those skilled in the art. The terms “pharmaceutical formulation” and “pharmaceutical composition” as used in the context of the present invention refer in particular to preparations which are in such a form as to permit biological activity of the active ingredient(s) to be unequivocally effective and which contain no additional component which would be toxic to subjects to which the said formulation would be administered. In some embodiments, the (pharmaceutical) composition does not contain a further active component (e.g., “active” regarding cancer treatment) in addition to the STING agonist and/or the complex (and/or an optional third component (other than the complex and the STING agonist)).

As a further ingredient, the (pharmaceutical) composition may in particular comprise a pharmaceutically acceptable carrier and/or vehicle. In the context of the present invention, a pharmaceutically acceptable carrier typically includes the liquid or non-liquid basis of the (pharmaceutical) composition. If the (pharmaceutical) composition is provided in liquid form, the carrier will typically be pyrogen-free water; isotonic saline or buffered (aqueous) solutions, e.g phosphate, citrate etc. buffered solutions. Particularly for injection of the (pharmaceutical) composition, water or preferably a buffer, more preferably an aqueous buffer, may be used, containing a sodium salt, preferably at least 30 mM of a sodium salt, a calcium salt, preferably at least 0.05 mM of a calcium salt, and optionally a potassium salt, preferably at least 1 mM of a potassium salt. According to a preferred embodiment, the sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₃, K₂SO₄, and examples of calcium salts include e.g. CaCl₂, CaI₂, CaBr₂, CaCO₃, CaSO₄, Ca(OH)₂. Furthermore, organic anions of the aforementioned cations may be contained in the buffer. According to a more preferred embodiment, the buffer suitable for injection purposes as defined above, may contain salts selected from sodium chloride (NaCl), calcium chloride (CaCl₂) and optionally potassium chloride (KCl), wherein further anions may be present additional to the chlorides. CaCl₂ can also be replaced by another salt like KCl. Typically, the salts in the injection buffer are present in a concentration of at least 30 mM sodium chloride (NaCl), at least 1 mM potassium chloride (KCl) and at least 0.05 mM calcium chloride (CaCl₂)). The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects. Reference media are e.g. liquids occurring in “in vivo” methods, such as blood, lymph, cytosolic liquids, or other body liquids, or e.g. liquids, which may be used as reference media in “in vitro” methods, such as common buffers or liquids. Such common buffers or liquids are known to a skilled person. Saline (0.9% NaCl) and Ringer-Lactate solution are particularly preferred as a liquid basis. In some embodiments, the (pharmaceutical) composition further comprises arginine, such as L-arginine.

However, one or more compatible solid or liquid fillers or diluents or encapsulating compounds may be used as well for the (pharmaceutical) composition, which are suitable for administration to a subject to be treated. The term “compatible” as used herein means that these constituents of the (pharmaceutical) composition are capable of being mixed with the complex as defined above in such a manner that no interaction occurs which would substantially reduce the pharmaceutical effectiveness of the (pharmaceutical) composition under typical use conditions. Pharmaceutically acceptable carriers, fillers and diluents must, of course, have sufficiently high purity and sufficiently low toxicity to make them suitable for administration to a subject to be treated. Some examples of compounds which can be used as pharmaceutically acceptable carriers, fillers or constituents thereof are sugars, such as, for example, lactose, glucose and sucrose; starches, such as, for example, corn starch or potato starch; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

In some embodiments, the (pharmaceutical) composition may be administered by injection or via infusion techniques. Sterile injectable forms of the (pharmaceutical) compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation of the (pharmaceutical) composition.

For parenteral injection, the active ingredient will preferably be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

The (pharmaceutical) composition as described herein may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

The (pharmaceutical) composition may also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the (pharmaceutical) composition may be formulated in a suitable ointment, wherein the active ingredients are suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the (pharmaceutical) composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

In this context, prescription of treatment, e.g. decisions on dosage etc. when using the above (pharmaceutical) composition is typically within the responsibility of general practitioners and other medical doctors, and typically takes account of the disorder to be treated, the condition of the individual patient, the site of delivery, the method of administration and other factors known to practitioners.

Accordingly, the (pharmaceutical) composition typically comprises a therapeutically effective amount of the components of the (pharmaceutical) composition, in particular of the complex and/or of the STING agonist. The (pharmaceutical) composition may be used for human and also for veterinary medical purposes, preferably for human medical purposes, as a (pharmaceutical) composition in general or as a vaccine.

(Pharmaceutical) compositions, in particular vaccine compositions, or formulations may be administered as a pharmaceutical formulation which can contain the complex as described herein and/or the STING agonist as described herein in any form described herein. For example, (pharmaceutical) compositions, in particular vaccine compositions, or formulations may also be administered as a pharmaceutical formulation which can contain antigen presenting cells (e.g., dendritic cells) loaded with the complex as described herein in any form described herein.

The vaccine and/or the composition may also be formulated as (pharmaceutical) compositions and unit dosages thereof, in particular together with a conventionally employed adjuvant, immunomodulatory material, carrier, diluent or excipient as described above and below, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous and intradermal) use by injection or continuous infusion.

In the context of the present invention, in particular in the context of a (pharmaceutical) composition and vaccines, injectable compositions may be based upon injectable sterile saline or phosphate-buffered saline or other injectable carriers known in the art. Such (pharmaceutical) compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Examples of suitable adjuvants and/or immunomodulatory materials in the context of the present invention include MPL® (Corixa), aluminum-based minerals including aluminum compounds (generically called Alum), ASO1-4, MF59, CalciumPhosphate, Liposomes, Iscom, polyinosinic:polycytidylic acid (polyIC), including its stabilized form poly-ICLC (Hiltonol), CpG oligodeoxynucleotides, Granulocyte-macrophage colony-stimulating factor (GM-CSF), lipopolysaccharide (LPS), Montanide, polylactide co-glycolide (PLG), Flagellin, Soap Bark tree saponins (QS21), amino alkyl glucosamide compounds (e.g. RC529), two component antibacterial peptides with synthetic oligodeoxynucleotides (e.g. IC31), Imiquimod, Resiquimod, Immunostimulatory sequences (ISS), monophosphoryl lipid A (MPLA), and Fibroblast-stimulating lipopeptide (FSL1).

Compositions, in particular pharmaceutical compositions and vaccines, may be liquid formulations including, but not limited to, aqueous or oily suspensions, solutions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Preservatives include, but are not limited to, methyl or propyl p-hydroxybenzoate and sorbic acid. Dispersing or wetting agents include but are not limited to poly(ethylene glycol), glycerol, bovine serum albumin, Tween®, Span®.

Compositions, in particular pharmaceutical compositions and vaccines, may also be formulated as a depot preparation, which may be administered by implantation or by intramuscular injection.

Compositions, in particular pharmaceutical compositions and vaccines, may also be solid compositions, which may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, acacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

Compositions, in particular pharmaceutical compositions and vaccines, may also be administered in sustained release forms or from sustained release drug delivery systems.

Moreover, the compositions, in particular pharmaceutical compositions and vaccines, may be adapted for delivery by repeated administration.

Further materials as well as formulation processing techniques and the like, which are useful in the context of compositions, in particular pharmaceutical compositions and vaccines, or in the context of their preparation are known to the skilled artisan.

Preferably, the composition is a vaccine. As used in the context of the present invention, the term “vaccine” refers to a biological preparation that provides innate and/or adaptive immunity, typically to a particular disease, preferably cancer. Thus, a vaccine supports in particular an innate and/or an adaptive immune response of the immune system of a subject to be treated. For example, the antigen or antigenic epitope of the complex as described herein typically leads to or supports an adaptive immune response in the patient to be treated, and the TLR peptide agonist of the complex as described herein may lead to or support an innate immune response.

The vaccine may also comprise a pharmaceutically acceptable carrier, adjuvant, and/or vehicle as defined above for the (pharmaceutical) composition. In the specific context of the vaccine, the choice of a pharmaceutically acceptable carrier is determined in principle by the manner in which the vaccine is administered. The vaccine can be administered, for example, systemically or locally as described above. More preferably, vaccines may be administered by an intravenous, intratumoral, intradermal, subcutaneous, or intramuscular route. The vaccine is therefore preferably formulated in liquid (or sometimes in solid) form. The suitable amount of the vaccine to be administered can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the vaccine is to be administered orally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

The vaccine can additionally contain one or more auxiliary substances in order to further increase its immunogenicity. A synergistic action of the STING agonist and/or the complex as defined above and of an auxiliary substance, which may be optionally contained in the vaccine as described above, is preferably achieved thereby. Depending on the various types of auxiliary substances, various mechanisms can come into consideration in this respect. For example, compounds that permit the maturation of dendritic cells (DCs), for example lipopolysaccharides or TNF-alpha, form a first class of suitable auxiliary substances. In general, it is possible to use as auxiliary substance any agent that influences the immune system in the manner of a “danger signal” (LPS, GP96, etc.) or cytokines, such as GM-CSF, which allow an immune response produced by the STING agonist or the complex to be enhanced and/or influenced in a targeted manner. Particularly preferred auxiliary substances are cytokines, such as monokines, lymphokines, interleukins or chemokines, that further promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta or TNF-alpha, growth factors, such as hGH.

In general, the (pharmaceutical) composition, in particular the vaccine, as described herein may be used in medicine as described for the medical use above. In particular, it may be used in the prevention and/or treatment of diseases or disorders including for example cancers, hematological disorders, infectious diseases, autoimmunity disorders and transplant rejections, whereby cancer is preferred, as described above.

### Kits

In a further aspect, the present invention also provides a kit, in particular a kit of parts, comprising

(i) a STING agonist and
(ii) a complex comprising:
a) a cell penetrating peptide;
b) at least one antigen or antigenic epitope; and
c) a TLR peptide agonist,
   wherein the components a)-c) comprised by the complex are covalently linked.

In particular, such a kit according to the present invention comprises (i) the STING agonist as described above (in the context of the combination according to the present invention) and (ii) the complex as described above (in the context of the combination according to the present invention). In other words, preferred embodiments of the STING agonist as described above (in the context of the combination according to the present invention) are also preferred in the kit according to the present invention. Accordingly, preferred embodiments of the complex as described above (in the context of the combination according to the present invention) are also preferred in the kit according to the present invention.

The various components of the kit may be packaged in one or more containers. The above components may be provided in a lyophilized or dry form or dissolved in a suitable buffer. For example, the kit may comprise a (pharmaceutical) composition comprising the STING agonist as described above and a (pharmaceutical) composition comprising the complex as described above, e.g. with each composition in a separate container.

As described above, (i) the STING agonist and (ii) the complex may be comprised in the same container (e.g., a syringe). In some embodiments, (i) the STING agonist and (iii) an optional third component (other than the complex and the STING agonist) may be comprised in the same container (e.g., a syringe). In some embodiments, (ii) the complex and (iii) an optional third component (other than the complex and the STING agonist) may be comprised in the same container (e.g., a syringe). For example, (i) the STING agonist; (ii) the complex and (iii) an optional third component (other than the complex and the STING agonist) may be comprised in the same container (e.g., a syringe).

In some embodiments, (i) the STING agonist and (ii) the complex may be provided in distinct containers (e.g., distinct syringes). In some embodiments, (i) the STING agonist and (iii) an optional third component (other than the complex and the STING agonist) may be provided in distinct containers (e.g., distinct syringes). In some embodiments, (ii) the complex and (iii) an optional third component (other than the complex and the STING agonist) may be provided in distinct containers (e.g., distinct syringes). For example, (i) the STING agonist; (ii) the complex and (iii) an optional third component (other than the complex and the STING agonist) may be provided in distinct containers (e.g., distinct syringes).

The kit may also comprise additional reagents including, for instance, preservatives, growth media, and/or buffers for storage and/or reconstitution of the above-referenced components, washing solutions, and the like.

In addition, the kit-of-parts according to the present invention may optionally contain instructions of use. Preferably, the kit further comprises a package insert or label with directions to treat a disease as described herein, for example cancer.

Such a kit may preferably be for use in medicine as described herein, in particular for use in the prevention and/or treatment of cancer as described herein.

### BRIEF DESCRIPTION OF THE FIGURES

In the following a brief description of the appended figures will be given. The figures are intended to illustrate the present invention in more detail. However, they are not intended to limit the subject matter of the invention in any way.

Throughout the figure legends, the letter ‘K’ stands for the complex comprising a cell penetrating peptide, at least one antigen or antigenic epitope and a TLR peptide agonist, such as Z13Mad25Anaxa (SEQ ID NO: 55) or ATP128 (SEQ ID NO: 54), as indicated in the respective Examples sections.

FIG. 1A-1M shows for Example 1 that combined administration of a complex comprising a cell penetrating peptide, at least one antigen or antigenic epitope and a TLR peptide agonist (K) with a STING agonist (STINGa) modulate both CD4 and CD8 T cell peripheral responses in tumor-free mice. C57BL/6 mice were treated with two administrations of a complex comprising a cell penetrating peptide, at least one antigen or antigenic epitope and a TLR peptide agonist (K), STING agonist (STINGa) or a combination of the two at two weeks interval. (A) Vaccination schedule. (B) Serum IFN-a level measured 4 and 24 hours post first vaccination. (C) Circulating HPV-E7-specific CD8 T cells measured by multimer staining one week after the second vaccination. Mice were sacrificed one week after the third vaccination and CD8 (D-E) or CD4 (F-K) T cell responses were analyzed by flow cytometry. (D) Frequency of CD8 T cells among splenocytes. (E) Percentage of cytokine-producing PMA-restimulated CD8 T cells. (F) Frequency of CD4 T cells among splenocytes. Frequency of Treg (G), Th17 (H), Th1 (I) and Th2 (J) among splenic CD4 T cells. (K) Ratio of Th1/Th2 splenic CD4 T cells. (L) In vivo cytotoxicity of RAHYNIVTF-specific CD8 T cells as measured by transfer of RAHYNIVTF peptide loaded splenocytes. (M) RAHYNIVTF-specific CD8 T cell TCR avidity as measured by ex vivo ELIspot.

FIG. 2A-2B shows for Example 2 that combined administration of a complex comprising a cell penetrating peptide, at least one antigen or antigenic epitope and a TLR peptide agonist (K) with a STING agonist (STINGa) is well tolerated by tumor bearing mice. (A-B) 10⁵ TC-1 cells were implanted on the back of C57BL/6 mice. When tumors were visible, mice were treated with two administrations of a complex comprising a cell penetrating peptide, at least one antigen or antigenic epitope and a TLR peptide agonist (K), a STING agonist (STINGa) or a combination thereof at one week interval. Mouse temperature (A) and weight (B) were measured at the indicated time points.

FIG. 3A-3B shows for Example 3 the phenotype of circulating HPV-specific CD8 T cells in TC-1 tumor bearing mice. 10⁵ TC-1 cells were implanted on the back of C57BL/6 mice. When tumors were visible, mice were treated with two administrations of a complex comprising a cell penetrating peptide, at least one antigen or antigenic epitope and a TLR peptide agonist (K), STING agonist (STINGa) or a combination of the two at one week interval. One week after the last treatment HPV-specific CD8 T cell responses were analysed in mouse blood. Frequency (A) and number (B) of circulating HPV-specific CD8 T cells as measured by flow cytometry.

FIG. 4A-4D shows for Example 3 the effects of combined administration of a complex comprising a cell penetrating peptide, at least one antigen or antigenic epitope and a TLR peptide agonist (K) with a STING agonist (STINGa) on CD8 T cells. 10⁵ TC-1 cells were implanted on the back of C57BL/6 mice. When tumors were visible, mice were treated with two administrations of the complex comprising a cell penetrating peptide, at least one antigen or antigenic epitope and a TLR peptide agonist (K), STING agonist (STINGa) or a combination of the two at one week interval. One week after the last treatment, mice were sacrificed, tumor harvested, and CD8 T cells presence and phenotype was analyzed by FACS staining. Frequency and total number of total (A-B) and HPV-specific (C-D) CD8 T cells among tumor infiltrating leukocytes are shown.

FIG. 5A-5B shows for Example 3 the functionality of tumor-infiltrating HPV-specific CD8 T cells as monitored by measuring IFNγ, TNFα and degranulating marker CD107α expression after ex vivo stimulation with HPV peptide-loaded bone marrow derived dendritic cells (BMDCs). Tumor infiltrating CD45+ cells were co-cultured ex vivo with HPV peptide-loaded BMDCs for 6 hours. Antigen-specific cytokine production was measured by intracellular staining; representative FACS plots and frequency of cytokine-producing among CD8 T cells are shown.

FIG. 6A-6D shows for Example 3 the intracellular production of Granzyme B (GzB) following brief ex vivo TILs culture in presence of Golgi inhibitor. CD45+ tumor infiltrating cells were cultured ex vivo with Golgi inhibitor for 4 hours. Granzyme B production was monitored by intracellular staining; frequency and total number of granzyme B-producing total (A-B) and HPV-specific (C-D) CD8 T cells are depicted.

FIG. 7A-7B shows for Example 3 the phenotype of circulating HPV-specific CD8 T cells in TC-1 tumor bearing mice, namely, that a very low frequency of cytokine- or GzB-producing splenic HPV-specific CD8 T cells was observed in all the different treatments. To this end, splenocytes were restimulated ex vivo with HPV-derived peptides. Frequency of cytokine-producing (A) and Granzyme B secreting (B) HPV-specific CD8 T cells are shown.

FIG. 8A-8I shows for Example 3 the expression of activation and exhaustion markers by total (A) or HPV-specific (B) CD8 T cells was measured by flow cytometry. Frequency of CD38 (C-D), NKg2 Da (E-F) or TCF-1 (G-H) expression by total (C-E-G) or HPV-specific (D-F-H) CD8 T cells. Co-expression of PD-1, Granzyme B and TCF-1 on HPV-specific CD8 T cells (I).

FIG. 9 shows for Example 3 the phenotype of circulating HPV-specific CD8 T cells in TC-1 tumor bearing mice, namely, the expression of activation/exhaustion markers by circulating HPV-specific CD8 T cells as measured by flow cytometry.

FIG. 10A-10L shows for Example 4 that combined administration of a complex comprising a cell penetrating peptide, at least one antigen or antigenic epitope and a TLR peptide agonist (K) with a STING agonist (STINGa) modulates intra-tumoral CD4 T cells in TC-1 model. 10⁵ TC-1 cells were implanted on the back of C57BL/6 mice. When tumors were visible, mice were treated with 2 administrations of the complex comprising a cell penetrating peptide, at least one antigen or antigenic epitope and a TLR peptide agonist (K), STING agonist (STINGa) or a combination of the two at one week interval. One week post the last treatment, mice were sacrificed, blood and tumor harvested, and CD4 T cells presence and phenotype was analyzed by FACS staining. Frequency and total number of total (A-B) and Treg (C-D) CD4 T cells among tumor infiltrating leukocytes. Ratio between tumor infiltrating CD8 T cells and total (E) or Treg (F) CD4 T cells. (G) Frequency of Treg and non-Treg among tumor infiltrating CD4 T cells. Frequency of Th1 (H), Th2 (I) and Th17 (K) among tumor infiltrating CD4 T cells. (J) ratio between Th1 and Th2 tumor infiltrating CD4 T cells. (L) Tumor infiltrating CD45+ cells were co-cultured ex vivo with HPV peptide-loaded BMDCs for 6 hours. Antigen-specific cytokine production was measured by intracellular staining; frequency of cytokine-producing among CD4 T cells is shown.

FIG. 11A-11H shows for Example 5 that combined administration of a complex comprising a cell penetrating peptide, at least one antigen or antigenic epitope and a TLR peptide agonist (K) with a STING agonist (STINGa) modulates the tumor microenvironment (TME). 10⁵ TC-1 cells were implanted on the back of C57BL/6 mice. When tumors were visible, mice were treated with 2 administrations of the complex comprising a cell penetrating peptide, at least one antigen or antigenic epitope and a TLR peptide agonist (K), STING agonist (STINGa) or a combination of the two at one week interval. One week post the last treatment, mice were sacrificed, tumor harvested, and tumor microenvironment was analyzed by FACS staining. (A) Proportion of different cell populations among CD45+ tumor-infiltrating cells, every circle represent 1% of the CD45+ population. (B) Proportion of different dendritic cell populations. Proportion of type 1 (C) or type 2 (D) tumor associate macrophages (TAM) among CD45+ tumor-infiltrating cells. (E) Ratio between TAM1 and TAM2. Proportion of monocytic myeloid-derived suppressor cells mMDSC (F), granulocytic MDSC (G) and neutrophils (H) among CD45+ tumor-infiltrating cells.

FIG. 12A-12H shows for Example 6 that combined administration of a complex comprising a cell penetrating peptide, at least one antigen or antigenic epitope and a TLR peptide agonist (K) with a STING agonist (STINGa) modulates intra-tumoral expression of PD-L1 and MHC-I. 10⁵ TC-1 cells were implanted on the back of C57BL/6 mice. When tumors were visible, mice were treated with 2 administrations of the complex comprising a cell penetrating peptide, at least one antigen or antigenic epitope and a TLR peptide agonist (K), STING agonist (STINGa) or a combination of the two at one week interval. One week post the last treatment, mice were sacrificed, tumor harvested, and tumor microenvironment was analyzed by FACS staining. Expression level (mean MFI) of PD-L1 on CD45− (A) and CD45+(B). % of PD-L1 among infiltrating TAM1 (C) and TAM2 (D). Expression level of H2-Kb (E) and H2-db (F) on CD45− tumor infiltrating cells. Expression level (G) and frequency (H) of MHC-II^{hi} among CD11b+ cells. A pool of two independent experiment is shown (n=7), Mann-Whitney test *p<0.05, **p<0.01, ***p<0.001.

FIG. 13A-13B shows for Example 7 the antitumoral effect of the combined administration of a complex comprising a cell penetrating peptide, at least one antigen or antigenic epitope and a TLR peptide agonist (K) with a STING agonist (STINGa). 10⁵ TC-1 cells (A-B) were implanted on the back of C57BL/6 mice. When tumors were visible, mice were treated twice at one week interval and tumor growth (A) and mouse survival (B) were monitored.

FIG. 14A-14B shows for Example 8 the anti-tumoral effect of the combined administration of a complex comprising a cell penetrating peptide, at least one antigen or antigenic epitope and a TLR peptide agonist (K) with a STING agonist (STINGa 2). 10⁵ TC-1 cells were implanted on the back of C57BL/6 mice. When tumors were visible, mice were treated twice at one week interval with the complex comprising a cell penetrating peptide, at least one antigen or antigenic epitope and a TLR peptide agonist (K) and/or at days 6, 10, 13 and 17 with the STING agonist (STINGa 2) administered systemically. Tumor growth (A) and mouse survival (B) were monitored.

FIG. 15A-15B shows for Example 8 that combined administration of a complex comprising a cell penetrating peptide, at least one antigen or antigenic epitope and a TLR peptide agonist (K) with a STING agonist (STINGa 2) modulates CD8 T cell peripheral responses. Circulating HPV-specific CD8 T cells measured by multimer staining one week after the second vaccination are shown as % among CD8+ T cells (A) and number of HPV-specific CD8 T cells/ml blood (B).

FIG. 16A-16C shows for Example 9 the ATP128 immunogenicity tested in a mouse model, and that the combination of STINGa and ATP128 (K) induces a CEA-specific CD8 T cells response. Female C57BL/6J mice were implanted with 5*10⁵ MC38-CEA tumor cells subcutaneously on the back of the mouse. At day 6 and day 13 post tumor implantation, mice were vaccinated with 10 nmoles of ATP128, 25 μg of a STING agonist (ADU-S100) or a combination of the two. Both the ATP128 and the STING agonist were injected subcutaneously at the base of the tail. One week after the second vaccination, mouse blood was collected from the tail vein and the frequency and the total number of CEA-specific CD8 T cells was analyzed by flow cytometry using a custom designed multimer, wherein the specific epitope from CEA in C57BL/6 mice was predicted and designed. ATP128 vaccination elicits CEA-specific CD8 T cells, which can be monitored using a custom dextramer staining (A). Custom CEA-dextramer staining was performed 1 week after the 2nd vaccination. The combination of STINGa and ATP128 induces a CEA-specific CD8 T cells response (B,C).

FIG. 17A-17C shows for Example 10 that combined administration of a complex comprising a cell penetrating peptide, at least one antigen or antigenic epitope and a TLR peptide agonist (K) with a STING agonist (STINGa) enhances functionality of CD8 T and CD4 T cell peripheral responses in tumor-free mice. (A) C57BL/6 mice were treated with two administrations of Z13Mad39Anaxa vaccine, STING agonist or a combination of the two at two weeks interval. One week after the second vaccination, circulating (left) and splenic (right) SIINFEKL-specific CD8 T cells were measured by multimer staining. (B) SIINFEKL-specific CD8 T cell TCR avidity was measured by ex vivo ELIspot (upper graph). Antigen-specific cytokine production by CD8 T cells was measured by intracellular staining after ex vivo stimulation with SIINFEKL peptide (lower graph). (C) Frequency of Treg (FoxP3⁺), Th1 (T-bet⁺), Th2 (GATA-3⁺) among splenic CD4 T cells and Th1/Th2 ratio was measured by flow cytometry one week after the second vaccination. Antigen-specific cytokine production by CD4 T cells was measured by intracellular staining after ex vivo stimulation with ISQAVHAAHAEINEAGR (OVA-CD4) peptide. One representative experiment is shown (n=5), Mann-Whitney test or Two-way ANOVA *p<0.05, **p<0.01, ***p<0.001.

FIG. 18A-18D shows for Example 11 that the combination of a complex comprising a cell penetrating peptide, at least one antigen or antigenic epitope and a TLR peptide agonist (K) with a STING agonist (STINGa) inhibits B16-OVA tumor growth. 10⁵ B16-OVA cells were injected intravenously into C57BL/6 mice. At day 3 and 10 post tumor injection, mice were treated with two administrations of a complex comprising a cell penetrating peptide, at least one antigen or antigenic epitope and a TLR peptide agonist (K), STING agonist (STINGa) or a combination of the two. At day 20, lungs were perfused to eliminate blood, the number of tumor metastasis was counted and lung infiltrating lymphocytes were analysed. (A) Vaccination schedule. (B) Number of metastatic nodules per lung and representative pictures. (C) Frequency of SIINFEKL (OVA)-specific CD8 T cells among tumor infiltrating leukocytes and expression of Granzyme B was measured by flow cytometry. Antigen-specific cytokine production by CD8 T cells was measured by intracellular staining after ex vivo stimulation with SIINFEKL peptide (SEQ ID NO: 57) in presence of Golgi inhibitor. Antigen-specific cytokine production was measured by intracellular staining; frequency of cytokine-producing among CD8 T cells is shown. (D) Frequency of Treg (FoxP3⁺) and Th1/Th2 ratio were measured by flow cytometry. Antigen-specific cytokine production by CD4 T cells was measured by intracellular staining after ex vivo stimulation with ISQAVHAAHAEINEAGR (OVA-CD4) peptide (SEQ ID NO: 59) in presence of Golgi inhibitor. Antigen-specific cytokine production was measured by intracellular staining; frequency of cytokine-producing among CD4 T cells is shown. A pool of two independent experiments (B) or one representative experiment (C-D) are shown (n≥7), Mann-Whitney test *p<0.05, **p<0.01, ***p<0.001.

FIG. 19A-19C shows for Example 11 the phenotype and functionality of peripheral antigen-specific T cells in B16-OVA tumor bearing mice. One week after the last treatment, antigen-specific CD8 T cell responses were analyzed in mouse blood and spleen. (A) Frequency and number of circulating SIINFEKL (OVA)-specific CD8 T cells was measured by flow cytometry. (B) Splenocytes were stimulated ex vivo with (or without for granzyme B) SIINFEKL (OVA) peptide (SEQ ID NO: 57) and cytokine and granzyme B production by CD8 T cells was measured by intracellular staining. (C) Splenocytes were stimulated ex vivo with ISQAVHAAHAEINEAGR (OVA-CD4) peptide (SEQ ID NO: 59) and antigen-specific cytokine production was measured by intracellular staining. One representative experiment is shown (n=7) *p<0.05, **p<0.01, ***p<0.001.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Methods

### Mice

Female C57BL/6J mice were purchased from Charles River Laboratories (L'arbresles, France). All animals used were between 6 and 10 weeks old at the time of experiments. These studies have been reviewed and approved by the institutional and cantonal veterinary authorities in accordance with Swiss Federal law on animal protection.

### Vaccines

Vaccine constructs were designed in-house and produced in E. coli by Genscript. Vaccines were prepared by dilution in vaccine buffer and administered by subcutaneously (s.c.) injection of 10 nmoles in 100 μl volume. Z13Mad25Anaxa (SEQ ID NO: 55) contains CD4 and CD8 epitopes issued from HPV-16 and was utilized in TC-1 tumor model. ATP128 (SEQ ID NO: 54) contains CEA, survivin and ASCL2 epitopes and was utilized in a MC-38 CEA tumor model.

### STING Agonist

The following STING agonists were used: ADU-S100 (Aduro; also referred to as “STINGa”) in Examples 1-7 and 9; and a distinct STING agonist (referred to as “STINGa 2”) in Example 8.

STINGa (ADU-S100) has the following structural formula (III):

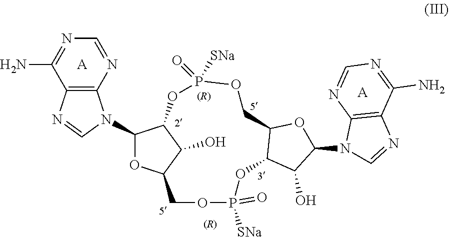

STINGa 2 has the following structural formula Ia.2:

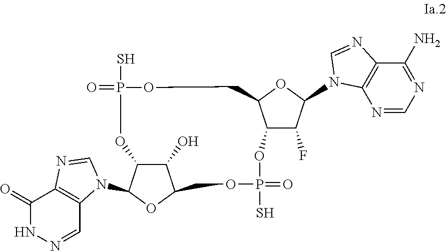 [SMILES: Nc1ncnc2c1ncn2[C@@H]1O[C@@H]2COP(=O)(S)O[C@@H]3[C@H](O)[C@@H](COP(=O)(S)O[C@H]2[C@H]1F)O[C@H]3n1cnc2c(=O)[nH]ncc21]

ADU-S100 (Aduro) was resuspended in DMSO and diluted in 1× phosphate buffer saline (PBS, Gibco) prior to injection. STINGa 2 was resuspended in 1× phosphate buffer saline (PBS, Gibco) prior to injection.

### Cell Lines

TC-1 cells, a cell line derived from lung epithelial cells transfected with HPV16 E6/E7 and c-H-ras oncogenes, were maintained in RPMI 1640 Glutamax™ supplemented with 10% heat-inactivated fetal calf serum (FCS), 100 U/ml Penicillin/Streptomycin (P/S), 1 mM Sodium Pyruvate, MEM NEAA and 0.4 mg/ml geneticin G418.

The MC-38 C57BL/6 mouse colon adenocarcinoma cell line has been transduced with a retroviral construct containing cDNA encoding the human carcinoembryonic antigen (CEA) gene. The CEA expressed by the MC-38-cea-1 clone had a molecular mass of 180 kDa, similar to that of native CEA. This MC-38-cea-1 clone, used here, expresses high levels of CEA on their cell surface (Hand et al. 1993, Cancer Immunol Immunother. 36:65-75). MC-38-CEA-1 cells were cultured in DMEM Medium 1640 Glutamax (Life Technology) with 10% high inactivated fetal calf serum (Life technologies) using standard laboratory techniques (MC-38-CEA-1 culture for tumor implantation_190115).

The B16-OVA cell line was provided by Bertrand Huard, University of Grenoble-Alpes, France). This cell line derived from mouse melanoma cells transfected with OVA, was maintained in RPMI 1640 Glutamax™ supplemented with 10% heat-inactivated fetal calf serum (FCS), 100 U/ml Penicillin/Streptomycin (P/S), 1 mM Sodium Pyruvate, MEM NEAA and 1 mg/ml geneticin G418.

### In Vivo Tumor Experiments

C57BL/6 mice were implanted s.c. with 1×10⁵ TC-1 tumor cells in the back and mice were stratified according to tumor size on day 6 tumor implantation. Alternatively, C57BL/6J mice were injected i.v. with 1×10⁵ B16-OVA cells. Mice were vaccinated two times (at day 6 and 13 post tumor implantation) by s.c. injection of 10 nmoles of vaccine at the tail base. At the same time of vaccination, mice received 25 μg of STING agonist administered via 2×50 μl s.c. injections in each side of the low back. Alternatively, at the same time of vaccination, mice received 10 μg of STING agonist 2 administered via 2×100 μl s.c. injections in each side of the low back.

Alternatively, female C57BL/6J mice were implanted with 5×10⁵ MC38-CEA tumor cells subcutaneously on the back of the mouse and vaccinated twice at the base of the tail (at day 6 and 13 post tumor implantation) by s.c. injection of 10 nmoles of vaccine, 25 μg of STINGa (ADU-S100) or a combination of the two.

Tumor size was measured with a caliper and mice were euthanized when tumor reached a volume of 1000 mm³. Tumor volume was calculated with the following formula:

V=length×length×width×Pi/6

B16-OVA tumor bearing mice were sacrificed at day 20, lungs were perfused with a saline solution and the number of lung metastasis was counted.

### Cell Preparation

Bone marrow derived DCs (BMDCs) were prepared from C57BL/6 mice by extracting bone marrow from tibias and femurs and culturing DCs in BMDC medium (DMEM Glutamax supplemented with 10% FCS, 100 U/ml P/S, 50 μM β-Mercaptoethanol, 10 mM HEPES, 0.116 mg/ml of L-Arginine, MEM NEAA and 10 ng/ml of GM-CSF). After 3 days at 37° C., 5% CO₂, half a volume of fresh medium was added. At day 6, floating cells were recovered, resuspended in BMDCs medium and cultured separately. BMDCs were harvested at day 9 and used for ex vivo T cells stimulation.

TC-1 tumors were harvested at day 20 post implantation and tumor-infiltrating leucocytes (TIL) were purified using Miltenyi tumor dissociation kit following manufacturer instruction. Briefly, tumor tissues were cut into small pieces, and resuspended in DMEM medium containing tumor dissociating enzymes (Miltenyi). Tumors were digested on a Gentle MACS with heating system (Miltenyi) using solid tumor program. Enzymatic digestion was stopped by adding cold PBS 0.5% BSA solution and keeping cells on ice. Digested tumors were passed through a 70 μm to eliminate remaining undigested tissue. CD45+ cells were purified using CD45 TIL microbeads (Miltenyi) following manufacturer protocol. Purified CD45+ cells were used for flow cytometry staining or ex vivo T cells stimulation.

B16-OVA tumor bearing mice were perfused with a saline solution to eliminate blood from the lungs before their collection. Lung-infiltrating leucocytes (LILs) were purified using mouse tumor dissociation kit from Miltenyi, following manufacturer instruction.

Peripheral blood and spleen mononuclear cell suspensions from mice were isolated using Ficoll-Paque gradient (GE Healthcare) before flow cytometry analysis, ex vivo stimulation or TCR avidity assay.

### Ex Vivo T Cell Restimulation

TILs, LILs or splenocytes were numerated and 1×10⁵ or 2×10⁶ cells were plated per condition, respectively. Cells were incubated with HPV-CD4, HPV-CD8, OVA-CD8 or OVA-CD4 epitope peptide, with PMA/ionomycin as a positive control or without any stimulant as a negative control, in presence of Golgi stop (BD biosciences) and anti-CD107a for 6 hours. After washing, cells were stained for cells surface antigens and fixable viability dye, then, after fixation and permeabilization according to manufacturer's instructions (BD biosciences), cells were stained for intracellular cytokines.

### In Vivo Cytotoxicity Assay

Naive splenocytes were harvested and incubated for 1.5 h in DMEM complete medium at 37° C. with or without HPV-E7 CD8 epitope peptide (SEQ ID NO: 56). Then, loaded and non-loaded splenocytes were stained with cell tracer violet (CTV) or CFSE (both from ThermoFisher Scientific), respectively, following manufacturer instruction. Splenocytes were then mixed at a 1:1 ratio and a total of 5×10⁶ cells were transferred by intravenous injection into previously vaccinated mice. 20 hours post cell transfer, splenocytes were harvested and the survival of CTV or CFSE stained cells was assessed by flow cytometry. The percentage of antigen-specific killing was calculated with the following formula: % antigen-specific killing=(1-(ratio peptide⁺:peptide⁻ vaccinated/ratio peptide⁺:peptide⁻ naive))*100.

### Ex Vivo TCR Avidity Assay

One week after the second vaccination, spleens were harvested and splenocytes isolated (see above). 1×10⁶ cells/well were seeded in a IFN-γ ELIspot plate and stimulated 0/N with decreasing concentrations of RAHYNIVTF (SEQ ID NO: 56) or SIINFEKL (SEQ ID NO: 57) peptide. ELIspot plates were then revealed following manufacturer instruction and the percentage of maximal response calculated relatively to the highest concentration of stimulating peptide.

### Antibodies and Flow Cytometry

The following antibodies were used: CD45 (30-F11), CD11b (M1/70), KLRG1 (2F1), CD103 (M290), NKg2a (20d5), Ly6C (AL-21), Ly6G (1A8), PD-L1 (MIH5), I-A/I-E (M5/114), CD11c (HL3), PDCA1 (927), CD64 (X54-5/7.1), B220 (RA3-6B2), CD24 (M1/69), CD4 (GK1.5), CD25 (3C7), CD3 (500A2), NKp46 (29A1.4), TNF-α (MP6-XT22), IFN-γ (XMG1.2), H2-Kb (AF6-88.5) and H2-db (28-14-8) were from BD Biosciences; Tim3 (RMT3-23), PD-1 (29F.1A12), CD38 (90), Gr-1 (RB6-8C5), CD206 (C068C2), CD68 (FA-11) were from BioLegend; Ki67 (solA15), FoxP3 (FJK-16s), T-bet (4B10), GATA-3 (TWAJ) and RORγt (AFKJS-9) were from ThermoFisher Scientific; Granzyme B (REA226) was from Miltenyi; CD8 (KT15) was from MBL. Dead cells were stained with LIVE/DEAD Yellow or Aqua fluorescent reactive dye (Life Technologies) and excluded from analyses. Murine MHC-peptide multimers were from Immudex (Copenhagen, Denmark). Cells were analyzed using an Attune NxT flow cytometer (ThermoFisher Scientific) Kaluza (Beckman Coulter) software.

### Quantification of Serum Interferon-α

Blood was collected from mouse tail vein and serum was isolated by centrifugation using Starstedt tubes. The concentration of IFN-α cytokine was measured using commercial ELISA kits according to the manufacturer's recommendations (PBL Assay Science).

### Statistical Analysis

Statistical analyses were performed using Prism software (GraphPad) and considered statistically significant if p<0.05.

### Example 1: Combinations of STING Agonists and Vaccine Complexes Modulate T Cell Responses

In preclinical tumor model and on-going clinical trials, STING agonists are usually administered by intra-tumoral (i.t.) injection in order to inflame the tumor microenvironment (TME). In contrast thereto, in the present experiments systemic administration of the STING agonist in combination with a complex comprising a cell penetrating peptide, at least one antigen or antigenic epitope, and at least one TLR peptide agonist was investigated.

In order to evaluate the immunogenicity of the combination, tumor-free C57BL/6 mice were vaccinated twice at 2 weeks interval (at day 0 and 14) by s.c. injection of 10 nmoles of Z13Mad25Anaxa (an exemplified a complex comprising a cell penetrating peptide, at least one antigen or antigenic epitope, and a TLR peptide agonist, which contains human papilloma virus (HPV)-derived CD4 and CD8 epitopes. At about the same time of vaccination, mice received 25 μg of STING agonist ADU-S100 administered via 2×50 μl s.c. injections in each side of the low back. Serum was collected 4 and 24 hours after the first vaccination and IFN-α concentration was measured by ELISA. Whole blood was collected at day 21 and used for antigen-specific CD8 T cells measurement by multimer flow cytometry staining. At the same time, spleens were harvested and splenocytes used for ex vivo stimulation and intracellular cytokine production was analyzed by flow cytometry. Alternatively, splenocytes were used for TCR avidity assay.

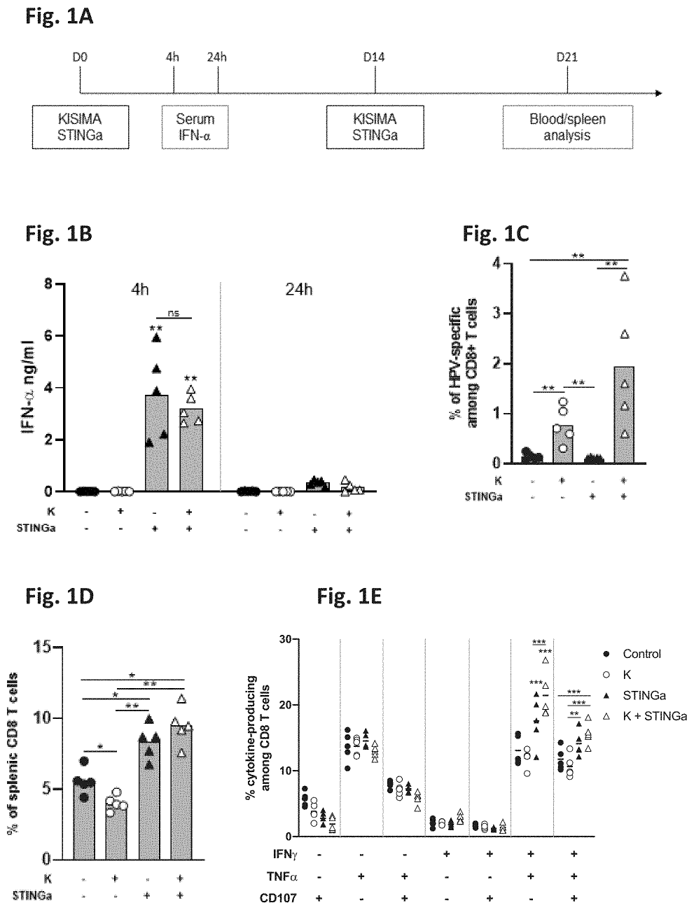

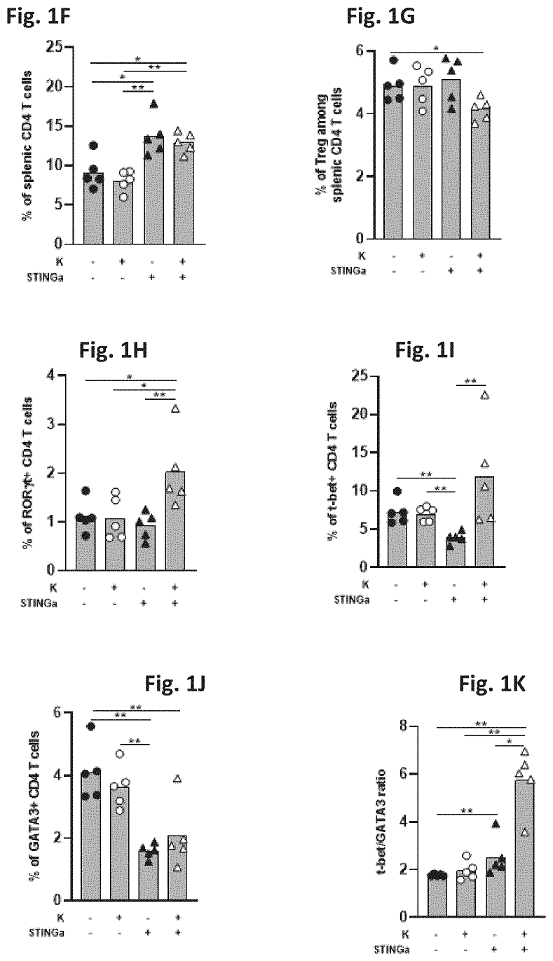

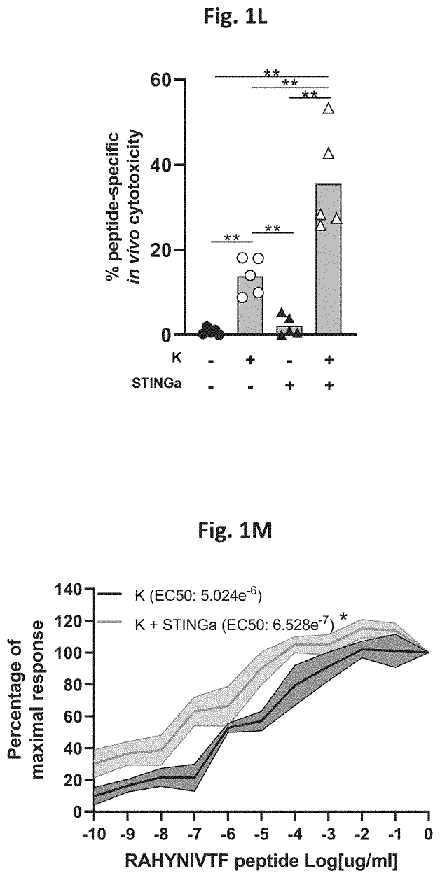

The timeline and results are shown in FIG. 1. FIG. 1A illustrates the timeline of the experiment. Differently from vaccination with the complex comprising a cell penetrating peptide, at least one antigen or antigenic epitope, and a TLR peptide agonist, which induces only local inflammation, systemic treatment with the STINGa induced a potent but short lived systemic type I interferon response, characterized by high IFN-α serum levels peaking 4 hours post injection and decreasing already 24 hours later (FIG. 1B). This systemic response was not affected by concomitant injection of the complex comprising a cell penetrating peptide, at least one antigen or antigenic epitope, and a TLR peptide agonist. While vaccination with Z13Mad25Anaxa is able to elicit circulating HPV-E7-specific CD8 T cells, the data show that combination with STINGa treatment further increases the frequency of antigen-specific CD8 T cells (FIG. 1C). In addition, STINGa treatment resulted in higher proportion of splenic CD8 T cells and increased cytokine secretion after ex vivo PMA/Ionomycin restimulation, indicating a better cell functionality (FIG. 1 D-E). Moreover, combination of a complex comprising a cell penetrating peptide, at least one antigen or antigenic epitope, and a TLR peptide agonist with a STING agonist modulated also CD4 T cells response, slightly increasing splenic proportion and deeply changing their polarization. In fact, a significantly higher proportion of T helper 1 (Th1) and Th17 and lower proportion of Treg and Th2 CD4 T cells was found in combination treated mice, resulting in positive Th1/Th2 and Th17/Th2 ratios (FIG. 1 F-K). Altogether, combination of a complex comprising a cell penetrating peptide, at least one antigen or antigenic epitope, and a TLR peptide agonist with STING agonist improves both CD4 and CD8 T cells response boosting antigen-specific CD8 T cells. In addition to their frequency, combination treatment of a complex comprising a cell penetrating peptide, at least one antigen or antigenic epitope, and a TLR peptide agonist with STINGa also highly enhanced the effector function of antigen-specific CD8 T cells. In vivo killing assay performed one week after vaccination revealed a significant 2.5-fold increase of antigen-specific cytotoxicity in mice treated with a combination of a complex comprising a cell penetrating peptide, at least one antigen or antigenic epitope, and a TLR peptide agonist with STINGa (FIG. 1 L). Furthermore, ex vivo stimulation with decreasing concentration of HPV-CD8 peptide showed significantly higher TCR avidity on complex—STINGa primed T cells (FIG. 1 M).

### Example 2: Safety and Tolerability of Combined Administration of STING Agonists and Vaccine Complexes

Systemic injections of a STING agonist lead to a potent systemic type I interferon response. As this may result in undesired side effects, safety and tolerability of combined administration of STING agonists and vaccine complexes were investigated.

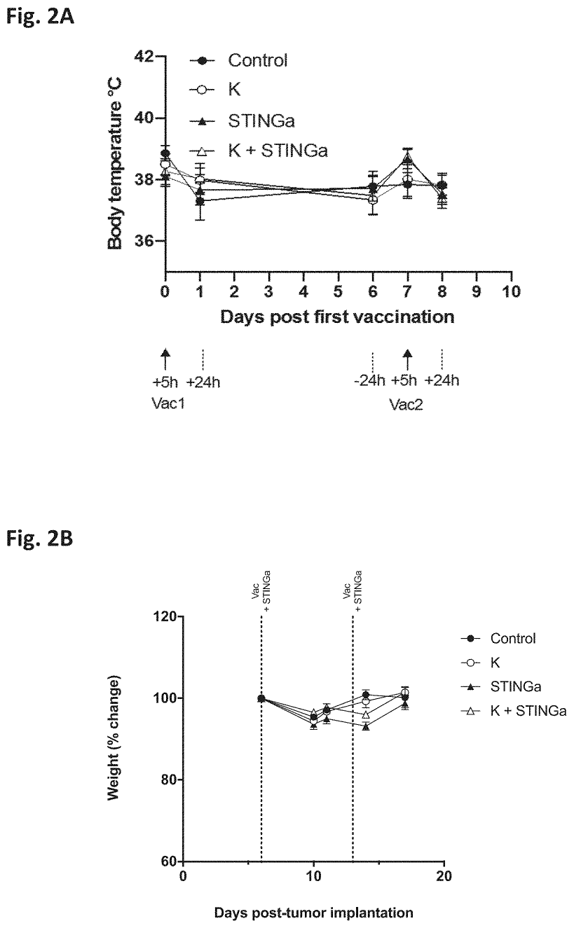

To this end, 10⁵ TC-1 tumor cells were implanted on the back of C57BL/6 mice. When tumors were visible, mice were treated with two administrations of (i) a complex comprising a cell penetrating peptide, at least one antigen or antigenic epitope, and a TLR peptide agonist (Z13Mad25Anaxa); (ii) a STING agonist; or (iii) a combination of both at one week interval, essentially as described in Example 1. Mouse temperature and weight were measured at the time points indicated in FIG. 2.

Results are shown in FIG. 2. Neither single nor combination treatment caused significant variation of body temperature (FIG. 2A) or weight (FIG. 2B) shortly after administration to TC-1 tumor-bearing mice, confirming the safety and tolerability of combined administration of a STING agonist and a complex comprising a cell penetrating peptide, at least one antigen or antigenic epitope, and a TLR peptide agonist.

### Example 3: Combinations of STING Agonists and Vaccine Complexes Improve Antigen-Specific CD8 T Cell Responses in TC-1 Tumor Bearing Mice

TC-1 is a well-known cold tumor model, which is characterized by very low CD4 and CD8 T cells infiltration. To investigate the effects of a combined administration of a STING agonist and a complex comprising a cell penetrating peptide, at least one antigen or antigenic epitope, and a TLR peptide agonist (Z13Mad25Anaxa), antigen-specific CD8 T cell responses were assessed in the TC-1 cold tumor model.

To this end, 10⁵ TC-1 tumor cells were implanted on the back of C57BL/6 mice. When tumors were visible, mice were treated with two administrations of (i) a complex comprising a cell penetrating peptide, at least one antigen or antigenic epitope, and a TLR peptide agonist (Z13Mad25Anaxa); (ii) a STING agonist; or (iii) a combination thereof at one week interval, essentially as described in Example 1. One week after the last treatment, mice were sacrificed, tumor harvested, and CD8 T cells presence and phenotype was analyzed by FACS staining.

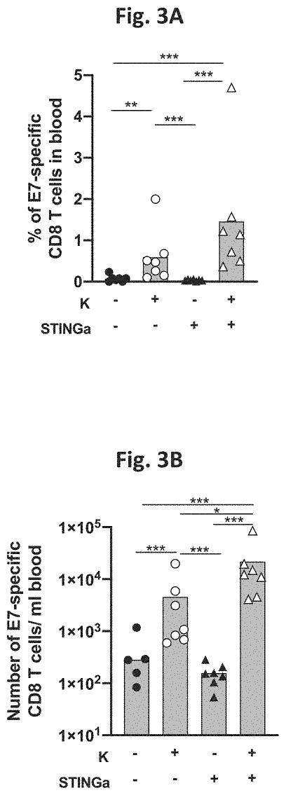

TC-1 tumor cells being lowly immunogenic, very low proportion and number of circulating HPV-specific CD8 T cells were found in vehicle treated mice (FIG. 3). Similarly to the observation in tumor-free mice, vaccination with a complex comprising a cell penetrating peptide, at least one antigen or antigenic epitope, and a TLR peptide agonist significantly increases peripheral HPV-specific response, and while STINGa monotherapy had no effect, combination treatment increases antigen-specific CD8 T cells number.

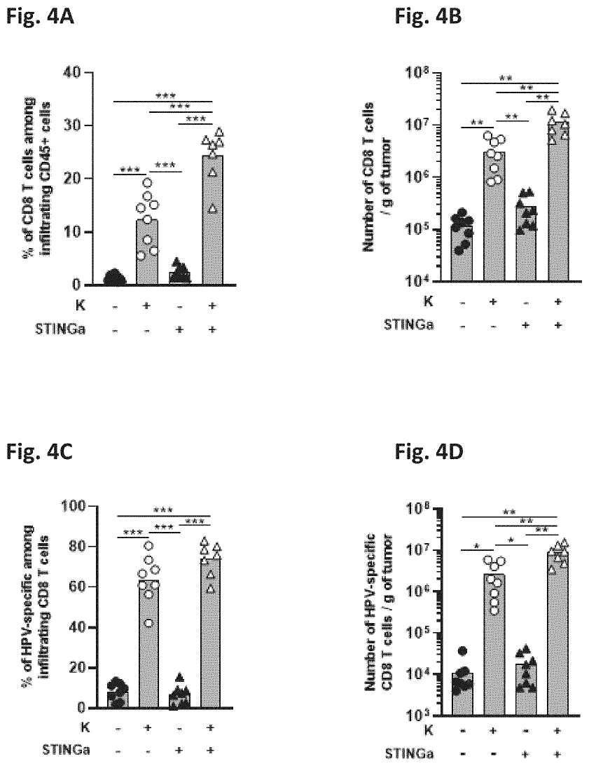

Next, the ability of HPV-specific CD8 T cells to infiltrate TC-1 tumors was investigated. TC-1 being a well-known cold tumor model, very few total or HPV-specific CD8 T cells were found within control tumors, either taking into account proportion—they represent less than 1% of tumor infiltrating leukocytes—or total number (FIG. 4A-D). Vaccination with the complex comprising a cell penetrating peptide, at least one antigen or antigenic epitope, and a TLR peptide agonist induced a significant increase of CD8 T cells tumor infiltration, of which over 50% were HPV-specific. Of note, HPV-specific CD8 T cells are massively present within the tumor despite the rather low percentage in the blood, suggesting that measurement of peripheral responses can only partially predict the intra-tumoral outcome (FIG. 3A-B). STINGa monotherapy did not modulate CD8 T cells tumor infiltration nor the proportion of HPV-specific, thus differing from the observation in tumor-free mouse spleen (FIG. 1). Interestingly, combination of a complex comprising a cell penetrating peptide, at least one antigen or antigenic epitope, and a TLR peptide agonist with a STING agonist showed a synergic effect, increasing both CD8 T cells infiltration and HPV-specific proportion. This confirms that systemic administration of a STING agonist can modulate the intra-tumoral effect of the complex comprising a cell penetrating peptide, at least one antigen or antigenic epitope, and a TLR peptide agonist.

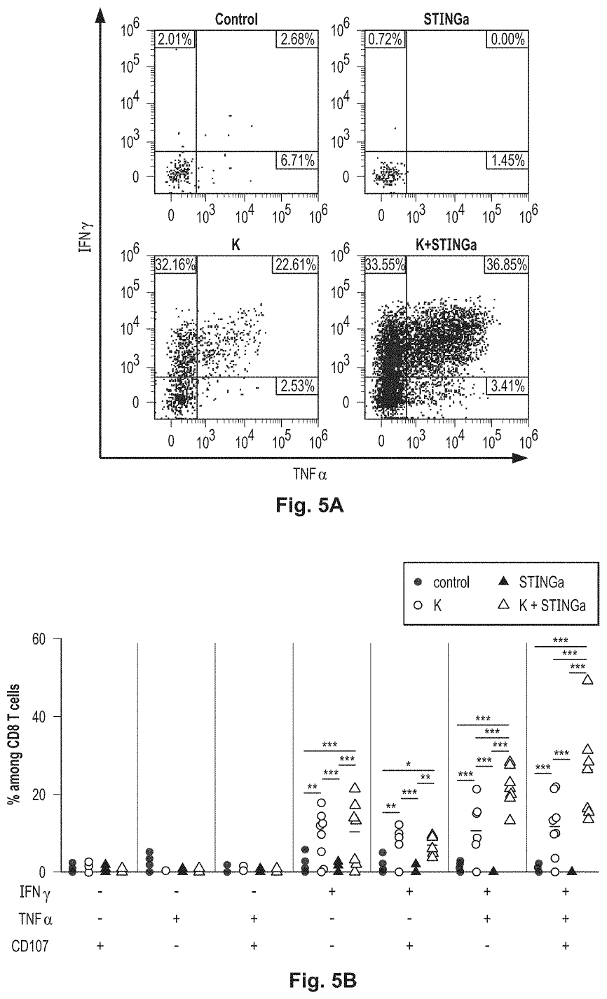

In addition, the functionality of tumor-infiltrating HPV-specific CD8 T cells was monitored by measuring IFNγ, TNFα and degranulating marker CD107α expression after ex vivo stimulation with HPV peptide-loaded bone marrow derived dendritic cells (BMDCs), and a significant increase of HPV-specific cytokine-producing and degranulating CD8 T cells was found in mice treated with the complex comprising a cell penetrating peptide, at least one antigen or antigenic epitope, and a TLR peptide agonist compared to control or STINGa monotherapy group (FIG. 5). Interestingly, vaccination with the complex comprising a cell penetrating peptide, at least one antigen or antigenic epitope, and a TLR peptide agonist particularly increased the proportion of multifunctional CD8 T cells, able to simultaneously produce IFNγ, TNFα and/or CD107α. Combination with STINGa further increased CD8 T cells functionality, and importantly the frequency of multifunctional cells.

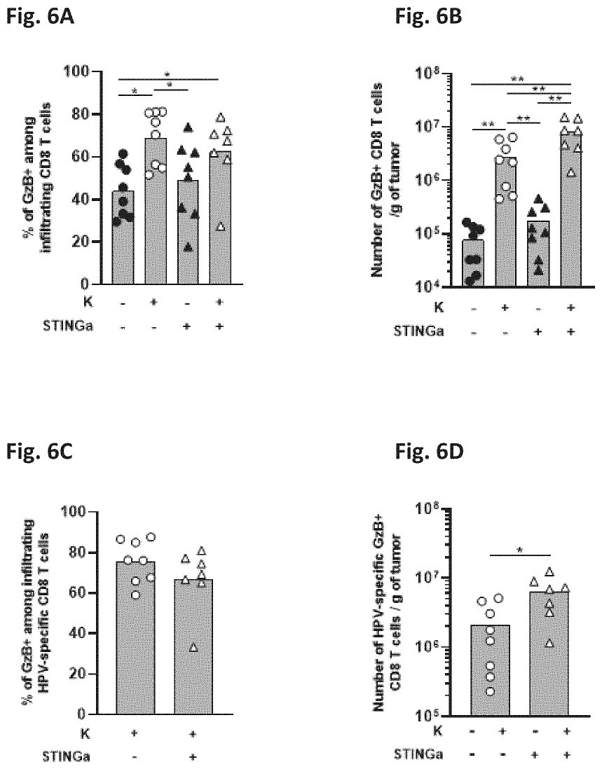

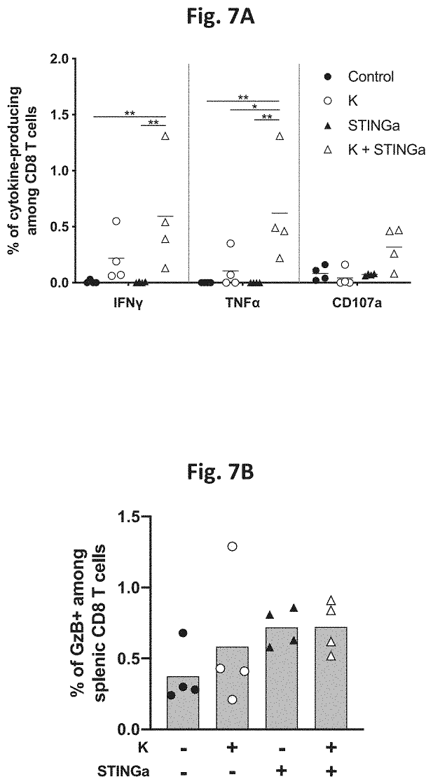

To further characterize tumor infiltrating T cells functionality, also intracellular production of Granzyme B (GzB) was measured, following brief ex vivo TILs culture in presence of Golgi inhibitor, as GzB is one of the main weapons used by CD8 T cells to eliminate cancer cells. Significantly higher frequency and number of GzB-producing total or HPV-specific CD8 T cells were found in mice vaccinated with a complex comprising a cell penetrating peptide, at least one antigen or antigenic epitope, and a TLR peptide agonist, compared to vehicle or STINGa treated (FIG. 6). Despite not changing the frequency of GzB-positive among HPV-specific CD8 T cells, combination with STINGa further increased their total number. Importantly, contrarily to the intra-tumoral compartment, very low frequency of cytokine- or GzB-producing splenic HPV-specific CD8 T cells was observed in all the different treatments (FIG. 7), demonstrating that CD8 T cells are not systemically activated.

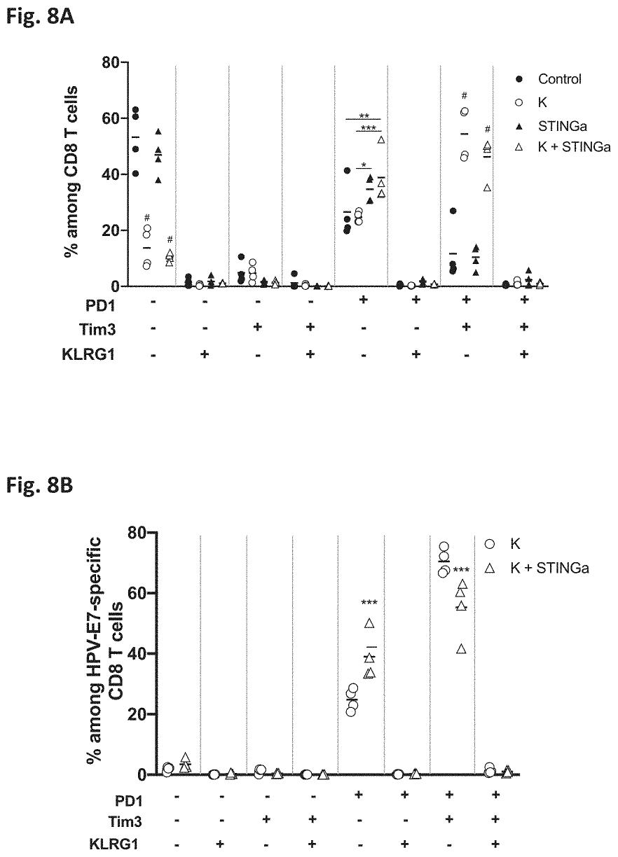

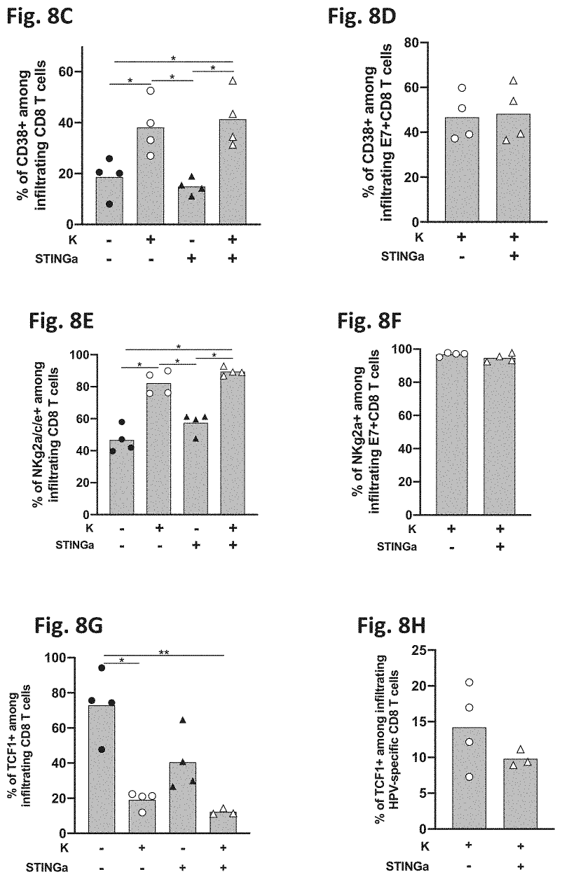

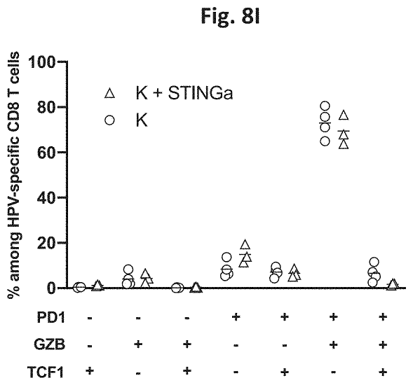

The efficacy of cancer-specific T cells is often limited by tumor induced exhaustion. Therefore, the expression of activation and exhaustion markers on intra-tumoral and peripheral CD8 T cells was analyzed next. T cells exhaustion is a gradual process eventually resulting in a loss of cell functionality, which can be monitored by the progressive expression of exhaustion markers. While most of tumor infiltrating CD8 T cells in control and STINGa single treated mice expressed only PD-1 or no exhaustion marker at all, in mice vaccinated with the complex comprising a cell penetrating peptide, at least one antigen or antigenic epitope, and a TLR peptide agonist or in combination treated mice the majority of CD8 T cells, and in particular HPV-specific cells, expressed exhaustion markers PD-1 and Tim-3 (FIG. 8A-B). Interestingly, in the combination group a lower proportion of CD8 T cells co-express PD-1 and Tim-3, suggesting a less exhausted phenotype, which correlate with the higher proportion of cytokine-secreting cells. In addition, a higher proportion of mice vaccinated with complex comprising a cell penetrating peptide, at least one antigen or antigenic epitope, and a TLR peptide agonist showed CD8 T cells, which also expressed CD38 (FIG. 8C-D), NKg2a (FIG. 8E-F), and TCF-1 (FIG. 8G-H), which are other markers associated with reduced T cell functionality. FIG. 81 shows the co-expression of PD1, Granzyme B and TCF-1 on HPV-specific CD8 T cells.

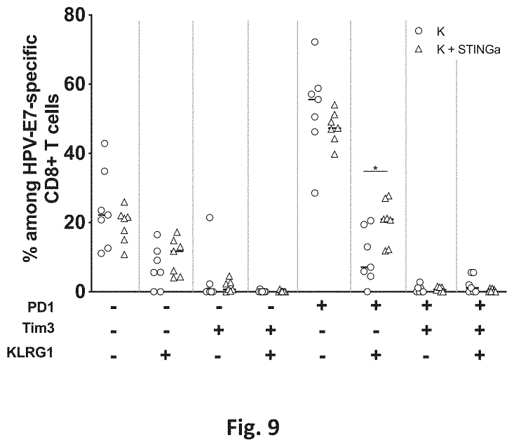

Similarly to functionality analysis, peripheral CD8 T cells showed a less-exhausted phenotype, with the majority of cells that expressed only PD-1 and some cells still expressing the early activation marker KLRG1 (FIG. 9), suggesting that exhaustion is acquired within the TME.

In summary, vaccination with the complex comprising a cell penetrating peptide, at least one antigen or antigenic epitope, and a TLR peptide agonist highly increases HPV-specific CD8 T cells tumor infiltration and functionality, and while STINGa monotherapy has no effect, and combination treatment further enhances the efficacy of the complex comprising a cell penetrating peptide, at least one antigen or antigenic epitope, and a TLR peptide agonist. Nevertheless, intra-tumoral CD8 T cells have a partially exhausted phenotype, which is less advanced in combination treated mice compared to mice vaccinated with the complex comprising a cell penetrating peptide, at least one antigen or antigenic epitope, and a TLR peptide agonist alone.

### Example 4: Combinations of STING Agonists and Vaccine Complexes Modulate Intra-Tumoral CD4 T Cell Responses

Immunotherapy research has widely focused on CD8 T cells, neglecting CD4 T cells, which are often only considered as immune-suppressives due to the regulatory T cells (Treg). However, recent studies highlight the importance of CD4 T cells, in particular the Th1 and Th17 polarized, for the development of a proper anti-tumoral CD8 T cells response (Meissen, M. and C. L. Slingluff, Jr. (2017). “Vaccines targeting helper T cells for cancer immunotherapy.” Curr Opin Immunol 47: 85-92; Muranski, P., et al. (2008). “Tumor-specific Th17-polarized cells eradicate large established melanoma.” Blood 112(2): 362-373).

In view thereof, intra-tumoral CD4 T cells were monitored next. To this end, the TC-1 tumor model, as described in Example 3, was used.

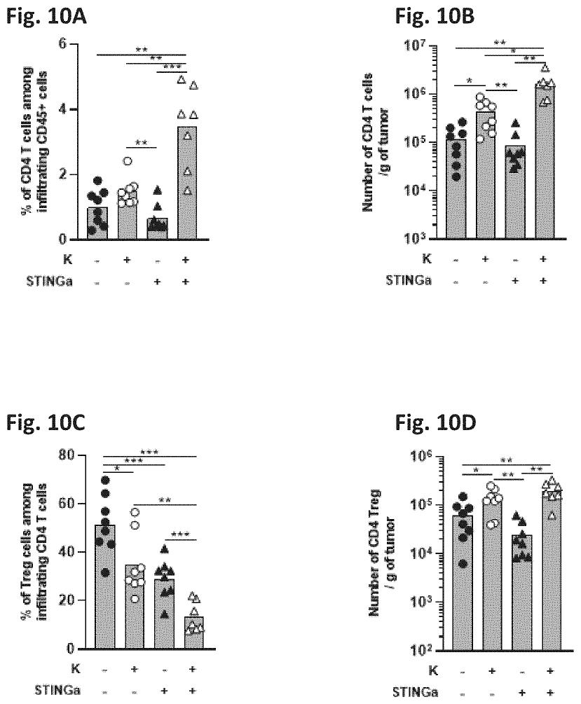

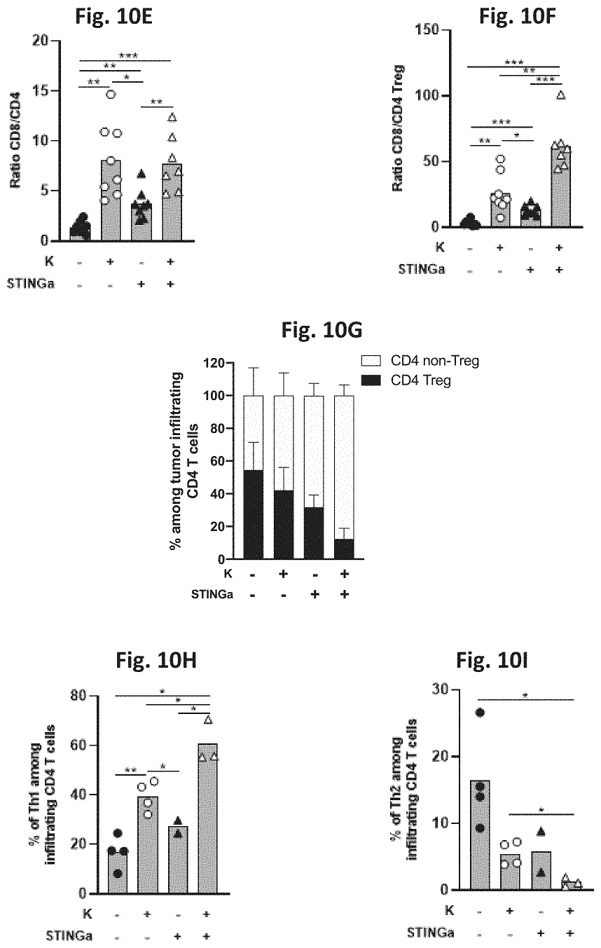

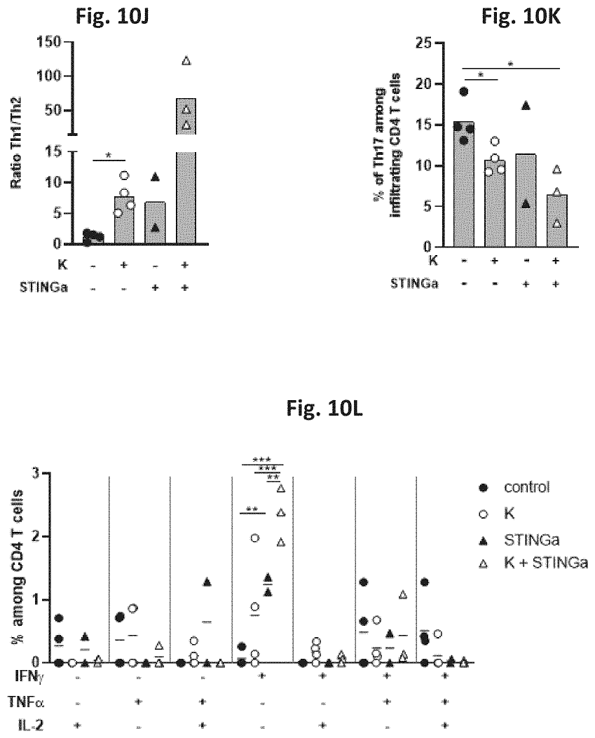

Results are shown in FIG. 10. The data show a significantly increased tumor infiltration by CD4 T cells in mice treated with a combination of complex comprising a cell penetrating peptide, at least one antigen or antigenic epitope, and a TLR peptide agonist (Z13Mad25Anaxa) and a STING agonist (STINGa), as compared to all other groups (FIG. 10A-B). Differently form the observation in the spleen, STINGa monotherapy had no effect on intra-tumoral CD4 T cells recruitment. Interestingly, this increased CD4 T cell infiltration was not led by Treg, as their percentage was highly reduced in combination treated mice, but rather by effector CD4 T cells (FIGS. 10C, D and G). The ratio between intra-tumoral CD8 and total or regulatory CD4 T cells is often used as a predictive value for the immunological state of TME. Vaccination with the complex comprising a cell penetrating peptide, at least one antigen or antigenic epitope, and a TLR peptide agonist induced a higher CD8/CD4 T cells ratio compared to vehicle or STINGa treated groups (FIG. 10E). While combination treatment resulted in a CD8/CD4 T cells ratio similar to vaccination with the complex comprising a cell penetrating peptide, at least one antigen or antigenic epitope, and a TLR peptide agonist, it induced a significantly higher CD8/Treg ratio (FIG. 10F), highlighting a less immunosuppressive TME. Further analysis revealed that in combination treated mice most of intra-tumoral CD4 T cells are T-bet+ Th1, while only a minimal part are GATA-3+Th2 cells, resulting in a positive Th1/Th2 ratio (FIG. 10H-J). Contrarily from the spleen, a slight decrease in intra-tumoral Th17 CD4 T cells was observed in combination treated mice (FIG. 10K). As Th1 CD4 T cells are usually characterized by the production of IFN-γ and TNF-α production of cytokines was measured by flow cytometry after ex vivo restimulation with HPV peptide-loaded BMDCs. However, contrarily to CD8 T cells, neither IFN-γ nor TNF-α production by intra-tumoral CD4 T cells (FIG. 10L) was detected.

### Example 5: Combinations of STING Agonists and Vaccine Complexes Modulate Tumor Microenvironment (TME)

Despite T cells being the principal target of immunotherapy, due to their ability to directly kill cancer cells, the TME is a very complex network constituted by different immune cell types able to promote or inhibit cancer growth. In view thereof, the composition of TME was deeply dissected in order to obtain a complete overview of its immunological status. To this end, the TC-1 tumor model was used, as described above.

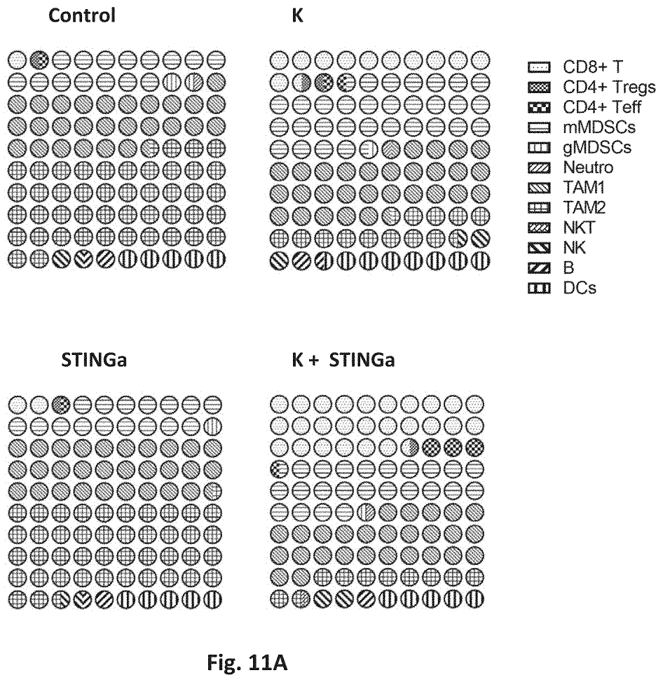

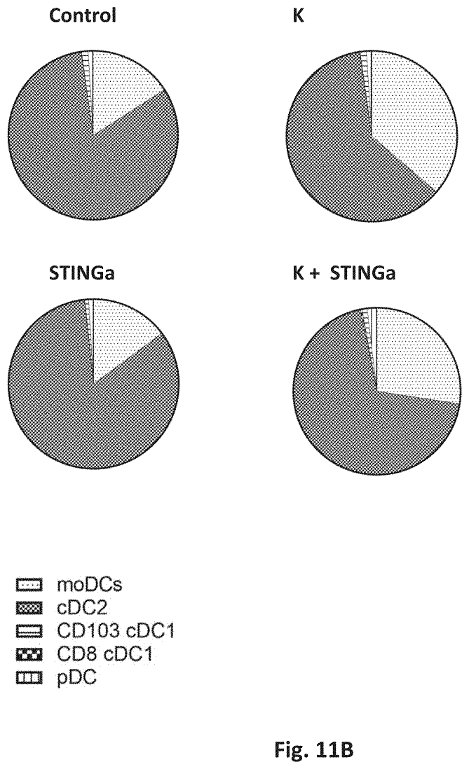

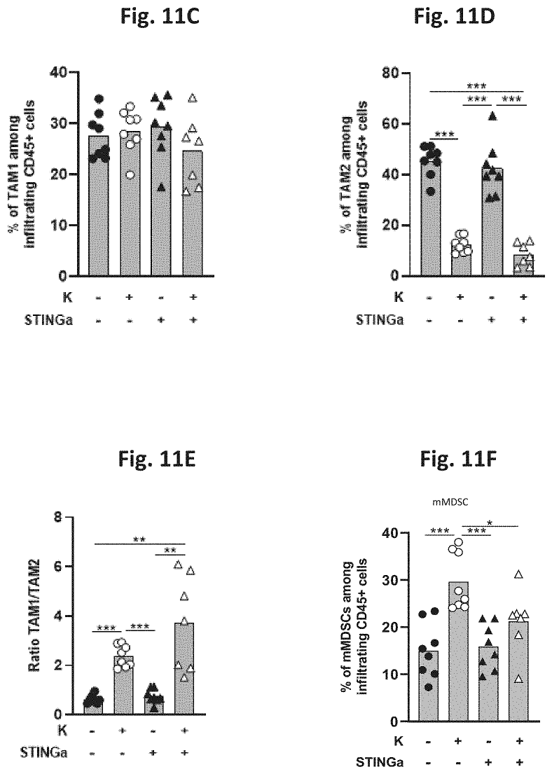

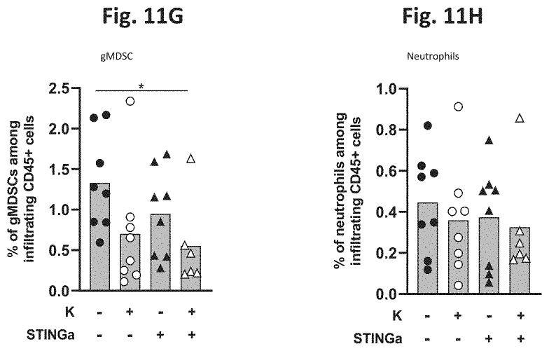

Results are shown in FIG. 11. As previously mentioned, TC-1 is a cold tumor model, characterized by very low CD4 and CD8 T cells infiltration that combined represent less than 2% of tumor infiltrating CD45+ cells in vehicle treated mice (FIG. 11A). The most prominent cell type are tumor associated macrophages (TAM), representing up to 75% of the infiltrate, and in particular the immunosuppressive TAM2, which have been associated to promotion of tumor growth in different cancer types. Myeloid derived suppressor cells (MDSC)—whose role is less clear and have been associated to tumor promotion or control depending on cancer type—represent another 15%, with the monocytic type (mMDSC) being prevalent. Other cell types found with lower frequency were dendritic cells (DCs, 7%), B cells (2%), NK and NKT cells (1.5%) and neutrophils (1%). Vaccination with the complex comprising a cell penetrating peptide, at least one antigen or antigenic epitope, and a TLR peptide agonist induced a profound modification of the TME, characterized by a strong increase in CD8 T cells and DCs frequency and the appearance of non-Treg CD4 T cells. Interestingly, the increase of DCs infiltration is also characterized by an increase of monocytic DCs (moDCs) proportion (FIG. 11B), a particular DC phenotype which has been described to differentiate only in inflammatory conditions and has been shown to activate anti-tumoral T cell responses (Kuhn, S., et al. (2015). “Monocyte-Derived Dendritic Cells Are Essential for CD8(+) T Cell Activation and Antitumor Responses After Local Immunotherapy.” Front Immunol 6: 584). While the TAM1 compartment remains mostly unaltered, TAM2 frequency is strongly decreased resulting in a higher TAM1/TAM2 ratio (FIG. 11C-E). Contrarily, the frequency of mMDSC is increased by vaccination with the complex comprising a cell penetrating peptide, at least one antigen or antigenic epitope, and a TLR peptide agonist, while granulocytic MDSC remains mostly unchanged (FIG. 11F-G). The inverse effect on TAM2 and mMDSC, suggests a possible cell re-polarization, as both population of monocytic origins are known for their plasticity and ability to change differentiation status depending on the environment.

Similarly to the observation on CD8 T cells infiltration and phenotype, systemic administration of STINGa alone does not affect the composition of TME, which is essentially identical to vehicle treated mice. However, in combination treatment, the STING agonist showed a synergic effect with vaccination with the complex comprising a cell penetrating peptide, at least one antigen or antigenic epitope, and a TLR peptide agonist, further expanding CD8 and non-Treg CD4 T cells infiltration by 2.5 fold, while decreasing TAM2 frequency, thus resulting in an even more inflammatory environment.

### Example 6: Combinations of STING Agonists and Vaccine Complexes Modulate Intra-Tumoral Expression of PD-L1 and MHC

The PD-1/PD-L1 axis is the major pathway leading to T cells exhaustion, thus inhibiting the anti-tumoral effect of antigen-specific CD8 T cells, and PD-L1 expression has been shown to be up-regulated on tumoral cells upon intra-tumoral treatment with STING agonist. Furthermore, down-regulation of MHC-I expression on tumor cells is one of the main mechanism of immune evasion. Therefore, the intra-tumoral expression of PD-L1 and MHC-I as well as MHC-II was also monitored in the TC-1 tumor model as described above.

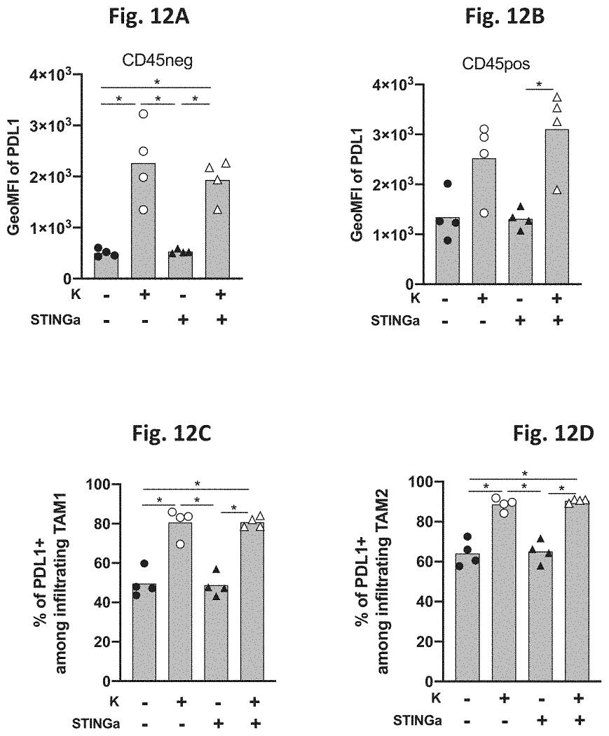

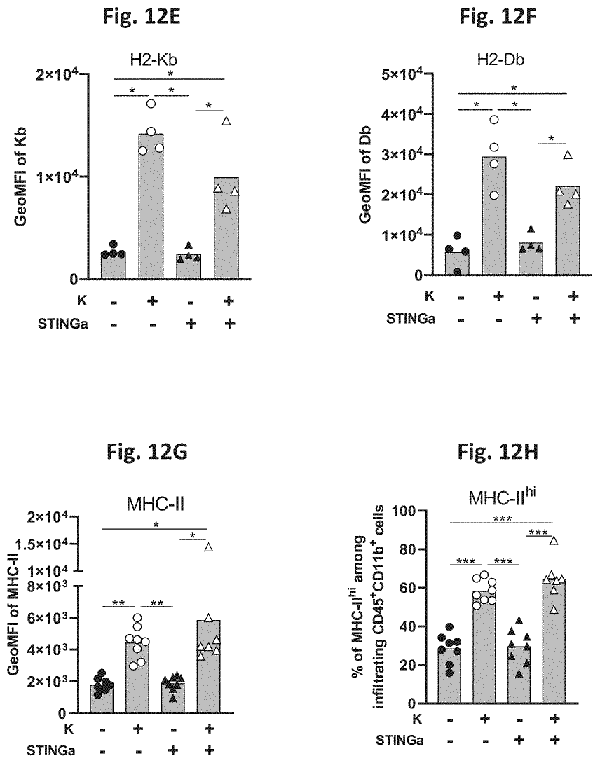

Results are shown in FIG. 12. Increased PD-L1 expression was found upon treatment with the complex (Z13Mad25Anaxa) comprising a cell penetrating peptide, at least one antigen or antigenic epitope, and a TLR peptide agonist alone or in combination with the STINGa, as compared to vehicle or STINGa s.c. monotherapy (FIG. 12A-B). PD-L1 expression was increased on both CD45− and CD45+ cell compartments, highlighting that both, tumoral and immune cells, could promote T cell exhaustion. The main immune cell population to express PD-L1 was identified as TAMs of both types (FIG. 12C-D). Furthermore, with regard to MHC-I expression on tumor cells, both, H2-Kb and H2-db alleles expression, was up-regulated by tumor cells upon treatment with the complex comprising a cell penetrating peptide, at least one antigen or antigenic epitope, and a TLR peptide agonist alone or in combination with the STINGa, as compared to both vehicle and STINGa monotherapy (FIG. 12E-F), excluding this mechanism of immune evasion and suggesting that vaccination with the complex comprising a cell penetrating peptide, at least one antigen or antigenic epitope, and a TLR peptide agonist could even promote epitope presentation by tumor cells. At the same time, vaccination with the complex comprising a cell penetrating peptide, at least one antigen or antigenic epitope, and a TLR peptide agonist alone or in combination with the STINGa also increased MHC-II expression on CD11b+ cells (FIG. 12G-H) as compared to both vehicle and STINGa monotherapy, thus promoting the presentation of epitopes to CD4 T cells.

Altogether, these results highlight the profound modulation of TME induced by vaccination with the complex comprising a cell penetrating peptide, at least one antigen or antigenic epitope, and a TLR peptide agonist, which is able to turn a cold tumor into hot, and the synergistic effect of STING agonist treatment combined with the vaccine complex, which further increases intra-tumoral immunogenicity, although no effect was observed with STING agonist monotherapy.

### Example 7: Antitumoral Effects of the Combination of STING Agonists and Vaccine Complexes

Next, the anti-tumoral effect of therapeutic of the combination of the complex comprising a cell penetrating peptide, at least one antigen or antigenic epitope, and a TLR peptide agonist with a STING agonist was evaluated in the TC-1 tumor model.

10⁵ TC-1 cells were implanted on the back of C57BL/6 mice. When tumors were visible, mice were treated twice at one-week interval as described above in therapeutic settings of the TC-1 tumor model with (i) the complex comprising a cell penetrating peptide, at least one antigen or antigenic epitope, and a TLR peptide agonist (Z13Mad25Anaxa); (ii) the STING agonist; or (iii) a combination of both.

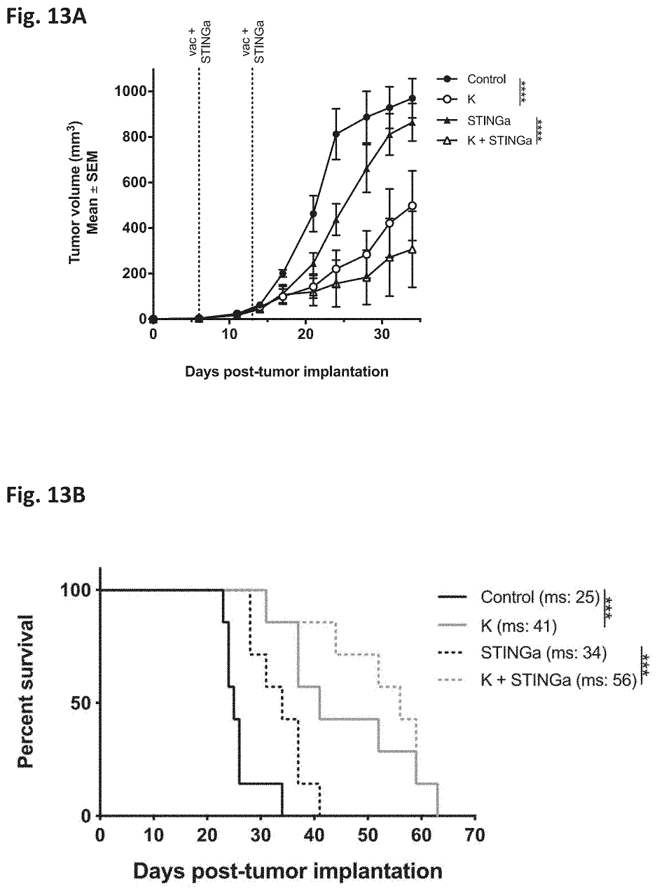

Results are shown in FIG. 13. In the TC-1 model, two vaccinations with the complex comprising a cell penetrating peptide, at least one antigen or antigenic epitope, and a TLR peptide agonist resulted in a significant delay of tumor development and an increased median survival, (FIG. 13A-B). While STINGa monotherapy had only a small effect on tumor growth, its combination with the complex comprising a cell penetrating peptide, at least one antigen or antigenic epitope, and a TLR peptide agonist considerably increased this effect, thereby significantly delaying tumor development and enhancing median survival.

### Example 8: Antitumoral Effects of the Combination of the Vaccine Complex with a Distinct STING Agonist

Next, the anti-tumoral effect of therapeutic of the combination of the complex comprising a cell penetrating peptide, at least one antigen or antigenic epitope, and a TLR peptide (Z13Mad25Anaxa) agonist with a distinct STING agonist was evaluated in the TC-1 tumor model. Instead of STING agonist ADU-S100 (Aduro), as used in the experiments described above, STING agonist STINGa 2 was used.

Similarly as described above, 10⁵ TC-1 cells were implanted on the back of C57BL/6 mice and assigned to distinct groups (control (no treatment); complex comprising a cell penetrating peptide, at least one antigen or antigenic epitope, and a TLR peptide agonist (Z13Mad25Anaxa); STING agonist STINGa 2; and a combination thereof). For the treatment with the complex comprising a cell penetrating peptide, at least one antigen or antigenic epitope, and a TLR peptide agonist (Z13Mad25Anaxa), mice were vaccinated by s.c. injection of 10 nmoles of the complex comprising a cell penetrating peptide, at least one antigen or antigenic epitope, and a TLR peptide agonist (Z13Mad25Anaxa) at day 6 (when tumors were visible) and day 13 post tumor implantation. For the treatment with the STING agonist STINGa 2, mice received 10 μg of STING agonist STINGa 2 administered systemically (s.c.) at days 6, 10, 13 and 17. Whole blood was collected at day 20 and used for antigen-specific CD8 T cells measurement by multimer flow cytometry staining.

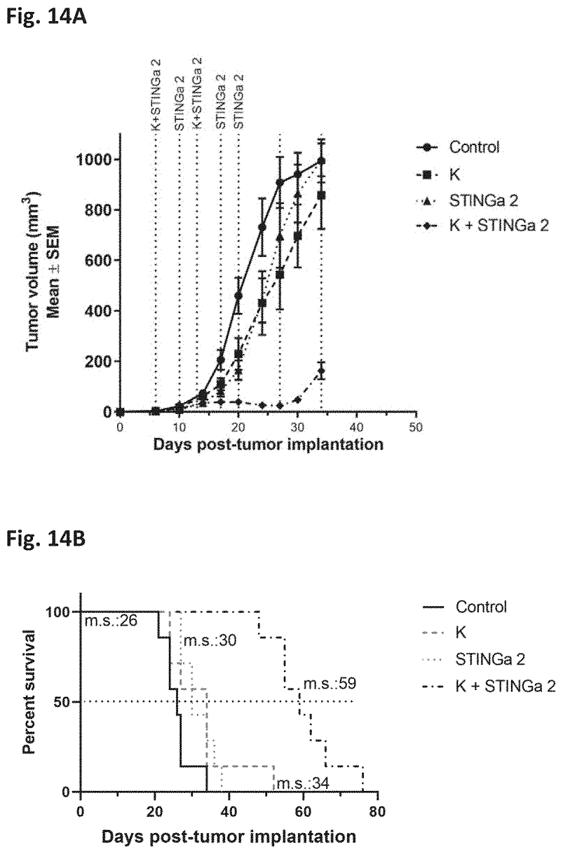

As shown in FIG. 14, monotherapy with the complex comprising a cell penetrating peptide, at least one antigen or antigenic epitope, and a TLR peptide agonist increased survival and reduced tumor growth, while STING agonist monotherapy only provided a slight improvement. However, combination of both showed a synergistic effect with considerably increased survival and reduced tumor growth. This confirms the results described in Example 7 above with a systemic administration of a distinct STING agonist.

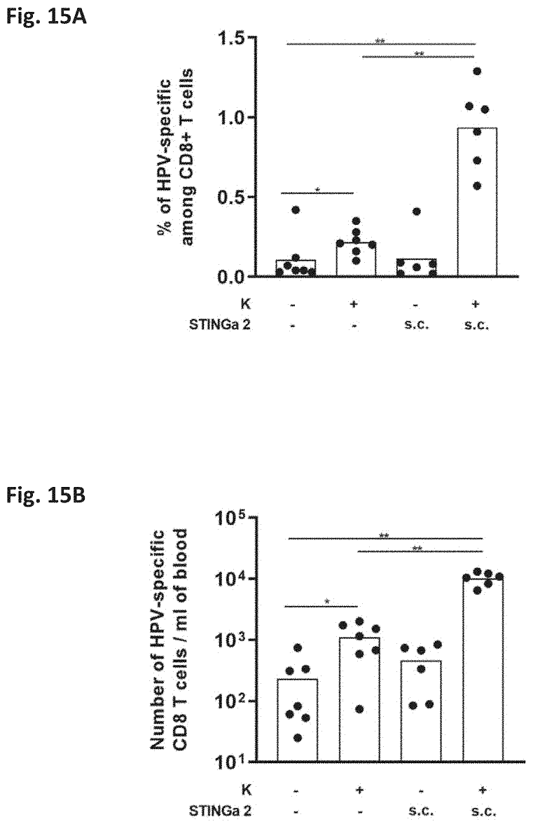

As shown in FIG. 15, vaccination with the complex comprising a cell penetrating peptide, at least one antigen or antigenic epitope, and a TLR peptide agonist is able to elicit circulating HPV-specific CD8 T cells. However, combination with STING agonist treatment further increased the frequency of antigen-specific CD8 T cells, thereby confirming the results described in Example 1 for a distinct STING agonist.

### Example 9: ATP128 Immunogenicity in Mouse; the Combination of STINGa and ATP128 Induces a CEA-Specific CD8 T Cells Response

Female C57BL/6J mice were implanted with 5*10⁵ MC38-CEA tumor cells subcutaneously on the back of the mouse. At day 6 and day 13 post tumor implantation, mice were vaccinated with 10 nmol of ATP128, 25 μg of a STING agonist (ADU-S100) or a combination of the two. Both the ATP128 vaccine and the STING agonist were injected subcutaneously at the base of the tail. One week after the second vaccination, mouse blood was collected from the tail vein and the frequency and the total number of CEA-specific CD8 T cells was analyzed by flow cytometry using a custom designed multimer.

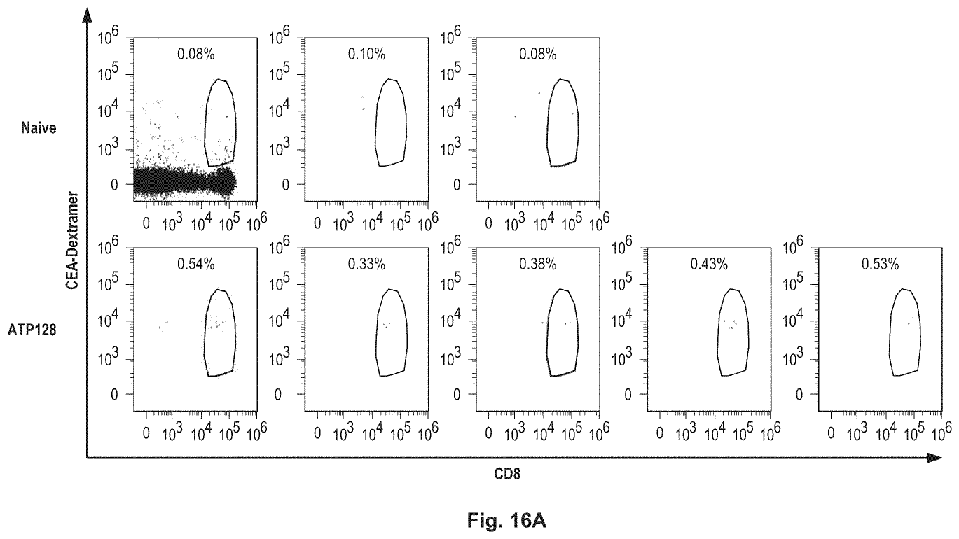

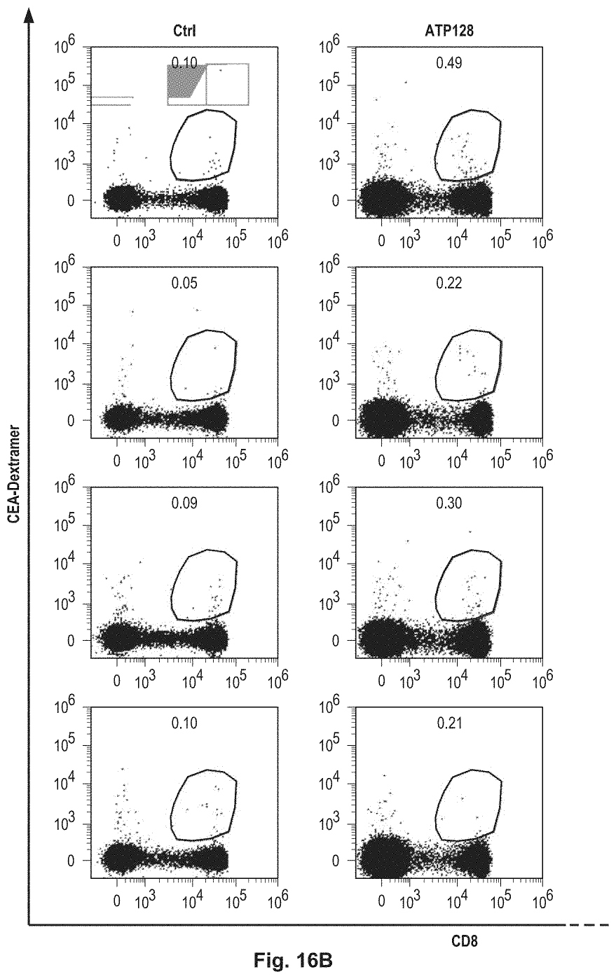

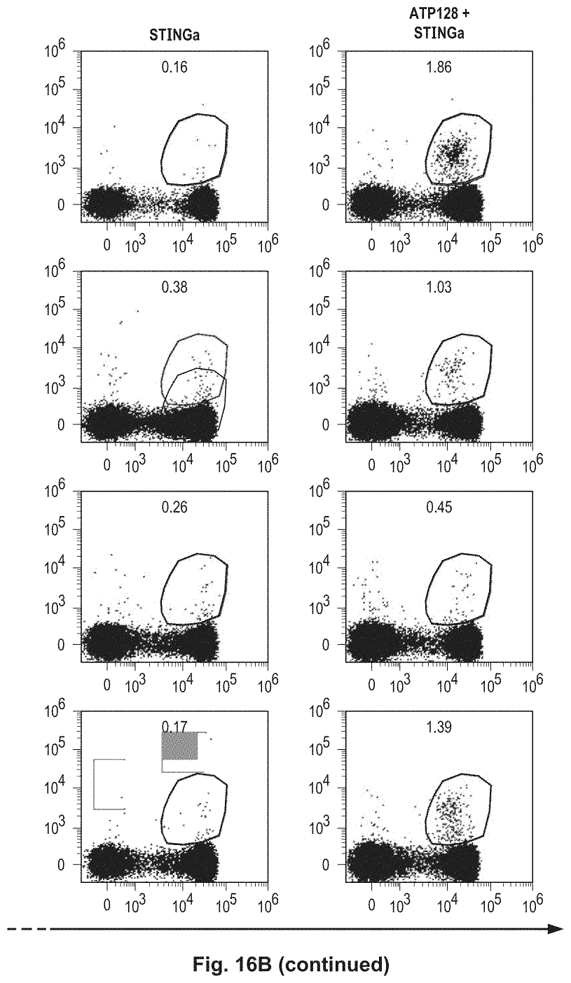

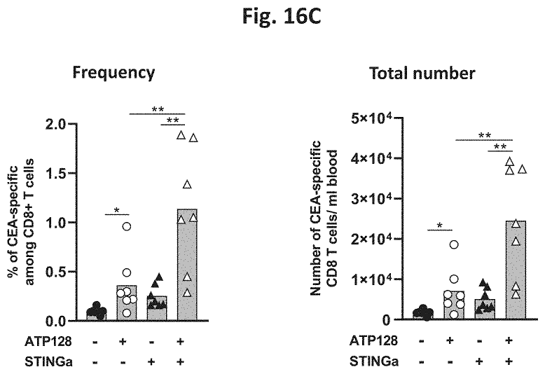

Results are shown in FIG. 16. An IFN-g Elispot assay was carried out one week after the third vaccination with ATP128. ATP128 vaccination elicits CEA-specific CD8 T cells, which can be monitored by multimer staining (FIG. 16A). Multimer staining was performed one week after the second vaccination. The data show that the addition of the STING agonist to ATP128 enhances CEA-specific CD8 T cell responses (FIG. 16 B,C).

### Example 10: Combination of STING Agonist and Vaccine Complex Enhances Functionality of CD8 T and CD4 T Cell Peripheral Responses in Tumor-Free Mice

Similarly to Example 1, the immunogenicity of the combination was evaluated in tumor-free C57BL/6 mice, but using a different complex (Z13Mad39Anaxa; SEQ ID NO: 58). Briefly, tumor-free C57BL/6 mice were vaccinated twice at one week interval (at day 0 and 7) by s.c. injection of 10 nmoles of Z13Mad39Anaxa (SEQ ID NO: 58; an exemplified a complex comprising a cell penetrating peptide, at least one antigen or antigenic epitope, and a TLR peptide agonist, which contains CD4 and CD8 epitopes derived from ovalbumin (OVA; SEQ ID NOs 59 and 57, respectively). At about the same time of vaccination, mice received 25 μg of STING agonist ADU-S100 administered via 2×50 μl s.c. injections in each side of the low back. Serum was collected 4 and 24 hours after the first vaccination and IFN-α concentration was measured by ELISA. Whole blood was collected at day 14 and used for antigen-specific CD8 T cells measurement by multimer flow cytometry staining. At the same time, spleens were harvested and splenocytes used for ex vivo stimulation and intracellular cytokine production was analyzed by flow cytometry. Alternatively, splenocytes were used for TCR avidity assay.

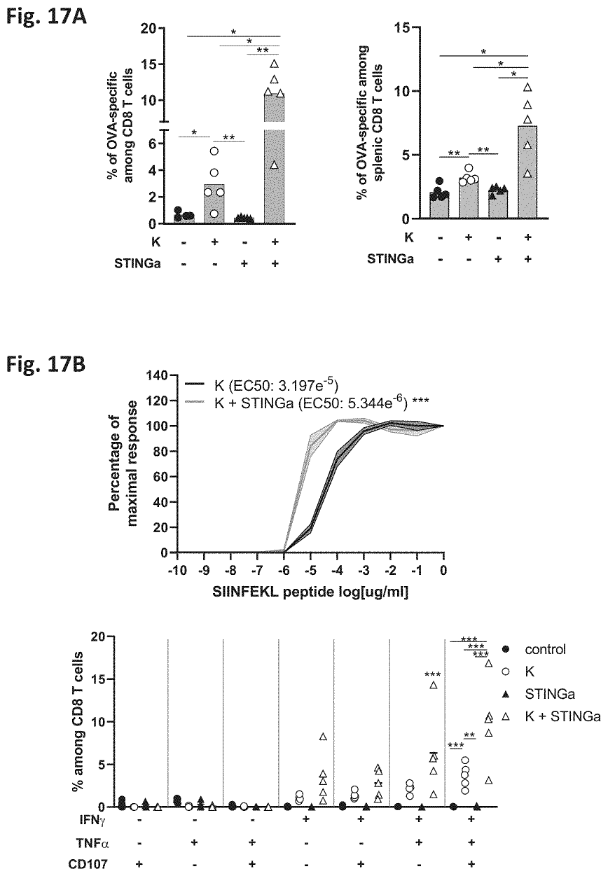

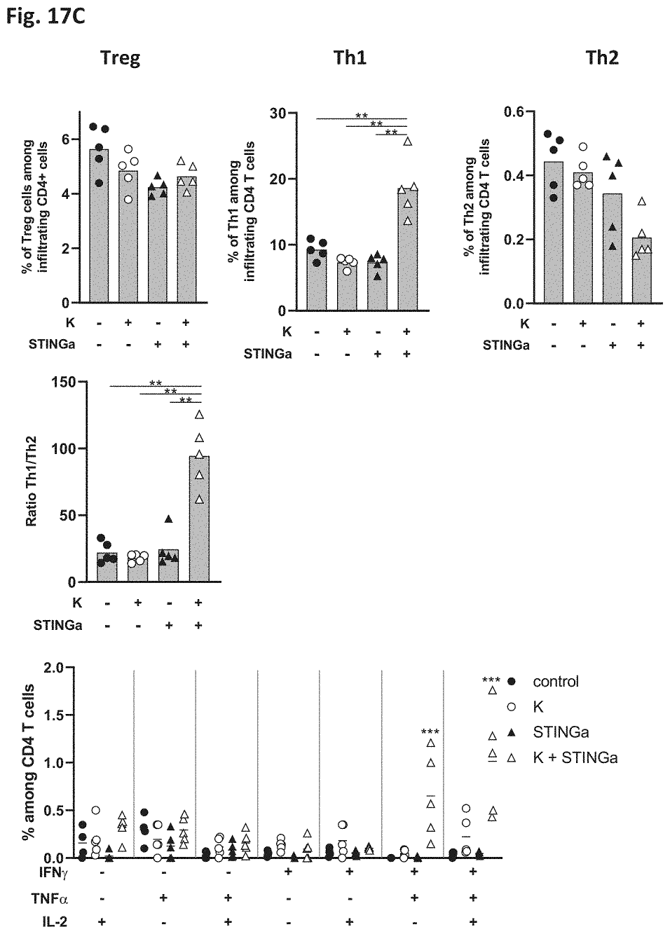

The results are shown in FIG. 17. As shown in FIG. 17A, one week after the second vaccination, circulating (left) and splenic (right) SIINFEKL-specific CD8 T cells were measured by multimer staining. FIG. 17B shows SIINFEKL-specific CD8 T cell TCR avidity measured by ex vivo ELIspot (upper panel) and antigen-specific cytokine production by CD8 T cells measured by intracellular staining after ex vivo stimulation with SIINFEKL peptide (lower panel). FIG. 17C shows the frequency of Treg (FoxP3⁺), Th1 (T-bet⁺), and Th2 (GATA-3⁺) among splenic CD4 T cells as well as the Th1/Th2 ratio (measured by flow cytometry one week after the second vaccination). In addition, antigen-specific cytokine production by CD4 T cells is shown, which was measured by intracellular staining after ex vivo stimulation with ISQAVHAAHAEINEAGR (OVA-CD4) peptide (SEQ ID NO: 59).

In summary, similar modulation of CD8 and CD4 T cell response was observed as in Example 1 using a different complex (Z13Mad39Anaxa containing CD4 and CD8 epitopes derived from ovalbumin (OVA) in the present case vs. HPV-E7-epitope containing Z13Mad25Anaxa in Example 1). This confirms that the modulation of the T cell response does not depend on the antigenic cargo. Z13Mad39Anaxa vaccination elicited polyfunctional CD8 and CD4 antigen-specific T cells, which produced IFNγ and TNFα following ex vivo stimulation with the specific peptide (fig. S2). Altogether, addition of the STING agonist to a complex comprising a cell penetrating peptide, at least one antigen or antigenic epitope, and a TLR peptide agonist profoundly impact frequency and quality of CD8 T cell response along with polarization of CD4 T cell toward Th1.

### Example 11: Combination of STING Agonist and Vaccine Complex Inhibits B16-OVA Tumor Growth

The anti-tumoral efficacy of the combination of the complex comprising a cell penetrating peptide, at least one antigen or antigenic epitope, and a TLR peptide (Z13Mad39Anaxa) agonist with the STING agonist (STINGa) was then evaluated in the B16-OVA pulmonary metastases tumor model.

Briefly, 10⁵ B16-OVA cells were injected intravenously into C57BL/6 mice. Starting three days post tumor cell intravenous injection, mice were vaccinated twice at one-week interval with an exemplified a complex comprising a cell penetrating peptide, at least one antigen or antigenic epitope, and a TLR peptide agonist (Z13Mad39Anaxa; SEQ ID NO: 58), STING agonist (STINGa) or a combination of the two. At day 20 (10 days after the last vaccination), lungs were perfused to eliminate blood, the number of pulmonary metastasis was counted, and lung infiltrating lymphocytes (LILs) were analyzed.

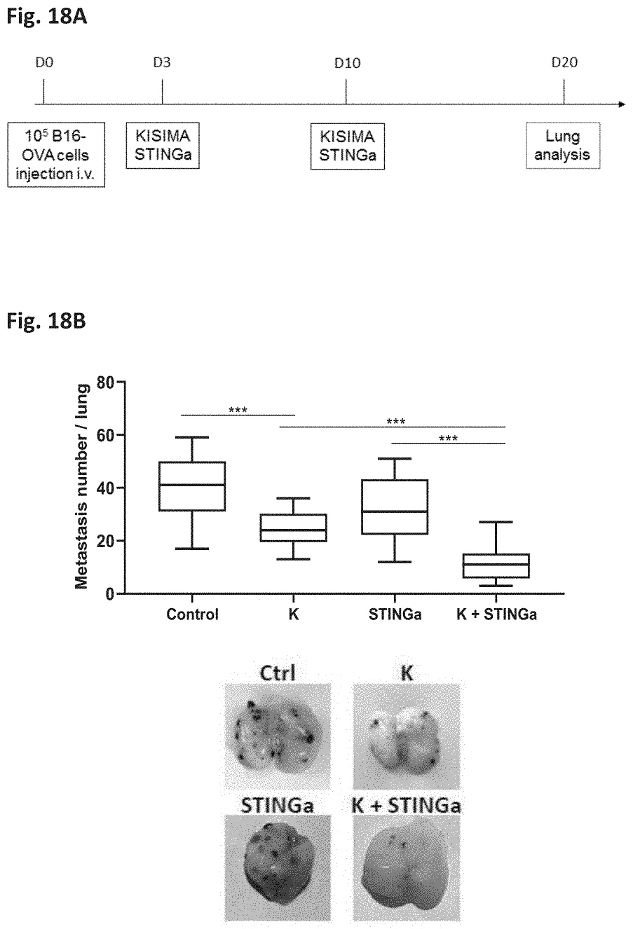

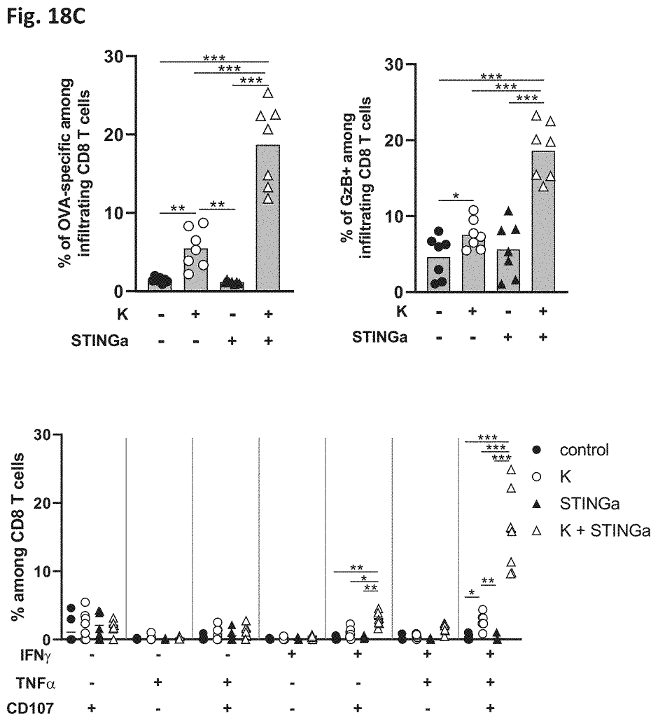

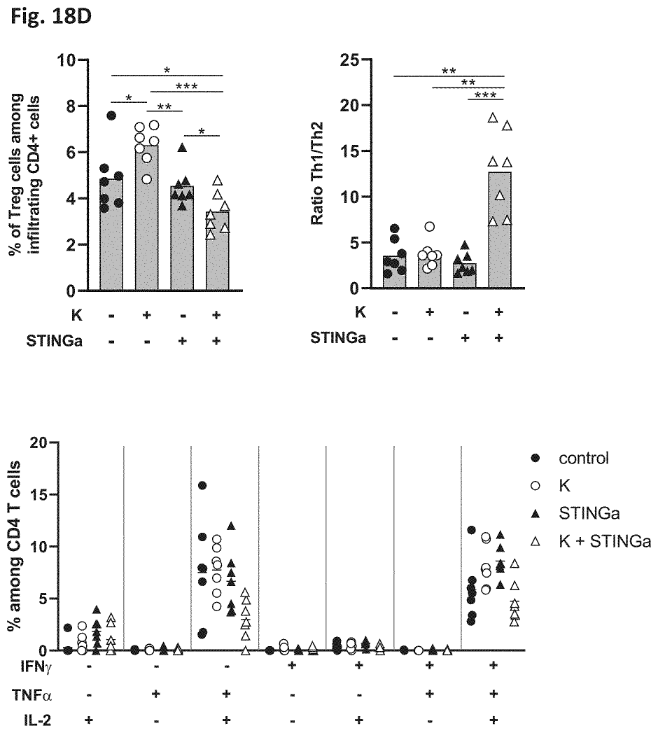

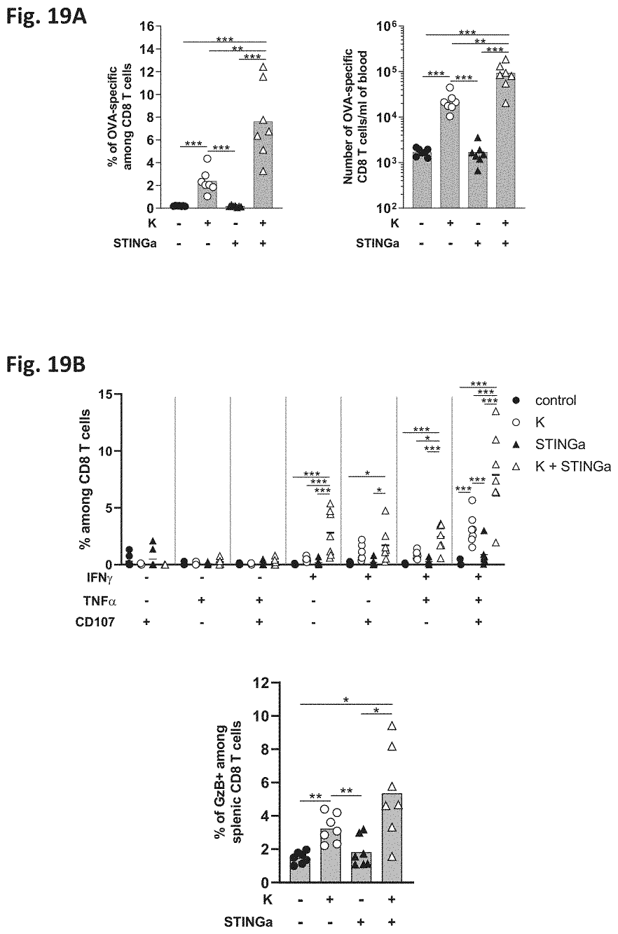

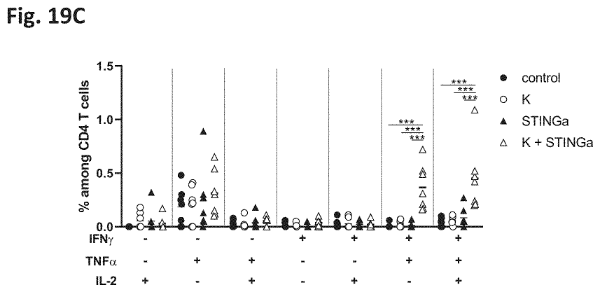

Results are shown in FIGS. 18 and 19. FIG. 18A shows the experimental schedule. The number of metastatic nodules per lung shown in FIG. 18B demonstrate that Z13Mad39Anaxa vaccination resulted in a significant reduction of the number of metastasis and while STINGa monotherapy had no effect, in combination with KISIMA it significantly further lowered the number of metastasis. In addition, the presence and functionality of lung infiltrating lymphocytes (LILs) was analyzed by flow cytometry. The vaccination induced polyfunctional OVA-specific CD8 T cells infiltration, characterized by the expression of granzyme B (GzB), IFNγ and TNFα (FIG. 18C), which were significantly increased with STINGa combination. Similar increase in T cells phenotype and functionality was observed in the periphery (blood and spleen) with a lower magnitude, suggesting that antigen-specific T cells are prevalently recruited to the tumor site, as shown in FIGS. 19A and B. As observed in tumor-free mice (Example 10), the combination of the complex comprising a cell penetrating peptide, at least one antigen or antigenic epitope, and a TLR peptide agonist (Z13Mad39Anaxa; SEQ ID NO: 58) and the STING agonist (STINGa) modulated the polarization of intratumoral CD4 T cells, decreasing the presence of Tregs while increasing the Th1/Th2 ratio (FIG. 18D). Ex vivo stimulation with OVA peptide highlighted the presence of functional antigen-specific CD4 T cells in the spleen but not in the lungs, suggesting that the helping to CD8 T cell response is prevalently happening in the secondary lymphoid organ (FIGS. 18D and 19C).

Taken together these results show that combination treatment of a complex comprising a cell penetrating peptide, at least one antigen or antigenic epitope, and a TLR peptide agonist with a STING agonist promotes both intratumoral infiltration of antigen-specific effector CD8 T cells and the functionality of peripheral CD4 T cells, resulting in the inhibition of B16-OVA tumor growth.

TABLE OF SEQUENCES AND SEQ ID NUMBERS (SEQUENCE LISTING):

| SEQ ID NO | Sequence | Remarks |
|---|---|---|
| SEQ ID NO: 1 | RQIKIYFQNRRMKWKK | CPP: Penetratin |
| SEQ ID NO: 2 | YGRKKRRQRRR | CPP: TAT minimal |
| | | domain |
| SEQ ID NO: 3 | MMDPNSTSEDVKFTPDPYQVPFVQAFDQATRVYQDLG | ZEBRA amino acid |
| | GPSQAPLPCVLWPVLPEPLPQGQLTAYHVSTAPTGSWF | sequence (natural |
| | SAPQPAPENAYQAYAAPQLFPVSDITQNQQTNQAGGE | sequence from |
| | APQPGDNSTVQTAAAVVFACPGANQGQQLADIGVPQ | Epstein-Barr virus |
| | PAPVAAPARRTRKPQQPESLEECDSELEIKRYKNRVASRK | (EBV)) (YP_401673) |
| | CRAKFKQLLQHYREVAAAKSSENDRLRLLLKQMCPSLDV | |
| | DSIIPRTPDVLHEDLLNF | |
| SEQ ID NO: 4 | KRYKNRVASRKCRAKFKQLLQHYREVAAAKSSENDRLR | CPP1 (Z11) |
| | LLLKQMC | |
| SEQ ID NO: 5 | KRYKNRVASRKCRAKFKQLLQHYREVAAAKSSENDRLR | CPP2 (Z12) |
| | LLLK | |
| SEQ ID NO: 6 | KRYKNRVASRKSRAKFKQLLQHYREVAAAKSSENDRLRL | CPP3 (Z13) |
| | LLK | |
| SEQ ID NO: 7 | KRYKNRVASRKSRAKFKQLLQHYREVAAAK | CPP4 (Z14) |
| SEQ ID NO: 8 | KRYKNRVASRKSRAKFK | CPP5 (Z15) |
| SEQ ID NO: 9 | QHYREVAAAKSSEND | CPP6 (Z16) |
| SEQ ID NO: 10 | QLLQHYREVAAAK | CPP7 (Z17) |
| SEQ ID NO: 11 | REVAAAKSSENDRLRLLLK | CPP8 (Z18) |
| SEQ ID NO: 12 | KRYKNRVA | CPP9 (Z19) |
| SEQ ID NO: 13 | VASRKSRAKFK | CPP10 (Z20) |
| SEQ ID NO: 14 | MPLEQRSQHCKPEEGLEARGEALGLVGAQAPATEEQEA | MAGE-A3 |
| | ASSSSTLVEVTLGEVPAAESPDPPQSPQGASSLPTTMNY | |
| | PLWSQSYEDSSNQEEEGPSTFPDLESEFQAALSRKVAEL | |
| | VHFLLLKYRAREPVTKAEMLGSVVGNWQYFFPVIFSKAF | |
| | SSLQLVFGIELMEVDPIGHLYIFATCLGLSYDGLLGDNQI | |
| | MPKAGLLIIVLAIIAREGDCAPEEKIWEELSVLEVFEGRED | |
| | SILGDPKKLLTQHFVQENYLEYRQVPGSDPACYEFLWGP | |
| | RALVETSYVKVLHHMVKISGGPHISYPPLHEWVLREGEE | |
| SEQ ID NO: 15 | MALPTARPLLGSCGTPALGSLLFLLFSLGWVQPSRTLAG | mesothelin |
| | ETGQEAAPLDGVLANPPNISSLSPRQLLGFPCAEVSGLST | |
| | ERVRELAVALAQKNVKLSTEQLRCLAHRLSEPPEDLDALP | |
| | LDLLLFLNPDAFSGPQACTRFFSRITKANVDLLPRGAPER | |
| | QRLLPAALACWGVRGSLLSEADVRALGGLACDLPGRFV | |
| | AESAEVLLPRLVSCPGPLDQDQQEAARAALQGGGPPYG | |
| | PPSTWSVSTMDALRGLLPVLGQPIIRSIPQGIVAAWRQR | |
| | SSRDPSWRQPERTILRPRFRREVEKTACPSGKKAREIDES | |
| | LIFYKKWELEACVDAALLATQMDRVNAIPFTYEQLDVLK | |
| | HKLDELYPQGYPESVIQHLGYLFLKMSPEDIRKWNVTSLE | |
| | TLKALLEVNKGHEMSPQAPRRPLPQVATLIDRFVKGRG | |
| | QLDKDTLDTLTAFYPGYLCSLSPEELSSVPPSSIWAVRPQ | |
| | DLDTCDPRQLDVLYPKARLAFQNMNGSEYFVKIQSFLG | |
| | GAPTEDLKALSQQNVSMDLATFMKLRTDAVLPLTVAEV | |
| | QKLLGPHVEGLKAEERHRPVRDWILRQRQDDLDTLGLG | |
| | LQGGIPNGYLVLDLSMQEALSGTPCLLGPGPVLTVLALLL | |
| | ASTLA | |
| SEQ ID NO: 16 | MGAPTLPPAWQPFLKDHRISTFKNWPFLEGCACTPER | survivin |
| | MAEAGFIHCPTENEPDLAQCFFCFKELEGWEPDDDPIEE | |
| | HKKHSSGCAFLSVKKQFEELTLGEFLKLDRERAKNKIAKE | |
| | TNNKKKEFEETAKKVRRAIEQLAAMD | |
| SEQ ID NO: 17 | RISTFKNWPF | survivin epitope |
| SEQ ID NO: 18 | APTLPPAWQPFLKDHRISTFKNWPFLEGSAVKKQFEELT | survivin fragment |
| | LGEFLKLDRER | |
| SEQ ID NO: 19 | MQAEGRGTGGSTGDADGPGGPGIPDGPGGNAGGPGE | NY-ESO-1 |
| | AGATGGRGPRGAGAARASGPGGGAPRGPHGGAASGL | |
| | NGCCRCGARGPESRLLEFYLAMPFATPMEAELARRSLA | |
| | QDAPPLPVPGVLLKEFTVSGNILTIRLTAADHRQLQLSISS | |
| | CLQQLSLLMWITQCFLPVFLAQPPSGQRR | |
| SEQ ID NO: 20 | MERRRLWGSIQSRYISMSVWTSPRRLVELAGQSLLKDE | PRAME |
| | ALAIAALELLPRELFPPLFMAAFDGRHSQTLKAMVQAW | |
| | PFTCLPLGVLMKGQHLHLETFKAVLDGLDVLLAQEVRPR | |
| | RWKLQVLDLRKNSHQDFWTVWSGNRASLYSFPEPEAA | |
| | QPMTKKRKVDGLSTEAEQPFIPVEVLVDLFLKEGACDEL | |
| | FSYLIEKVKRKKNVLRLCCKKLKIFAMPMQDIKMILKMV | |
| | QLDSIEDLEVTCTWKLPTLAKFSPYLGQMINLRRLLLSHIH | |
| | ASSYISPEKEEQYIAQFTSQFLSLQCLQALYVDSLFFLRGRL | |
| | DQLLRHVMNPLETLSITNCRLSEGDVMHLSQSPSVSQLS | |
| | VLSLSGVMLTDVSPEPLQALLERASATLQDLVFDECGITD | |
| | DQLLALLPSLSHCSQLTTLSFYGNSISISALQSLLQHLIGLS | |
| | NLTHVLYPVPLESYEDIHGTLHLERLAYLHARLRELLCELG | |
| | RPSMVWLSANPCPHCGDRTFYDPEPILCPCFMPN | |
| SEQ ID NO: 21 | MDGGTLPRSAPPAPPVPVGCAARRRPASPELLRCSRRR | ASCL2 |
| | RPATAETGGGAAAVARRNERERNRVKLVNLGFQALRQ | |
| | HVPHGGASKKLSKVETLRSAVEYIRALQRLLAEHDAVRN | |
| | ALAGGLRPQAVRPSAPRGPPGTTPVAASPSRASSSPGR | |
| | GGSSEPGSPRSAYSSDDSGCEGALSPAERELLDFSSWLG | |
| | GY | |
| SEQ ID NO: 22 | SAVEYIRALQ | ASCL2 epitope |
| SEQ ID NO: 23 | ERELLDFSSW | ASCL2 epitope |
| SEQ ID NO: 24 | AAVARRNERERNRVKLVNLGFQALRQHVPHGGASKKLS | ASCL2 fragment |
| | KVETLRSAVEYIRALQRLLAEHDAVRNALAGGLRPQAVR | |
| | PSAPRGPSEGALSPAERELLDFSSWLGGY | |
| SEQ ID NO: 25 | MTPGTQSPFFLLLLLTVLTVVTGSGHASSTPGGEKETSAT | MUC-1 |
| | QRSSVPSSTEKNAVSMTSSVLSSHSPGSGSSTTQGQDVT | |
| | LAPATEPASGSAATWGQDVTSVPVTRPALGSTTPPAHD | |
| | VTSAPDNKPAPGSTAPPAHGVTSAPDTRPAPGSTAPPA | |
| | HGVTSAPDTRPAPGSTAPPAHGVTSAPDTRPAPGSTAP | |
| | PAHGVTSAPDTRPAPGSTAPPAHGVTSAPDTRPAPGST | |
| | APPAHGVTSAPDTRPAPGSTAPPAHGVTSAPDTRPAPG | |
| | STAPPAHGVTSAPDTRPAPGSTAPPAHGVTSAPDTRPA | |
| | PGSTAPPAHGVTSAPDTRPAPGSTAPPAHGVTSAPDTR | |
| | PAPGSTAPPAHGVTSAPDTRPAPGSTAPPAHGVTSAPD | |
| | TRPAPGSTAPPAHGVTSAPDTRPAPGSTAPPAHGVTSA | |
| | PDTRPAPGSTAPPAHGVTSAPDTRPAPGSTAPPAHGVT | |
| | SAPDTRPAPGSTAPPAHGVTSAPDTRPAPGSTAPPAHG | |
| | VTSAPDTRPAPGSTAPPAHGVTSAPDTRPAPGSTAPPA | |
| | HGVTSAPDTRPAPGSTAPPAHGVTSAPDTRPAPGSTAP | |
| | PAHGVTSAPDTRPAPGSTAPPAHGVTSAPDTRPAPGST | |
| | APPAHGVTSAPDTRPAPGSTAPPAHGVTSAPDTRPAPG | |
| | STAPPAHGVTSAPDTRPAPGSTAPPAHGVTSAPDTRPA | |
| | PGSTAPPAHGVTSAPDTRPAPGSTAPPAHGVTSAPDTR | |
| | PAPGSTAPPAHGVTSAPDTRPAPGSTAPPAHGVTSAPD | |
| | TRPAPGSTAPPAHGVTSAPDTRPAPGSTAPPAHGVTSA | |
| | PDTRPAPGSTAPPAHGVTSAPDTRPAPGSTAPPAHGVT | |
| | SAPDTRPAPGSTAPPAHGVTSAPDTRPAPGSTAPPAHG | |
| | VTSAPDTRPAPGSTAPPAHGVTSAPDTRPAPGSTAPPA | |
| | HGVTSAPDTRPAPGSTAPPAHGVTSAPDNRPALGSTAP | |
| | PVHNVTSASGSASGSASTLVHNGTSARATTTPASKSTPF | |
| | SIPSHHSDTPTTLASHSTKTDASSTHHSSVPPLTSSNHSTS | |
| | PQLSTGVSFFFLSFHISNLQFNSSLEDPSTDYYQELQRDIS | |
| | EMFLQIYKQGGFLGLSNIKFRPGSVVVQLTLAFREGTINV | |
| | HDVETQFNQYKTEAASRYNLTISDVSVSDVPFPFSAQSG | |
| | AGVPGWGIALLVLVCVLVALAIVYLIALAVCQCRRKNYG | |
| | QLDIFPARDTYHPMSEYPTYHTHGRYVPPSSTDRSPYEK | |
| | VSAGNGGSSLSYTNPAVAATSANL | |
| SEQ ID NO: 26 | GSTAPPVHN | MUC-1 epitope |
| SEQ ID NO: 27 | TAPPAHGVTS | MUC-1 epitope |
| SEQ ID NO: 28 | MGRGLLRGLWPLHIVLWTRIASTIPPHVQKSVNNDMIV | TGFPR2 |
| | TDNNGAVKFPQLCKFCDVRFSTCDNQKSCMSNCSITSIC | |
| | EKPQEVCVAVWRKNDENITLETVCHDPKLPYHDFILEDA | |
| | ASPKCIMKEKKKPGETFFMCSCSSDECNDNIIFSEEYNTS | |
| | NPDLLLVIFQVTGISLLPPLGVAISVIIIFYCYRVNRQQKLSS | |
| | TVVETGKTRKLMEFSEHCAIILEDDRSDISSTCANNINHNT | |
| | ELLPIELDTLVGKGRFAEVYKAKLKQNTSEQFETVAVKIFP | |
| | YEEYASWKTEKDIFSDINLKHENILQFLTAEERKTELGKQY | |
| | WLITAFHAKGNLQEYLTRHVISWEDLRKLGSSLARGIAHL | |
| | HSDHTPCGRPKMPIVHRDLKSSNILVKNDLTCCLCDFGLS | |
| | LRLDPTLSVDDLANSGQVGTARYMAPEVLESRMNLENV | |
| | ESFKQTDVYSMALVLWEMTSRCNAVGEVKDYEPPFGSK | |
| | VREHPCVESMKDNVLRDRGRPEIPSFWLNHQGIQMVC | |
| | ETLTECWDHDPEARLTAQCVAERFSELEHLDRLSGRSCSE | |
| | EKIPEDGSLNTTK | |
| SEQ ID NO: 29 | MESPSAPPHRWCIPWQRLLLTASLLTFWNPPTTAKLTIE | CEA |
| | STPFNVAEGKEVLLLVHNLPQHLFGYSWYKGERVDGNR | |
| | QIIGYVIGTQQATPGPAYSGREIIYPNASLLIQNIIQNDTG | |
| | FYTLHVIKSDLVNEEATGQFRVYPELPKPSISSNNSKPVED | |
| | KDAVAFTCEPETQDATYLWWVNNQSLPVSPRLQLSNG | |
| | NRTLTLFNVTRNDTASYKCETQNPVSARRSDSVILNVLY | |
| | GPDAPTISPLNTSYRSGENLNLSCHAASNPPAQYSWFVN | |
| | GTFQQSTQELFIPNITVNNSGSYTCQAHNSDTGLNRTTV | |
| | TTITVYAEPPKPFITSNNSNPVEDEDAVALTCEPEIQNTTY | |
| | LWWVNNQSLPVSPRLQLSNDNRTLTLLSVTRNDVGPYE | |
| | CGIQNKLSVDHSDPVILNVLYGPDDPTISPSYTYYRPGVN | |
| | LSLSCHAASNPPAQYSWLIDGNIQQHTQELFISNITEKNS | |
| | GLYTCQANNSASGHSRTIVKTITVSAELPKPSISSNNSKP | |
| | VEDKDAVAFTCEPEAQNTTYLWWVNGQSLPVSPRLQL | |
| | SNGNRTLTLFNVTRNDARAYVCGIQNSVSANRSDPVTL | |
| | DVLYGPDTPIISPPDSSYLSGANLNLSCHSASNPSPQYSW | |
| | RINGIPQQHTQVLFIAKITPNNNGTYACFVSNLATGRNN | |
| | SIVKSITVSASGTSPGLSAGATVGIMIGVLVGVAL | |
| SEQ ID NO: 30 | YLSGANLNLS | CEA epitope |
| SEQ ID NO: 31 | SWRINGIPQQ | CEA epitope |
| SEQ ID NO: 32 | NRTLTLFNVTRNDARAYVSGIQNSVSANRSDPVTLDVLP | CEA fragment |
| | DSSYLSGANLNLSCHSASPQYSWRINGIPQQHTQVLFIA | |
| | KITPNNNGTYACFVSNLATGRNNSIVKSITVSASGTSPGL | |
| | SA | |
| SEQ ID NO: 33 | MEEPQSDPSVEPPLSQETFSDLWKLLPENNVLSPLPSQA | P53 |
| | MDDLMLSPDDIEQWFTEDPGPDEAPRMPEAAPPVAP | |
| | APAAPTPAAPAPAPSWPLSSSVPSQKTYQGSYGFRLGFL | |
| | HSGTAKSVTCTYSPALNKMFCQLAKTCPVQLWVDSTPP | |
| | PGTRVRAMAIYKQSQHMTEVVRRCPHHERCSDSDGLA | |
| | PPQHLIRVEGNLRVEYLDDRNTFRHSVVVPYEPPEVGSD | |
| | CTTIHYNYMCNSSCMGGMNRRPILTIITLEDSSGNLLGR | |
| | NSFEVRVCACPGRDRRTEEENLRKKGEPHHELPPGSTKR | |
| | ALPNNTSSSPQPKKKPLDGEYFTLQIRGRERFEMFRELNE | |
| | ALELKDAQAGKEPGGSRAHSSHLKSKKGQSTSRHKKLM | |
| | FKTEGPDSD | |
| SEQ ID NO: 34 | MTEYKLVVVGAGGVGKSALTIQLIQNHFVDEYDPTIEDS | KRas |
| | YRKQVVIDGETCLLDILDTAGQEEYSAMRDQYMRTGEG | |
| | FLCVFAINNTKSFEDIHHYREQIKRVKDSEDVPMVLVGN | |
| | KCDLPSRTVDTKQAQDLARSYGIPFIETSAKTRQRVEDAF | |
| | YTLVREIRQYRLKKISKEEKTPGCVKIKKCIIM | |
| SEQ ID NO: 35 | VVVGAGGVG | KRas epitope |
| SEQ ID NO: 36 | MASSVGNVADSTEPTKRMLSFQGLAELAHREYQAGDF | OGT |
| | EAAERHCMQLWRQEPDNTGVLLLLSSIHFQCRRLDRSA | |
| | HFSTLAIKQNPLLAEAYSNLGNVYKERGQLQEAIEHYRH | |
| | ALRLKPDFIDGYINLAAALVAAGDMEGAVQAYVSALQY | |
| | NPDLYCVRSDLGNLLKALGRLEEAKACYLKAIETQPNFAV | |
| | AWSNLGCVFNAQGEIWLAIHHFEKAVTLDPNFLDAYINL | |
| | GNVLKEARIFDRAVAAYLRALSLSPNHAVVHGNLACVYY | |
| | EQGLIDLAIDTYRRAIELQPHFPDAYCNLANALKEKGSVA | |
| | EAEDCYNTALRLCPTHADSLNNLANIKREQGNIEEAVRL | |
| | YRKALEVFPEFAAAHSNLASVLQQQGKLQEALMHYKEAI | |
| | RISPTFADAYSNMGNTLKEMQDVQGALQCYTRAIQINP | |
| | AFADAHSNLASIHKDSGNIPEAIASYRTALKLKPDFPDAY | |
| | CNLAHCLQIVCDWTDYDERMKKLVSIVADQLEKNRLPS | |
| | VHPHHSMLYPLSHGFRKAIAERHGNLCLDKINVLHKPPY | |
| | EHPKDLKLSDGRLRVGYVSSDFGNHPTSHLMQSIPGMH | |
| | NPDKFEVFCYALSPDDGTNFRVKVMAEANHFIDLSQIPC | |
| | NGKAADRIHQDGIHILVNMNGYTKGARNELFALRPAPI | |
| | QAMWLGYPGTSGALFMDYIITDQETSPAEVAEQYSEKL | |
| | AYMPHTFFIGDHANMFPHLKKKAVIDFKSNGHIYDNRIV | |
| | LNGIDLKAFLDSLPDVKIVKMKCPDGGDNADSSNTALN | |
| | MPVIPMNTIAEAVIEMINRGQIQITINGFSISNGLATTQI | |
| | NNKAATGEEVPRTIIVTTRSQYGLPEDAIVYCNFNQLYKI | |
| | DPSTLQMWANILKRVPNSVLWLLRFPAVGEPNIQQYA | |
| | QNMGLPQNRIIFSPVAPKEEHVRRGQLADVCLDTPLCN | |
| | GHTTGMDVLWAGTPMVTMPGETLASRVAASQLTCLG | |
| | CLELIAKNRQEYEDIAVKLGTDLEYLKKVRGKVWKQRISS | |
| | PLFNTKQYTMELERLYLQMWEHYAAGNKPDHMIKPVE | |
| | VTESA | |
| SEQ ID NO: 37 | MAEDSGKKKRRKNFEAMFKGILQSGLDNFVINHMLKN | CASP5 |
| | NVAGQTSIQTLVPNTDQKSTSVKKDNHKKKTVKMLEYL | |
| | GKDVLHGVFNYLAKHDVLTLKEEEKKKYYDTKIEDKALIL | |
| | VDSLRKNRVAHQMFTQTLLNMDQKITSVKPLLQIEAGP | |
| | PESAESTNILKLCPREEFLRLCKKNHDEIYPIKKREDRRRLA | |
| | LIICNTKFDHLPARNGAHYDIVGMKRLLQGLGYTVVDEK | |
| | NLTARDMESVLRAFAARPEHKSSDSTFLVLMSHGILEGIC | |
| | GTAHKKKKPDVLLYDTIFQIFNNRNCLSLKDKPKVIIVQA | |
| | CRGEKHGELWVRDSPASLALISSQSSENLEADSVCKIHEE | |
| | KDFIAFCSSTPHNVSWRDRTRGSIFITELITCFQKYSCCCH | |
| | LMEIFRKVQKSFEVPQAKAQMPTIERATLTRDFYLFPGN | |
| SEQ ID NO: 38 | MSSPLASLSKTRKVPLPSEPMNPGRRGIRIYGDEDEVDM | COA-1 |
| | LSDGCGSEEKISVPSCYGGIGAPVSRQVPASHDSELMAF | |
| | MTRKLWDLEQQVKAQTDEILSKDQKIAALEDLVQTLRP | |
| | HPAEATLQRQEELETMCVQLQRQVREMERFLSDYGLQ | |
| | WVGEPMDQEDSESKTVSEHGERDWMTAKKFWKPGD | |
| | SLAPPEVDFDRLLASLQDLSELVVEGDTQVTPVPGGARL | |
| | RTLEPIPLKLYRNGIMMFDGPFQPFYDPSTQRCLRDILDG | |
| | FFPSELQRLYPNGVPFKVSDLRNQVYLEDGLDPFPGEGR | |
| | VVGRQLMHKALDRVEEHPGSRMTAEKFLNRLPKFVIRQ | |
| | GEVIDIRGPIRDTLQNCCPLPARIQEIVVETPTLAAERERS | |
| | QESPNTPAPPLSMLRIKSENGEQAFLLMMQPDNTIGDV | |
| | RALLAQARVMDASAFEIFSTFPPTLYQDDTLTLQAAGLV | |
| | PKAALLLRARRAPKSSLKFSPGPCPGPGPGPSPGPGPGP | |
| | SPGPGPGPSPCPGPSPSPQ | |
| SEQ ID NO: 39 | MAFVCLAIGCLYTFLISTTFGCTSSSDTEIKVNPPQDFEIV | IL13Ralpha2 |
| | DPGYLGYLYLQWQPPLSLDHFKECTVEYELKYRNIGSET | |
| | WKTIITKNLHYKDGFDLNKGIEAKIHTLLPWQCTNGSEV | |
| | QSSWAETTYWISPQGIPETKVQDMDCVYYNWQYLLCS | |
| | WKPGIGVLLDTNYNLFYWYEGLDHALQCVDYIKADGQ | |
| | NIGCRFPYLEASDYKDFYICVNGSSENKPIRSSYFTFQLQN | |
| | IVKPLPPVYLTFTRESSCEIKLKWSIPLGPIPARCFDYEIEIR | |
| | EDDTTLVTATVENETYTLKTTNETRQLCFVVRSKVNIYCS | |
| | DDGIWSEWSDKQCWEGEDLSKKTLLRFWLPFGFILILVIF | |
| | VTGLLLRKPNTYPKMIPEFFCDT | |
| SEQ ID NO: 40 | LPFGFIL | IL13Ralpha2 epitope |
| SEQ ID NO: 41 | MNKLYIGNLSENAAPSDLESIFKDAKIPVSGPFLVKTGYA | KOC1 |
| | FVDCPDESWALKAIEALSGKIELHGKPIEVEHSVPKRQRI | |
| | RKLQIRNIPPHLQWEVLDSLLVQYGVVESCEQVNTDSET | |
| | AVVNVTYSSKDQARQALDKLNGFQLENFTLKVAYIPDE | |
| | MAAQQNPLQQPRGRRGLGQRGSSRQGSPGSVSKQKP | |
| | CDLPLRLLVPTQFVGAIIGKEGATIRNITKQTQSKIDVHRK | |
| | ENAGAAEKSITILSTPEGTSAACKSILEIMHKEAQDIKFTE | |
| | EIPLKILAHNNFVGRLIGKEGRNLKKIEQDTDTKITISPLQE | |
| | LTLYNPERTITVKGNVETCAKAEEEIMKKIRESYENDIAS | |
| | MNLQAHLIPGLNLNALGLFPPTSGMPPPTSGPPSAMTP | |
| | PYPQFEQSETETVHLFIPALSVGAIIGKQGQHIKQLSRFA | |
| | GASIKIAPAEAPDAKVRMVIITGPPEAQFKAQGRIYGKIK | |
| | EENFVSPKEEVKLEAHIRVPSFAAGRVIGKGGKTVNELQ | |
| | NLSSAEVVVPRDQTPDENDQVVVKITGHFYACQVAQRK | |
| | IQEILTQVKQHQQQKALQSGPPQSRRK | |
| SEQ ID NO: 42 | MAPKFPDSVEELRAAGNESFRNGQYAEASALYGRALRV | TOMM34 |
| | LQAQGSSDPEEESVLYSNRAACHLKDGNCRDCIKDCTSA | |
| | LALVPFSIKPLLRRASAYEALEKYPMAYVDYKTVLQIDDN | |
| | VTSAVEGINRMTRALMDSLGPEWRLKLPSIPLVPVSAQK | |
| | RWNSLPSENHKEMAKSKSKETTATKNRVPSAGDVEKAR | |
| | VLKEEGNELVKKGNHKKAIEKYSESLLCSNLESATYSNRAL | |
| | CYLVLKQYTEAVKDCTEALKLDGKNVKAFYRRAQAHKAL | |
| | KDYKSSFADISNLLQIEPRNGPAQKLRQEVKQNLH | |
| SEQ ID NO: 43 | MSGGHQLQLAALWPWLLMATLQAGFGRTGLVLAAAV | RN F-43 |
| | ESERSAEQKAIIRVIPLKMDPTGKLNLTLEGVFAGVAEITP | |
| | AEGKLMQSHPLYLCNASDDDNLEPGFISIVKLESPRRAPR | |
| | PCLSLASKARAGERGASAVLFDITEDRAAAEQLQQPLGLT | |
| | WPVVLIWGNDAEKLMEFVYKNQKAHVRIELKEPPAWP | |
| | DYDVWILMTVVGTIFVIILASVLRIRCRPRHSRPDPLQQR | |
| | TAWAISQLATRRYQASCRQARGEWPDSGSSCSSAPVCA | |
| | ICLEEFSEGQELRVISCLHEFHRNCVDPWLHQHRTCPLC | |
| | MFNITEGDSFSQSLGPSRSYQEPGRRLHLIRQHPGHAHY | |
| | HLPAAYLLGPSRSAVARPPRPGPFLPSQEPGMGPRHHRF | |
| | PRAAHPRAPGEQQRLAGAQHPYAQGWGLSHLQSTSQ | |
| | HPAACPVPLRRARPPDSSGSGESYCTERSGYLADGPASD | |
| | SSSGPCHGSSSDSVVNCTDISLQGVHGSSSTFCSSLSSDF | |
| | DPLVYCSPKGDPQRVDMQPSVTSRPRSLDSVVPTGETQ | |
| | VSSHVHYHRHRHHHYKKRFQWHGRKPGPETGVPQSRP | |
| | PIPRTQPQPEPPSPDQQVTRSNSAAPSGRLSNPQCPRAL | |
| | PEPAPGPVDASSICPSTSSLFNLQKSSLSARHPQRKRRGG | |
| | PSEPTPGSRPQDATVHPACQIFPHYTPSVAYPWSPEAHP | |
| | LICGPPGLDKRLLPETPGPCYSNSQPVWLCLTPRQPLEPH | |
| | PPGEGPSEWSSDTAEGRPCPYPHCQVLSAQPGSEEELEE | |
| | LCEQAV | |
| SEQ ID NO: 44 | MAPPQVLAFGLLLAAATATFAAAQEECVCENYKLAVNC | EpCAM |
| | FVNNNRQCQCTSVGAQNTVICSKLAAKCLVMKAEMNG | |
| | SKLGRRAKPEGALQNNDGLYDPDCDESGLFKAKQCNGT | |
| | SMCWCVNTAGVRRTDKDTEITCSERVRTYWIIIELKHKA | |
| | REKPYDSKSLRTALQKEITTRYQLDPKFITSILYENNVITIDL | |
| | VQNSSQKTQNDVDIADVAYYFEKDVKGESLFHSKKMDL | |
| | TVNGEQLDLDPGQTLIYYVDEKAPEFSMQGLKAGVIAVI | |
| | VVVVIAVVAGIVVLVISRKKRMAKYEKAEIKEMGEMHRE | |
| | LNA | |
| SEQ ID NO: 45 | GLKAGVIAV | EpCAM epitope |
| SEQ ID NO: 46 | MELAALCRWGLLLALLPPGAASTQVCTGTDMKLRLPAS | Her2/neu |
| | PETHLDMLRHLYQGCQVVQGNLELTYLPTNASLSFLQDI | |
| | QEVQGYVLIAHNQVRQVPLQRLRIVRGTQLFEDNYALA | |
| | VLDNGDPLNNTTPVTGASPGGLRELQLRSLTEILKGGVLI | |
| | QRNPQLCYQDTILWKDIFHKNNQLALTLIDTNRSRACHP | |
| | CSPMCKGSRCWGESSEDCQSLTRTVCAGGCARCKGPLP | |
| | TDCCHEQCAAGCTGPKHSDCLACLHFNHSGICELHCPAL | |
| | VTYNTDTFESMPNPEGRYTFGASCVTACPYNYLSTDVGS | |
| | CTLVCPLHNQEVTAEDGTQRCEKCSKPCARVCYGLGME | |
| | HLREVRAVTSANIQEFAGCKKIFGSLAFLPESFDGDPASN | |
| | TAPLQPEQLQVFETLEEITGYLYISAWPDSLPDLSVFQNL | |
| | QVIRGRILHNGAYSLTLQGLGISWLGLRSLRELGSGLALIH | |
| | HNTHLCFVHTVPWDQLFRNPHQALLHTANRPEDECVG | |
| | EGLACHQLCARGHCWGPGPTQCVNCSQFLRGQECVEE | |
| | CRVLQGLPREYVNARHCLPCHPECQPQNGSVTCFGPEA | |
| | DQCVACAHYKDPPFCVARCPSGVKPDLSYMPIWKFPDE | |
| | EGACQPCPINCTHSCVDLDDKGCPAEQRASPLTSIISAVV | |
| | GILLVVVLGVVFGILIKRRQQKIRKYTMRRLLQETELVEPL | |
| | TPSGAMPNQAQMRILKETELRKVKVLGSGAFGTVYKGI | |
| | WIPDGENVKIPVAIKVLRENTSPKANKEILDEAYVMAGV | |
| | GSPYVSRLLGICLTSTVQLVTQLMPYGCLLDHVRENRGR | |
| | LGSQDLLNWCMQIAKGMSYLEDVRLVHRDLAARNVLV | |
| | KSPNHVKITDFGLARLLDIDETEYHADGGKVPIKWMALE | |
| | SILRRRFTHQSDVWSYGVTVWELMTFGAKPYDGIPAREI | |
| | PDLLEKGERLPQPPICTIDVYMIMVKCWMIDSECRPRFR | |
| | ELVSEFSRMARDPQRFVVIQNEDLGPASPLDSTFYRSLLE | |
| | DDDMGDLVDAEEYLVPQQGFFCPDPAPGAGGMVHHR | |
| | HRSSSTRSGGGDLTLGLEPSEEEAPRSPLAPSEGAGSDVF | |
| | DGDLGMGAAKGLQSLPTHDPSPLQRYSEDPTVPLPSET | |
| | DGYVAPLTCSPQPEYVNQPDVRPQPPSPREGPLPAARP | |
| | AGATLERPKTLSPGKNGVVKDVFAFGGAVENPEYLTPQ | |
| | GGAAPQPHPPPAFSPAFDNLYYWDQDPPERGAPPSTF | |
| | KGTPTAENPEYLGLDVPV | |
| SEQ ID NO: 47 | MGSDVRDLNALLPAVPSLGGGGGCALPVSGAAQWAP | WT1 |
| | VLDFAPPGASAYGSLGGPAPPPAPPPPPPPPPHSFIKQE | |
| | PSWGGAEPHEEQCLSAFTVHFSGQFTGTAGACRYGPFG | |
| | PPPPSQASSGQARMFPNAPYLPSCLESQPAIRNQGYSTV | |
| | TFDGTPSYGHTPSHHAAQFPNHSFKHEDPMGQQGSLG | |
| | EQQYSVPPPVYGCHTPTDSCTGSQALLLRTPYSSDNLYQ | |
| | MTSQLECMTWNQMNLGATLKGVAAGSSSSVKWTEG | |
| | QSNHSTGYESDNHTTPILCGAQYRIHTHGVFRGIQDVRR | |
| | VPGVAPTLVRSASETSEKRPFMCAYPGCNKRYFKLSHLQ | |
| | MHSRKHTGEKPYQCDFKDCERRFSRSDQLKRHQRRHT | |
| | GVKPFQCKTCQRKFSRSDHLKTHTRTHTGKTSEKPFSCR | |
| | WPSCQKKFARSDELVRHHNMHQRNMTKLQLAL | |
| SEQ ID NO: 48 | NRTLTLFNVTRNDARAYVSGIQNSVSANRSDPVTLDVLP | antigenic cargo of |
| | DSSYLSGANLNLSCHSASPQYSWRINGIPQQHTQVLFIA | ATP128 |
| | KITPNNNGTYACFVSNLATGRNNSIVKSITVSASGTSPGL | |
| | SAAPTLPPAWQPFLKDHRISTFKNWPFLEGSAVKKQFEE | |
| | LTLGEFLKLDRERAAVARRNERERNRVKLVNLGFQALRQ | |
| | HVPHGGASKKLSKVETLRSAVEYIRALQRLLAEHDAVRN | |
| | ALAGGLRPQAVRPSAPRGPSEGALSPAERELLDFSSWLG | |
| | GY | |
| SEQ ID NO: 49 | STVHEILCKLSLEGDHSTPPSAYGSVKPYTNFDAE | TLR2 peptide agonist |
| | | Anaxa |
| SEQ ID NO: 50 | STVHEILSKLSLEGDHSTPPSAYGSVKPYTNFDAE | TLR peptide agonist |
| | | ″Anaxa″ sequence |
| | | variant |
| SEQ ID NO: 51 | MGKGDPKKPRGKMSSYAFFVQTCREEHKKKHPDASVN | TLR2 agonist mo- |
| | FSEFSKKCSERWKTMSAKEKGKFEDMAKADKARYEREM | HMGB1 |
| | KTYIPPKGETKKKFKDPNAPKRPPSAFFLFCSEYRPKIKGE | |
| | HPGLSIGDVAKKLGEMWNNTAADDKQPYEKKAAKLKEK | |
| | YEKDIAAYRAKGKPDAAKKGVVKAEKSKKKK | |
| SEQ ID NO: 52 | NIDRPKGLAFTDVDVDSIKIAWESPQGQVSRYRVTYSSPE | EDA |
| | DGIRELFPAPDGEDDTAELQGLRPGSEYTVSVVALHDD | |
| | MESQPLIGIQST | |
| SEQ ID NO: 53 | DPNAPKRPPSAFFLFCSEKRYKNRVASRKSRAKFKQLLQH | Hp91 |
| | YREVAAAKSSENDRLRLLLKESLKISQAVHAAHAEINEAG | |
| | REVVGVGALKVPRNQDWLGVPRFAKFASFEAQGALANI | |
| | AVDKANLDVEQLESIINFEKLTEWTGS | |
| SEQ ID NO: 54 | KRYKNRVASRKSRAKFKQLLQHYREVAAAKSSENDRLRL | ATP128 |
| | LLKNRTLTLFNVTRNDARAYVSGIQNSVSANRSDPVTLD | |
| | VLPDSSYLSGANLNLSCHSASPQYSWRINGIPQQHTQVL | |
| | FIAKITPNNNGTYACFVSNLATGRNNSIVKSITVSASGTSP | |
| | GLSAAPTLPPAWQPFLKDHRISTFKNWPFLEGSAVKKQF | |
| | EELTLGEFLKLDRERAAVARRNERERNRVKLVNLGFQAL | |
| | RQHVPHGGASKKLSKVETLRSAVEYIRALQRLLAEHDAV | |
| | RNALAGGLRPQAVRPSAPRGPSEGALSPAERELLDFSSW | |
| | LGGYSTVHEILSKLSLEGDHSTPPSAYGSVKPYTNFDAE | |
| SEQ ID NO: 55 | KRYKNRVASRKSRAKFKQLLQHYREVAAAKSSENDRLRL | Z13Mad25Anaxa |
| | LLKQAEPDRAHYNIVTFSSKSSTVHEILSKLSLEGDHSTPP | |
| | SAYGSVKPYTNFDAE | |
| SEQ ID NO: 56 | RAHYNIVTF | HPV-E7 CD8 epitope |
| SEQ ID NO: 57 | SIINFEKL | OVA CD8 epitope |
| SEQ ID NO: 58 | KRYKNRVASRKSRAKFKQLLQHYREVAAAKSSENDRLRL | Z13Mad39Anaxa |
| | LLKESLKISQAVHAAHAEINEAGREVVGVGALKVPRNQD | |
| | WLGVPRFAKFASFEAQGALANIAVDKANLDVEQLESIIN | |
| | FEKLTEWTGSSTVHEILSKLSLEGDHSTPPSAYGSVKPYT | |
| | NFDAE | |
| SEQ ID NO: 59 | ISQAVHAAHAEINEAGR | OVA CD4 epitope |

1. A combination of
(i) a Stimulator of Interferon Genes (STING) agonist; and
(ii) a complex comprising:
a) a cell penetrating peptide;
b) at least one antigen or antigenic epitope; and
c) a Toll Like Receptor (TLR) peptide agonist,
wherein the components a)-c) comprised by the complex are covalently linked.

2. The combination according to claim 1, wherein the complex is a recombinant polypeptide or a recombinant protein.

3. The combination according to claim 1, wherein the cell penetrating peptide
(1) has a length of the amino acid sequence of said peptide of 15 to 45 amino acids in total; and/or
(2) has an amino acid sequence comprising a fragment of the minimal domain of ZEBRA, said minimal domain extending from residue 170 to residue 220 of the ZEBRA amino acid sequence according to SEQ ID NO: 3, wherein, optionally, 1, 2, 3, 4, or 5 amino acids have been substituted, deleted, and/or added without abrogating said peptide's cell penetrating ability.

4.-6. (canceled)

7. The combination according to claim 6, wherein the cell penetrating peptide has an amino acid sequence comprising or consisting of an amino acid sequence according to SEQ ID NO: 6 (CPP3/Z13), SEQ ID NO: 7 (CPP4/Z14), SEQ ID NO: 8 (CPP5/Z15), or SEQ ID NO: 11 (CPP8/Z18), or sequence variants thereof having at least 70%, 75%, 80%, 85%, 90% or 95% sequence identity without abrogating said peptide's cell penetrating ability.

8.-11. (canceled)

12. The combination according to claim 1, wherein the at least one antigen or antigenic epitope comprises or consists of at least one tumor or cancer epitope.

13. The combination according to claim 12, wherein the at least one tumor epitope is selected from the group of tumors comprising endocrine tumors, gastrointestinal tumors, genitourinary and gynecologic tumors, breast cancer, head and neck tumors, hematopoietic tumors, skin tumors, and thoracic and respiratory tumors.

14. The combination according to claim 12, wherein the at least one tumor or cancer epitope is selected from the group of tumors or cancers of: gastrointestinal tumors comprising anal cancer, appendix cancer, cholangiocarcinoma, carcinoid tumor, gastrointestinal colon cancer, extrahepatic bile duct cancer, gallbladder cancer, gastric (stomach) cancer, gastrointestinal carcinoid tumor, gastrointestinal stromal tumor (GIST), hepatocellular cancer, pancreatic cancer, rectal cancer, colorectal cancer, and metastatic colorectal cancer.

15. The combination according to claim 12, wherein the at least one antigen or antigenic epitope is selected from a tumor associated antigen, tumor-specific antigen, and tumor neoantigen.

16. The combination according to claim 12, wherein the at least one tumor or cancer epitope is an epitope of an antigen selected from the group consisting of EpCAM, HER-2, MUC-1, TOMM34, RNF 43, KOC1, VEGFR, βhCG, survivin, CEA, TGFβR2, p53, KRas, OGT, CASP5, COA-1, MAGE, SART, IL13Ralpha2, ASCL2, NY-ESO-1, MAGE-A3, PRAME, and WT1.

17. (canceled)

18. The combination according to claim 1, wherein the complex comprises a multi-antigenic domain, which comprises epitopes of at least two distinct antigens.

19.-43. (canceled)

44. The combination according to claim 18, wherein the multi-antigenic domain comprises
one or more epitopes of survivin or sequence variants thereof;
one or more epitopes of CEA or sequence variants thereof; and
one or more epitopes of ASCL2 or sequence variants thereof.

45.-47. (canceled)

48. The combination according to claim 18, wherein the multi-antigenic domain comprises a peptide consisting of an amino acid sequence according to SEQ ID NO: 32 or a sequence variant thereof having at least 70%, 75%, 80%, 85%, 90% or 95% sequence identity; a peptide consisting of an amino acid sequence according to SEQ ID NO: 18 or a sequence variant thereof having at least 70%, 75%, 80%, 85%, 90% or 95% sequence identity; and a peptide consisting of an amino acid sequence according to SEQ ID NO: 24 or a sequence variant thereof having at least 70%, 75%, 80%, 85%, 90% or 95% sequence identity.

49. The combination according to claim 18, wherein the multi-antigenic domain of the complex comprises a peptide consisting of an amino acid sequence according to SEQ ID NO: 48 or a sequence variant thereof having at least 70%, 75%, 80%, 85%, 90% or 95% sequence identity.

50. (canceled)

51. The combination according to claim 1, wherein the TLR peptide agonist is a TLR2 peptide agonist and/or a TLR4 peptide agonist.

52. (canceled)

53. The combination according to claim 1, wherein the TLR peptide agonist comprises or consists of an amino acid sequence according to SEQ ID NO: 49 or 50; or a sequence variant thereof having at least 70%, 75%, 80%, 85%, 90% or 95% sequence identity.

54.-59. (canceled)

60. The combination according to claim 1, wherein the complex is a polypeptide or protein, wherein
a) the cell penetrating peptide has an amino acid sequence comprising or consisting of an amino acid sequence according to SEQ ID NO: 6 (CPP3/Z13), SEQ ID NO: 7 (CPP4/Z14), SEQ ID NO: 8 (CPP5/Z15), or SEQ ID NO: 11 (CPP8/Z18), or a sequence variant thereof having at least 70%, 75%, 80%, 85%, 90% or 95% sequence identity (without abrogating said peptide's cell penetrating ability;
b) the at least one antigen or antigenic epitope is a peptide, polypeptide or protein; and
c) the TLR peptide agonist is a TLR2 peptide agonist and/or a TLR4 peptide agonist.

61. (canceled)

62. The combination according to claim 1, wherein the complex comprises or consists of an amino acid sequence according to SEQ ID NO: 54, or a sequence variant thereof having at least 70%, 75%, 80%, 85%, 90% or 95% sequence identity.

63. The combination according to claim 1, wherein the STING agonist is a cyclic-dinucleotide (CDN) based STING agonist.

64. The combination according to claim 1, wherein the STING agonist is selected from the group consisting of ADU-S100, MK-1454, E-7766, MK-2118, BMS-986301, IMSA-101, SB-11285, SYNB-1891, GSK-3745417, TAK-676, and TTI-10001.

65. The combination according to claim 1, wherein the STING agonist is a compound of formula I 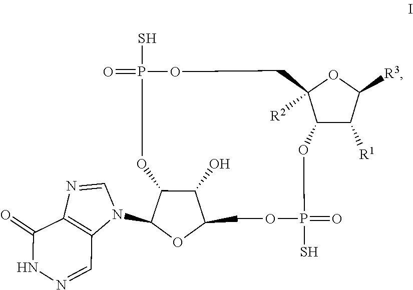
wherein
R¹ is selected from the group consisting of H, F, —O—C₁₋₃ alkyl and OH, and
R² is H, or
R² is —CH₂— and R¹ is —O—, forming together a —CH₂—O— bridge, and
R³ is a purine nucleobase selected from the group consisting of purine, adenine, guanine, xanthine, and hypoxanthine, connected through its N⁹ nitrogen;
or a salt thereof.

66. The combination according to claim 65, wherein the STING agonist is a compound of formula Ia 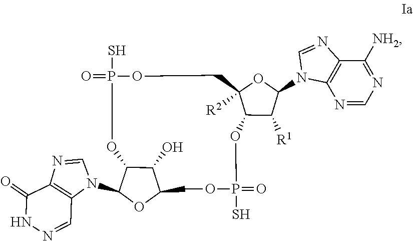
or a salt thereof;
or
a compound of formula Ib 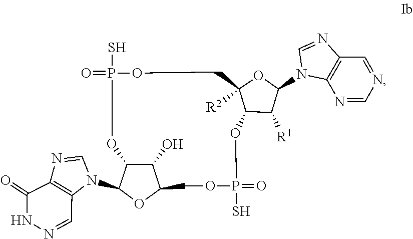
or a salt thereof.

67. (canceled)

68. The combination according to claim 66, wherein the STING agonist is a compound of formula Ia.1 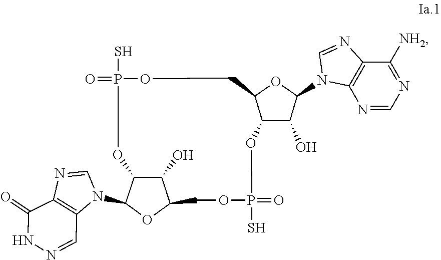 [SMILES: Nc1ncnc2c1ncn2[C@@H]1O[C@@H]2COP(=O)(S)O[C@@H]3[C@H](O)[C@@H](COP(=O)(S)O[C@H]2[C@H]1O)O[C@H]3n1cnc2c(=O)[nH]ncc21]
or a salt thereof
or
a compound of formula Ia.2 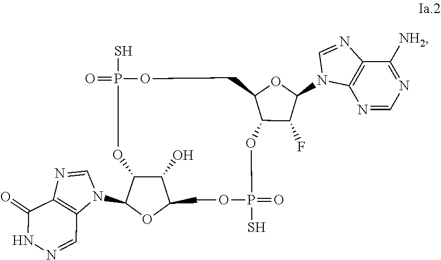 [SMILES: Nc1ncnc2c1ncn2[C@@H]1O[C@@H]2COP(=O)(S)O[C@@H]3[C@H](O)[C@@H](COP(=O)(S)O[C@H]2[C@H]1F)O[C@H]3n1cnc2c(=O)[nH]ncc21]
or a salt thereof;
or
a compound of formula Ia.3 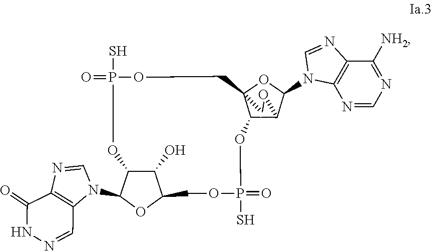 [SMILES: Nc1ncnc2c1ncn2[C@@H]1O[C@]23CO[C@@H]1[C@@H]2OP(=O)(S)OC[C@H]1O[C@@H](n2cnc4c(=O)[nH]ncc42)[C@H](OP(=O)(S)OC3)[C@@H]1O]
or a salt thereof;
or
a compound of formula Ib.1 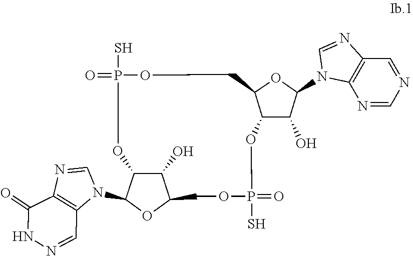 [SMILES: O=c1[nH]ncc2c1ncn2[C@@H]1O[C@@H]2COP(=O)(S)O[C@H]3[C@@H](O)[C@H](n4cnc5cncnc54)O[C@@H]3COP(=O)(S)O[C@@H]1[C@@H]2O]
or a salt thereof.

69. (canceled)

70. (canceled)

71. (canceled)

72. The combination according to claim 1, wherein the STING agonist is a compound of formula II: 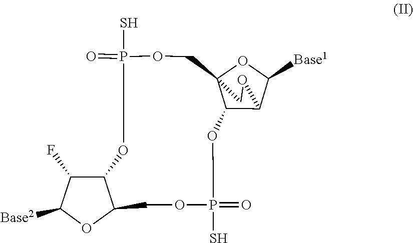
wherein
Base¹ and Base² are independently selected from the group consisting of purine, adenine, guanine, xanthine, and hypoxanthine, connected through their N⁹ nitrogen atoms;
or a salt thereof.

73. The combination according to claim 72, wherein the STING agonist is a compound of formula II-1 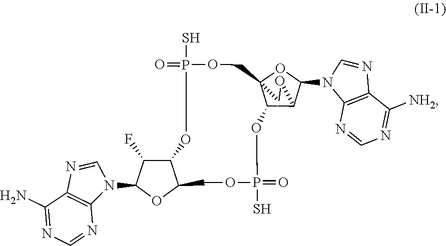 [SMILES: Nc1ncnc2c1ncn2[C@@H]1O[C@@H]2COP(=O)(S)O[C@H]3[C@H]4OC[C@@]3(COP(=O)(S)O[C@H]2[C@H]1F)O[C@H]4n1cnc2c(N)ncnc21]
or a salt thereof;
or
a compound of formula II-2 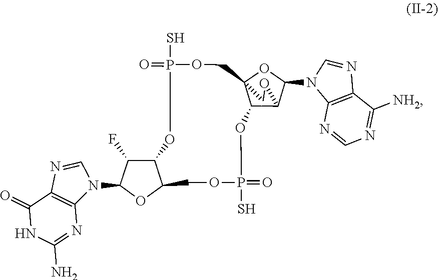 [SMILES: Nc1nc2c(ncn2[C@@H]2O[C@@H]3COP(=O)(S)O[C@H]4[C@H]5OC[C@@]4(COP(=O)(S)O[C@H]3[C@H]2F)O[C@H]5n2cnc3c(N)ncnc32)c(=O)[nH]1]
or a salt thereof;
or
a compound of formula II-3 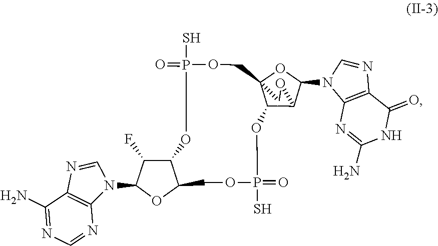 [SMILES: Nc1nc2c(ncn2[C@@H]2O[C@]34CO[C@@H]2[C@@H]3OP(=O)(S)OC[C@H]2O[C@@H](n3cnc5c(N)ncnc53)[C@H](F)[C@@H]2OP(=O)(S)OC4)c(=O)[nH]1]
or a salt thereof;
or
a compound of formula II-4 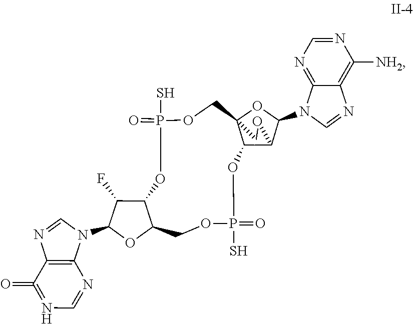
or a salt thereof.

74. (canceled)

75. (canceled)

76. (canceled)

77. The combination according to claim 1, wherein the STING agonist is a substantially pure (Sp,Sp), (Rp,Rp), (Sp,Rp), or (Rp,Sp) stereoisomer of a compound selected from the group of compounds represented by any one of formula I, Ia, Ia.1, Ia.2, Ia.3, Ib, Ib.1, II, II-1, II-2, II-3 and II-4, which is at least 90% pure relative to the other possible diastereomers, or a salt thereof.

78. The combination according to claim 1, wherein the STING agonist is a substantially pure (Rp,Rp) stereoisomer of a compound selected from the group of compounds represented by any one of formula I, Ia, Ia.1, Ia.2, Ia.3, Ib, Ib.1, II, II-1, II-2, II-3 and II-4, which is at least 90% pure relative to the other possible diastereomers, or a salt thereof.

79. The combination according to claim 1, wherein the STING agonist is a pharmaceutically acceptable salt of a compound selected from the group of compounds represented by any one of formula I, Ia, Ia.1, Ia.2, Ia.3, Ib, Ib.1, II, II-1, II-2, II-3 and II-4, or a substantially pure (Sp,Sp), (Rp,Rp), (Sp,Rp), or (Rp,Sp) stereoisomer thereof, which is at least 90% pure relative to the other possible diastereomers.

80. The combination according to claim 1, wherein the STING agonist is a sodium salt of a compound selected from the group of compounds represented by any one of formula Ia.1, Ia.2, Ia.3, Ib.1, II-1, II-2, II-3 and II 4.

81. (canceled)

82. (canceled)

83. The combination according to claim 1, wherein the complex comprises or consists of an amino acid sequence according to SEQ ID NO: 55 or a sequence variant thereof having at least 70%, 75%, 80%, 85%, 90% or 95% sequence identity, and wherein the STING agonist is ADU-S100.

84. The combination according to claim 1, wherein the complex comprises or consists of an amino acid sequence according to SEQ ID NO: 55 or a sequence variant thereof having at least 70%, 75%, 80%, 85%, 90% or 95% sequence identity, and wherein the STING agonist is one compound selected from the group of compounds represented by formula Ia.1, Ia.2, Ia.3, Ib.1, II-1, II-2, II-3 and II 4, or a salt thereof.

85. The combination according to claim 1, wherein the complex comprises or consists of an amino acid sequence according to SEQ ID NO: 54 or a sequence variant thereof having at least 70%, 75%, 80%, 85%, 90% or 95% sequence identity, and wherein the STING agonist is ADU-S100.

86. The combination according to claim 1, wherein the complex comprises or consists of an amino acid sequence according to SEQ ID NO: 54 or a sequence variant thereof having at least 70%, 75%, 80%, 85%, 90% or 95% sequence identity, and wherein the STING agonist is one compound selected from the group of compounds represented by formula Ia.1, Ia.2, Ia.3, Ib.1, II-1, II-2, II-3 and II-4, or a salt thereof.

87.-96. (canceled)

97. A kit comprising
(i) a STING agonist and
(ii) a complex comprising:
a) a cell penetrating peptide;
b) at least one antigen or antigenic epitope; and
c) a TLR peptide agonist,
wherein the components a)-c) are covalently linked.

98. The kit according to claim 97, wherein the complex comprises or consists of an amino acid sequence according to SEQ ID NO: 54 or a sequence variant thereof having at least 70%, 75%, 80%, 85%, 90% or 95% sequence identity, and wherein the STING agonist is one compound selected from the group of compounds represented by formula Ia.1, Ia.2, Ia.3, Ib.1, II-1, II-2, II-3 and II-4, or a salt thereof.

99.-102. (canceled)

103. A composition comprising
(i) a STING agonist and
(ii) a complex comprising:
a) a cell penetrating peptide;
b) at least one antigen or antigenic epitope; and
c) a TLR peptide agonist,
wherein the components a)-c) are covalently linked.

104.-109. (canceled)

110. A method for treating cancer or initiating, enhancing or prolonging an anti-tumor-response in a subject in need thereof comprising administering to the subject an effective amount of
(i) a STING agonist and
(ii) a complex comprising:
a) a cell penetrating peptide;
b) at least one antigen or antigenic epitope; and
c) a TLR peptide agonist,
wherein the components a)-c) are covalently linked.

111. A method for increasing the infiltration of a tumor with tumor antigen-specific T-cells in a patient, the method comprising administering to a patient afflicted with a tumor or cancer
(i) a STING agonist and
(ii) a complex comprising:
a) a cell penetrating peptide;
b) at least one antigen or antigenic epitope; and
c) a TLR peptide agonist,
wherein the components a)-c) are covalently linked.

112. A combination therapy for preventing and/or treating cancer, wherein the combination therapy comprises administration of
(i) a STING agonist and
(ii) a complex comprising:
a) a cell penetrating peptide;
b) at least one antigen or antigenic epitope; and
c) a TLR peptide agonist,
wherein the components a)-c) are covalently linked.

113. The method of claim 110, wherein the subject suffers from cancer or a tumor.

114. The method of claim 113, wherein the subject suffers from an endocrine tumor, a gastrointestinal tumor, a genitourinary or gynecologic tumor, breast cancer, head and neck tumor, hematopoietic tumor, skin tumor, or thoracic or respiratory tumor.

115.-118. (canceled)

119. The combination according to claim 15, wherein the at least one antigen or antigenic epitope is selected from the group of tumor associated antigens, tumor-specific antigens, or tumor neoantigens of colorectal cancer, or metastatic colorectal cancer.

120. The combination according to claim 60, wherein the at least one antigen or antigenic epitope comprises or consists of at least one cancer epitope.

121. The method of claim 114, wherein the subject suffers from colorectal cancer.

122. The method of claim 121, wherein the subject suffers from metastatic colorectal cancer.


SEQUENCE LISTING (59 sequences)

SEQ ID NO: 1 - protein, 16 aa, Artificial Sequence
  CPP penetratin
RQIKIYFQNRRMKWKK

SEQ ID NO: 2 - protein, 11 aa, Artificial Sequence
  CPP TAT minimal domain
YGRKKRRQRRR

SEQ ID NO: 3 - protein, 245 aa, Artificial Sequence
  ZEBRA
MMDPNSTSEDVKFTPDPYQVPFVQAFDQATRVYQDLGGPSQAPLPCVLWPVLPEPLPQGQLTAYHVSTAPTGSWFSAPQPAPENAYQAYAAPQLFPVSDITQNQQTNQAGGEAPQPGDNSTVQTAAAVVFACPGANQGQQLADIGVPQPAPVAAPARRTRKPQQPESLEECDSELEIKRYKNRVASRKCRAKFKQLLQHYREVAAAKSSENDRLRLLLKQMCPSLDVDSIIPRTPDVLHEDLLNF

SEQ ID NO: 4 - protein, 45 aa, Artificial Sequence
  CPP1 (Z11)
KRYKNRVASRKCRAKFKQLLQHYREVAAAKSSENDRLRLLLKQMC

SEQ ID NO: 5 - protein, 42 aa, Artificial Sequence
  CPP2 (Z12)
KRYKNRVASRKCRAKFKQLLQHYREVAAAKSSENDRLRLLLK

SEQ ID NO: 6 - protein, 42 aa, Artificial Sequence
  CPP3 (Z13)
KRYKNRVASRKSRAKFKQLLQHYREVAAAKSSENDRLRLLLK

SEQ ID NO: 7 - protein, 30 aa, Artificial Sequence
  CPP4 (Z14)
KRYKNRVASRKSRAKFKQLLQHYREVAAAK

SEQ ID NO: 8 - protein, 17 aa, Artificial Sequence
  CPP5 (Z15)
KRYKNRVASRKSRAKFK

SEQ ID NO: 9 - protein, 15 aa, Artificial Sequence
  CPP6 (Z16)
QHYREVAAAKSSEND

SEQ ID NO: 10 - protein, 13 aa, Artificial Sequence
  CPP7 (Z17)
QLLQHYREVAAAK

SEQ ID NO: 11 - protein, 19 aa, Artificial Sequence
  CPP8 (Z18)
REVAAAKSSENDRLRLLLK

SEQ ID NO: 12 - protein, 8 aa, Artificial Sequence
  CPP9 (Z19)
KRYKNRVA

SEQ ID NO: 13 - protein, 11 aa, Artificial Sequence
  CPP10 (Z20)
VASRKSRAKFK

SEQ ID NO: 14 - protein, 314 aa, Artificial Sequence
  MAGE-A3
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKAFSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 15 - protein, 630 aa, Artificial Sequence
  mesothelin
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 16 - protein, 142 aa, Artificial Sequence
  survivin
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 17 - protein, 10 aa, Artificial Sequence
  survivin epitope
RISTFKNWPF

SEQ ID NO: 18 - protein, 50 aa, Artificial Sequence
  survivin fragment
APTLPPAWQPFLKDHRISTFKNWPFLEGSAVKKQFEELTLGEFLKLDRER

SEQ ID NO: 19 - protein, 180 aa, Artificial Sequence
  NY-ESO-1
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 20 - protein, 509 aa, Artificial Sequence
  PRAME
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 21 - protein, 193 aa, Artificial Sequence
  ASCL2
MDGGTLPRSAPPAPPVPVGCAARRRPASPELLRCSRRRRPATAETGGGAAAVARRNERERNRVKLVNLGFQALRQHVPHGGASKKLSKVETLRSAVEYIRALQRLLAEHDAVRNALAGGLRPQAVRPSAPRGPPGTTPVAASPSRASSSPGRGGSSEPGSPRSAYSSDDSGCEGALSPAERELLDFSSWLGGY

SEQ ID NO: 22 - protein, 10 aa, Artificial Sequence
  ASCL2 epitope
SAVEYIRALQ

SEQ ID NO: 23 - protein, 10 aa, Artificial Sequence
  ASCL2 epitope
ERELLDFSSW

SEQ ID NO: 24 - protein, 106 aa, Artificial Sequence
  ASCL2 fragment
AAVARRNERERNRVKLVNLGFQALRQHVPHGGASKKLSKVETLRSAVEYIRALQRLLAEHDAVRNALAGGLRPQAVRPSAPRGPSEGALSPAERELLDFSSWLGGY

SEQ ID NO: 25 - protein, 1255 aa, Artificial Sequence
  MUC-1
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 26 - protein, 9 aa, Artificial Sequence
  MUC-1 epitope
GSTAPPVHN

SEQ ID NO: 27 - protein, 10 aa, Artificial Sequence
  MUC-1 epitope
TAPPAHGVTS

SEQ ID NO: 28 - protein, 567 aa, Artificial Sequence
  TGFbetaR2
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 29 - protein, 701 aa, Artificial Sequence
  CEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVAL

SEQ ID NO: 30 - protein, 10 aa, Artificial Sequence
  CEA epitope
YLSGANLNLS

SEQ ID NO: 31 - protein, 10 aa, Artificial Sequence
  CEA epitope
SWRINGIPQQ

SEQ ID NO: 32 - protein, 120 aa, Artificial Sequence
  CEA fragment
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSA

SEQ ID NO: 33 - protein, 393 aa, Artificial Sequence [sequence omitted]

SEQ ID NO: 34 - protein, 189 aa, Artificial Sequence [sequence omitted]

SEQ ID NO: 35 - protein, 9 aa, Artificial Sequence
  KRas epitope
VVVGAGGVG

SEQ ID NO: 36 - protein, 1046 aa, Artificial Sequence
  OGT
MASSVGNVADSTEPTKRMLSFQGLAELAHREYQAGDFEAAERHCMQLWRQEPDNTGVLLLLSSIHFQCRRLDRSAHFSTLAIKQNPLLAEAYSNLGNVYKERGQLQEAIEHYRHALRLKPDFIDGYINLAAALVAAGDMEGAVQAYVSALQYNPDLYCVRSDLGNLLKALGRLEEAKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 37 - protein, 434 aa, Artificial Sequence
  CASP5
MAEDSGKKKRRKNFEAMFKGILQSGLDNFVINHMLKNNVAGQTSIQTLVPNTDQKSTSVKKDNHKKKTVKMLEYLGKDVLHGVFNYLAKHDVLTLKEEEKKKYYDTKIEDKALILVDSLRKNRVAHQMFTQTLLNMDQKITSVKPLLQIEAGPPESAESTNILKLCPREEFLRLCKKNHDEIYPIKKREDRRRLALIICNTKFDHLPARNGAHYDIVGMKRLLQGLGYTVVDEKNLTARDMESVLRAFAARPEHKSSDSTFLVLMSHGILEGICGTAHKKKKPDVLLYDTIFQIFNNRNCLSLKDKPKVIIVQACRGEKHGELWVRDSPASLALISSQSSENLEADSVCKIHEEKDFIAFCSSTPHNVSWRDRTRGSIFITELITCFQKYSCCCHLMEIFRKVQKSFEVPQAKAQMPTIERATLTRDFYLFPGN

SEQ ID NO: 38 - protein, 520 aa, Artificial Sequence
  COA-1
MSSPLASLSKTRKVPLPSEPMNPGRRGIRIYGDEDEVDMLSDGCGSEEKISVPSCYGGIGAPVSRQVPASHDSELMAFMTRKLWDLEQQVKAQTDEILSKDQKIAALEDLVQTLRPHPAEATLQRQEELETMCVQLQRQVREMERFLSDYGLQWVGEPMDQEDSESKTVSEHGERDWMTAKKFWKPGDSLAPPEVDFDRLLASLQDLSELVVEGDTQVTPVPGGARLRTLEPIPLKLYRNGIMMFDGPFQPFYDPSTQRCLRDILDGFFPSELQRLYPNGVPFKVSDLRNQVYLEDGLDPFPGEGRVVGRQLMHKALDRVEEHPGSRMTAEKFLNRLPKFVIRQGEVIDIRGPIRDTLQNCCPLPARIQEIVVETPTLAAERERSQESPNTPAPPLSMLRIKSENGEQAFLLMMQPDNTIGDVRALLAQARVMDASAFEIFSTFPPTLYQDDTLTLQAAGLVPKAALLLRARRAPKSSLKFSPGPCPGPGPGPSPGPGPGPSPGPGPGPSPCPGPSPSPQ

SEQ ID NO: 39 - protein, 380 aa, Artificial Sequence
  IL13Ralpha2
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 40 - protein, 7 aa, Artificial Sequence
  IL13Ralpha2 epitope
LPFGFIL

SEQ ID NO: 41 - protein, 579 aa, Artificial Sequence
  KOC1
MNKLYIGNLSENAAPSDLESIFKDAKIPVSGPFLVKTGYAFVDCPDESWALKAIEALSGKIELHGKPIEVEHSVPKRQRIRKLQIRNIPPHLQWEVLDSLLVQYGVVESCEQVNTDSETAVVNVTYSSKDQARQALDKLNGFQLENFTLKVAYIPDEMAAQQNPLQQPRGRRGLGQRGSSRQGSPGSVSKQKPCDLPLRLLVPTQFVGAIIGKEGATIRNITKQTQSKIDVHRKENAGAAEKSITILSTPEGTSAACKSILEIMHKEAQDIKFTEEIPLKILAHNNFVGRLIGKEGRNLKKIEQDTDTKITISPLQELTLYNPERTITVKGNVETCAKAEEEIMKKIRESYENDIASMNLQAHLIPGLNLNALGLFPPTSGMPPPTSGPPSAMTPPYPQFEQSETETVHLFIPALSVGAIIGKQGQHIKQLSRFAGASIKIAPAEAPDAKVRMVIITGPPEAQFKAQGRIYGKIKEENFVSPKEEVKLEAHIRVPSFAAGRVIGKGGKTVNELQNLSSAEVVVPRDQTPDENDQVVVKITGHFYACQVAQRKIQEILTQVKQHQQQKALQSGPPQSRRK

SEQ ID NO: 42 - protein, 309 aa, Artificial Sequence
  TOMM34
MAPKFPDSVEELRAAGNESFRNGQYAEASALYGRALRVLQAQGSSDPEEESVLYSNRAACHLKDGNCRDCIKDCTSALALVPFSIKPLLRRASAYEALEKYPMAYVDYKTVLQIDDNVTSAVEGINRMTRALMDSLGPEWRLKLPSIPLVPVSAQKRWNSLPSENHKEMAKSKSKETTATKNRVPSAGDVEKARVLKEEGNELVKKGNHKKAIEKYSESLLCSNLESATYSNRALCYLVLKQYTEAVKDCTEALKLDGKNVKAFYRRAQAHKALKDYKSSFADISNLLQIEPRNGPAQKLRQEVKQNLH

SEQ ID NO: 43 - protein, 782 aa, Artificial Sequence
  RNF-43
MSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 44 - protein, 314 aa, Artificial Sequence
  EpCAM
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 45 - protein, 9 aa, Artificial Sequence
  EpCAM epitope
GLKAGVIAV

SEQ ID NO: 46 - protein, 1255 aa, Artificial Sequence [sequence omitted]

SEQ ID NO: 47 - protein, 449 aa, Artificial Sequence
  WT1
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 48 - protein, 276 aa, Artificial Sequence
  antigenic cargo of ATP128
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSAAPTLPPAWQPFLKDHRISTFKNWPFLEGSAVKKQFEELTLGEFLKLDRERAAVARRNERERNRVKLVNLGFQALRQHVPHGGASKKLSKVETLRSAVEYIRALQRLLAEHDAVRNALAGGLRPQAVRPSAPRGPSEGALSPAERELLDFSSWLGGY

SEQ ID NO: 49 - protein, 35 aa, Artificial Sequence
  TLR2 peptide agonist Anaxa
STVHEILCKLSLEGDHSTPPSAYGSVKPYTNFDAE

SEQ ID NO: 50 - protein, 35 aa, Artificial Sequence
  TLR peptide agonist “Anaxa” sequence variant
STVHEILSKLSLEGDHSTPPSAYGSVKPYTNFDAE

SEQ ID NO: 51 - protein, 185 aa, Artificial Sequence
  TLR2 agonist ?30-HMGB1
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKK

SEQ ID NO: 52 - protein, 90 aa, Artificial Sequence
  EDA
NIDRPKGLAFTDVDVDSIKIAWESPQGQVSRYRVTYSSPEDGIRELFPAPDGEDDTAELQGLRPGSEYTVSVVALHDDMESQPLIGIQST

SEQ ID NO: 53 - protein, 145 aa, Artificial Sequence
  Hp91
DPNAPKRPPSAFFLFCSEKRYKNRVASRKSRAKFKQLLQHYREVAAAKSSENDRLRLLLKESLKISQAVHAAHAEINEAGREVVGVGALKVPRNQDWLGVPRFAKFASFEAQGALANIAVDKANLDVEQLESIINFEKLTEWTGS

SEQ ID NO: 54 - protein, 353 aa, Artificial Sequence
  ATP128
KRYKNRVASRKSRAKFKQLLQHYREVAAAKSSENDRLRLLLKNRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSAAPTLPPAWQPFLKDHRISTFKNWPFLEGSAVKKQFEELTLGEFLKLDRERAAVARRNERERNRVKLVNLGFQALRQHVPHGGASKKLSKVETLRSAVEYIRALQRLLAEHDAVRNALAGGLRPQAVRPSAPRGPSEGALSPAERELLDFSSWLGGYSTVHEILSKLSLEGDHSTPPSAYGSVKPYTNFDAE

SEQ ID NO: 55 - protein, 95 aa, Artificial Sequence
  Z13Mad25Anaxa
KRYKNRVASRKSRAKFKQLLQHYREVAAAKSSENDRLRLLLKQAEPDRAHYNIVTFSSKSSTVHEILSKLSLEGDHSTPPSAYGSVKPYTNFDAE

SEQ ID NO: 56 - protein, 9 aa, Artificial Sequence
  HPV-E7 CD8 epitope
RAHYNIVTF

SEQ ID NO: 57 - protein, 8 aa, Artificial Sequence
  OVA CD8 epitope
SIINFEKL

SEQ ID NO: 58 - protein, 162 aa, Artificial Sequence
  Z13Mad39Anaxa
KRYKNRVASRKSRAKFKQLLQHYREVAAAKSSENDRLRLLLKESLKISQAVHAAHAEINEAGREVVGVGALKVPRNQDWLGVPRFAKFASFEAQGALANIAVDKANLDVEQLESIINFEKLTEWTGSSTVHEILSKLSLEGDHSTPPSAYGSVKPYTNFDAE

SEQ ID NO: 59 - protein, 17 aa, Artificial Sequence
  OVA CD4 epitope
ISQAVHAAHAEINEAGR